(19) United States
(12) Patent Application Publication
(10) Pub. No.: US 2009/0311714 A1
(43) Pub. Date: Dec. 17, 2009

(54) Methods for the Screening of Antibacterial Substances

(75) Inventors: Jean-Luc Mainardi, Chaville (FR); Laurent Gutmann, Paris (FR); Michel Arthur, Arcueil (FR); Samuel Bellais, Chennevieres Sur Marne (FR); Jean Emmanuel Hugonnet, Arcueil (FR); Claudine Mayer, Strasbourg (FR); Sabrina Biarotte-Sorin, Rocquencourt (FR)

Correspondence Address: FULBRIGHT & JAWORSKI L.L.P., 600 CONGRESS AVE., SUITE 2400, AUSTIN, TX, 78701, US

(73) Assignees: UNIVERSITE RENE DESCARTES, Paris (FR); INSERM (Institut National de la Sante et de la Recherche Medicale), Paris (FR)

(21) Appl. No.: 11/997,705

(22) PCT Filed: Aug. 1, 2006
(86) PCT No.: PCT/EP2006/064903

§ 371 Date: Mar. 5, 2009

(30) Foreign Application Priority Data
Aug. 2, 2005 (EP) 05300644.1

(51) Int. Cl. G01N 33/53; C12Q 1/37; C12Q 1/25; C12N 9/80; C40B 30/04; C07H 21/04; C07K 16/40

(52) U.S. Cl. 435/7.1; 435/24; 435/4; 435/228; 506/9; 536/23.2; 530/387.9

(57) ABSTRACT

The present invention concerns a method for the screening of antibacterial substances comprising a step of determining the ability of a candidate substance to inhibit the activity of a purified enzyme selected from the group consisting of: (i) a D-aspartate ligase comprising a polypeptide having an amino acid sequence possessing at least 50% amino acid identity with an amino acid sequence selected from the group consisting of SEQ ID No 1 to SEQ ID No 10, or a biologically active fragment thereof; and (ii) a L,D-transpeptidase comprising a polypeptide having an amino acid sequence possessing at least 50% amino acid identity with the amino acid sequence of SEQ ID No 11, or a biologically active fragment thereof.

### FIELD OF THE INVENTION

The present invention relates to the field of anti-microbial therapy, and more precisely to methods for the screening of antimicrobial substances active against bacteria possessing a cell wall comprising peptidoglycan.

### BACKGROUND OF THE INVENTION

Bacterial infections remain among the most common and deadly causes of human disease. Unfortunately, the overuse of antibiotics has led to antibiotic resistant pathogenic strains of bacteria. Indeed, bacterial resistance to the new chemical analogs of these drugs appears to be out-pacing the development of such analogs. For example, life-threatening strains of three species of bacteria (Enterococcus faecalis, Mycobacterium tuberculosis, and Pseudomonas aeruginosa) have evolved to be resistant against all known antibiotics. [Stuart B. Levy, “The Challenge of Antibiotic Resistance”, in Scientific American, pgs. 46-53 (March 1998)].

Antibacterial substances that have already been identified include low-molecular weight substances that are produced as secondary metabolites by certain groups of micro-organisms, especially Streptomyces, Bacillus, and a few molds (Penicillium and Cephalosporium) that are inhabitants of soils. These antibacterial substances may have a bactericidal effect or a static effect on a range of micro-organisms.

Antibacterial substances that have already been identified also include chemotherapeutic agents which are chemically synthesized, as well as semi-synthetic antibiotics, wherein an antibacterial substance that is naturally produced by a micro-organism is subsequently modified by chemical methods to achieve desired properties.

Antibiotics effective against prokaryotes which kill or inhibit a wide range of Gram-positive and Gram-negative bacteria are said to be of broad spectrum. If effective against Gram-positive or Gram-negative bacteria, they are of narrow spectrum. If effective against a single organism or disease, they are referred to as limited spectrum.

Antibacterial substances achieve their bactericidal or static effects by altering various metabolic pathways of the target micro-organisms.

Several antibacterial substances act as cell membrane inhibitors that disorganise the structure or inhibit the function of bacterial membranes, like polymixin B, which binds to membrane phospholipids and thereby interferes with membrane function, mainly against Gram-negative bacteria.

Several other antibacterial substances act as protein synthesis inhibitors, like tetracyclines, chloramphenicol, macrolides and aminoglycosides.

Still other antibacterial substances affect the synthesis of DNA or DNA, or can bind to DNA or RNA, like quinolones and rifamycins.

Yet other antibacterial substances act as competitive inhibitors of essential metabolites or growth factors, like sulfonamides.

Important antibacterial substances act as inhibitors of the cell wall synthesis, and more specifically as inhibitors of the synthesis of the bacterial peptidoglycan. The peptidoglycan is a macromolecular structure found on the outer face of the cytoplasmic membrane of almost all bacteria. This structure is of importance for the maintenance of the integrity of the bacteria and for the cell division process. The basic unit of the peptidoglycan is a disaccharide peptide assembled by a series of cytoplasmic and membrane reactions. The resulting unit is composed of N-acetylglucosamine (GlcNAc) linked to N-acetylmuramic acid (MurNAc) substituted by a stem peptide. In the majority of pathogenic Gram positive bacteria such as Staphylococcus, Streptococcus and Enterococcus, the stem peptide consists in a conserved L-alanyl-γ-D-glutamyl-L-lysyl-D-alanyl-D-alanine pentapeptide and variable side chains linked to the ε-amino group of the third residue (L-Lys₃). The structure of the side chain conserved in the members of the same species consists of glycines or various L-amino acids added by the transferases which used the corresponding specific aminoacyl-tRNAs as substrates. Once this basic unit have been transferred through the cytoplasmic membrane, the final steps of peptidoglycan synthesis involve its polymerization to glycan strands by glycosyltransferases and the cross-linking of the stem peptides by multiple D,D-transpeptidases. In Enterococcus faecium peptidoglycan, the side chain consists of one D-Asp or one D-Asn which is linked by its β-carboxyl group to the ε-amino group of L-Lys₃. The resulting unit is composed of GlcNAc-MurNAc substituted by an L-alanyl-γ-D-glutamyl-L-(N^{ε}-D-isoaspartyl)lysyl-D-alanyl-D-alanine or an L-alanyl-γ-D-glutamyl-L-(N^{ε}-D-isoasparaginyl)lysyl-D-alanyl-D-alanine stem hexapeptide (D-Asx-pentapeptide)(4-6). At the late stage of the polymerisation, the interpeptide bridge synthesized by the D,D-transpeptidases consist in a peptide bond between the carboxyl group of D-Ala at position 4 of a donor stem peptide and the amino group of the D-Asn or D-Asp (D-Asx) linked to the L-Lys₃ of an acceptor peptide stem.

Peptidoglycan synthesis inhibitors exert their selective toxicity against eubacteria, since mammal cells lack peptidoglycan. All beta lactams have a common mechanism of action and act as suicide substrates of the D,D-transpeptidase catalytic domain of the penicillin binding proteins (PBPs) responsible for the last cross-linking step of the cell wall assembly.

The main inhibitors of the cell wall synthesis are those of the beta lactam family, which include penicillins and cephalosporins. The beta lactam antibiotics are stereochemically related to D-alanyl-D-alanine which is a substrate for the last step in peptidoglycan synthesis, i.e. the final cross-linking between peptide side chains. Beta lactam compounds include natural and semi-synthetic penicillins, clavulanic acid, cephalosporins, carbapenems and monobactams. Other inhibitors also encompass glycopeptides such as vancomycin.

Over the past three decades, there has been an increasing use of beta lactams, which have entered clinical use since 1965. Unfortunately, the widespread use of these antibacterial substances has resulted in an alarming increase in the number of resistant strains, especially among clinically important bacteria such as the genera Salmonella, Enterobacteriacae, Pseudomonas and Staphylococcus.

Generally, bacterial resistance to beta lactams occurs primarily through three mechanisms: (i) destruction of the antibiotic by beta-lactamases, (ii) decreased penetration due to changes in bacterial outer membrane composition and (iii) alteration in penicillin-binding proteins (PBPs) resulting in interference with beta lactam binding. The latter pathway is especially important, as the binding of beta lactams to PBPs is essential for inhibiting peptidoglycan biosynthesis. For glycopeptides, increasing numbers of Vancomycin-resistant strains of enterococci have been found since 1988. Vancomycin-resistant enterococci exhibit changes in the cell wall production.

Overuse of antibiotics, non-compliance with a full course of antibiotic treatment, routine prophylactic use and sub-therapeutic drug levels all contribute to the development of resistant strains of bacteria.

There is thus a need in the art for identifying novel antibacterial substances exerting an inhibiting effect on the peptidoglycan biosynthesis, as well as for novel methods for their screening.

Notably, there is a need in the art for identifying inhibitors of peptidoglycan biosynthesis that are active against antibiotic-resistant bacteria, including beta lactams-resistant bacteria.

This need in the art includes identifying novel bacterial target proteins that are involved in peptidoglycan biosynthesis that will allow performing screening methods of active antibacterial substances. Such screening methods encompass in vitro screening methods wherein inhibitory activity of candidate substances against newly identified bacterial target protein(s) is assayed. Such screening methods also encompass in silico screening methods wherein blocking biological activity of newly identified bacterial target protein(s) can be assayed, once said target protein(s) is (are) identified and its (their) tridimensional structure deciphered.

### SUMMARY OF THE INVENTION

The present invention relates primarily to a method for the screening of antibacterial substances comprising a step of determining the ability of a candidate substance to inhibit the activity of a purified enzyme selected from the group consisting of:
(i) a D-aspartate ligase comprising a polypeptide having an amino acid sequence possessing at least 50% amino acid identity with an amino acid sequence selected from the group consisting of SEQ ID No 1 to SEQ ID No 10, or a biologically active fragment thereof; and
(ii) a L,D-transpeptidase comprising a polypeptide having an amino acid sequence possessing at least 50% amino acid identity with the amino acid sequence of SEQ ID No 11, or a biologically active fragment thereof.

This invention also pertains to a method for the screening of antibacterial substances, wherein said method comprises the steps of:
a) providing a candidate substance;
b) assaying said candidate substance for its ability to bind to a D-aspartate ligase or to a L,D-transpeptidase as defined herein.

This invention also concerns a crystallized L,D-transpeptidase having the amino acid sequence starting at the amino acid located in position 119 and ending at the amino acid located in position 466 of the amino acid sequence of SEQ ID No 13 defined herein.

This invention also pertains to a method for selecting a compound that interacts with the catalytic site of the L,D-transpeptidase defined herein, wherein said method comprises the steps of:
a) generating a three-dimensional model of said catalytic site using a set of data corresponding to the relative structural coordinates according to Table 3; and
b) employing said three-dimensional model to design or select a compound, from a serial of compounds, that interacts with said catalytic site of the L,D-transpeptidase defined herein.

The present invention also relates to various other methods for the screening of an antibiotic candidate substance that take benefit from the availability of the three-dimensional structure of the L,D-transpeptidase that is defined in detail in the present specification.

This invention also concerns computer systems and methods that are useful for performing methods for the screening of antibiotic candidate substances acting on the target L,D-transpeptidase that is defined in detail in the present specification.

### BRIEF DESCRIPTION OF THE FIGURES

FIG. 1. Schematic representation of peptidoglycan cross-linking in Enterococcus faecium. Peptidoglycan is polymerized from a subunit comprising a disaccharide composed of β-1-4-linked N-acetylglucosamine (GlcNAc) and N-acetylmuramic acid (MurNAc), a conserved pentapeptide stem (L-Ala-D-iGln-L-Lys-D-Ala-D-Ala) and a side chain which consists of D-Asp or D-Asn (D-Asx).

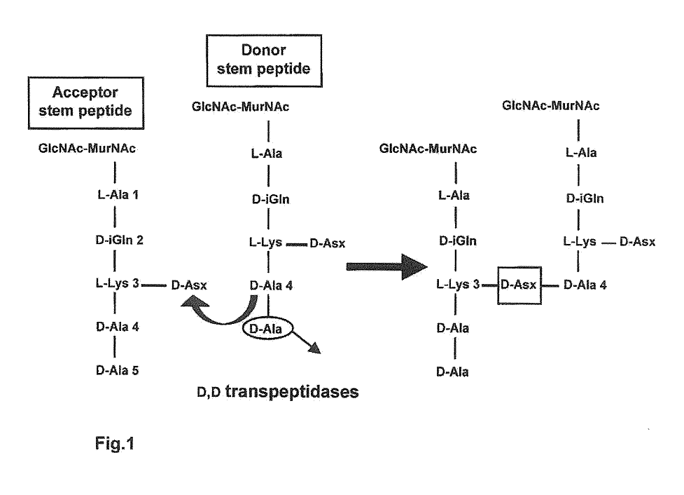

FIG. 2. Radiochromatogram revealing the D-aspartate ligase assay using crude cytoplasmic extracts. Crude cytoplasmic extract (60 μg of protein) of E. faecium D359V8 were incubated for 2 h with UDP-MurNac-pentapeptide (0.8 mM), D-[¹⁴C]aspartic acid (0.11 mM, 55 mCi/mmol), ATP (20 mM) and MgCl₂ (50 mM). D-[¹⁴C]aspartic acid was separated from [¹⁴C]UDP-MurNac-hexapeptide by descending paper chromatography. The chromatogram was revealed after 4 days exposure. (+) presence of cytoplasmic extracts; (−) absence of crude extracts.

FIG. 3. D-aspartate activity of the purified protein fusion produced in E. coli. The assay was performed as described in FIG. 2 using 2 μg of purified protein. A, separation of D-[¹⁴C]aspartic acid (peak A) and [¹⁴C] UDP-MurNac-hexapeptide (peak B) were obtained by HPLC with isocratic elution (10 mM ammonium acetate, pH 5.0) at a flow rate of 0.5 ml/min. B, MS analysis of UDP-MurNac-hexapeptide showing peaks at m/z 1265.4, 633.2, 644.2 and 652.2, which were assigned to be [M+H]⁺, [M+2H]²⁺, [M+H+Na]²⁺ and [M+H+K]²⁺ ions, respectively. C, MS/MS analysis of peak at m/z 1265.3. D, MS/MS analysis of peak at m/z 676.3 assigned to be the lactyl-hexapeptide moieties of the UDP-MurNac-hexapeptide

FIG. 4. HPLC muropeptide profiles of JH2-2/pJEH11 (A) and JH2-2/pSJL2(Asl_{fm}) grown in presence of 50 mM of D-Asp (B). Purified peptidoglycan was digested with lysozyme and mutanolysine, treated with ammonium hydroxide to produce D-lactoyl peptide fragments which were separated by reversed-phase HPLC. Absorbance was monitored at 210 nm (mAU, absorbance unit×10³). Numbering of the peaks (1 to 10) in A and B is the same as in Arbeloa et al (Arbeloa, A., Segal, H., Hugonnet, J. E., Josseaume, N., Dubost, L., Brouard, J. P., Gutmann, L., Mengin-Lecreuix, D. & Arthur, M. (2004) J Bacteriol 186, 1221-8) Letters present in B represent new peaks. The structure of the muropeptides present in the peaks is described in Table 1.

FIG. 5. Analysis of the main monomer from JH2-2/pSJL2(Asl_{fm}) by tandem mass spectrometry. A, fragmentation was performed on the ion at m/z 675.3 corresponding to the [M+H]¹⁺ from the lactoyl peptide peptidoglycan fragment from the major monomer C. B, structure of the major monomer and inferred fragmentation pattern. The m/z values in A originate from cleavage at single peptide bonds as represented in B. Peaks at m/z 560.3 matched the predicted value for loss of one N-terminal D-aspartate residue. Loss of one and two D-Ala from the C-terminus of the pentapeptide stem gave ions at m/z 586.2 and 515.2. The peak at m/z 532.2 matched the predicted value for loss of D-Lac-L-Ala. From this ion, further loss of one and two D-Ala from the C-terminus gave ions at m/z 443.2 and 372.1. Cleavage of the same peptide bond also produced peaks at m/z 144.0 corresponding to the D-Lac-L-Ala moiety of the molecule. Fragmentation at the D-iGln-L-Lys peptide bond produced ions at 272.1 and 404.2. Additional ions could be accounted for by combinations of the fragmentation described above.

FIG. 6. Alignment of the Asl_{fm} with identified homologs from different bacterial species. Multiple sequence alignment was performed using the BLAST and FASTA softwares available over the Internet at the National Center for Biotechnology Information Web site (htt://www.ncib.nim.nih.gov/). *: conserved residues which in the ATP-grasp proteins interact with ATP. (Galperin, M. Y. & Koonin, E. V. (1997) Protein Sci 6, 2639-43.), (Eroglu, B. & Powers-Lee, S. G. (2002) Arch Biochem Biophys 407, 1-9), (Stapleton, M. A., Javid-Majd, F., Harmon, M. F., Hanks, B. A., Grahmann, J. L., Mullins, L. S. & Raushel, F. M. (1996) Biochemistry 35, 14352-61). abbreviations: Ent Face, Enterococcus faecium; Ltc lact, Lactococcus lactis subsp. Lactis IL1403; Ltc crem, Lactococcus lactis subsp cremoris SK11; Ltb gass, Lactobacillus gasseri ATCC 333323; Ltb john, Lactobacillus johnsonii NCC 533; Ltb delb, Lactobacillus delbrueckii subsp bulgaricus ATCC BAA-365; Ltb brev, Lactobacillus brevis ATCC 367; Ltb casei, Lactobacillus casei ATCC 334; Ped pent, Pediococcus pentosaceus ATCC 24745.

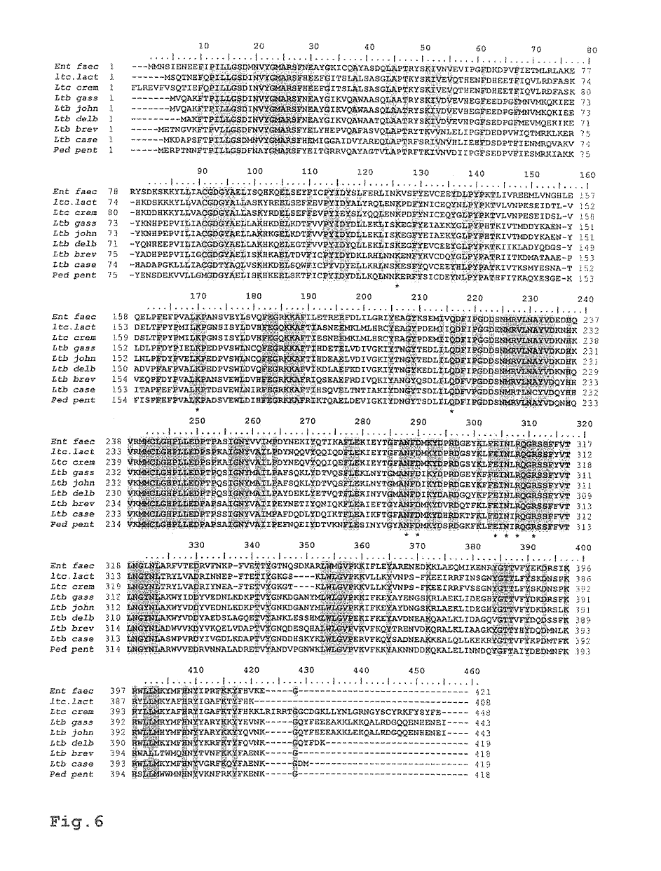

FIG. 7. Proposed catalytic mechanism of the D-asparte ligase. In the first step, the D-aspartate ligase couple ATP hydrolysis to activation of an acyl group to form the D-aspartyl-phosphate intermediate before linkage to the ε-amino group of the L-Lys₃ of the stem peptide.

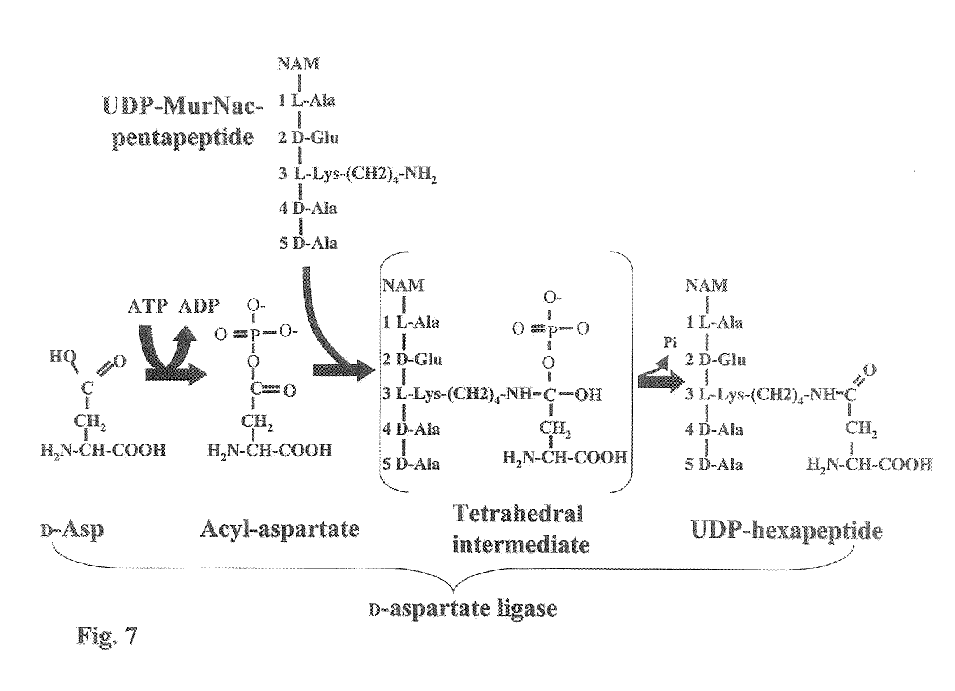

FIG. 8 (Ex FIG. 1). Cross-links generated by the D,D-transpeptidase activity of the penicillin binding proteins (PBPs) and the β-lactam insensitive L,D-transpeptidase. The cell wall of most bacteria is stabilized by an exoskeleton made of the cross-linked heteropolymer called peptidoglycan. The peptidoglycan subunit is a disaccharide-peptide which is assembled by a series of cytoplasmic and membrane reactions (J. van Heijenoort, Nat. Prod. Rep. 18, 503 (2001).). In E. faecium, the resulting subunit is composed of β,1-4-linked N-acetylglucosamine and N-acetylmuramic acid (GlcNAc-MurNAc, not represented) substituted by a branched stem pentapeptide containing a D-isoasparagine residue (iAsn) linked to the c-amino group of L-Lys₃ [L-alanyl₁-D-isoglutamyl₂-L-(N^{ε}-D-isoasparaginyl)lysyl₃-D-alanyl₄-D-alanine₅ stem peptide] (J. L. Mainardi et al., J. Biol. Chem. 275, 16490 (2000).). The final steps of peptidoglycan synthesis involve transfer of the unit through the cytoplasmic membrane, formation of glycan strands by glycosyltransferases, and cross-linking of stem peptides by D,D-transpeptidases. The latter enzymes cleave the C-terminal residue (D-Ala₅) of the first substrate (pentapeptide donor), and link the carboxyl of the penultimate residue (D-Ala₄) to the amino group of the second substrate (the acceptor) resulting in the formation of a D-Ala₄⋄D-iAsn-L-Lys₃ cross-link (C. Goffin, J. M. Ghuysen, Microbiol. Mol. Biol. Rev. 62, 1079 (1998).; J. L. Mainardi et al., J. Biol. Chem. 275, 16490 (2000).). The D,D-transpeptidases belong to the penicillin-binding protein (PBP) family and are the essential targets of β-lactams. Bypass of the β-lactam-sensitive D,D-transpeptidase in the mutant E. faecium M512 highly resistant to ampicillin (minimal inhibitory concentration>2000 μg/ml) requires the production of a D,D-carboxypeptidase, which cleaves the C-terminal D-alanine residue (D-Ala₅) of the pentapeptide stem, to generate the tetrapeptide donor of the L,D-transpeptidase. The latter enzyme cleaves the L-Lys₃-D-Ala₄ bond and links the carboxyl of L-Lys₃ to the side chain of the acceptor (L-Lys₃⋄D-iAsn-L-Lys₃ cross-link).

FIG. 9 (Ex FIG. 2). Identification and characterization of the L,D-transpeptidase of E. faecium M512. (A) Purification of the L,D-transpeptidase from an E. faecium M512 extract (lane 1) led to partial purification of a 48-kDa protein (lane 2). (B) The open reading frame for the 48-kDa protein was identified based on N-terminal sequencing (AEKQEIDPVSQNHQKLDTTV [SEQ ID No 20], underlined) and similarity searches in the partial genome sequence of E. faecium. The partially purified 48-kDa protein corresponded to a proteolytic fragment since the sequence encoding its N-terminus was not preceded by a translation initiation codon. The upstream sequence contained a single likely translation initiation site (ACTTAAggagTTGTCGATatg [SEQ ID No 21]), consisting of an ATG initiation codon preceded by a putative ribosome binding site (lower case). The proteolytic cleavage removed the first 118 residues of the protein, including a cluster of hydrophobic residues at positions 13 to 28 (italicized), which could correspond to a membrane anchor. The C-terminus of Ldt_{fm} (positions 340 to 466; bold) was related to a family of 341 sequences from eubacteria appearing in the Protein Families Database of Alignments under the pfam accession number PF03734. Ser439 and Cys442 (asterisks) are potential catalytic residues. (C) The portion of the open reading frame encoding the soluble protein partially purified form the E. faecium extract (positions 119 to 466) was cloned into E. coli and Ldt_{fm} was purified with an overall yield of 3 mg per liter of culture. (D) The purified protein was active in an exchange reaction which assays for the capacity of the enzyme to catalyze cleavage of the L-Lys-D-Ala peptide bond of the model donor dipeptide substrate N^{α},N^{ε}-diacetyl-L-lysyl-D-alanine (Ac₂-L-Lys-D-Ala) and formation of a peptide bond between Ac₂-L-Lys and D-[¹⁴C]Ala. (E) Ldt_{fm} (3 μg) was incubated with Ac₂-L-Lys-D-Ala (0.3 mM), D-[¹⁴C]Ala (0.15 mM), and various concentration of ampicillin showing the absence of inhibition. (F) The exchange assay was also performed with non-radioactive 2-amino and 2-hydroxy acids based on detection of the products by mass spectrometry and determination of their structure by tandem mass spectrometry, as exemplified by the fragmentation of Ac₂-L-Lys-D-Met (m/z of 362.24). Loss of a C-terminal D-Met and of additional H₂O led to ions at m/z 213.14 and 185.14, respectively. Ions at m/z 84.10 and 126.11 correspond to the immonium of L-Lys and its acetylated form, respectively.

FIG. 10 (Ex FIG. 3). In vitro formation of dimers by Ldt_{fm}. (A) Ldt_{fm} was incubated with a pool of three monomeric muropeptides containing the disaccharide GlcNAc-MurNAc substituted by three different stem peptides. Formation of dimers was observed by mass spectrometry for four of the six possible combinations of donors and acceptors. Tetrapeptide-iAsn, L-Ala-D-iGln-L-(N^{ε}-D-iAsn)Lys-D-Ala; Tripeptide-iAsn, L-Ala-D-iGln-L-(N^{ε}-D-iAsn)Lys; Tetrapeptide, L-Ala-D-iGln-L-Lys-D-Ala. (B) Fragmentation was performed on the muropeptide lactoyl dimer at m/z 1118.5 which was obtained by ammonium hydroxide treatment of the dimer with a monoisotopic mass of 1927.88. The treatment cleaved off the disaccharide and converted D-iAsn into D-iAsp. (C) Structure of the dimer and inferred fragmentation pattern.

FIG. 11. Alignment of the deduced sequence of L,D-transpeptidase from E. faecium (Ldt_{fm}) with close homologs from Gram-positive bacteria. L. plant, Lactobacillus plantarum WCFS1; C. aceto, Clostridium acetobutylicum ATCC:824; E. faeca, Enterococcus faecalis V583; B. anthr, Bacillus anthracis Ames.

FIG. 12: molecular surface of the L,D-transpeptidase (FIG. 12A). Zoom of the hole of domain 2 (FIG. 12B), the histidines are shown in cyan, the cysteine in orange, and the serine in green. An uncharacterized ion bridges the two histidines (FIG. 12C).

### DETAILED DESCRIPTION OF THE INVENTION

According to the invention, it has been characterised two proteins that have been found to be both involved in the bacterial cell wall peptidoglycan biosynthesis.

The findings of the invention according to which these two proteins are involved in the peptidoglycan biosynthesis has allowed the inventors to design various methods for the screening of candidate antibacterial substances, the effect of which is targeted against these two proteins.

More precisely, the two proteins that have been identified according to the invention consist of enzymes, thus target proteins for which alterations in their biological activity by candidate antibacterial substances may be easily detected.

Further, one of these two enzymes has been crystallized and its spatial conformation deciphered, including the spatial conformation of its catalytic site, thus allowing the design of in silico screening methods for substances that can enter the catalytic site and prevent availability of said catalytic site for natural substrate(s). In silico methods for screening antibiotics have already proved their efficiency, for instance in the case of screening for aminoglycoside complexing with RNA, using bacterial ribosomal RNA crystal structure as the antibiotics target.

The first enzyme, the involvement of which in the peptidoglycan biosynthesis has been found according to the present invention, consists of a D-aspartate ligase. It is to be noticed that a D-aspartic activating enzyme activity was previously described as being present in enzyme preparations from Streptococcus faecalis (in fact probably from Enterococcus faecium), but without any structural characterization of the corresponding protein(s) (See Staudenbauer and Strominger, 1972, The Journal of Biological Chemistry, Vol. 247(17): 5289-5296).

Said D-aspartate ligase catalyses incorporation of D-aspartate on UDP-MurNac pentapeptide to form the side chain of peptidoglycan precursor. It has been found according to the invention that recombinant expression of the gene encoding said D-aspartate ligase, in a host organism wherein this gene is not naturally present, induces the recombinant host organism to synthesise a cell wall peptidoglycan wherein D-aspartate residues are linked to the c-amino group of L-Lys3 of the main monomers of the peptidoglycan, which shows that said D-aspartate ligase characterised according to the invention is functional in various bacteria that do not naturally express said enzyme.

Further, it has been found structurally similar D-aspartate ligases in various bacteria for which existing data show that they produce D-aspartate-containing branched cell wall precursors of the peptidoglycan, including bacteria from the Lactobacilli species, Lactococci species and Pediococci species.

The second enzyme, the involvement of which in the peptidoglycan biosynthesis has been found according to the present invention, consists of a L,D-transpeptidase. It is to be noticed that a beta lactam-insensitive L,D-transpeptidase activity was previously described as being present in membrane preparations from Enterococcus faecium bacteria, but without any structural characterization of the corresponding protein(s) (See Mainardi et al., 2002, The Journal of Biological Chemistry, Vol. 277(39): 35801-35807).

Said L,D-transpeptidase catalyses the L,D transpeptidation of peptidoglycan subunits containing a tetrapeptide stem.

The L,D-transpeptidase characterised according to the present invention has a high value as a target protein for the screening of novel antibacterial substances.

Further, the catalytic site of the L,D-transpeptidase characterised according to the present invention has been identified, both (i) biologically, through directed mutagenesis experiments, and (ii) structurally, through the characterisation of the three-dimensional structure of this enzyme, including the characterisation of the three dimensional structure of its active site, after crystallisation of this enzyme.

Thus, according to the invention, the biological effectors for the previously known bacterial D-aspartate ligase activity and L,D-transpeptidase activity in certain bacteria have been characterized, isolated and recombinantly produced for the first time.

These findings have allowed the inventors to design methods for the screening of antibacterial substances having the ability to cause disorders in the bacterial peptidoglycan normal biosynthesis.

Thus, a first object of the invention consists of a method for the screening of antibacterial substances comprising a step of determining the ability of a candidate substance to inhibit the activity of a purified enzyme selected from the group consisting of:
(i) a D-aspartate ligase comprising a polypeptide having an amino acid sequence possessing at least 50% amino acid identity with an amino acid sequence selected from the group consisting of SEQ ID No 1 to SEQ ID No 10, or a biologically active fragment thereof; and
(ii) a L,D-transpeptidase comprising a polypeptide having an amino acid sequence possessing at least 50% amino acid identity with the amino acid sequence of SEQ ID No 11, or a biologically active fragment thereof.

According to the invention, it has been found that the D-aspartate ligase of SEQ ID No 1 originating from Enterococcus faecium bacteria, that has been newly characterised and isolated, possesses structural and functional similarities with proteins characterized herein as consisting of D-aspartate ligases originating from various other bacteria, including those originating from, respectively, Lactococcus lactis (SEQ ID No 2), Lactococcus cremoris SK111 (SEQ ID No 3), Lactobacillus gasseri (SEQ ID No 4), Lactobacillus johnosonii NCC 533 (SEQ ID No 5), Lactobacillus delbruckei Subsp. bulgaricus (SEQ ID No 6), Lactobacillus casei (SEQ ID No 7), Lactobacillus acidophilus (SEQ ID No 8), Lactobacillus brevis (SEQ ID No 9) and Pediococcus pentosaceus (SEQ ID No 10).

More specifically, beyond their amino acid sequence similarity with the D-aspartate ligase of SEQ ID No 1, the D-aspartate ligases of SEQ ID No 2 to SEQ ID No 10 all originate from bacteria which produce D-Asp-containing branched cell wall peptidoglycan precursors. Conversely, no nucleic acid sequences encoding proteins having similarities with the D-aspartate ligase of SEQ ID No 1 are found in the genome of bacteria having cell wall peptidoglycan with either (i) direct crosslinks or (ii) crosslinks containing glycine or L-amino acids.

The amino acid sequence of SEQ ID No 11 consists of the C-terminal end located from the amino acid residue in position 340 and ending at the amino acid residue in position 466 of the L,D-transpeptidase originating from Enterococcus faecium bacteria of SEQ ID No 13, that catalyses the L,D transpeptidation of peptidoglycan subunits containing a tetrapeptide stem. More precisely, it has been found according to the invention that the C-terminal portion of SEQ ID No 11 of said L,D-transpeptidase comprises the catalytic site of said enzyme, both by directed mutagenesis experiments and by crystallisation of this protein. Notably, it has been found that important amino acid residues comprised in the catalytic site of said L,D-transpeptidase include the Serine residue located at position 439 of SEQ ID No 13 and the Cysteine residue located at position 442 of SEQ ID No 13. From crystallisation data, it has further been found that the Histidine residue located at position 421 of SEQ ID No 13 and the Histidine residue located at position 440 of SEQ ID No 13 both form part of the catalytic site of said L,D-transpeptidase. More generally, the catalytic site of said L,D-transpeptidase of SEQ ID No 13 is comprised in the amino acid sequence beginning at the Isoleucine residue located at position 368 of SEQ ID No 13 and ending at the Methionine residue located at position 450 of SEQ ID No 13.

The L,D-transpeptidase notably comprises a C-terminal portion of SEQ ID No 12, which includes the amino acid sequence of SEQ ID No 11 at its C-terminal end. The L,D-transpeptidase C-terminal portion of SEQ ID No 12 forms a protein domain that is also found in proteins originating from various other bacteria, notably Gram-positive bacteria. Proteins having strong amino acid sequence identity with the L,D-transpeptidase comprising SEQ ID No 11 or 12 are found in proteins originating from Lactobacillus plantarum, Clostridium acetobutylicum, Enterococcus faecalis and Bacillus anthracis.

The complete amino acid sequence of the L,D-transpeptidase that has been characterised according to the invention consists of the amino acid sequence of SEQ ID No 13.

As intended herein, a D-aspartate ligase or a L,D-transpeptidase characterized according to the invention, or any biologically active peptide thereof, “comprises” a polypeptide as defined above because, in certain embodiments, said D-aspartate ligase or said L,D-transpeptidase may not simply consist of said polypeptide defined above. Illustratively, a D-aspartate ligase or a L,D-transpeptidase characterized according to the invention, or any biologically active peptide thereof, may comprise, in addition to a polypeptide as defined above, additional amino acid residues that are located (i) at the N-terminal end, (ii) at the C-terminal end or (iii) both at the N-terminal end and at the C-terminal end of said polypeptide above. Generally, at the N-terminal end or at the C-terminal end of a polypeptide defined above, there is no more than 30 additional amino acid residues and often no more than 29, 28, 27, 26, 25, 24, 23, 22, 21, 20, 19, 18, 17, 16, 15, 14, 13, 12, 11, 10, 9, 8, 7, 6, 5 or 4 additional amino acid residues. Illustratively, a polypeptide as defined above that possesses a D-aspartate ligase or a L,D-transpeptidase activity possesses, at its C-terminal end, six additional Histidine amino acid residues.

As intended herein, a polypeptide or a protein having at least 50% amino acid identity with a reference amino acid sequence possesses at least 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.5% amino acid identity with said reference amino acid sequence.

For the purpose of determining the percent of identity of two amino acid sequences according to the present invention, the sequences are aligned for optimal comparison purposes. For example, gaps can be introduced in one or both of a first and a second amino acid sequence for optimal alignment and non-homologous sequences can be disregarded for comparison purposes.

For optimal comparison purposes, the percent of identity of two amino acid sequences can be achieved with CLUSTAL W (version 1.82) with the following parameters: (1) CPU MODE=ClustalW mp; (2) ALIGNMENT=<<full>>; (3) OUTPUT FORMAT=<<aln w/numbers>>; (4) OUTPUT ORDER=<<aligned>>; (5) COLOR ALIGNMENT=<<no>>; (6) KTUP (word size)=<<default>>; (7) WINDOW LENGTH=<<default>>; (8) SCORE TYPE=<<percent>>; (9) TOPDIAG=<<default>>; (10) PAIRGAP=<<default>>; (11) PHYLOGENETIC TREE/TREE TYPE=<<none>>; (12) MATRIX=<<default>>; (13) GAP OPEN=<<default>>; (14) END GAPS=<<default>>; (15) GAP EXTENSION=<<default>>; (16) GAP DISTANCES=<<default>>; (17) TREE TYPE=<<cladogram>>; et (18) TREE GRAP DISTANCES=<<hide>>.

By a “biologically active fragment” of a D-aspartate ligase or of a L,D-transpeptidase that are defined above, it is intended herein a polypeptide having an amino acid length that is shorter than the amino acid length of the enzyme polypeptide of reference, while preserving the same D-aspartate ligase or of a L,D-transpeptidase activity, that is the same specificity of catalytic activity and an activity of at least the same order of magnitude than the activity of the parent enzyme polypeptide.

A biologically active fragment of a D-aspartate ligase characterized according to the invention possesses a D-aspartate ligase activity that is assessed, using, as substrates, D-aspartate and a compound selected from the group consisting of UDP-MurNac pentapeptide and UDP-MurNac tetrapeptide, and then quantifying the UDP-MurNac pentapeptide-Asp or the UDP-MurNac tetrapeptide-Asp that is produced. Said fragment consists of a biologically active fragment of a D-aspartate ligase according to the invention if the rate of production of UDP-MurNac tetrapeptide-Asp is at least 0.1 the rate of the D-aspartate ligase of SEQ ID No 1.

A biologically active fragment of a L,D-transpeptidase characterised according to the invention possesses a L,D-transpeptidase activity that is assessed using, as substrates, (i) a donor compound consisting of a tetrapeptide preferably selected from the group consisting of L-Ala-D-Glu-L-Lys-D-Ala, Ac₂-L-Lys-D-Ala and disaccharide-tetrapeptide(iAsn) and (ii) an acceptor compound selected from the group consisting of a D-amino acid or a D-hydroxy acid. Said fragment consists of a biologically active fragment of a L,D-transpeptidase according to the invention if the rate of production of the final dimer product is at least 0.1 the rate of the L,D-transpeptidase of SEQ ID No 12, or of the L,D-transpeptidase of SEQ ID No 13.

Generally, a biologically active fragment of a D-aspartate ligase or of a L,D-transpeptidase according to the invention has an amino acid length of at least 100 amino acid residues. Usually, a biologically active fragment of a D-aspartate ligase or of a L,D-transpeptidase according to the invention comprises at least 100 consecutive amino acid residues of a D-aspartate ligase or of a L,D-transpeptidase as defined above.

Advantageously, a biologically active fragment of a D-aspartate ligase as defined above comprises, or consists of, a polypeptide consisting of 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439 or 440 consecutive amino acid residues of a D-aspartate ligase as defined above, it being understood that the amino acid length of said biologically active peptide fragment is necessary limited by the amino acid length of the D-aspartate ligase from which said biologically active peptide fragment derives.

Advantageously, a biologically active fragment of a L,D-transpeptidase as defined above comprises, or consists of, a polypeptide consisting of 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459 or 461 consecutive amino acid residues of a L,D-transpeptidase as defined above, it being understood that the amino acid length of said biologically active peptide fragment is necessary limited by the amino acid length of the L,D-transpeptidase from which said biologically active peptide fragment derives.

In a preferred embodiment of the method for the screening of antibacterial substances that is defined above, said method comprises the steps of:
a) providing a composition comprising said purified D-aspartate ligase or said L,D-transpeptidase, and a substrate thereof;
b) adding the candidate substance to be tested to the composition provided at step a), whereby providing a test composition; and
c) comparing the activity of said enzyme in said test composition with the activity of the same D-aspartate ligase or the same L,D-transpeptidase in the absence of said candidate substance;
d) selecting positively the candidate substance that inhibits the catalytic activity of said enzyme.

As intended herein, a candidate substance to be tested inhibits the catalytic activity of said D-aspartate ligase or of said L,D-transpeptidase if the activity of said enzyme, when the candidate substance is present, is lower than when said enzyme is used without the candidate substance under testing.

Preferably, the candidate substances that are positively selected at step d) of the method above are those that cause a decrease of the production rate of the final product by said D-aspartate ligase or by said L,D-transpeptidase that leads to less than 0.5 times the production rate of the same enzyme in the absence of the candidate substance, more preferably a decrease that leads to less 0.3, 0.2, 0.1, 0.05 or 0.025 times the production rate of the same enzyme in the absence of the candidate substance. The most active candidate substances that may be positively selected at step d) of the method above may completely block the catalytic activity of said enzyme, which leads to a production rate of the final product by said D-aspartate ligase or by said L,D-transpeptidase which is undetectable, i.e. zero, or very close to zero.

In a preferred embodiment of the screening method above, said enzyme consists of a D-aspartate ligase comprising a polypeptide having an amino acid sequence possessing at least 60% amino acid identity with an amino acid sequence selected from the group consisting of SEQ ID No 1 to SEQ ID No 10, or a biologically active fragment thereof.

In another preferred embodiment of the screening method above, said enzyme consists of a D-aspartate ligase comprising a polypeptide having an amino acid sequence possessing at least 90% amino acid identity with the amino acid sequence of SEQ ID No 1 to SEQ ID No 10, or a biologically active fragment thereof.

In a further preferred embodiment of the screening method above, said enzyme consists of a D-aspartate ligase comprising a polypeptide having an amino acid sequence possessing at least 90% amino acid identity with the amino acid sequence of SEQ ID No 1, or a biologically active fragment thereof.

In still a further embodiment, said enzyme consists of the D-aspartate ligase comprising a polypeptide having the amino acid sequence of SEQ ID No 1, or a biologically active fragment thereof.

In yet a further embodiment, said enzyme consists of the D-aspartate ligase of SEQ ID No 1, or a biologically active fragment thereof.

In one preferred embodiment of the screening method above, the D-aspartate ligase activity is assessed using, as substrates, D-aspartate and a compound selected from the group consisting of UDP-MurNac pentapeptide and UDP-MurNac tetrapeptide.

Preferably, radioactively labeled D-aspartate is used, such as D-[¹⁴C] aspartate or D-[³H] aspartate.

Usually, the reaction mixture comprising (i) labeled D-aspartate, (ii) UDP-MurNac pentapeptide or UDP-MurNac tetrapeptide and (iii) optionally the candidate inhibitor compound is incubated in the suitable reaction medium during a time period of from 1 h to 3 h, advantageously from 1.5 h to 2.5 h, at a temperature ranging from 35° C. to 39° C., advantageously from 36° C. to 38° C. and most preferably at 37° C., before the reaction is stopped. Usually, the reaction is stopped by boiling the resulting reaction mixture during the appropriate time period, which may be 3 min.

Then, the remaining labeled D-aspartate is separated from the reaction product consisting of labeled UDP-MurNac hexapeptide or UDP-MurNac pentapeptide, e.g. [¹⁴C]UDP-MurNac hexapeptide or [¹⁴C]UDP-MurNac pentapeptide, depending of the substrate which is used, preferably by performing a chromatography separation step. Usually, non-reacted labeled D-aspartate is separated from the other reaction products by descending paper chromatography, such as disclosed in the examples.

Then, the reaction products are further separated, preferably by performing a subsequent chromatography step, such as a step of reverse phase high-pressure liquid chromatography (rpHPLC), such as disclosed in the examples.

In order to confirm the structure of the final product, the reaction step described above may be performed with non-radioactive D-aspartate and samples of UDP-MurNac-peptide products may be isolated by rpHPLC and then lyophilized. Said lyophilized product may then be resuspended, for example in water, and analyzed by Mass spectrometry (MS) and MS/MS, as disclosed in the examples herein, for instance by performing the technique previously described by Bouhss et al. (2002).

Detection of the labeled reaction product resulting from the D-aspartate ligase catalytic activity may be performed simultaneously with said chromatographic step. For example, if the initial substrate, and thus also the reaction product, are radioactively labeled, then the detection of the reaction product, or the detection and the quantification, of the reaction product, may be performed with a suitable radioactivity detector that is coupled to the chromatography device, such as disclosed in the examples.

Thus, in one preferred embodiment of the screening method above, the D-aspartate ligase activity is assessed by quantifying the UDP-MurNac pentapeptide-Asp or the UDP-MurNac tetrapeptide-Asp that is produced, as it is detailed above and is fully described in the examples.

In another preferred embodiment of the screening method above, said enzyme consists of a L,D-transpeptidase comprising a polypeptide having an amino acid sequence possessing at least 60% amino acid identity with the amino acid sequence of SEQ ID No 11, or a biologically active fragment thereof.

In a further preferred embodiment of the screening method above, said enzyme consists of a L,D-transpeptidase comprising a polypeptide having an amino acid sequence possessing at least 90% amino acid identity with the amino acid sequence of SEQ ID No 11, or a biologically active fragment thereof.

In a still further preferred embodiment of the screening method above, said enzyme consists of a L,D-transpeptidase comprising a polypeptide having the amino acid sequence of SEQ ID No 11, or a biologically active fragment thereof.

It is reminded here that the amino acid sequence of SEQ ID No 11 comprises the C-terminal part of the L,D-transpeptidase of SEQ ID No 13, said amino acid sequence of SEQ ID No 11 comprising the important amino acid residues that form part of the active site of said enzyme, including HIS421, S439, HIS440 and CYS442.

Thus, in another preferred embodiment of the screening method above, said enzyme consists of a L,D-transpeptidase comprising a polypeptide having at least 90% amino acid identity with the amino acid sequence possessing at least 90% amino acid identity with the amino acid sequence of SEQ ID No 12, or a biologically active fragment thereof. The amino acid sequence of SEQ ID No 12 consists of a C-terminal portion of the L,D-transpeptidase of SEQ ID No 13. The amino acid sequence of SEQ ID No 12 is longer than, and comprises SEQ ID No 11. The amino acid sequence of SEQ ID No 12 also comprises the important amino acid residues that form part of the active site of said enzyme, including HIS421, S439, HIS440 and CYS442. It has been shown according to the invention that the L,D-transpeptidase consisting of SEQ ID No 12 has the same catalytic activity than the L,D-transpeptidase consisting of SEQ ID No 13, despite it lacks the N-terminal end of the L,D-transpeptidase of SEQ ID No 13.

Thus, in one preferred embodiment of the screening method above, said enzyme consists of a L,D-transpeptidase comprising a polypeptide having at least 90% amino acid identity with the amino acid sequence of SEQ ID No 12, or a biologically active fragment thereof

According to further preferred embodiment of the screening method above, said enzyme consists of a L,D-transpeptidase having at least 90% amino acid identity with the amino acid sequence of SEQ ID No 13, or a biologically active peptide fragment thereof.

In yet a further embodiment of said screening method, said enzyme consists of the L,D-transpeptidase comprising a polypeptide consisting of the amino acid sequence of SEQ ID No 13, or a biologically active peptide fragment thereof.

In still a further embodiment of said screening method, said enzyme consists of the L,D-transpeptidase consisting of the amino acid sequence of SEQ ID No 13, or a biologically active peptide fragment thereof.

Preferably, any one of the biologically active peptide fragments of the polypeptide of SEQ ID No 13 comprises at least 100 consecutive amino acids of SEQ ID No 13 and comprises the amino acid residues SER439 and CYS442.

Preferably, any one of the biologically active peptide fragments of the polypeptide of SEQ ID No 13 comprises at least 100 consecutive amino acids of SEQ ID No 13 and comprises the amino acid residues HIS421, SER439, HIS440 and CYS442.

Preferably, any one of the biologically active peptide fragments of the polypeptide of SEQ ID No 13 comprises the amino acid sequence beginning at the Isoleucine amino acid residue located at position 368 and ending at the Methionine amino acid residue located at position 450 of the L,D-transpeptidase of SEQ ID No 13.

A specific embodiment of a biologically active peptide fragment of the polypeptide of SEQ ID No 13 consists of a polypeptide comprising, or consisting of, the amino acid sequence beginning at the amino acid residue located at position 119 and ending at the amino acid residue located at position 466 of SEQ ID No 13.

In a preferred embodiment of the screening method above, the L,D-transpeptidase activity is assessed using, as substrates, (i) a donor compound consisting of a tetrapeptide preferably selected from the group consisting of L-Ala-D-Glu-L-Lys-D-Ala, Ac₂-L-Lys-D-Ala and disaccharide-tetrapeptide(iAsn) and (ii) an acceptor compound selected from the group consisting of a D-amino acid or a D-hydroxy acid.

In certain embodiments of the method above, said D-amino acid is selected from the group consisting of D-methionine, D-asparagine and D-serine.

In certain other embodiments of the method above, said D-hydroxy acid is selected from the group consisting of D-2-hydroxyhexanoic acid and D-lactic acid.

Preferably, the L,D-transpeptidase activity is assessed by performing a standard exchange assay that is based on icubation of non-radioactive Ac₂-L-Lys-D-Ala and D-[¹⁴C]Ala and determination of Ac₂-L-Lys-D[¹⁴C]Ala formed by the L,D-transpeptidase catalytic activity, such as disclosed by Mainardi et al. (J. L. Mainardi et al., J. Biol. Chem. 277, 35801 (2002)) as well as in the examples herein.

In an illustrative embodiment of said standard exchange assay, a reaction mixture is provided, which reaction mixture contains (i) purified L,D-transpeptidase, (ii) Ac₂-L-Lys-D-Ala, (iii) D-[¹⁴C]Ala and (iv) optionally the candidate inhibitor compound. Then the enzyme reaction is performed until completion, generally during a time period of from 1.5 h to 2.5 h, most preferably 1 h, at a temperature range comprised between 36° C. and 38° C., advantageously between 36.5° C. and 37.5° C., most preferably at 37° C. Then, the enzyme reaction is stopped, for example by boiling the resulting reaction product mixture for a time period sufficient to inactivate the enzyme, such as for a period of time ranging from 3 min to 20 min, most preferably a period of time of about 15 min.

Then, the resulting reaction product mixture is centrifuged and a sample collected from the supernatant of centrifugation is analysed by chromatography, preferably by carrying out a reverse phase high-pressure liquid chromatography (rpHPLC), most preferably with isocratic elution.

Detection of the labeled reaction product resulting from the L,D-transpeptidase catalytic activity may be performed simultaneously with said chromatographic step. For example, if the initial substrate, and thus also the reaction product, are radioactively labeled, then the detection, or the detection and the quantification, of the reaction product may be performed with a suitable radioactivity detector that is coupled to the chromatography device, such as disclosed in the examples.

To assay for in vitro transpeptidation, the one skilled in the art may prepare a reaction mixture comprising (i) purified L,D-transpeptidase, (ii) GlcNAc-MurNAc-L-Ala-D-iGln-L-(N^{ε}-D-iAsn)Lys-D-Ala, GlcNAc-MurNAc-L-Ala-D-iGln-L-(N^{ε}-D-iAsn)Lys and GlcNAc-MurNAc-L-Ala-D-iGln-L-Lys-D-Ala and (iii) optionally the inhibitor candidate compound, in a suitable reaction buffer. Then, the transpeptidation reaction is allowed to proceed during a time period preferably ranging from 1.5 h to 2.5 h, most preferably of about 2 h, at a preferred temperature range between 36.5° C. and 37.5° C., most preferably of about 37° C. Then, when brought to completion, the transpeptidation reaction is stopped, for example by boiling for a time period sufficient to inactivate the L,D-transpeptidase, e.g. for a period of time ranging from 3 min to 20 min, most preferably a period of time of about 15 min.

Then, the resulting reaction product mixture is centrifuged and an aliquot sample is collected from the supernatant of centrifugation.

Said supernatant sample is then used to determine, and usually also quantify, the formation of dimers.

Preferably, the formation of dimers is determined, and usually quantified, by mass-spectrometry.

A tandem-mass spectrometry is usually also performed after having cleaved the ether link internal to MurNac by treatment of a sample from the supernatant resulting product reaction mixture with ammonium hydroxide, such as disclosed by Arbeloa et al. (A. Arbeloa et al., J. Biol. Chem. 279, 41546 (2004)). Then, the resulting lactoyl-peptides are fragmented using N₂ as the collision gas, such as disclosed by Arbeloa et al. (A. Arbeloa et al., J. Biol. Chem. 279, 41546 (2004)). Any of the D-aspartate ligases or of the L,D-transpeptidases that are defined throughout the present specification can be produced by performing various techniques of protein synthesis that are well known by the one skilled in the art, including chemical synthesis and genetic engineering methods for producing recombinant proteins.

Preferably, any one of the D-aspartate ligases and any one of the L,D-transpeptidases that are defined throughout the present specification are produced as recombinant proteins.

### Production of the D-aspartate Ligases or of the L,D-transpeptidases

The description below relates primarily to production of the D-aspartate ligases or of the L,D-transpeptidases according to the invention by culturing cells transformed or transfected with a vector containing nucleic acid encoding corresponding polypeptides. It is, of course, contemplated that alternative methods that are well known in the art may be employed to prepare the polypeptides of interest according to the invention. For instance, the polypeptide sequence of interest, or portions thereof, may be produced by direct peptide synthesis using solid-phase techniques. See, e.g., Stewart et al., Solid-Phase Peptide Synthesis (W.H. Freeman Co.: San Francisco, Calif., 1969); Merrifield, J. Am. Chem. Soc., 85: 2149-2154 (1963). In vitro protein synthesis may be performed using manual techniques or by automation. Automated synthesis may be accomplished, for instance, with an Applied Biosystems Peptide Synthesizer (Foster City, Calif.) using manufacturer's instructions. Various portions of the polypeptide of interest may be chemically synthesized separately and combined using chemical or enzymatic methods to produce the full-length polypeptide of interest

### Isolation of DNA Encoding the D-Aspartate Ligases or the L,D-transpeptidases of Interest

DNA encoding the polypeptide of interest may be obtained from a cDNA library prepared from tissue believed to possess the mRNA encoding it and to express it at a detectable level. Accordingly, DNAs encoding the D-aspartate ligases or the L,D-transpeptidases can be conveniently obtained from cDNA libraries prepared from bacteria.

Generally, a DNA encoding a D-aspartate ligase or a L,D-transpeptidase as defined herein may be obtained by amplification of bacterial genomic DNA or bacterial cDNA by a specific pair of primers. A specific pair of primers can be easily designed by the one skilled in the art who has the knowledge of the nucleic acid sequence that encodes the enzyme of interest.

The nucleic acid sequences that encode the D-aspartate ligases of SEQ ID No 1 to 10 consist of the polynucleotides of SEQ ID No 22 to 31, respectively. The nucleic acid sequences that encode the L,D-transpeptidase of SEQ ID No 13 consists of the polynucleotide of SEQ ID No 32.

Illustratively, a DNA encoding a D-aspartate ligase of SEQ ID No 1 may be easily obtained by amplifying bacterial DNA with the pair of primers of SEQ ID No 14 and 15, as shown in the examples.

Illustratively, a DNA encoding a L,D-transpeptidase of SEQ ID No 13 may be easily obtained by amplifying bacterial DNA with the pair of primers of SEQ ID No 18 and 19, as shown in the examples.

### Selection and Transformation of Host Cells

Host cells are transfected or transformed with expression or cloning vectors described herein for polypeptide of interest production and cultured in conventional nutrient media modified as appropriate for inducing promoters, selecting transformants, or amplifying the genes encoding the desired sequences. The culture conditions, such as media, temperature, pH, and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991).

Methods of transfection are known to the ordinarily skilled artisan, for example, CaPO₄ treatment and electroporation. Depending on the host cell used, transformation is performed using standard techniques appropriate to such cells. The calcium treatment employing calcium chloride, as described in Sambrook et al., supra, or electroporation is generally used for prokaryotes or other cells that contain substantial cell-wall barriers. For mammalian cells without such cell walls, the calcium phosphate precipitation method of Graham and van der Eb, Virology, 52:456-457 (1978) can be employed. General aspects of mammalian cell host system transformations have been described in U.S. Pat. No. 4,399,216. Transformations into yeast are typically carried out according to the method of Van Solingen et al., J. Bact., 130: 946 (1977) and Hsiao et al., Proc. Natl. Acad. Sci. (USA), 76: 3829 (1979). However, other methods for introducing DNA into cells, such as by nuclear microinjection, electroporation, bacterial protoplast fusion with intact cells, or polycations, e.g., polybrene or polyornithine, may also be used. For various techniques for transforming mammalian cells, see, Keown et al., Methods in Enzymology, 185: 527-537 (1990) and Mansour et al., Nature, 336: 348-352 (1988).

Suitable host cells for cloning or expressing the DNA in the vectors herein include prokaryote, yeast, or higher eukaryote cells. Suitable prokaryotes include, but are not limited to, eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325); and K5772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan.sup.r; E. coli W3110 strain 37D6, which has the complete genotype tona ptr3 phoA E15 (argF-lac)169 degP ompT rbs7 ilvG kan.sup.r; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for vectors encoding the polypeptide of interest. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9: 968-975 (1991)) such as, e.g., K. lactis (MW98-8C, CBS683, CBS4574; Louvencourt et al., J. Bacteriol., 737 [1983]), K. fragilis (ATCC 12,424), K. bulgaricus (ATCC 16,045), K. wickeramii(ATCC 24,178), K. waltii(ATCC 56,500), K. drosophilarum(ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28: 265-278 [1988]); Candida; Trichoderma reesia (EP 244,234); Neurospora crassa (Case et al., Proc. Natl. Acad. Sci. USA, 76: 5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112: 284-289 [1983]; Tilburn et al., Gene, 26: 205-221 [1983]; Yelton et al., Proc. Natl. Acad. Sci. USA, 81: 1470-1474 [1984]) and A. niger (Kelly and Hynes, EMBO J., 4: 475-479 [1982]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of nucleic acid encoding glycosylated polypeptides of interest are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen. Virol., 36: 59 (1977)); Chinese hamster ovary cells/−DHFR(CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

### Selection and Use of a Replicable Vector

The nucleic acid (e.g., cDNA or genomic DNA) encoding the polypeptide of interest may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence if the sequence is to be secreted, an origin of replication, one or more marker genes, an enhancer element, a promoter, and a transcription termination sequence. Construction of suitable vectors containing one or more of these components employs standard ligation techniques that are known to the skilled artisan.

The polypeptide of interest may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the DNA encoding the polypeptide of interest that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces .alpha.-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2.mu. plasmid origin is suitable for yeast, and various viral origins (SV40, polyoma, adenovirus, VSV, or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the nucleic acid encoding the polypeptide of interest such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77: 4216 (1980). A suitable selection gene for use in yeast is the trp 1 gene present in the yeast plasmid YRp7. Stinchcomb et al., Nature, 282: 39 (1979); Kingsman et al., Gene, 7: 141 (1979); Tschemper et al, Gene, 10: 157 (1980). The trp1 gene provides a selection marker for a mutant strain of yeast lacking the ability to grow in tryptophan, for example, ATCC No. 44076 or PEP4-1. Jones, Genetics, 85:12 (1977).

Expression and cloning vectors usually contain a promoter operably linked to the nucleic acid sequence encoding the polypeptide of interest to direct mRNA synthesis. Promoters recognized by a variety of potential host cells are well known. Promoters suitable for use with prokaryotic hosts include the .beta.-lactamase and lactose promoter systems (Chang et al., Nature, 275: 615 (1978); Goeddel et al., Nature, 281: 544 (1979)), alkaline phosphatase, a tryptophan (trp) promoter system (Goeddel, Nucleic Acids Res., 8:4057 (1980); EP 36,776), and hybrid promoters such as the tac promoter (deBoer et al., Proc. Natl. Acad. Sci. USA, 80: 21-25 (1983)). promoters for use in bacterial systems also will contain a Shine-Dalgarno (S.D.) sequence operably linked to the DNA encoding the polypeptide of interest.

Examples of suitable promoting sequences for use with yeast hosts include the promoters for 3-phosphoglycerate kinase (Hitzeman et al., J. Biol. Chem., 255: 2073 (1980)) or other glycolytic enzymes (Hess et al., J. Adv. Enzyme Reg., 7: 149 (1968); Holland, Biochemistry, 17: 4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters that are inducible promoters having the additional advantage of transcription controlled by growth conditions are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Nucleic acid of interest transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus, and Simian Virus 40 (SV40); by heterologous mammalian promoters, e.g., the actin promoter or an immunoglobulin promoter; and by heat-shock promoters, provided such promoters are compatible with the host cell systems.

Transcription of a DNA encoding the polypeptide of interest by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, .alpha.-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the sequence coding for polypeptides of interest, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding polypeptide of interest.

Still other methods, vectors, and host cells suitable for adaptation to the synthesis of the of interest in recombinant vertebrate cell culture are described in Gething et al., Nature, 293: 620-625 (1981); Mantei et al., Nature, 281: 40-46 (1979); EP 117,060; and EP 117,058.

### Purification of the Polypeptides of Interest

Forms of polypeptides of interest may be recovered from culture medium or from host cell lysates. If membrane-bound, it can be released from the membrane using a suitable detergent solution (e.g., TRITON-X™ 100) or by enzymatic cleavage. Cells employed in expression of nucleic acid encoding the polypeptide of interest can be disrupted by various physical or chemical means, such as freeze-thaw cycling, sonication, mechanical disruption, or cell-lysing agents. It may be desired to purify the polypeptide of interest from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; Protein A Sepharose columns to remove contaminants such as IgG; and metal chelating columns to bind epitope-tagged forms of the polypeptide of interest. Various methods of protein purification may be employed and such methods are known in the art and described, for example, in Deutscher, Methods in Enzymology, 182 (1990); Scopes, Protein Purification: Principles and Practice (Springer-Verlag: New York, 1982). The purification step(s) selected will depend, for example, on the nature of the production process used and the particular polypeptide produced.

Finally, specific embodiments for obtaining a nucleic acid encoding a D-aspartate ligase or a L,D-transpeptidase as defined throughout the present specification, inserting said nucleic acid in a suitable expression vector, and transfecting host cells with said vector in order to produce the corresponding protein are disclosed in the examples herein.

### Other In Vitro Screening Methods According to the Invention

As detailed previously in the specification, this invention encompasses methods for the screening of candidate antibacterial substances that inhibit the activity of a D-aspartate ligase or a L,D-transpeptidase as defined herein.

However, this invention also encompasses methods for the screening of candidate antibacterial substances, that are based on the ability of said candidate substances to bind to a D-aspartate ligase or to a L,D-transpeptidase as defined herein, thus methods for the screening of potentially antibacterial substances

The binding assays can be performed in a variety of formats, including protein-protein binding assays, biochemical screening assays, immunoassays, and cell-based assays, which are well characterized in the art.

All binding assays for the screening of candidate antibacterial substances are common in that they comprise a step of contacting the candidate substance with a D-aspartate ligase or with a L,D-transpeptidase as defined herein, under conditions and for a time sufficient to allow these two components to interact.

These screening methods also comprise a step of detecting the formation of complexes between said D-aspartate ligase or said L,D-transpeptidase and said candidate antibacterial substances.

Thus, screening for antibacterial substances include the use of two partners, through measuring the binding between two partners, respectively (i) a D-aspartate ligase or a L,D-transpeptidase as defined herein and (ii) the candidate compound.

In binding assays, the interaction is binding and the complex formed between a D-aspartate ligase or a L,D-transpeptidase as defined above and the candidate substance that is tested can be isolated or detected in the reaction mixture. In a particular embodiment, (i) the D-aspartate ligase or the L,D-transpeptidase as defined above or (ii) the antibacterial candidate substance is immobilized on a solid phase, e.g., on a microtiter plate, by covalent or non-covalent attachments. Non-covalent attachment generally is accomplished by coating the solid surface with a solution of the D-aspartate ligase or the L,D-transpeptidase as defined above and drying. Alternatively, an immobilized antibody, e.g., a monoclonal antibody, specific for the D-aspartate ligase or for the L,D-transpeptidase as defined above to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

The binding of the antibacterial candidate substance to a D-aspartate ligase or to a L,D-transpeptidase as defined above may be performed through various assays, including traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340: 245-246 (1989); Chien et al., Proc. Natl. Acad. Sci. USA, 88: 9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for .beta.-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Thus, another object of the invention consists of a method for the screening of antibacterial substances, wherein said method comprises the steps of:
a) providing a candidate substance;
b) assaying said candidate substance for its ability to bind to a D-aspartate ligase or to a L,D-transpeptidase as defined above;

The same method may also be defined as a method for the screening of antibacterial substances, wherein said method comprises the steps of:
a) contacting a candidate substance with a D-aspartate ligase or a L,D-transpeptidase as defined herein, or with a biologically active fragment thereof;
b) detecting the complexes eventually formed between (i) said D-aspartate ligase or said L,D-transpeptidase as defined herein, or with said biologically active fragment thereof and (ii) said candidate substance.

The candidate substances, which may be screened according to the screening method above, may be of any kind, including, without being limited to, natural or synthetic compounds or molecules of biological origin such as polypeptides.

### Binding Assays Based on Enzyme Peptide Mapping

According to one embodiment of the screening method above, step b) comprises a step of proteolysis of said D-aspartate ligase or of said L,D-transpeptidase prior to the detection of a binding between the candidate inhibitor substance and said enzyme.

More precisely, according to this specific embodiment of step b) of the screening method described above, said D-aspartate ligase or said L,D-transpeptidase is incubated with a protease during a time period sufficient to generate a plurality of peptide fragments. Then, a step of detection of formation of eventual complexes between at least one of these peptide fragments and the candidate inhibitor compound is performed.

According to this specific embodiment of step b) of the screening method above, said step b) of assaying for the binding of said candidate substance to a D-aspartate ligase or to a L,D-transpeptidase as defined above comprises the following steps:
b1) subjecting said D-aspartate ligase or said L,D-transpeptidase to proteolysis, so as to generate a plurality of peptide fragments;
b2) separating the peptide fragments obtained at the end of step ci); and
b3) detecting the complexes eventually formed between one or more of the peptide fragments separated at step b2) and the inhibitor candidate substance.

At step b1), any one of the proteases known in the art may be used. However, the most preferred protease consists of trypsin.

Trypsin digestion of said D-aspartate ligase or said L,D-transpeptidase is performed according to methods well known in the art.

Typically, said purified D-aspartate ligase or said purified L,D-transpeptidase in a suitable liquid buffer is subjected to trypsin digestion at 37° C. for a time period ranging from 1 h to 24 h, depending on the respective concentrations of said purified enzyme and of trypsin, respectively. Illustratively, said purified D-aspartate ligase or said purified L,D-transpeptidase is present in a suitable buffer selected from the group consisting of (i) a 1% (w/v) ammonium bicarbonate buffer, a 25 mM potassium buffer and (iii) a 50 mM Tris-HCl buffer at pH 8.0. Then, the proteolysis reaction is stopped, for example by adding (i) 1% trifluoroacetic acid solution or (ii) phenylmethyl sulfonyl fluoride (PMSF) solution to the resulting proteolysis mixture.

Then, at step b2), the various peptide fragments that are generated by trypsin proteolysis are subjected to a separation step.

In certain embodiments, said separation step may consist of an electrophoresis gel separation of the peptide fragments, using conventional electrophoresis conditions that are well known when performing classical Western blotting peptide separation.

In certain other embodiments, said separation step consists of a step of High Pressure Liquid Chromatography (HPLC), for example using a LC-Packing® system that is disclosed in the examples herein.

Then, at step b3), detection of the complexes eventually formed between one or more of the peptide fragments separated at step b2) and the inhibitor candidate substance is performed.

In most embodiments of step b3), detection of the complexes eventually formed between one or more of the peptide fragments separated at step b2) and the inhibitor candidate substance is performed by:
b3-a) comparing (i) the peptide separation pattern from said D-aspartate ligase or from said L,D-transpeptidase in the absence of the inhibitor candidate substance with (ii) the peptide separation pattern from said D-aspartate ligase or from said L,D-transpeptidase when said inhibitor candidate substance has previously been contacted with the enzyme of interest;
b3-b) detecting differences between the two peptide separation patterns (i) and (ii), which differences, when present, are indicative of the binding of said inhibitor candidate compound to said D-aspartate ligase or to said L,D-transpeptidase.

When step b2) consists of a conventional gel electrophoresis separation step, the differences between the two peptide separation patterns (i) and (ii) that are detected at step b3) consist of differences in the migration location on the gel of one or more peptide fragments onto which said inhibitor candidate compound is bound. Illustratively, the one or more peptides that are bound to the candidate substance generally migrate faster in the gel than the same unbound peptide(s).

When step b2) consists of an HPLC step, the differences between the two peptide separation patterns (i) and (ii) that are detected at step c3) consist of differences in the elution time of the one or more peptide fragments onto which said inhibitor candidate compound is bound.

In certain embodiments, said screening method may also comprises an additional step b4) of identification of the peptide fragment(s) onto which is bound said inhibitor candidate substance.

Usually, step b4) is performed by subjecting the peptide fragment(s) onto which is bound said inhibitor candidate substance to identification by mass spectrometry, for example by using an ion trap mass spectrometer as it is disclosed in the examples. Performing step b4) allows to identify precisely the binding location of said inhibitor candidate substance onto said D-aspartate ligase or onto said L,D-transpeptidase, so as to determine, notably, if said inhibitor candidate compound binds to the active site or close to the active site of the enzyme, or conversely binds at a protein location which is distant of the active site of said enzyme. This will allow to discriminate, notably, between competitive and non-competitive candidate inhibitor substances.

### Two Hybrid Screening System

Two-hybrid screening methods are performed for the screening of candidate substances that consist of candidate polypeptides.

In a preferred embodiment, of the screening method, the candidate polypeptide is fused to the LexA binding domain, the D-aspartate ligase or the L,D-transpeptidase as defined above is fused to Gal 4 activator domain and step (b) is carried out by measuring the expression of a detectable marker gene placed under the control of a LexA regulation sequence that is responsive to the binding of a complete protein containing both the LexA binding domain and the Gal 4 activator domain. For example, the detectable marker gene placed under the control of a LexA regulation sequence can be the β-galactosidase gene or the HIS3 gene, as disclosed in the art.

In a particular embodiment of the screening method, the candidate compound consists of the expression product of a DNA insert contained in a phage vector, such as described by Parmley and Smith (1988). Specifically, random peptide libraries are used. The random DNA inserts encode for peptides of 8 to 20 amino acids in length (Oldenburg et al., 1992, Proc. Natl. Acad. Sci. USA, 85(8): 2444-2448; Valadon et al., 1996, J Mol Biol, 261: 11-22; Lucas, 1994, In: Development and Clinical Uses of Haemophilus b Conjugate; Westerink, 1995, Proc. Natl. Acad. Sci. USA, 92: 4021-4025; Felici et al., 1991, J Mol Biol, 222: 301-310). According to this particular embodiment, the recombinant phages expressing a polypeptide that specifically binds to a D-aspartate ligase or to a L,D-transpeptidase as defined above, are retained as expressing a candidate substance for use in the screening method above.

More precisely, In a first preferred embodiment of the screening method above, the screening system used in step (b) includes the use of a Two-hybrid screening assay. The yeast two-hybrid system is designed to study protein-protein interactions in vivo and relies upon the fusion of a bait protein to the DNA binding domain of the yeast Gal4 protein. This technique is described in the U.S. Pat. No. 5,667,973.

The general procedure of the two-hybrid assay is described hereafter. In an illustrative embodiment, the polynucleotide encoding the D-aspartate ligase or to the L,D-transpeptidase as defined above is fused to a polynucleotide encoding the DNA binding domain of the Gal4 protein, the fused protein being inserted in a suitable expression vector, for example pAS2 or pM3.

Then, the polynucleotide encoding the candidate polypeptide is fused to a nucleotide sequence in a second expression vector that encodes the activation domain of the Gal4 protein.

The two expression plasmids are transformed into yeast cells and the transformed yeast cells are plated on a selection culture medium which selects for expression of selectable markers on each of the expression vectors as well as GAL4 dependent expression of the HIS3 gene. Transformants capable of growing on medium lacking histidine are screened for gal4 dependent LacZ expression. Those cells which are positive in the histidine selection and the Lac Z assay denote the occurrence of an interaction between the D-aspartate ligase or the L,D-transpeptidase as defined above and the candidate polypeptide and allow to quantify the binding of the two protein partners.

Since its original description, the yeast two-hybrid system has been used extensively to identify protein-protein interactions from many different organisms. Simultaneously, a number of variations on a theme based on the original concept have been described. The original configuration of the two-hybrid fusion proteins was modified to expand the range of possible protein-protein interactions that could be analyzed. For example, systems were developed to detect trimeric interactions. Finally, the original concept was turned upside down and ‘reverse n-hybrid systems’ were developed to identify peptides or small molecules that dissociate macromolecular interactions (Vidal et al., 1999, Yeast forward and reverse ‘n’-hybrid systems. Nucleic Acids Res. 1999 Feb. 15; 27(4):919-29). These variations in the two-hybrid system can be applied to the disruption of the interaction between candidates antibacterial polypeptides and a D-aspartate ligase a L,D-transpeptidase as defined above and enters in the scope of the present invention.

### Western Blot

In another preferred embodiment, of the screening method according to the invention, step (b) consists of subjecting to a gel migration assay the mixture obtained at the end of step (a) and then measuring the binding of the candidate polypeptide with the D-aspartate ligase or with the L,D-transpeptidase as defined above by performing a detection of the complexes formed between the candidate polypeptide and said D-aspartate ligase or said L,D-transpeptidase as defined above.

The gel migration assay can be carried out by conventional widely used western blot techniques that are well known from the one skilled in the art.

The detection of the complexes formed between the candidate polypeptide and the D-aspartate ligase or the L,D-transpeptidase as defined above can be easily observed by determining the stain position (protein bands) corresponding to the proteins analysed since the apparent molecular weight of a protein changes if it is in a complex.

On one hand, the stains (protein bands) corresponding to the proteins submitted to the gel migration assay can be detected by specific antibodies for example antibodies specifically directed against the D-aspartate ligase or the L,D-transpeptidase as defined above or against the candidate polypeptide, if the latter are available. Alternatively, the candidate polypeptide or the D-aspartate ligase or the L,D-transpeptidase as defined above can be tagged for an easier revelation of the gel, for example by fusion to GST, HA, poly Histidine chain, or other probes in order to facilitate the identification of the different protein on the gel, according to widely known techniques.

### Biosensor

In another preferred embodiment of the screening method above, the screening system used in step (b) includes the use of an optical biosensor such as described by Edwards and Leatherbarrow (1997, Analytical Biochemistry, 246: 1-6) or also by Szabo et al. (1995, Curr. Opinion Struct. Biol., 5(5): 699-705). This technique permits the detection of interactions between molecule in real time, without the need of labelled molecules. This technique is based on the surface plasmon resonance (SPR) phenomenon. Briefly, a first protein partner molecule, for example the candidate polypeptide, is attached to a surface (such as a carboxymethyl dextran matrix). Then, the second protein partner molecule, in this case the D-aspartate ligase or the L,D-transpeptidase as defined above, is incubated with the first partner, in the presence or in the absence of the candidate compound to be tested and the binding, including the binding level, or the absence of binding between the first and second protein partner molecules is detected. For this purpose, a light beam is directed towards the side of the surface area of the substrate that does not contain the sample to be tested and is reflected by said surface. The SPR phenomenon causes a decrease in the intensity of the reflected light with a specific combination of angle and wavelength. The binding of the first and second protein partner molecules causes a change in the refraction index on the substrate surface, which change is detected as a change in the SPR signal.

According to the preferred embodiment of the screening method cited above, the “first partner” of the screening system consists of the substrate onto which the first protein partner molecule is immobilised, and the “second partner” of the screening system consists of the second partner protein molecule itself.

### Affinity Chromatography

Candidate compounds for use in the screening method above can also be selected by any immunoaffinity chromatography technique using any chromatographic substrate onto which (i) the candidate polypeptide or (ii) the D-aspartate ligase or the L,D-transpeptidase as defined above, have previously been immobilised, according to techniques well known from the one skilled in the art.

In a preferred embodiment of the invention, the screening method includes the use of affinity chromatography.

The a D-aspartate ligase or the L,D-transpeptidase as defined above may be attached to a column using conventional techniques including chemical coupling to a suitable column matrix such as agarose, Affi Gel®, or other matrices familiar to those of skill in the art. In some embodiment of this method, the affinity column contains chimeric proteins in which the D-aspartate ligase or the L,D-transpeptidase as defined above, is fused to glutathion-s-transferase (GST). Then a candidate compound is applied to the affinity column. The amount of the candidate compound retained by the immobilized D-aspartate ligase or L,D-transpeptidase as defined above allows measuring the binding ability of said candidate compound on the enzyme and thus allows to assess the potential antibacterial activity of said candidate compound.

### High Throughput Screening

In another preferred embodiment of the screening method according to the invention, at step (b), the candidate substance and the D-aspartate ligase or the L,D-transpeptidase as defined above are labelled by a fluorophore. The measurement of the binding of the candidate compound to the D-aspartate ligase or to the L,D-transpeptidase as defined above, at step (b) consists of measuring a fluorescence energy transfer (FRET). Disruption of the interaction by a candidate compound is then followed by decrease or absence of fluorescence transfer. As an example, the one skilled in the art can make use of the TRACE technology of fluorescence transfer for Time Resolved Amplified Cryptate Emission developed by Leblanc V, et al. for measuring the FRET. This technique is based on the transfer of fluorescence from a donor (cryptate) to an acceptor of energy (XL665), when the two molecules are in close proximity in cell extracts.

Generally, the method for the screening of antibacterial substance that binds to a D-aspartate ligase or to a L,D-transpeptidase as defined above comprises further steps wherein the candidate substances that bind to the enzyme and which are thus positively selected at the end of step (b) of the screening method, are then assayed for their ability to actually inhibit said enzyme activity, by performing, as step (c) of said method, the corresponding screening method comprising a step of determining the ability of said candidate substances to inhibit the activity of a purified enzyme selected from the group consisting of:
(i) a D-aspartate ligase comprising a polypeptide having an amino acid sequence possessing at least 50% amino acid identity with an amino acid sequence selected from the group consisting of SEQ ID No 1 to SEQ ID No 10, or a biologically active fragment thereof; and
(ii) a L,D-transpeptidase comprising a polypeptide having an amino acid sequence possessing at least 50% amino acid identity with the amino acid sequence of SEQ ID No 11, or a biologically active fragment thereof.

### Crystallized L,D-Transpeptidase According to the Invention and Methods of Screening Using the Same

### Crystallized L-D, Transpeptidase

As shown in the examples herein, a L,D-transpeptidase as defined above has been crystallized.

More precisely, it has been obtained a high quality crystal of the L,D-transpeptidase consisting of the amino acid sequence beginning at the amino acid residue located at position 119 and ending at the amino acid residue located at position 466 of the L,D-transpeptidase of SEQ ID No 13.

Said amino acid sequence 119-466 portion of SEQ ID No 13 may also be termed SEQ ID No 33 throughout the present specification. Usually, for the amino acid residue numbering of SEQ ID No 33 herein, it is referred to the numbering of the same amino acid residue found in the complete amino acid sequence of said L,D-transpeptidase of SEQ ID No 13, without any indication to the contrary.

A method for preparing said crystallized L,D-transpeptidase is fully disclosed in the examples herein.

Most preferably, for crystallization, said L,D-transpeptidase is equilibrated against a reservoir containing 12.5% PEG 2000®, 100 mM ammonium sulfate, 300 mM NaCl and 100 mM sodium acetate trihydrate at pH 4.6.

Thus, another object of the invention consists of crystallized L,D-transpeptidase having the amino acid sequence of SEQ ID No 33.

It has been found according to the invention that the crystallized L,D-transpeptidase having the amino acid sequence of SEQ ID No 33 belong to the space group P3₁21 (a=b=115.976 and c=68.275) with one molecule per asymmetric unit and a solvent content of 64%.

### Three-Dimensional Structure of the Crystallized L,D-Transpeptidase of SEQ ID No 13.

Using a grown crystal of the L,D-transpeptidase according to the present invention, X-ray diffraction data can be collected by a variety of means in order to obtain the atomic coordinates of the molecules in the crystallized L,D-transpeptidase. In the examples herein, X-ray diffraction data were collected at the European Synchrotron Radiation Facility (ESRF) with the ESRF FIP-BM30A beamline. Then, the X-ray diffraction data were processed with the CCP4 program suite (containing the softwares named MOSFLM and SCALA).

With the aid of specifically designed computer software, such crystallographic data can be used to generate a three dimensional structure of the L,D-transpeptidase molecule. Various methods used to generate and refine a three dimensional structure of a molecular structure are well known to those skilled in the art, and include, without limitation, multiwavelength anomalous dispersion (MAD), single wavelength anomalous dispersion (SAD), multiple isomorphous replacement, reciprocal space solvent flattening, molecular replacement, and single isomorphous replacement with anomalous scattering (SIRAS).

The method for determining the structure of the L,D-transpeptidase disclosed in the examples herein consists of the single wavelength anomalous dispersion (SAD). The position of the three ordered Se atoms (out of a possible 5) were found using the CNS (Crystallography & NMR Software) software.

After density modification using the CNS SAD phase, the three-dimensional model of the L,D-transpeptidase was manually built using the program O described by Jones et al. (Jones, T. A., Zou, J. Y., Cowan, S. W. and Kjeldgaard, M. (1991); Improved methods for building protein models in electron density maps and the location of errors in these models. Acta Cryst A47, 110-119).

Most preferably, the structure is refined at 2.4 Å resolution using CNS, such as described by Brunger et al. (Brunger, A., Adams, P., Clore, G., DeLano, W., Gros, P., Grosse-Kunstleve, R., Jiang, J.-S., Kuszewski, J., Nilges, N., Pannu, N., et al. 1998. Crystallography and NMR system (CNS): A new software system for macromolecular structure determination, Acta Crystallogr. D 54: 905-921), with 20838 unique reflections (99.2% completeness).

The final three-dimensional model (Rcryst=22.0% and Rfree=25.7%; test set: 5% of the reflections) consists of residues 217-398 and 400 466 of SEQ ID No 13, one sulfate ion, one zinc ion and 295 water molecules. The 97 amino acid residues beginning at the amino acid residue located at position 119 and ending at the amino acid residue located at position 216 of SEQ ID No 13 could not be located in the map.

Most preferably, the final model of the three-dimensional structure of said L,D-transpeptidase, or of the 217-466 amino acid sequence thereof, is validated using the PROCHECK® software described by Laskowski et al. (Laskowski, R. A., McArthur, M. W., Moss, D. S., & Thornton, J. M. (1993). PROCHECK: A program to check the stereochemical quality of protein structures. J. Appl. Cryst 26, 283-291).

Ramachandran analysis has indicated that, for the three-dimensional model of the L,D-transpeptidase that is disclosed herein, 83.3% of the amino acid residues are in the most favored region, 15.3% of the amino acid residues are additionally allowed, and 1.4% of the amino acid residues are generously allowed.

The pertinency of the three-dimensional structure of the LD-transpeptidase (217-466 of SEQ ID No 13) has been performed by comparing the covalent bond distances and angles found from the X-ray diffraction data with standard values of covalent bond distances and angles for proteins, such as those standard values found in the book of Engh and Huber (Engh R. A. and Huber R., <<accurate Bond and Angle Parameters for X-ray Protein structure refinement>>, Acta Crytsallogr, A47 (1991): 392-400).

It was found that the three-dimensional model of the crystallized L,D-transpeptidase of the invention (i) has a root mean square deviation of bonds of 0.008 Å in respect to standard values and (ii) has a root mean square deviation of angles of 1.2° in respect to standard values, which are the total average deviation values that are found in standard dictionaries, including that of Engh and Huber that is referred to above.

As it can be noticed, the structural coordinates of the crystallized L,D-transpeptidase (217-466 of SEQ ID No 13) begin, in Table 3, with the amino acid residue LYS217, because of too much poor structural data concerning the amino acid residues 119-216 of said crystallized enzyme.

The X-ray diffraction data generated from the crystallized L,D-transpeptidase of SEQ ID No 33 has allowed to determine the spatial location of every atom of the polypeptide having the amino acid sequence beginning at the amino acid residue located at position 217 and ending at the amino acid residue located at position 466 of the L,D-transpeptidase of SEQ ID No 13

The cartesian coordinates which define one and every structural conformation feature of the L,D-transpeptidase (217-466 of SEQ ID No 13) of the invention are listed in Table 3.

In Table 3:
first column designates the nature of the information given in the corresponding line;
second column represents a single increment numbering of the lines of Table 3;
third column refers to a specific atom of the considered amino acid;
fourth column designates the specific amino acid of the peptide fragment which is considered;
fifth column refers to the peptide chain to which a specific amino acid belongs;
sixth column specifies the amino acid position of the amino acid which is considered, as regards the numbering of the amino acid sequence of the L,D-transpeptidase (119-466 of SEQ ID No 13)
seventh, eighth and ninth columns specify the Cartesian coordinates of the atom which is considered along, respectively, the x, y and z axis;
tenth column specifies the occupancy of the considered position by the considered atom;
eleventh column specifies the B factor characterizing the thermal motion of the considered atom;
twelfth column refers to the peptide chain to which a specific amino acid belongs;

As used herein, “structural coordinates” are the Cartesian coordinates corresponding to an atom's spatial relationship to other atoms in a molecule or molecular complex. Various software programs allow for the graphical representation of a set of structural coordinates of the present invention may be modified from the original sets provided in Table 3 by mathematical manipulation, such as by inversion or integer additions or subtractions. As such, it is recognised that the structural coordinates of the present invention are relative, and are in no way specifically limited by the actual x, y, z coordinates in Table 3.

As used herein, “Root mean square deviation” is the square root of the arithmetic mean of the squares of the deviations from the mean, and is a way of expressing deviation or variation from the structural coordinates described herein. The present invention includes all embodiments comprising conservative substitutions of the noted amino acid residues resulting in the same structural coordinates within the stated root mean square deviation.

It will be obvious to the one skilled in the art that the numbering of the amino acid residues of the crystallized L,D-transpeptidase defined herein may be different than set forth herein, and may contain certain conservative amino acid substitutions that yield similar three-dimensional structures as those defined in Table 3 herein. Corresponding amino acids and conservative substitutions are easily identified by visual inspection of the relevant amino acid sequences or by using commercially available homology modelling software programs, such as MODELLER (MSI, San Diego, Calif., USA).

As used herein, “conservative substitutions” are those amino acid substitutions which are functionally equivalent to the substituted amino acid residue, either by way of having similar polarity, steric arrangement, or by belonging to the same class as the substituted residue (e.g. hydrophobic, acidic or basic), and includes substitutions having an inconsequential effect on the three dimensional structure of the crystallized protein complex of the invention with respect to the use of said structures for the identification of ligand compounds which interact with the catalytic site of the L,D-transpeptidase of SEQ iD No 33 or of SEQ ID No 13, more particularly, inhibitor compounds, for molecular replacement analyses and/or for homology modelling.

As shown in the examples, the various amino acid residues from the catalytic site of the L,D-transpeptidase of SEQ ID No 33 or of SEQ ID No 13 that delineate the inner space area of said catalytic site have been determined, using the structural coordinates of the crystallized protein complex which are set forth in Table 3.

The crystallized L,D-transpeptidase of SEQ ID No 33, and more specifically the inner space area of its catalytic site, can also be defined exclusively as respect to the various amino acid residues which are involved in delineating it.

From the three-dimensional structure of the crystallized L,D-transpeptidase of SEQ ID No 33 that can be determined from the structure coordinates of (217-466 of SEQ ID No 13) found in Table 3, the structure of the catalytic site of said enzyme has been deciphered. The structural data strictly corroborate the biological data found by directed mutagenesis.

It has been found that the most important amino acid residues contained in the active site are SER439 and CYS442, respectively, which are phylogenetically conserved residues on the basis of which a specific protein family can be defined.

It has also been found that two additional amino acid residues are important in the active site, respectively HIS421 and HIS440.

Another object of the invention consists of a crystallized L,D-transpeptidase of SEQ ID No 33, a three-dimensional atomic structure of the catalytic sites is defined by a set of structure coordinates having a root mean square deviation of not more than 1.5 Å from the set of structure coordinates corresponding to amino acid residues HIS421, SER439, HIS440 and CYS442 according to Table 3.

It has also been found according to the invention that the whole amino acid residues that delineate the catalytic site of the L,D-transpeptidase of SEQ ID No 13 or of SEQ ID No 33 are encompassed within the polypeptide beginning at the amino acid residue ILE368 and ending at the amino acid residue MET450 of SEQ ID No 13.

Thus, the three-dimensional structure of the catalytic site of the crystallized L,D-transpeptidase of SEQ ID No 13 or of SEQ ID No 33 comprises, in addition to the set of data corresponding to the relative structural coordinates of amino acid residues HIS421, SER439, HIS440 and CYS442 according to Table 3, equally a set of data corresponding to the relative structural coordinates of one or more of the following amino acid residues: ILE368, VAL369, SER370, GLY371, LYS372, PRO373, THR374, THR375, PRO376, THR377, PRO378; ALA379, GLY380, VAL381, PHE382, TYR383, VAL384, TRP385, ASN386, LYS387, GLU388, GLU389, ASP390, ALA391, THR392, LEU393, LYS394, GLY395, THR396, ASN397, ASP398, ASP399, GLY400, THR401, PRO402, TYR403, GLU404, SER405, PRO406, VAL407, ASN408, TYR409, TRP410, MET411, PRO412, ILE413, ASP414, TRP415, THR416, GLY417, VAL418, GLY419, ILE420, ASP422, SER423, ASP424, TRP425, GLN426, PRO427, GLU428, TYR429, GLY430, GLY431, ASP432, LEU433, TRP434, LYS435, THR436, ARG437, GLY438, GLY441, ILE443, ASN444, THR445, PRO446, PRO447, SER448, VAL449, MET450, LYS451, GLU452, LEU453, PHE454, GLY455, MET456, VAL457, GLU458, LYS459, GLY460, THR461, PRO462, VAL463, LEU464, VAL465 and PHE466.

### Methods for the Screening of Compounds Inhibiting the L,D-Transpeptidase of the Invention, Using the Three-Dimensional Structure of Said L,D-Transpeptidase.

The availability, according to the present invention, of the whole structural coordinates of the 217-466 portion of the L,D-transpeptidase of SEQ ID No 13 described above, and specifically of the structural coordinates of the various amino acid residues which are involved for forming the catalytic site, allows the one skilled in the art to generate models of docking compounds of a known chemical structure within said catalytic site and select those compounds that are potential or actual antibacterial compounds, that is compounds that potentially inhibit said L,D-transpeptidase.

More particularly, according to the invention, a compound which will behave as an inhibitor of the L,D-transpeptidase of SEQ ID No 13 or of SEQ id No 33 consists of a compound that, when docked in its catalytic site, either:
(i) said compound induces steric constraints onto one or several chemical groups, including lateral chains, of one or several of the amino acid residues which are involved in delineating said catalytic site, so that said compound causes a spatial change, namely a deformation, of said catalytic site leading potentially to an inhibition of said L,D-transpeptidase;
(ii) said compound forms one or more non covalent bonds with one or several chemical groups, including lateral chains, of one or several of the amino acid residues which are involved in delineating said catalytic site, so that availability of the catalytic site of said L,D-transpeptidase for its substrate(s) is potentially reduced or blocked; or
(iii) said compound forms one or more covalent bonds with one or several chemical groups, including lateral chains, of one or several of the amino acid residues which are involved in delineating said catalytic site, so that availability of the catalytic site of said L,D-transpeptidase for its substrate(s) is blocked.

In another aspect, the present invention is directed to a method for identifying a ligand compound, more particularly an inhibitor of the L,D-transpeptidase of SEQ ID No 13 or of SEQ ID No 33, said method comprising a step of docking or fitting the three-dimensional structure of a candidate compound with the three-dimensional structure of the catalytic site of the L,D-transpeptidase of Seq ID No 13 or of SEQ ID No 33.

Thus, another object of the invention consists of a method for selecting a compound that fits in the catalytic site of the L,D-transpeptidase of SEQ ID No 13 or of SEQ ID No 33, wherein said method comprises the steps of:
a) generating a three-dimensional model of the L,D-transpeptidase (217-466 of SEQ ID No 13) using a set of data corresponding to the relative structural coordinates according to Table 3, and
b) employing said three-dimensional model to design or select a compound, from a serial of compounds, that interacts with said catalytic site.

A further object of the invention consists of a method for selecting an inhibitor compound for the L,D-transpeptidase of SEQ ID No 13 or of SEQ ID No 33, wherein said method comprises the steps of:
a) generating a three-dimensional model of the catalytic site of the L,D-transpeptidase (119-466 or 217-466 of SEQ ID No 13) using a set of data corresponding to the relative structural coordinates of amino acid residues HIS421, SER439, HIS440 and CYS442 according to Table 3±a root mean square deviation from the backbone atoms of said amino acids of not more than 1.5 Å; and
b) performing, for each candidate compound, a computer fitting analysis of said candidate inhibitor compound with three-dimensional model generated at step a); and
c) selecting, as an inhibitor compound either:
   (i) every candidate compound having a chemical structure inducing hydrogen bonds with at least two of the HIS421, SER439, HIS440 and CYS442 amino acid residues of the catalytic site of the L,D-transpeptidase (119-466 or 217-466 of SEQ ID No 13); and
   (ii) every candidate compound having a chemical structure inducing steric constraints with at least one of the amino acid residues comprised in the 368-450 polypeptide portion of the L,D-transpeptidase of SEQ ID No 13.
   (iii) every candidate compound having a chemical structure such that one or more covalent bonds are formed between said candidate compound and one or more chemical groups, including lateral chains, of one or several amino acid residues which are involved in delineating the catalytic site of the L,D-transpeptidase (119-466 or 217-466 of SEQ ID No 13).
      Candidate Compounds that May be Designed or Selected at Step (B) of the Screening Method.

In order to further precise the class of compounds to which the selected ligand belongs, step b) may further comprise specific sub-steps wherein it is determined whether the compound, which has been primarily selected for its ability to interact with the catalytic site of the L,D-transpeptidase of SEQ ID No 13 or of SEQ ID No 33, further induces stabilisation or, in contrast, steric constraints onto chemical groups belonging to the amino acid residues involved in said catalytic site so as to stabilise the spatial conformation of the catalytic site or, in contrast, cause a change in the spatial conformation of the catalytic site that reduces or even blocks the catalytic activity of the L,D-transpeptidase.

According to a first aspect of the screening method above, the candidate ligand compound, more particularly the candidate inhibitor compound, is selected from a library of compounds previously synthesised.

According to a second aspect of the screening method above, the candidate ligand compound, more particularly the candidate inhibitor compound, is selected from compounds, the chemical structure of which is defined in a database, for example an electronic database.

According to a third embodiment of the screening method above, the candidate ligand compound, more particularly the candidate inhibitor compound, is conceived de novo, by taking into account the spatial conformation stabilisation or, in contrast, the spatial conformation changes, that chemical group(s) of said compound may cause, when docked within the catalytic site of the L,D-transpeptidase of SEQ ID No 33. Indeed, after its de novo conception, and if positively selected, said candidate ligand compound, more particularly said candidate inhibitor compound, can be actually chemically synthesised.

Generally, computational methods for designing an inhibitor of the L,D-transpeptidase of SEQ ID No 13 or of SEQ ID No 33 determine which amino acid or which amino acids of the catalytic site interact with a chemical moiety (at least one) of the ligand compound using a three dimensional model of the crystallized protein complex of the invention, the structural coordinates of which are set forth in Table 3.

These computational methods are particularly useful in designing an inhibitor of the L,D-transpeptidase of SEQ ID No 13 or of SEQ ID No 33, wherein said inhibitor compound has a chemical moiety, or chemical group(s) that allow the formation of hydrogen bonds with the side chains of the amino acid residues that are involved in the catalytic site, and more particularly the side chains of HIS421 (NH group), SER439 (OH group), HIS440 (NH group) and CYS442 (SH group).

Methods for Docking or Fitting Candidate Compounds with the Catalytic Site of Said L,D-Transpeptidase.

The three-dimensional structure of the L,D-transpeptidase of SEQ ID No 33 will greatly aid in the development of inhibitors of L,D transpeptidases that can be used as antibacterial substances. In addition, said L,D-transpeptidase is overall well suited to modern methods including three dimensional structure elucidation and combinatorial chemistry such as those disclosed in the European patent No EP 335 628 and the U.S. Pat. No. 5,463,564, which are incorporated herein by reference. Computer programs that use crystallographic data when practising the present invention will enable the rational design of ligand to, particularly inhibitor of, the L,D-transpeptidase of SEQ ID No 13 or of SEQ ID No 33.

Generally, the computational method of designing a synthetic ligand to, particularly a synthetic inhibitor of, the L,D-transpeptidase of SEQ ID No 13 or of SEQ ID No 33 comprises two steps:
1) determining which amino acid or amino acids of the L,D-transpeptidase (217-466 of SEQ ID No 13) interacts with a first chemical moiety (at least one) of the ligand using a three dimensional model of a crystallized protein comprising the catalytic site with a bound ligand; and
2) selecting a chemical modification (at least one) of the first chemical moiety to produce a second chemical moiety with a structure to either increase or decrease an interaction between the interacting amino acid and the second chemical moiety compared to the interaction between the interacting amino acid and the first chemical moiety.

As shown herein, interacting amino acids form contacts with the ligand and the center of the atoms of the interacting amino acids are usually 2 to 4 angstroms away from the center of the atoms of the ligand. Generally these distances are determined by computer as discussed herein and as it is described by Mc Ree (1993), however distances can be determined manually once the three dimensional model is made. Also, it has been described how performing stereochemical figures of three dimensional models using for instance the program Bobscript on the following website: http://www.strubi.ox.ac.uk/bobscript/doc24.html#StereoPS.

More commonly, the atoms of the ligand and the atoms of interacting amino acids are 3 to 4 angstroms apart. The invention can be practiced by repeating step 1 and 2 above to refine the fit of the ligand to the catalytic site of the L,D-transpeptidase (119-466 or 217-466 of SEQ ID No 13) and to determine a better ligand, specifically an inhibitor compound. The three dimensional model of the L,D-transpeptidase (119-466 or 217-466 of SEQ ID No 13) can be represented in two dimensions to determine which amino acids contact the ligand and to select a position on the ligand for chemical modification and changing the interaction with a particular amino acid compared to that before chemical modification. The chemical modification may be made using a computer, manually using a two dimensional representation of the three dimensional model or by chemically synthesizing the ligand. The ligand can also interact with distant amino acids after chemical modification of the ligand to create a new ligand. Distant amino acids are generally not in contact with the ligand before chemical modification. A chemical modification can change the structure of the ligand to make a new ligand that interacts with a distant amino acid usually at least 4.5 angstroms away from the ligand, preferably wherein said first chemical moiety is 6 to 12 angstroms away from a distant amino acid. Often distant amino acids will not line the surface of the binding activity for the ligand, they are too far away from the ligand to be part of a pocket or binding cavity. The interaction between a catalytic site amino acid and an atom of a ligand can be made by any force or attraction described in nature. Usually the interaction between the atom of the amino acid and the ligand will be the result of a hydrogen bonding interaction, charge interaction, hydrophobic effect, van der Waals interaction or dipole interaction. In the case of the hydrophobic effect it is recognized that is not a per se interaction between the amino acid and ligand, but rather the usual result, in part, of the repulsion of water or other hydrophilic group from a hydrophobic surface. Reducing or enhancing the interaction of the catalytic site of the L,D-transpeptidase (119-466 or 217-466 of SEQ ID No 13) and a ligand can be measured by calculating or testing binding energies, computationally or using thermodynamic or kinetic methods as known in the art.

Chemical modifications will often enhance or reduce interactions of an atom of a catalytic site amino acid and an atom of the ligand. Steric hindrance will be a common means of changing the interaction of the catalytic cavity with the ligand.

However, as will be understood by those of skill in the art upon this disclosure, other structure based design methods can be used. Various computational structure based design methods have been disclosed in the art.

For example, a number computer modeling systems are available in which the sequence of the L,D-transpeptidase (119-466 or 217-466 of SEQ ID No 13) structure, particularly of the catalytic site of the L,D-transpeptidase (119-466 or 217-466 of SEQ ID No 13) structure (i.e., atomic coordinates of the catalytic site, the bond and dihedral angles, and distances between atoms in the active site such as provided in Table 3) can be input. This computer system then generates the structural details of the site in which a potential ligand compound binds so that complementary structural details of the potential modulators can be determined. Design in these modelling systems is generally based upon the compound being capable of physically and structurally associating with the catalytic site of the L,D-transpeptidase (119-466 or 217-466 of SEQ ID No 13). In addition, the compound must be able to assume a conformation that allows it to associate with said catalytic site.

Methods for screening chemical entities or fragments for their ability to associate with the L,D-transpeptidase (119-466 or 217-466 of SEQ ID No 13), and more particularly the catalytic site of the L,D-transpeptidase (119-466 or 217-466 of SEQ ID No 13), are also well known. Often these methods begin by visual inspection of the active site on the computer screen. Selected fragments or chemical entities are then positioned with the catalytic site of the L,D-transpeptidase (119-466 or 217-466 of SEQ ID No 13). Docking is accomplished using software such as QUANTA and SYBYL, followed by energy minimization and molecular dynamics with standard molecular mechanic forcefields such as CHARMM and AMBER. Examples of computer programs which assist in the selection of chemical fragment or chemical entities useful in the present invention include, but are not limited to, GRID (Goodford, P. J. J. Med. Chem. 1985 28: 849-857), AUTODOCK (Goodsell, D. S. and Olsen, A. J. Proteins, Structure, Functions, and Genetics 1990 8: 195-202), and DOCK (Kunts et al. J. Mol. Biol. 1982 161:269-288).

Upon selection of preferred chemical entities or fragments, their relationship to each other and the catalytic site of the L,D-transpeptidase (119-466 or 217-466 of SEQ ID No 13) can be visualized and then assembled into a single potential modulator. Programs useful in assembling the individual chemical entities include, but are not limited to, CAVEAT (Bartlett et al. Molecular Recognition in Chemical and Biological Problems Special Publication, Royal Chem. Soc. 78, 00. 182-196 (1989)) and 3D Database systems (Martin, Y. C. J. Med. Chem. 1992 35:2145-2154).

Alternatively, compounds may be designed de novo using either an empty active site or optionaly including some portion of a known inhibitor. Methods of this type of design include, but are not limited to LUDI (Bohm H-J, J. Comp. Aid. Molec. Design 1992 6:61-78) and LeapFrog (Tripos Associates, St. Louis Mo.).

For “fitting” or “docking” a ligand compound to the catalytic site of the L,D-transpeptidase (119-466 or 217-466 of SEQ ID No 13), starting from the structural coordinates of the protein complex of the invention which are set forth in Table 3, the one skilled in the art may use known techniques such as those reviewed by Sheridan et al. (1987), Goodford (1984), Beddell (1985), Hol (1986), Verlinde et al. (1994) and Blundell et al. (1987).

Fitting or docking a ligand compound to to the catalytic site of the L,D-transpeptidase (119-466 or 217-466 of SEQ ID No 13), starting from the structural coordinates of the protein complex of the invention which are set forth in Table 3, can also be performed using_software such as QUANTA and SYBYL, followed by energy minimisation and molecular dynamics with standard molecular mechanic force fields such as CHARMM and AMBER. Examples of computer programs which assist in the selection of chemical fragment or chemical entities useful in the present invention include, but are not limited to, GRID (Goodford, P. J. J. Med. Chem. 1985 28: 849-857), AUTODOCK (Goodsell, D. S. and Olsen, A. J. Proteins, Structure, Functions, and Genetics 1990 8: 195-202), and DOCK (Kunts et al. J. Mol. Biol. 1982 161:269-288).

Most preferably, according to the invention, the structure determination of a crystallized protein complex, whether free of a ligand compound or under the form of a complex with a ligand compound, is performed by molecular replacement using AMoRe, as described by Navaza et al. (1994) with the crystallized L,D-transpeptidase that is described herein as the search model.

Use of a computer program has two main goals: complex prediction and virtual screening.

### Complex Prediction

In the first approach (complex prediction), one starts from a small molecule selected on the basis of a visual examination of the ligand-binding pocket as revealed by X-ray crystallography or predicted from homology modelling. Indeed, the knowledge of the ligand-binding pocket gives indications about the size, the shape, and putative anchoring groups of the ligand. Once a suitable candidate is selected, its molecular model can be built thanks to modules of programs such as the QUANTA Molecular Modeling Package (Accelrys, San Diego, Calif., USA). Then the putative ligand is docked manually in the ligand-binding pocket by the one skilled in the art to evaluate its suitability as a candidate ligand, based on:
the absence of steric clashes with atoms from the protein residues forming the catalytic site of the L,D-transpeptidase (119-466 or 217-466 of SEQ ID No 13) (showing the physical possibility to be accommodated in the pocket),
the possibility to form favourable interactions with atoms from the catalytic site, such as salt bridges, hydrogen bonds, or van der Waals contacts (showing the potential for a high affinity for the catalytic site of the L,D-transpeptidase).

This procedure can be referred to as “manual” design.

In an improved procedure, the position of the manually docked ligand in the catalytic site is optimised through the use of an energy minimization algorithm such as the one provided in CNS (Brunger, A. T. et al. (1998) “Crystallography and NMR system (CNS): A new software system for macromolecular structure determination” Acta Cryst. D54: 905-921). In an even further improved procedure, docking programs are used to predict the geometry of the protein-ligand complex and estimates the binding affinity. Programs that perform flexible protein-ligand docking include GOLD (Jones et al. (1995) J. Mol. Biol. 245:43-53), FlexX (Rarey, M. et al. (1995) “Time-efficient docking of flexible ligands into active sites of proteins” Proc. Int. Conf. Intell. Syst. Mol. Biol. 3:300-308, AAAI Press, Menlo Park, Calif., USA), and Dock (Ewing, T. J. A. and Kuntz, I. D. (1997) “Critical evaluation of search algorithms for automated molecular docking and database screening” J. Comput. Chem. 18:1175-1189). The SuperStar program (Verdonk, M. L. et al. (1999) “A knowledge-based approach for identifying interaction sites in proteins” J. Mol. Biol. 289; 1093-1108) is used for the prediction of favourable interaction sites in proteins.

### Virtual Screening

In the second approach (virtual screening), a more advanced procedure, the computer program is used to search a whole small-molecule database (see for instance: Makino, S. and Kuntz, I. D. (1997) “Automated flexible ligand docking method and its application for database search” J. Comp. Chem. 18:1812-1825).

Further Characterization as L,D-Transpeptidase Inhibitors of the Compounds that are Positively Selected at the End of Step b) of the Method.

Once a ligand has been selected on the basis of its predicted binding to the receptor through docking studies as described above, it can be validated according to any of the methods below:

(i) Detecting of the direct binding of the ligand to the catalytic site of the L,D-transpeptidase of SEQ ID No 33, that can be demonstrated by electrospray ionisation mass spectrometry (ESI MS) under non-denaturing conditions, a technique allowing the detection of non-covalent compexes (Loo, J. A., (1997) “Studying noncovalent protein complexes by electrospray ionisation mass spectrometry” Mass Spectrom, Rev. 16: 1-23);

(ii) Measuring the L,D-transpeptidase activity in the presence of the candidate ligand.

In order to further characterise the biological activity of the compound which has been positively selected by performing steps (a) and (b) of the screening method above, it may be required to assay for the actual biological activity of said positively selected compound, in respect to the catalytic activity of the L,D-transpeptidase of SEQ ID No 13, or of the SEQ ID No 33 polypeptide portion thereof.

According to a first aspect, a further biological assay using said positively selected compound will confirm that said candidate compound that is positively selected at the end of step (b) of the method effectively reduces or blocks the catalytic activity of the L,D-transpeptidase.

Thus, in a further embodiment, the screening method above, said method further comprises the steps of:
c) obtaining the compound designed or selected at step b); and
d) contacting the compound obtained at step c) with a L,D-transpeptidase as defined in the present specification in order to determine the effect the compound has on the activity of said L,D-transpeptidase.

In a most preferred embodiment, step d) of the screening method above consists of performing the screening method which has been previously described in detail in the present specification, which screening method makes use of a L,D-transpeptidase comprising a polypeptide having an amino acid sequence possessing at least 50% amino acid identity with the amino acid sequence of SEQ ID No 13, or a biologically active fragment thereof.

In a preferred embodiment of said screening method, in step d), the compound which has been selected in step b) is used as the candidate inhibitor compound in step a) of the biological screening method which is used in step d).

Thus, from above, assays are known and available for determining whether a ligand identified or designed according to the present invention actually inhibits L,D-transpeptidase activity. High-affinity, high-specificity ligands found in this way can then be used for in vitro and in vivo assays aiming at determining the antibacterial properties of said ligand, including its spectrum of activity against various bacteria strains, species or genus.

Finally, from above, assays are available for determining whether these ligands may be useful therapeutically.

The present invention further relates to a method for selecting a compound that interacts with the catalytic site of the L,D-transpeptidase (119-466 or 217-466 of SEQ ID No 13), wherein said method consists in:
a) selecting or designing a candidate inhibitor compound for the L,D-transpeptidase (119-466 or 217-466 of SEQ ID No 13) by performing computer fitting analysis of said candidate inhibitor compound with the three-dimensional structure of the catalytic site of the L,D-transpeptidase (119-466 or 217-466 of SEQ ID No 13) that is disclosed in the present specification.

The selection or the design of said candidate inhibitor compound is carried out by one of the methods which are extensively described above.

Thus, in a further embodiment, the screening method above, said method further comprises the steps of:
b) obtaining the compound designed or selected at step a); and
c) contacting the compound obtained at step b) with a L,D-transpeptidase as defined herein in order to determine the effect the compound has on the catalytic activity of said L,D-transpeptidase.

In a preferred embodiment of said screening method, in step c), the compound which has been selected in step a) is used as the candidate inhibitor compound in step b) of the biological screening method which is described in the present specification and in the examples.

As already described previously in the present specification, an object of the present invention consists of a method for selecting an inhibitor compound for the L,D-transpeptidase (119-466 or 217-466 of SEQ ID No 13), wherein said method comprises the steps of:
a) generating a three-dimensional model of the catalytic site of the L,D-transpeptidase (119-466 or 217-466 of SEQ ID No 13) using a set of data corresponding to the relative structural coordinates of amino acid residues HIS421, SER439, HIS440 and CYS442 according to Table 3±a root mean square deviation from the backbone atoms of said amino acids of not more than 1.5 Å; and
b) performing, for each candidate compound, a computer fitting analysis of said candidate inhibitor compound with three-dimensional model generated at step a); and
c) selecting, as an inhibitor compound, every candidate compound having a chemical structure inducing either:
   (i) hydrogen bonds with at least two of the HIS421, SER439, HIS440 and CYS442 amino acid residues of the catalytic site of the L,D-transpeptidase (119-466 or 217-466 of SEQ ID No 13); or
   (ii) steric constraints with at least one amino acid residue comprised in the 368-450 polypeptide portion of the L,D-transpeptidase of SEQ ID No 13.

In a specific embodiment, the screening method above, said method further comprises the steps of:
d) obtaining the compound designed or selected at step c); and
e) contacting the compound obtained at step d) with a L,D-transpeptidase, particularly the L,D-transpeptidase (119-466 or 217-466 of SEQ ID No 13), in order to determine the effect the compound has on the catalytic activity of said L,D-transpeptidase.

In a preferred embodiment of said screening method, in step d), the compound which has been selected in step c) is used as the candidate inhibitor compound in step b) of the biological screening method which is disclosed in the present specification.

According to a first aspect of the screening method above, the candidate ligand compound, more particularly the candidate inhibitor compound, is selected from a library of compounds previously synthesised.

According to a second aspect of the screening method above, the candidate ligand compound, more particularly the candidate agonist or antagonist compound, is selected from compounds, the chemical structure of which is defined in a database, for example an electronic database.

According to a third embodiment of the screening method above, the candidate ligand compound, more particularly the candidate inhibitor compound, is conceived de novo, by taking into account the spatial conformation stabilisation or, in contrast, the spatial conformation changes, that chemical group(s) of said compound may cause, when docked within the catalytic site of the L,D-transpeptidase (119-466 or 217-466 of SEQ ID No 13). Indeed, after its de novo conception, and if positively selected, said candidate ligand compound, more particularly said candidate inhibitor compound, can be actually chemically synthesised.

### Molecular Models and Systems of the Invention

The present invention is also directed to a molecular model comprising:
(i) the catalytic site of the L,D-transpeptidase (119-466 or 217-466 of SEQ ID No 13) defined by a set of data corresponding to the structural coordinates of amino acid residues HIS421, SER439, HIS440 and CYS442 according to Table 3±a root mean square deviation from the backbone atoms of said amino acids of not more than 1.5 Å; and
(ii) a ligand for said catalytic site of the L,D-transpeptidase (119-466 or 217-466 of SEQ ID No 13).

The present invention is also directed to a machine-readable data storage medium, comprising a data storage material encoded with machine-readable data, wherein said machine-readable data consist of the X-ray structural coordinate data of the L,D-transpeptidase (119466 or 217466 of SEQ ID No 13) according to Table 3.

A used herein, a “machine-readable data storage medium” refers to any media which can be read and accessed directly by a computer. Such media include, but are not limited to, magnetic storage media such as floppy discs, hard disc storage medium and magnetic tape; optical storage media such as optical discs or CD-ROM; electrical storage media such as RAM and ROM; and hybrids of these categories such as magnetic/optical storage media.

This invention is also directed to a machine-readable data storage medium, comprising a data storage material encoded with machine-readable data which, when using a machine programmed with instructions for using said data, displays a graphical three-dimensional representation of a crystal of the catalytic site of the L,D-transpeptidase of SEQ ID No 33.

This invention is also directed to a machine-readable data storage medium, comprising a data storage material encoded with machine-readable data which, when using a machine programmed with instructions for using said data, displays a graphical three-dimensional representation of a crystal of the L,D-transpeptidase of SEQ ID No 33 that is complexed with one candidate inhibitor of the L,D-transpeptidase of SEQ ID No 33.

This invention is also directed to a system for generating a three-dimensional model of at least a portion of the L,D-transpeptidase of SEQ ID No 13, said system comprising:
a) a data storage device storing data comprising a set of structure coordinates defining at least a portion of the three-dimensional structure of said L,D-transpeptidase according to Table 3; and
b) a processing unit being for generating the three-dimensional model from said data stored in said data-storage device.

In preferred embodiments of the system above, said system further comprises a display device for displaying the three-dimensional model generated by said processing unit.

### Assessment of the Ex Vivo Activity of the Inhibitor Compounds Positively Selected by the In Vitro or in Silico Screening Methods Disclosed Above

Inhibitor substances that have been positively selected at the end of any one of the screening methods that are previously described in the present specification may then be assayed for their ex vivo antibacterial activity, in a further stage of their selection as a useful antibacterial active ingredient of a pharmaceutical composition.

By “ex vivo” antibacterial activity, it is intended herein the antibacterial activity of a positively selected candidate compound against bacteria cells that are cultured in vitro.

Thus, any substance that has been shown to behave like an inhibitor of a D-aspartate ligase or of a L,D-transpeptidase, after positive selection at the end of any one of the screening methods that are disclosed previously in the present specification, may be further assayed for his ex vivo antibacterial activity.

Consequently, any one of the screening methods that are described above may comprise a further step of assaying the positively selected inhibitor substance for its ex vivo antibacterial activity.

Usually, said further step consists of preparing in vitro bacterial cultures and then adding to said bacterial cultures the candidate compound to be tested, before determining the ability of said candidate compound to block bacterial growth or even most preferably kill the cultured bacterial cells.

For assaying the ex vivo antibacterial activity of a candidate compound that has previously been shown to affect the catalytic activity of a D-aspartate ligase encompassed by the present invention, bacteria cells that are cultured in vitro are preferably selected from the group consisting of Enterococcus faecium, Lactococcus lactis, Lactococcus cremoris SK111, Lactobacillus gasseri, Lactobacillus johnosonii NCC 533, Lactobacillus delbruckei Subsp. bulgaricus, Lactobacillus casei, Lactobacillus acidophilus, Lactobacillus brevis and Pediococcus pentosaceus.

Typically, bacterial cells are plated in Petri dishes containing the appropriate culture medium, generally in agar gel, at a cell number ranging from 10 to 10³ bacterial cells, including from 10 to 10² bacterial cells. In certain embodiments, serials of bacterial cultures are prepared with increasing numbers of seeded bacterial cells.

Typically, the candidate compound to be tested is then added to the bacterial cultures, preferably with a serial of amounts of said candidate compounds for each series of a given plated cell number of bacterial cultures.

Then, the bacterial cultures are incubated in the appropriate culture conditions, for instance in a cell incubator at the appropriate temperature, and for an appropriate time period, for instance a culture time period ranging from 1 day to 4 days, before counting the resulting CFUs (Colony Forming Units), either manually under a light microscope or binocular lenses, or automatically using an appropriate apparatus.

Generally, appropriate control cultures are simultaneously performed, i.e; negative control cultures without the candidate substance and positive control cultures with an antibiotic that is known to be toxic against the cultured bacterial cells.

Finally, said candidate compound is positively selected at the end of the method if it reduces the number of CFUs, as compared with the number of CFUs found in the corresponding negative control cultures.

Thus, another object of the present invention consists of a method for the ex vivo screening of a candidate antibacterial substance which comprises the steps of:
a) performing a method for the in vitro screening of a antibacterial substances as disclosed in the present specification, with a candidate substance; and
b) assaying a candidate substance that has been positively selected at the end of step a) for its ex vivo antibacterial activity.

### Assessment of the In Vivo Activity of the Inhibitor Compounds Positively Selected by the In Vitro, in Silico or Ex Vivo Screening Methods Disclosed Above

Inhibitor substances that have been positively selected at the end of any one of the screening methods that are previously described in the present specification may then be assayed for their in vivo antibacterial activity, in a further stage of their selection as a useful antibacterial active ingredient of a pharmaceutical composition.

Thus, any substance that has been shown to behave like an inhibitor of a D-aspartate ligase or a L,D-transpeptidase, after positive selection at the end of any one of the screening methods that are disclosed previously in the present specification, may be further assayed for his in vivo antibacterial activity.

Consequently, any one of the screening methods that are described above may comprise a further step of assaying the positively selected inhibitor substance for its in vivo antibacterial activity.

Usually, said further step consists of administering the inhibitor substance to a mammal and then determining the antibacterial activity of said substance.

Mammals are preferably non human mammals, at least at the early stages of the assessment of the in vivo antibacterial effect of the inhibitor compound tested. However, at further stages, human volunteers may be administered with said inhibitor compound to confirm safety and pharmaceutical activity data previously obtained from non human mammals.

Non human mammals encompass rodents like mice, rats, rabbits, hamsters, guinea pigs. Non human mammals and also cats, dogs, pigs, veals, cows, sheep, goats. Non human mammals also encompass primates like macaques and baboons.

Thus, another object of the present invention consists of a method for the in vivo screening of a candidate antibacterial substance which comprises the steps of:
a) performing a method for the in vitro screening of a antibacterial substances as disclosed in the present specification, with a candidate substance; and
b) assaying a candidate substance that has been positively selected at the end of step a) for its in vivo antibacterial activity.

Preferably, serial of doses containing increasing amounts of the inhibitor substance are prepared in view of determining the antibacterial effective dose of said inhibitor substance in a mammal subjected to a bacterial infection. Generally, the ED₅₀ dose is determined, which is the amount of the inhibitor substance that is effective against bacteria in 50% of the animals tested. In some embodiments, the ED₅₀ value is determined for various distinct bacteria species, in order to assess the spectrum of the antibacterial activity.

In certain embodiments, it is made use of serial of doses of the inhibitor substance tested ranging from 1 ng to 10 mg per kilogram of body weight of the mammal that is administered therewith.

Several doses may comprise high amounts of said inhibitor substance, so as to assay for eventual toxic or lethal effects of said inhibitor substance and then determine the LD₅₀ value, which is the amount of said inhibitor substance that is lethal for 50% of the mammal that has been administered therewith.

The inhibitor substance to be assayed may be used alone under the form of a solid or a liquid composition.

When the inhibitor substance is used alone, the solid composition is usually a particulate composition of said inhibitor substance, under the form of a powder.

When the inhibitor substance is used alone, the liquid composition is usually a physiologically compatible saline buffer, like Ringer's solution or Hank's solution, in which said inhibitor substance is dissolved or suspended.

In other embodiments, said inhibitor substance is combined with one or more pharmaceutically acceptable excipients for preparing a pre-pharmaceutical composition that is further administered to a mammal for carrying out the in vivo assay.

Before in vivo administration to a mammal, the inhibitor substances selected through any one of the in vitro screening methods above may be formulated under the form of pre-pharmaceutical compositions. The pre-pharmaceutical compositions can include, depending on the formulation desired, pharmaceutically acceptable, usually sterile, non-toxic carriers or diluents, which are defined as vehicles commonly used to formulate pharmaceutical compositions for animal or human administration. The diluent is selected so as not to affect the biological activity of the combination. Examples of such diluents are distilled water, physiological saline, Ringer's solutions, dextrose solution, and Hank's solution. In addition, the test composition or formulation may also include other carriers, adjuvants, or nontoxic, nontherapeutic, nonimmunogenic stabilizers and the like.

Compositions comprising such carriers can be formulated by well known conventional methods. These test compositions can be administered to the mammal at a suitable dose. Administration of the suitable compositions may be effected by different ways, e.g., by intravenous, intraperitoneal, subcutaneous, intramuscular, topical, intradermal, intranasal or intrabronchial administration. The dosage regimen will be determined by taking into account, notably, clinical factors. As is well known in the medical arts, dosages for any one mammal depends upon many factors, including the mammal's size, body surface area, age, the particular compound to be administered, sex, time and route of administration and general health. Administration of the suitable pre-pharmaceutical compositions may be effected by different ways, e.g., by intravenous, intraperitoneal, subcutaneous, intramuscular, topical or intradermal administration. If the regimen is a continuous infusion, it should also be in the range of 1 ng to 10 mg units per kilogram of body weight per minute, respectively. Progress can be monitored by periodic assessment. The pre-pharmaceutical compositions of the invention may be administered locally or systemically. Administration will generally be parenterally, e.g., intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, anti-oxidants, chelating agents, and inert gases and the like.

The inhibitor substances may be employed in powder or crystalline form, in liquid solution, or in suspension.

The injectable pre-pharmaceutical compositions may take such forms as suspensions, solutions, or emulsions in oily or aqueous vehicles, and may contain various formulating agents. Alternatively, the active ingredient may be in powder (lyophilized or non-lyophilized) form for reconstitution at the time of delivery with a suitable vehicle, such as sterile water. In injectable compositions, the carrier is typically comprised of sterile water, saline, or another injectable liquid, e.g., peanut oil for intramuscular injections. Also, various buffering agents, preservatives and the like can be included.

Topical applications may be formulated in carriers such as hydrophobic or hydrophilic base formulations to provide ointments, creams, lotions, in aqueous, oleaginous, or alcoholic liquids to form paints or in dry diluents to form powders.

Oral pre-pharmaceutical compositions may take such forms as tablets, capsules, oral suspensions and oral solutions. The oral compositions may utilize carriers such as conventional formulating agents and may include sustained release properties as well as rapid delivery forms.

In certain embodiments of the in vivo screening assay, the inhibitor substance is administered to a mammal which is the subject of a bacterial infection. For non human mammals, these animals have been injected with a composition containing bacteria prior to any administration of the inhibitor compound.

In certain other embodiments of the in vivo screening assay, non human animals are administered with the inhibitor compound to be tested prior to being injected with a composition containing bacteria.

For the in vivo assay, bacteria may be of various species, including Gram-positive and Gram-negative bacteria possessing a peptidoglycan cell wall. Bacteria of interest encompass streptococci, bacilli, micrococci, lactobacilli, lactococci, enterococci and pediococci.

Generally, non human mammals are injected with a number of bacteria cells ranging from 1×10² to 1×10¹² cells, including from 1×10⁶ to 1×10⁹ cells. Generally, bacteria cells that are injected to the non human mammals are contained in a physiologically acceptable liquid solution, usually a saline solution like Ringer's solution or Hank's solution.

Generally, in the embodiment wherein the inhibitor compound to be tested is administered subsequently to bacterial inoculation, said inhibitor compound is administered form 1 hour to 96 hours after bacterial injection, including from 6 hours to 48 hours after bacterial injection.

Generally, in the embodiment wherein the inhibitor compound to be tested is administered prior to bacterial injection, said inhibitor compound is administered from 1 min to 3 hours prior to bacterial injection.

Generally, all animals are sacrificed at the end of the in vivo assay.

For determining the in vivo antibacterial activity of the inhibitor compound that is tested, blood or tissue samples of the tested animals are collected at determined time periods after administration of said inhibitor compound and bacteria counts are performed, using standard techniques, such as staining fixed slices of the collected tissue samples or plating the collected blood samples and counting the bacterial colonies formed.

Then, the values of the bacteria counts found for animals having been administered with increasing amounts of the inhibitor compound tested are compared with the value(s) of bacteria count(s) obtained from animals that have been injected with the same number of bacteria cells but which have not been administered with said inhibitor compound.

### Polypeptides, Nucleic Acids and Antibodies of the Invention.

Another object of the invention consists of any one of the D-aspartate ligases that are disclosed in the present specification, including the D-aspartate ligases of SEQ ID No 1 to 10, as well as any one of the biologically active fragments thereof.

A further object of the invention consists of any one of the L,D-transpeptidases that are disclosed in the present specification, including the L,D-transpeptidase of SEQ ID No 13, as well as any one of the biologically active fragments thereof, including those fragments of SEQ ID No 11 and SEQ ID No 12.

A still further object of the present invention consists of a nucleic acid that encodes a D-aspartate ligase or any one of the biologically active fragments thereof, including the nucleic acids of SEQ ID No 22 to 31 that encode the D-aspartate ligases of SEQ ID No 1 to 10, respectively.

A yet further object of the present invention consists of a nucleic acid that encodes a L,D-transpeptidase or any one of the biologically active fragments thereof, including the nucleic acid of SEQ ID No 32 that encodes the L,D-transpeptidase of SEQ ID No 13.

Both polypeptides or nucleic acids of the invention are preferably under a purified form.

Nucleic acids of the invention may be inserted into suitable vectors, particularly expression vectors, such as those that are described elsewhere in the present specification. Recombinant vectors comprising a nucleic acid as defined above that is inserted therein are also part of the invention.

Host cells, particularly prokaryotic cells including yeast cells and cells from E. coli that have been transfected or transformed by a nucleic acid above or a recombinant vector above form also part of the present invention. Such recombinant host cells are for example those that are described elsewhere in the present specification.

Polypeptides of the invention are preferably recombinantly produced, illustratively according to any one of the techniques of production of recombinant proteins that are disclosed elsewhere in the present specification.

A yet further object of the present invention consists of an antibody directed against a D-aspartate ligase or a L,D-transpeptidase that is disclosed in the present specification, or to a biologically active peptide fragment thereof. Any one of these antibodies may be useful for purifying or detecting the corresponding D-aspartate ligase or the corresponding L,D-transpeptidase.

There is no particular limitation on the antibodies encompassed by the present invention, as long as they can bind specifically to the desired D-aspartate ligase or the desired biologically active fragment thereof, or to the desired L,D-transpeptidase or the desired biologically active fragment thereof. It is possible to use mouse antibodies, rat antibodies, rabbit antibodies, sheep antibodies, chimeric antibodies, humanized antibodies, human antibodies and the like, as appropriate. Such antibodies may be polyclonal or monoclonal, but are preferably monoclonal because uniform antibody molecules can be produced stably. Polyclonal and monoclonal antibodies can be prepared in a manner well known to those skilled in the art.

In principle, monoclonal antibody-producing hybridomas can be prepared using known techniques, as follows. Namely, the desired antigen or the desired antigen-expressing cell is used as a sensitizing antigen and immunized in accordance with conventional procedures for immunization. The resulting immunocytes are then fused with known parent cells using conventional procedures for cell fusion, followed by selection of monoclonal antibody-producing cells (hybridomas) through conventional screening procedures. Preparation of hybridomas may be accomplished according to, for example, the method of Milstein et al. (Kohler, G. and Milstein, C., Methods Enzymol. (1981) 73:3-46). If an antigen used is less immunogenic, such an antigen may be conjugated with an immunogenic macromolecule (e.g., albumin) before use in immunization.

In addition, antibody genes are cloned from hybridomas, integrated into appropriate vectors, and then transformed into hosts to produce antibody molecules using gene recombination technology. The genetically recombinant antibodies thus produced may also be used in the present invention (see, e.g., Carl, A. K. Borrebaeck, James, W. Larrick, <<Therapeutic monoclonal antibodies>>, Published in the United Kingdom by MacMillan Publishers Ltd, 1990). More specifically, cDNA of antibody variable domains (V domains) is synthesized from hybridoma mRNA using reverse transcriptase. Upon obtaining DNA encoding the target antibody V domains, the DNA is ligated to DNA encoding desired antibody constant domains (C domains) and integrated into an expression vector. Alternatively, the DNA encoding the antibody V domains may be integrated into an expression vector carrying the DNA of the antibody C domains. The DNA construct is integrated into an expression vector such that it is expressed under control of an expression regulatory region, e.g., an enhancer or a promoter. Host cells are then transformed with this expression vector for antibody expression.

In a case where antibody genes are isolated and then transformed into appropriate hosts to produce antibodies, any suitable combination of host and expression vector can be used for this purpose. When eukaryotic cells are used as hosts, animal cells, plant cells and fungal cells may be used. Animal cells known for this purpose include (1) mammalian cells such as CHO, COS, myeloma, BHK (baby hamster kidney), HeLa and Vero, (2) amphibian cells such as Xenopus oocytes, and (3) insect cells such as sf9, sf21 and Tn5. Plant cells include those derived from Nicotiana plants (e.g., Nicotiana tabacum), which may be subjected to callus culture. Fungal cells include yeasts such as Saccharomyces (e.g., Saccharomyces serevisiae) and filamentous fungi such as Aspergillus (e.g., Aspergillus niger). When prokaryotic cells are used, there are production systems employing bacterial cells. Bacterial cells known for this purpose are E. coli and Bacillus subtilis. Antibodies can be obtained by introducing target antibody genes into these cells via transformation and then culturing the transformed cells in vitro.

### Compositions or Kits for the Screening of Antibacterial Substances

The present invention also relates to compositions or kits for the screening of antibacterial substances.

In certain embodiments, said compositions or kits comprise a purified D-aspartate ligase or a purified L,D-transpeptidase, preferably under the form of a recombinant protein.

In said compositions or said kits, said D-aspartate ligase or said L,D-transpeptidase may be under a solid form or in a liquid form.

Solid forms encompass powder of said D-aspartate ligase or said L,D-transpeptidase under a lyophilized form.

Liquid forms encompass standard liquid solutions known in the art to be suitable for protein long time storage.

Preferably, said D-aspartate ligase or said L,D-transpeptidase is contained in a container such as a bottle, e.g. a plastic or a glass container.

In certain embodiments, each container comprises an amount of said D-aspartate ligase or said L,D-transpeptidase ranging from 1 ng to 10 mg, either in a solid or in a liquid form.

Further, said kits may comprise also one or more reagents, typically one or more substrate(s), necessary for assessing the enzyme activity of said D-aspartate ligase or of said L,D-transpeptidase.

Illustratively, if said kit comprises a container of D-aspartate ligase, then said kit may also comprise (i) a container comprising labeled aspartate such as [¹⁴C]aspartate or [³H] aspartate and/or (ii) a container comprising UDP-MurNac pentapeptide and UDP-MurNac tetrapeptide.

Illustratively, if said kit comprises a container of L,D-transpeptidase, then said kit may also comprise (i) a container comprising a donor compound consisting of a tetrapeptide preferably selected from the group consisting of L-Ala-D-Glu-L-Lys-D-Ala, Ac₂-L-Lys-D-Ala and disaccharide-tetrapeptide(iAsn) and (ii) a container comprising an acceptor compound selected from the group consisting of a D-amino acid or a D-hydroxy acid.

In certain embodiments, a kit according to the invention comprises one or more of each of the containers described above.

The present invention is further illustrated by, without in any way being limited to, the examples hereunder.

### EXAMPLES

### Examples 1 to 4 Related to the Characterization of a Bacterial D-Aspartate Ligase

### A. Material and Methods of Examples 1 to 4

A.1. Preparation of Cytoplasmic and Membrane Extracts.

Enterococcus faecium D359V8 was grown to an A₆₅₀ nm of 0.7 in 20 litters of BHI broth (Difco, Elancourt, France), harvested by centrifugation (6 000×g for 20 min at 4° C.), and washed twice in 50 mM sodium phosphate buffer (pH 7.0). Bacteria were disrupted with glass beads in a refrigerated cell disintegrator (B. Braun, Sartorius, Palaiseau, France) for 3×30 s. The extract was centrifuged (7 000×g for 10 min at 4° C.) to remove cell debris and the supernatant was ultracentrifuged at 100 000×g for 1 h at 4° C. The supernatant was saved (cytoplasmic fraction) and the pellet was washed twice in 50 mM sodium phosphate buffer (pH 7.0) (membrane fraction). The protein contents were determined with the Bio-Rad protein assay (Bio-Rad, Ivry-Sur-seine, France).

A.2 In Vitro Addition of D-Aspartate onto UDP-MurNac-pentapeptide-

The assay was performed in a total volume of 25 μl containing Tris-Hcl (100 mM, pH 8.5), MgCl₂ (50 mM), ATP (20 mM), D-[¹⁴C]aspartic acid (0.11 mM, 55 mCi/mmol, Isobio, Fleurus, Belgium), UDP-MurNac-pentapeptide (0.15 mM) purified from S. aureus as previously described (Billot-Klein et al., 1997), and membrane or cytoplasmic extracts (60 μg). The reaction mixture was incubated 2 h at 37° C. and the reaction was stopped by boiling the samples for 3 min. D-[¹⁴C]aspartic acid was separated from [¹⁴C]UDP-MurNac-hexapeptide by descending paper chromatography (Whatman no. 4 filter paper) with a mobile phase composed of isobutyric acid and 1 M ammonia (5:3, vol/vol). The products of the reaction were also separated by reverse phase high-pressure liquid chromatography (rpHPLC) on a Hypersil C18 column (3 □m, 4.6×250 nm, Interchrom, Montluçon, France) at a flow rate of 0.5 ml/min using isocratic elution (10 mM ammonium acetate, pH 5.0) and detected by the absorbance at 262 nm and liquid scintillation with a Radioflow Detector (LB508; Perkin Elmer, Courtaboeuf, France) coupled to the HPLC apparatus (L-62000A; Merck, Nogent-Sur-Mame, France).

A.3 Purification of the E. faecium D-Aspartate Ligase.

The D-aspartate ligase was partially purified from extracts of E. faecium D359V8 using three chromatographic steps and the D-aspartate ligase activity was detected in the fractions by the formation of [¹⁴C]UDP-MurNac-hexapeptide as described above. Briefly, soluble proteins from supernatant (1.3 g) were dialyzed against 50 mM phosphate buffer (pH 6.0) containing 200 mM NaCl (buffer A) and loaded onto a cation exchange HiLoad™ 26/10 SP Sepharose™ HP column (Amersham Pharmacia Biotech, Saclay, France) equilibrated in buffer A and elution was performed with a 0.2 to 2 M NaCl gradient in buffer A. Actives fractions, eluted between 0.8 and 0.9 M NaCl, were pooled (12 mg of proteins), concentrated with Polyethylene glycol (PEG), and loaded onto a gel filtration column (Superdex 75 HR26/60, Amersham Pharmacia Biotech) equilibrated with buffer A. Active fractions (1.8 mg of proteins) were loaded onto cation exchange HiTrap SP Sepharose Fastflow 1 ml column (Amersham Pharmacia Biotech) equilibrated in buffer A and elution was performed with a 0.2 to 2M NaCl gradient in buffer A. Proteins (200 μg), eluting between 0.8 and 0.95 M NaCl, were dialyzed against buffer A, concentrated by lyophilisation and deposited on a 12% SDS PAGE.

A.4. Protein Identification.

Candidate proteins were excised from the 12% SDS page, reduced with DTT (dithiothreitol, Sigma), alkylated with iodoacetamide and digested with trypsin (modified trypsin, sequencing grade, Roche) overnight at 37° C., using the automatic DIGESTPRO digester from ABIMED. Tryptic digests were dried under vacuum in a Speed-Vac. Samples were resuspended in 4 μl of 0.1% formic acid. They were then separated by HPLC in the LC-Packing system at a flow rate of 200 nl/min using a gradient starting at 2% acetonitril (AcCN) in 0.1% formic acid for 1 min, increasing to 50% AcCN over 40 min, and finally increasing to 90% AcCN over 10 minutes. The LC system is connected to an ion trap mass spectrometer (LCQ Deca, Finnigan Corp, San Jose, Calif.), running Excalibur. The spray voltage was set at 2.1 kV, the temperature of the ion transfer tube was set at 180° C. and the normalized collision energies were set at 35% for MS/MS. The sequences of the uninterpreted spectra were identified by correlation with the peptide sequences present in the NCBI non redundant protein database, using the SpectrumMill program (Millennium Pharmaceuticals).

A.5. Cloning and Purification of the Aspartate Ligase in E. coli.

The ORF coding for the putative aspartate ligase gene of E. faecium, designated hereafter Asl_{fm}, was amplified with primers Asl1 and Asl2. Primer Asl1 (GAGAGACCATGGTGAACAGTATTGAAAATGAAG—SEQ ID No 14) contained NcoI restriction site (bolded) and 21-bp of asl-5′ extremity. Primer Asl2 (CTCCATGGCTAGGATCCTTCTTTCACATGAAAATACTTTTTG—SEQ ID No 15) contained BamHI restriction site (bolded) and 25-bp of the asl-3′ end without stop codon. The asl_{fm} sequence was amplified using Pfu Turbo DNA polymerase (Stratagene, La Jolla, Calif., USA) and E. faecium chromosomal DNA as template (Williamson et al., 1985). The PCR product was cloned into NcoI-BamHI-restricted pET2818 plasmid, a derivative of pET2816 (Chastanet et al., 2005) generating pSJL1. This plasmid was introduced by electroporation into E. coli BL21 (DE3) harboring pREP4 plasmid (Amrein et al., 1995).

E. coli BL21(DE3) harboring pSJL1 was grown to an optical density at 600 nm of 0.7 under gentle shaking in 2 liters of BHI broth containing of kanamycin (50 μg/ml) and ampicillin (100 μg/ml). Isopropyl-β-D-thiogalactopyranoside (IPTG) was added (0.5 mM) and incubation was continued for 3.5 h. Bacteria were harvested by centrifugation (7 000×g for 20 min at 4° C.), washed in Tris-HCl 50 mM, pH 8.0 containing 150 mM of NaCl (buffer B) and resuspended in the same buffer. Bacteria were disrupted as previously described and the extract was centrifuged at 100 000×g for 1 h at 4° C. The supernatant was mixed with 1 ml of Ni2+-nitrilotriacetate-agarose resin (Qiagen, Courtabeuf, France) previously equilibrated with buffer B. After incubation overnight at 4° C., solution was loaded onto a poly-prep column (Bio-rad, Marnes-la-Coquette, France), resin was washed with 12 column volumes of buffer B and proteins were eluted with buffer B containing 250 mM of imidazole. Proteins eluted were dialyzed overnight at 4° C. against buffer A and loaded onto a HiTrap SP-sepharose fast flow (Pharmacia, Orsay, France) equilibrated with buffer A. Proteins were eluted with a gradient of NaCl (0.2-2M), concentrated against buffer B containing glycerol 50% and stored at −20° C. The purified protein was tested for the D-aspartate ligase activity as described above but using 2 μg of purified protein and 0.8 mM of UDP-MurNac-pentapeptide. To confirm its structure the synthesis of the hexapeptide was done in presence of non radio-active D-aspartate (3 mM) and samples of UDP-MurNAc-peptide products were isolated by rpHPLC, lyophilized, resuspended in water and analyzed by MS and MS/MS as previously described (Bouhss et al., 2002).

Antiserum against Asl_{fm} was obtained by injection subcutaneously of 200 μg of purified protein in a rabbit and used in Western blotting experiments carried out as previously described (Towbin et al., 1979).

A.6. Heterospecific Expression of the Asl_{fm} Gene in E. faecalis.

The shuttle vector (pJEH11) was constructed by amplification of the chloramphenicol acetyl transferase (CAT) gene from pNJ2 plasmid with primers pJE1 and pJE2 (Arbeloa et al., 2004). Primer pJE1 (GGGAGCTCAAGGAGGAGACTGACCATGGACTTTAATAAAATTGA—SEQ ID No 16) contained SacI restriction site (italicized), VanY Shine-Dagarno sequence (from E. faecium BM4107) underlined and NcoI restriction site bolded. Primer pJE2 (CATCTAGATTAAGATCTCAATGGTGATGATGGTGATGGGATCCCTA TTATAAAAGCCAGTCAT—SEQ ID No 17) contained a XbaI restriction site (italicized), a stop codon, a BglII restriction site (bolded), a stop codon, 6 histidine codons (underlined) and a BamHI restriction site (bolded and italicized). The PCR product was digested with SacI and XbaI enzymes and cloned into SacI-XbaI digested pAT392 vector generating pJEH11 plasmid. The NcoI-BamHI fragment of pSJL1 containing asl_{fm} open reading frame was cloned under the control of the p2 promoter into NcoI-BamHI restricted pJEH11 generating pSJL2 plasmid. This vector was introduced into E. faecalis JH2.2 by electroporation and clones were selected on BHI-agar plates containing 256 μg/ml of gentamicin.

A.7. Peptidoglycan Structure Analysis.

E. faecalis JH2-2/pSJL2asl_{fm} and of the parental strain JH2-2/pJEH11 were grown at 37° C. to an optical density of 0.7 in 250 ml of BHI broth, containing or not D-aspartate (50 mM) (Sigma-Aldrich). Peptidoglycan was extracted with 4% SDS and muropeptides obtained as previously described (Arbeloa et al., 2004; Mainardi et al., 1998). Lactoyl peptide peptidoglycan fragments were produced and separated by rp-HPLC as previously described (Arbeloa et al., 2004). The relative abundance of peptidoglycan fragments was estimated as the percentage of the total integrated area of the identified peaks. The peaks were individually collected, lyophilized and dissolved in 100 μl of water. The mass of the peptidoglycan fragments were determined using an electrospray time-of-flight mass spectrometer operating in positive mode (Qstar Pulsar I, Applied Biosystem, Courtaboeuf, France) (Arbeloa et al., 2004b). The determination of the structures of the muropeptides was performed by fragmentation. The ions were selected based on the m/z value ([M+H]¹⁺) in the high resolution mode, and fragmentation was performed with nitrogen as collision gas with an energy of 36-40 eV.

### B. Results of Examples 1 to 4

### Example 1

### Assay for UDP-MurNAc-Hexapeptide Synthesis

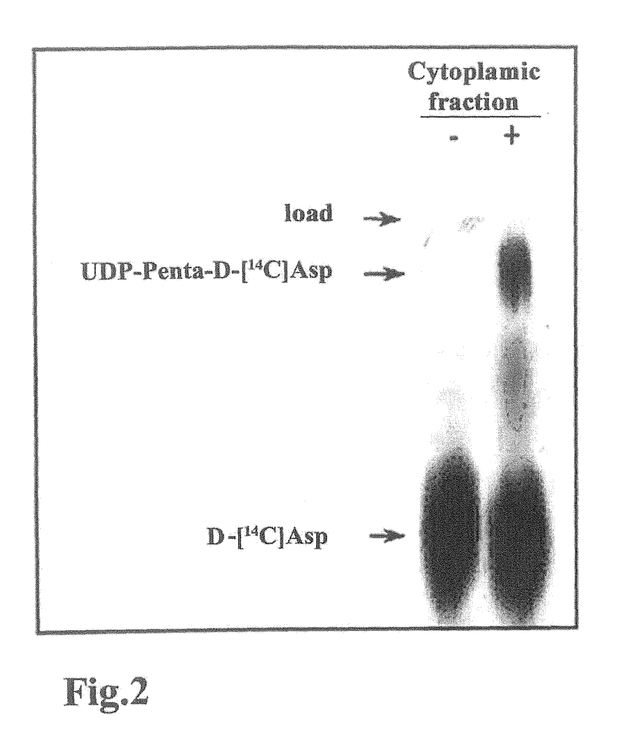

We first tested if the aspartate ligase activity was found in the membrane or the cytoplasmic extracts obtained from 20 liters culture of E. faecium D359V8. The assay was performed with 60 μg of membranes or cytoplasmic extracts in presence of D-[¹⁴C]aspartic acid, UDP-MurNac-pentapeptide, MgCl₂ and ATP. After 2 hours at 37° C., the percentage of conversion was about 5% in the different extracts and both paper (FIG. 2) and HPLC chromatographies (data not shown) revealed only two radioactive peaks corresponding to the labeled UDP-MurNac-hexapeptide and to the D-[¹⁴C]aspartate respectively. The incorporation of D-[¹⁴C]aspartate was not inhibited by addition of Rnase suggesting that the activity was not tRNA dependant. Omission of the divalent cation (MgCl₂), ATP, UDP-MurNAc-pentapeptide, cytoplasmic or membrane extracts resulted in absence of incorporation of D-[¹⁴C]aspartate. No hexapeptide was formed when D-[¹⁴C]aspartate was replaced by L-[¹⁴C]aspartate. These assays were subsequently used during the purification steps to identify the D-aspartate ligase from the cytoplasmic extract.

### Example 2

### Identification of the Gene Encoding the E. faecium D-Aspartate Ligase

Since the D-aspartate ligase activity present in the cytoplasmic extracts represented almost 50% of the total activity it was used for further purification of the enzyme. To overcome the precipitation of the protein, all the purification steps were performed at an ionic strength above 200 mM NaCl. A partially purified preparation enriched in D-aspartate ligase activity was obtained from 1.3 gram of soluble proteins by 3 chromatography steps. LC-MS-MS was performed on different candidate proteins excised from a 12% SDS page. Among them, a 50 kDa protein with a ATP grasp motif (Galperin et al., 1997) was identified as the most likely candidate for the D-aspartate ligase from the protein bank deduced from the incomplete genome of E. faecium (Enterococcus faecium at NCBI: Efae 03003049).

### Example 3

### Purification and Assay of the Activity of the Aspartate Ligase

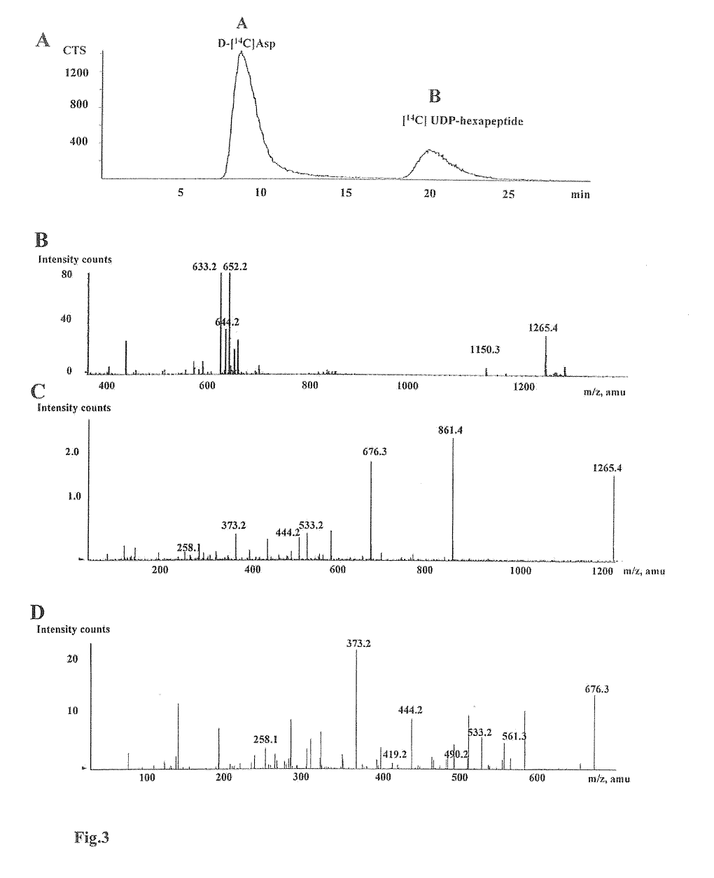

The gene (asl_{fm}) encoding the putative D-aspartate ligase was amplified, cloned and introduced into E. coli BL21 The presence of C-terminal six-His tag allowed the purification of the D-aspartate ligase in two steps after successive chromatography on a nickel column and a cation exchange column. SDS-page revealed the presence of the expected ca.49 kDa protein band estimated to be >95% pure (data not shown). Addition of 2 μg of purified protein in the D-aspartate ligase assay resulted in the formation of a radioactive product corresponding to the labeled hexapeptide (peak B in FIG. 3A) in addition to D-[¹⁴C]aspartate. To ensure that the labeled product in peak B was the expected hexapeptide (UDP-MurNAc-(D-Asp)pentapeptide), the D-aspartate ligase assay was scaled up for mass spectrometry and MS/MS analysis (FIGS. 3B, 3C and 3D). D-[¹⁴C]aspartic acid was replaced by D-aspartate (3 mM) and 8 μg of purified protein were used in the assay (200 μl). Peak B was purified by HPLC. The molecular mass of compound B was determined to be 1264.4 Da from the peaks at m/z 1265.4, 633.2, 644.2 and 652.2, which were assigned to be [M+H]⁺, [M+2H]²⁺, [M+H+Na]²⁺ and [M+H+K]²⁺ ions, respectively (FIG. 3B). These molecular masses match the predicted value of 1264.4 Da for UDP-MurNac-hexapeptide. The same analysis performed on the nucleotide substrate revealed the predicted value of 1149.3 for UDP-MurNac-pentapeptide. The MS/MS experiments performed on the peak at m/z 1265.4 (FIG. 3C) gave ions at m/z 861.4 corresponding to the loss of UDP residue (MurNAc-hexapeptide). Peak at m/z 533.3 matched the expected mass of the γ-D-Glu-L-Lys-(N^{ε}-D-Asp)-D-Ala-D-Ala. Further loss of two alanine residues from the C-terminus resulted in the peaks at m/z 444.2 and 373.2, respectively. From the ion 373.2 corresponding to γ-D-Glu-L-Lys-(N^{ε}-D-Asp), the loss of D-asp gave ion at m/z 258.1. This ion confirmed that one D-aspartate residue is branched to the L-lysyl residue. The peaks at m/z 676.3 matched the expected value of the 2-hydroxy propionyl (lactyl) hexapeptide moiety of the molecule. MS/MS experiments were also performed on this ion (FIG. 3 D). Peak at 561.3 matched the predicted value for loss of one D-aspartate residue linked to the ε-amino group of L-Lysine. Additional loss of one or two C-terminal D-alanine residues gave ions at m/z 490.2 and 419.2. Several other aspects of the fragmentation patterns of UDP-MurNac-hexapeptide were confirmed with the presence of peaks at 533.2, 444.2, 373.2 and 258.1.

### Example 4

### Heterologous Expression of Asl_{fm} and its Impact on the Peptidoglycan Structure

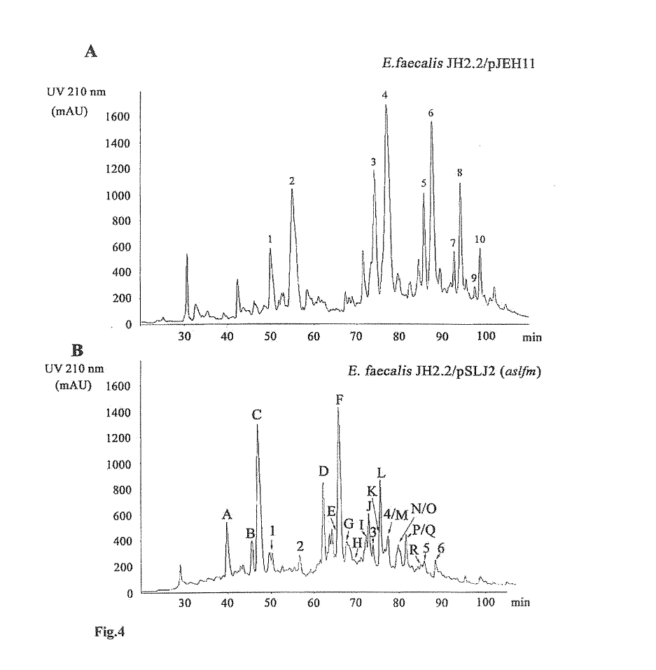

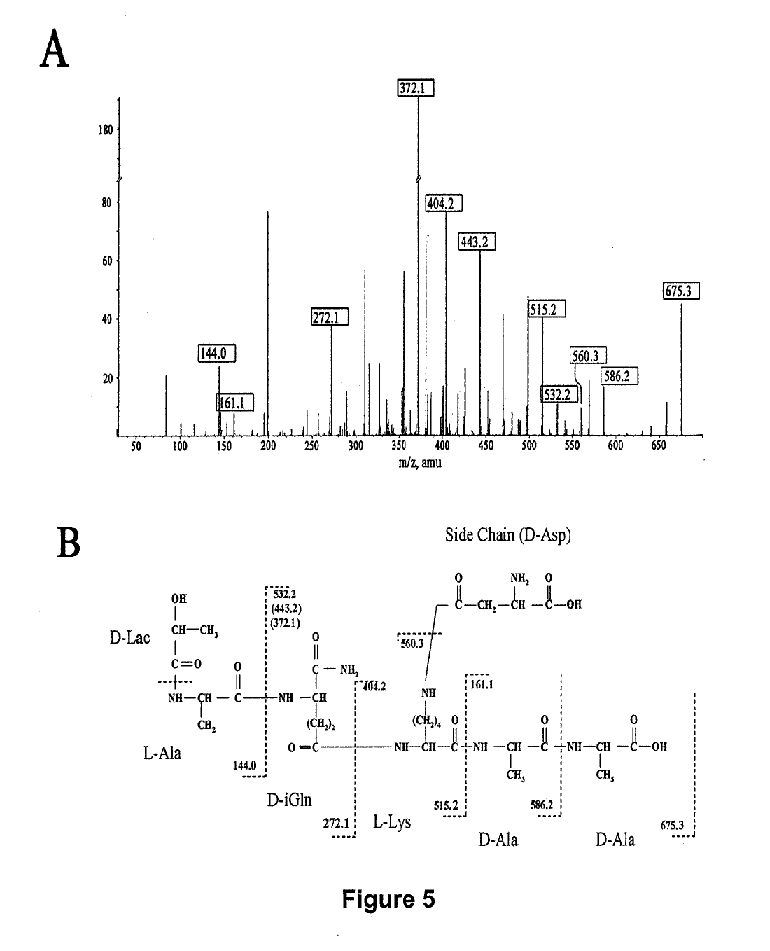

To assess the in vivo activity of the D-aspartate ligase, pSJL2(asl_{fm}) was introduced in the heterologous host E. faecalis JH2-2. The expression of D-aspartate ligase and its activity were detected in the cytoplasmic extracts by a Western blot assay using an anti-Asl_{fm} antiserum and the standard D-aspartate ligase assay respectively (data not shown). The peptidoglycan structure of E. faecalis JH2-2/pSJL2(asl_{fm}) and that of the parental strain JH2-2 containing the native plasmid pJEH11, were analyzed by liquid chromatography coupled to mass spectrometry. Since the structures of the muropeptides present in the peaks of JH2-2/pJEH11 peptidoglycan were identical to those found in JH2-2 (17), the same numbering was used (peak 1 to 10, FIG. 4A). All these muropeptides contained two L-alanyl residues either in the free N-terminal side chains of the stem peptide in the monomers (peaks 1 and 2, Table 1) or both in the side chain and the cross bridge in the multimers (Table 1). The same muropeptide profile was found in JH2-2/pJEH11 grown in presence of D-aspartate and in JH2-2/pSJL2(asl_{fm}) grown in absence of D-aspartate (data not shown). In contrast, the peptidoglycan of JH2-2/pSJL2(asl_{fm}) grown in presence of D-aspartate revealed the presence of new additional monomeric and multimeric structures (FIG. 4B and Table 1). The most abundant monomers of JH2-2/pSJL2(asl_{fm}) harbored a D-aspartate side chain (FIG. 4 and Table 1) and represented 87% of the monomers. The analysis of the lactoyl peptide peptidoglycan fragments from the main monomer of JH2-2/pSJL2(asl_{fm}) (peak C) showed a monoisotopic mass of 674.3 (FIG. 4B and Table I), which matched the calculated value for a D-lactoyl-pentapeptide stem substituted by a side chain consisting of one D-aspartate. The structure of this branched peptide was solved by MS/MS, based on the detection of specific ions generated by the loss of residues from the N terminus of the side chain and from the carboxyl or hydroxyl extremities of the lactoyl-pentapeptide stem (FIG. 5). Other monomeric structures harbouring a D-aspartate residue in the side chain were detected in peak A and B and their structures confirmed by MS/MS (data not shown). Provided that D-Asp was present in the medium, the presence of a D-aspartate linked to the c-amino group of L-Lys₃ of the main monomers indicated that the Asl_{fm} D-aspartate ligase of E. faecium was functional in the heterologous host E. faecalis. Beside these monomeric structures harbouring a D-aspartate residue, only 13% of the monomers with the usual L-Ala-L-Ala side chain generated by the natives BppA1 and BppA2 transferases present in E. faecalis (Bouhss et al., 2002) were produced (Table 1).

Similarly to what was observed for the monomers the concomitant expression of the Asl_{fm} ligase and of the BppA1 and BppA2 transferases in JH2-2/pSJL2(asl_{fm}), explains the polymorphism observed in the composition of the side chain and the cross-bridge of the multimers (Table 1). The sequence of the cross-bridge and of the side chain present in the multimers were determined by tandem mass spectrometry (data not shown). The first polymorphism was represented by novel dimers (peaks D, E and F), trimers (peak J, K and L) and tetramers (peak O and Q) which altogether represented 73% of all multimers and contained only one D-aspartate in the cross bridge and in the free N-terminal side chains of the stem peptide. The presence of D-aspartate at these positions in the multimers indicates that the D,D-transpeptidases of E. faecalis could cross-link D-aspartate-containing precursors and that peptide stems substituted by D-aspartate were used in the transpeptidation reaction both as acceptors and a donors. A second polymorphism was generated by the presence of dimers and trimers containing the usual L-Ala-L-Ala in the cross-bridge or in the free N-terminal side chain (peak 3, 4, 5, and 6). The third polymorphism was generated by the presence of dimers (peak G, H and I) and trimers (peak P and R) harboring the sequence L-Ala-L-Ala in the cross bridge and one D-aspartate residue in the side chain. The fourth polymorphism was generated by the presence of trimers (peak M and N) harboring one D-aspartate in one cross-bridge, the sequence L-Ala-L-Ala in the second cross-bridge and a D-Asp residue in the side chain. While in the side chains or cross-bridges L-Ala-L-Ala and D-Asp can be found in the same oligomer the absence of D-Asp-L-Ala or the L-Ala-D-Asp peptides suggested that the tRNA dependant transferases and the tRNA independent Asl_{fm} ligase cannot cooperate to form such a mosaic side chains in E. faecalis.

### Examples 5 to X Related to the Characterisation of a Bacterial L,D-Transpeptidase

### A. Material and Methods of Examples 5 to X

### Example 5

### Purification of the L,D-Transpeptidase from E. faecium and N-Terminal Sequencing

The L,D-transpeptidase was purified from E. faecium M512 (Mainardi et al., 2000)) in four chromatographic steps using the radioactive exchange assay (see below) to detect active fractions. Briefly, E. faecium M512 was grown to an OD₆₅₀ of 0.7 in 24 liters of brain heart infusion (BHI) broth (Difco, Elancourt, France), harvested by centrifugation, and washed twice in 10 mM sodium phosphate (pH 7.0). Bacteria were disrupted with glass beads in a cell disintegrator (The Mickle Laboratory Engineering Co, Gromshall, United Kingdom) for 2 h at 4° C. The extract was centrifuged (5000×g for 10 min at 4° C.) to remove cell debris and the supernatant was ultracentrifuged at 100,000×g for 30 min at 4° C. Soluble proteins (1 g) were loaded onto an anion exchange column (Hi-Load™ 26/10 Q Sepharose™ HP, Amersham Pharmacia Biotech, Saclay, France) equilibrated with 25 mM sodium cacodylate buffer (pH 7.86) (buffer A). Elution was performed with a linear 0 to 2M NaCl gradient in buffer A. Active fractions were pooled (30 mg of proteins), concentrated by ultrafiltration (Centricon YM10, Millipore, Saint-Quentin-en-Yvelines, France), and loaded onto a gel filtration column (Superdex 75 HR26/60, Amersham Pharmacia Biotech) equilibrated with buffer A containing 0.3M NaCl. Active fractions (1 mg of proteins) were loaded onto a weak anion exchange column (HiTrap™ DEAE fast Flow™ 1 ml, Amersham Pharmacia Biotech) equilibrated with buffer A. Proteins (300 μg) eluting between 0.2 and 0.3 M NaCl were concentrated by ultrafiltration (Amicon ultra-4, Millipore) and loaded onto a gel filtration column (Superdex 200 PC 3.2/30, Amersham Pharmacia Biotech) equilibrated with buffer A containing 0.3M NaCl. Active fractions (70 μg of proteins) were concentrated (Amicon ultra-4) and analyzed by SDS-PAGE revealing a major 48-kDa protein band which was transferred onto polyvinylidene difluoride membrane (Problott, Applied Biosystems, Framingham, Mass.) by passive adsorption (Messer et al., 1997). N-terminal Edman sequencing was performed on an Applied Biosystems Procise 494HT instrument with reagents and methods recommended by the manufacturer. The open reading frame for the L,D-transpeptidase was identified by similarity searches between the N-terminal sequence of the 48-kDa protein (AEKQEIDPVSQNHQKLDTTV [SEQ ID No 20]) and the partial genome sequence of E. faecium using the software tBLAST at the National Center for Biotechnology Information Web site (http://www.ncbi.nlm.nih.gov/).

### Example 6

### Production of the L,D-Transpeptidase in E. coli and Purification of the Protein

A portion of the ldt_{fm} open reading frame of E. faecium M512 was amplified with primers 5′-TTCCATGGCAGAAAAACAAGAAATAGATCC-3′ (SEQ ID No 18) and 5′-TTGGATCCGAAGACCAATACAGGCG-3′ (SEQ ID No 19). The PCR product digested with NcoI and BamHI (underlined) was cloned into pET2818, a derivative of pET2816 (Chastanet et al., 2003) lacking the sequence specifying the thrombin cleavage site (our laboratory collection). The resulting plasmid, pET2818 Ωldt_{fm}, encoded a fusion protein consisting of a methionine specified by the ATG initiation codon of pET2818, the sequence of the protein purified from E. faecium (residues 119 to 466), and a C-terminal polyhistidine tag GSH₆. E. coli BL21 (DE3) pREP4GroESL (Amrein et al., 1995) harboring pET2818 Ωldt_{fm} was grown at 37° C. to an OD₆₅₀ of 0.8 in three liters of BHI broth containing ampicillin (100 μg/ml). Isopropyl-D-thiogalactopyranoside was added to a final concentration of 0.5 mM and incubation was continued for 17 h at 16° C. Ldt_{fm} was purified from a clarified lysate by affinity chromatography on Ni²⁺-nitrilotriacetate-agarose resin (Qiagen GmbH, Hilden, Germany) followed by anion exchange chromatography (MonoQ HR5/5, Amersham Pharmacia Biotech, Uppsala Sweden) with a NaCl gradient in TrisHCl pH 7.5. An additional gel filtration was performed on a Superdex HR10/30 column (Amersham Pharmacia Biotech) equilibrated with 50 mM Tris-HCl (pH 7.5) containing 300 mM NaCl at a flow rate of 0.5 ml/min. Site-directed mutagenesis was performed according to the QuickChange procedure of Stratagene (La Jolla, Calif.).

### Example 7

### Peptide and Amino Acid Substrates of the L,D-Transpeptidase

The dipeptide N^{α},N^{ε}-diacetyl-L-lysyl-D-alanine (Ac₂-L-Lys-D-Ala) was prepared by coupling Boc₂-L-Lys p-nitrophenylester with D-Ala-Obenzyl p-toluenesulfonate (Novabiochem, Laufelfingen, Switzerland) in the presence of triethylamine followed by acetylation with acetic anhydride in the presence of pyridine as previously described (Mainardi et al., 2002). N^{α},N^{ε}-diacetyl-L-lysine-D-alanyl-D-alanine (Ac₂-L-Lys-D-Ala-D-Ala), L-Ala-D-iGlu-L-Lys-D-Ala-D-Ala (pentapeptide), and amino acids were purchased from Sigma-Aldrich (Saint-Quentin Fallavier, France). D-2-hydroxy acids were obtained from Acros Organics (Noisy-le-Grand, France). UDP-N-acetylmuramyl-L-Ala-D-iGlu-L-Lys-D-Ala-D-Ala (UDP-MurNAc-pentapeptide) was prepared from Staphylococcus aureus (Billot-Klein et al., 1997). The R39 D,D-carboxypeptidase was used to generate UDP-MurNAc-tetrapeptide and tetrapeptide from UDP-MurNAc-pentapeptide and pentapeptide, respectively (Billot-Klein et al., 1992). Disaccharide-peptide fragments of the peptidoglycan (muropeptides) were prepared by scaling up a previously published procedure (Arbeloa et al., 2004). Briefly, E. gallinarum strain SC1 (Grohs et al., 2000) was grown in 3 liters of BHI broth at 37° C. to an OD₆₅₀ of 0.7. Peptidoglycan was extracted with 4% sodium dodecyl sulfate at 100° C., treated overnight with pronase and trypsin, and digested with mutanolysin and lysozyme. Soluble disaccharide-peptides were purified by reversed-phase high pressure liquid chromatography (rp-HPLC) on a C₁₈ column, individually collected, lyophilized, and dissolved in water. The concentration of muropeptides was estimated after acidic hydrolysis with a Biotronik model LC2000 amino acid analyzer (Mengin-Lecreuix et al., 1999). The structure of the different substrates was confirmed by mass spectrometry and tandem mass spectrometry with an electrospray quadrupole time-of-flight mass spectrometer operated in the positive mode (Qstar Pulsar I, Applied Biosystems, Courtaboeuf, France), as previously described (Arbeloa et al., 2004).

### Example 8

### L,D-Transpeptidase Assays

The standard exchange assay was based on incubation of non-radioactive Ac₂-L-Lys-D-Ala and D-[¹⁴C]Ala and determination of Ac₂-L-Lys-D-[¹⁴C]Ala formed by the L,D-transpeptidase (Mainardi et al., 2002; Coyette et al., 1974). Briefly, the assay (50 μl) contained Ac₂-L-Lys-D-Ala (5 mM), D-[¹⁴C]Ala (0.15 mM; 2.0 GBq/mmol, ICN Pharmaceuticals, Orsay, France), 10 mM sodium cacodylate buffer (pH 6.0), and 0.1% triton X-100 (v/v). The reaction was allowed to proceed at 37° C. and stopped by boiling the samples for 3 min. After centrifugation (10,000×g, 2 min), 45 μl of the supernatant was analyzed by rpHPLC at 25° C. on a μ-Bondapak C₁₈ column (3.9 by 300 mm, Waters, Saint Quentin en Yvelines, France) with isocratic elution (0.05% TFA in water/methanol 9:1 per volume) at a flow rate of 0.5 ml/min. Products were detected by scintillation with a Radioflow Detector (LB508, Perkin Elmer) coupled to the HPLC device. To test different donors, 3 μg of Ldt_{fm} were incubated for 60 min in the same conditions, except that Ac₂-L-Lys-D-Ala was replaced by UDP-MurNAc-tetrapeptide (2.5 mM), UDP-MurNAc-pentapeptide (2.5 mM), tetrapeptide (2.5 mM), pentapeptide (2.5 mM), GlcNAc-MurNAc-tetrapeptide-iAsn (1 mM), and GlcNAc-MurNAc-pentapeptide-iAsn (1 mM).

To assay for in vitro transpeptidation, the L,D-transpeptidase (3 μg) was incubated with the monomeric muropeptides GlcNAc-MurNAc-L-Ala-D-iGln-L-(N^{ε}-D-iAsn)Lys-D-Ala (25 nmoles), GlcNAc-MurNAc-L-Ala-D-iGln-L-(N^{ε}-D-iAsn)Lys (5 nmoles) and GlcNAc-MurNAc-L-Ala-D-iGln-L-Lys-D-Ala (5 nmoles) for 2 h at 37° C. in 25 μl of 5 mM sodium phosphate buffer (pH 6.0). The reaction was stopped by boiling the sample for 3 min and the mixture was centrifuged (10,000×g, 2 min). The formation of dimers was determined by mass spectrometry on a 10-μl aliquot. For tandem mass spectrometry analysis, the remaining of the reaction mixture was treated with ammonium hydroxide to cleave the ether link internal to MurNAc (Arbeloa et al., 2004). The conditions for fragmentation of the resulting lactoyl-peptides with N₂ as the collision gas were as previously described (Arbeloa et al., 2004).

### Summary of the Results of Examples 5 to 8

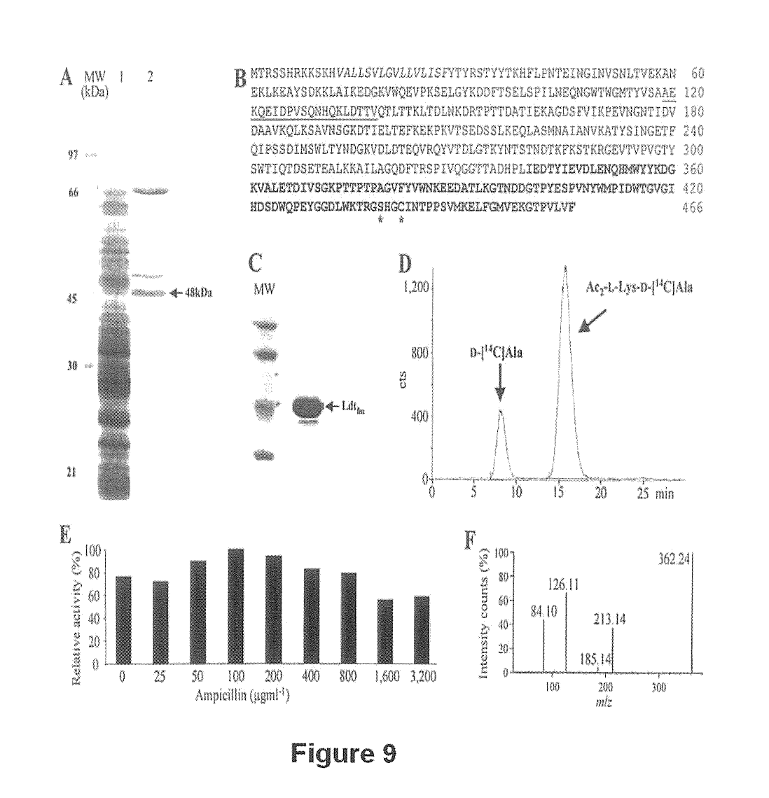

We identified the gene encoding the L,D-transpeptidase responsible for the formation of the L-Lys₃⋄D-iAsn-L-Lys₃ cross-links in E. faecium M512 by partial purification of the enzyme (FIG. 9A), sequencing of its N-terminus, and similarity searches in the partial genome sequence of E. faecium. The partially purified protein was a proteolytic fragment lacking the 118 N-terminal residues, including a putative membrane anchor (FIG. 9B). The portion of the open frame encoding the proteolytic fragment was expressed in Escherichia coli for large scale protein purification (FIG. 9C). The protein was active in an exchange assay (FIG. 9D) and was not inhibited by ampicillin (FIG. 9E), indicating that the gene encoding the L,D-transpeptidase of E. faecium M512 (Ldt_{fm}) had been successfully identified.

To gain insight in the activity of Ldt_{fm}, various 2-amino and 2-hydroxy acids were tested as potential acceptor substrates (Table 2) in an exchange reaction using the model dipeptide substrate Ac₂-L-Lys-D-Ala as the donor (FIG. 9F). Formation of depsipeptides with D-lactate, D-2-hydroxyhexanoic acid, and D-malic revealed that Ldt_{fm} can catalyze formation of ester bonds in addition to peptide bonds. Acceptors containing a relatively bulky side chain such as D-Met and D-2-hydroxyhexanoic acid were used as acceptors in the transpeptidation and transesterification reactions. Hydrolysis of the C-terminal D-Ala of Ac₂-L-Lys-D-Ala was not detected in the presence of a suitable acceptor substrate, indicating a biosynthetic function for Ldt_{fm}, in contrast to the previously characterized L,D-carboxypeptidase involved in peptidoglycan recycling in E. coli (Templin et al., 1999). Finally, Ldt_{fm} was stereo-specific since no product was detected when L-Met was used as donor (Table 2).

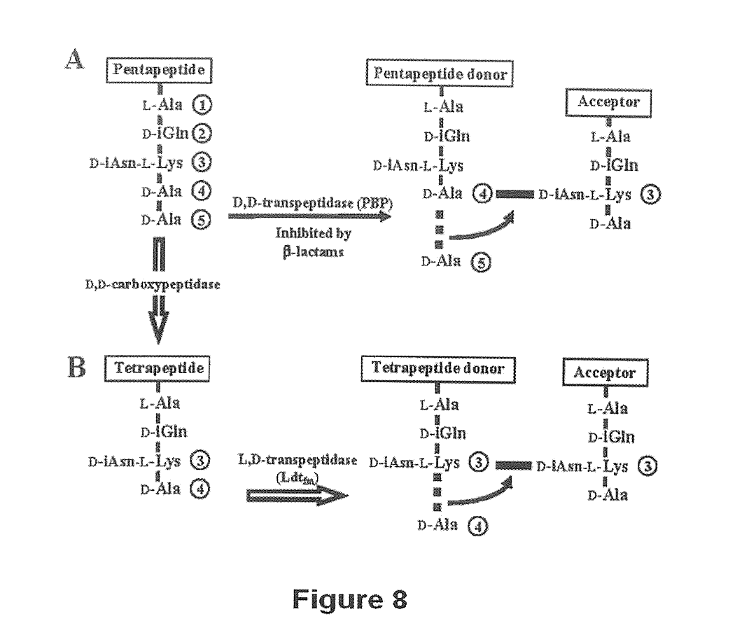

The Ldt_{fm} specificity for peptide donors was explored with the exchange assay using D-[¹⁴C]Ala as the acceptor. Formation of radioactive peptides was observed not only with Ac₂-L-Lys-D-Ala (FIG. 9D) but also with the complete disaccharide-tetrapeptide(iAsn) peptidoglycan unit and with other donors containing a tetrapeptide ending in D-Ala (Compounds used as donors by Ldt_{fm} in the radioactive exchange assay with D-¹⁴[Ala] as the acceptor included N^{α},N^{ε}-diacetyl-L-Lys-D-Ala (Ac₂-L-Lys-D-Ala), UDP-MurNAc-L-Ala-D-iGlu-L-Lys-D-Ala (UDP-MurNAc-tetrapeptide), L-Ala-D-iGlu-L-Lys-D-Ala (tetrapeptide), GlcNAc-MurNAc-L-Ala-D-iGlu-L-(N^{ε}-D-iAsn)Lys-D-Ala (GlcNAc-MurNAc-tetrapeptide-iAsn).). In contrast, no product was detected with Ac₂-L-Lys-D-Ala-D-Ala and compounds containing a pentapeptide ending in D-Ala-D-Ala (Formation of radioactive peptides was not detected with N^{α},N^{ε}-diacetyl-L-Lys-D-Ala-D-Ala (Ac₂-L-Lys-D-Ala-D-Ala), UDP-MurNAc-L-Ala-D-iGlu-L-Lys-D-Ala-D-Ala (UDP-MurNAc-pentapeptide), L-Ala-D-iGlu-L-Lys-D-Ala-D-Ala (pentapeptide), GlcNAc-MurNAc-L-Ala-D-iGlu-L-(N^{ε}-D-iAsn)Lys-D-Ala-D-Ala (GlcNAc-MurNAc-pentapeptide-iAsn).). Thus, Ldt_{fm} catalyzes peptidoglycan cross-linking exclusively with tetrapeptide-containing donors which are formed in vivo by the β-lactam insensitive D,D-carboxypeptidase according to the pathway depicted in FIG. 8. Strikingly, the specificity of Ldt_{fm} for a tetrapeptide donor ending in L-Lys₃-D-Ala₄ accounts for the lack of inhibition by β-lactams (FIG. 9E) since the drugs are structural analogs of the D-Ala₄-D-Ala₅ extremity of the pentapeptide stem of peptidoglycan precursors.

We have previously detected similar Ldt_{fm} activity in crude extracts from the ampicillin-resistant E. faecium mutant M512 and from the susceptible parental strain D344S (Mainardi et al., 2002). The identification of the corresponding gene, ldt_{fm}, allowed us to confirm that its sequence was identical in both strains and in the E. faecium genome data base. These observations indicate that activation of the L,D-transpeptidation pathway (FIG. 8) does not involve modification of the activity of Ldt_{fm} per se but that of the supply of the appropriate tetrapeptide donor substrate for the cross-linking reaction. The physiological role of the L,D-transpeptidase in β-lactam-susceptible E. faecium is unknown. Previous analyses of peptidoglycan structure in E. coli revealed that a small proportion of the cross-links is generated by L,D-transpeptidation during the exponential phase of growth (ca. 5.8%) (Pisabarro et al., 1985). An increase of their abundance during the stationary phase (ca. 11.3%) was attributed to a short supply of peptidoglycan subunits containing the pentapeptide required for D,D-transpeptidation (Pisabarro et al., 1985). Since mature peptidoglycan of E. faecium contains virtually no pentapeptide stems (Mainardi et al., 2000), we propose that Ldt_{fm} may have a role in the maintenance of peptidoglycan structure since the enzyme can catalyze new cross-links without de novo incorporation of pentapeptide-containing subunits.

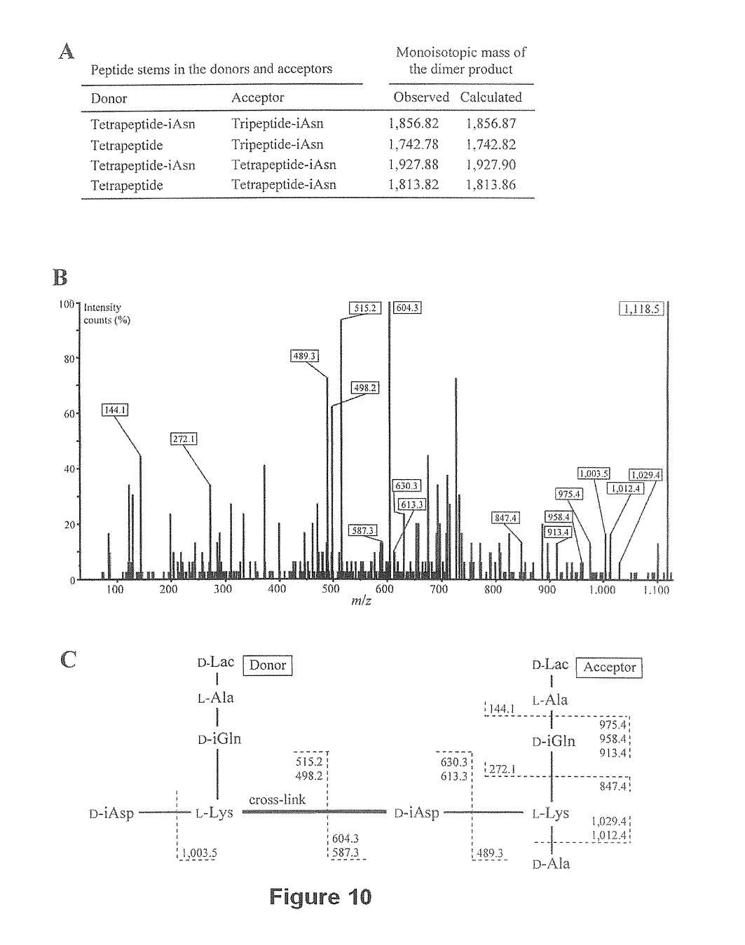

Since Ldt_{fm} had all the characteristics expected for a peptidoglycan cross-linking enzyme, we investigated the formation of L-Lys₃→D-iAsn-L-Lys₃ cross-links with substrates closely mimicking the natural peptidoglycan precursors. Such substrates were prepared from the peptidoglycan of Enterococcus gallinarum, as it contains large amounts of uncross-linked monomers containing a tetrapeptide-iAsn stem (Grohs et al., 2000). L,D-transpeptidation was assayed with a reconstituted pool of three muropeptides to simultaneously test six combinations of donors and acceptors (FIG. 10A). Mass spectrometric analysis of the reaction products revealed formation of dimers with two types of donors (tetrapeptide and tetrapeptide-iAsn) and two types of acceptors (tetrapeptide-iAsn and tripeptide-iAsn) in the four possible combinations. The muropeptide containing an unsubstituted tetrapeptide stem was not used as an acceptor, indicating that the side chain iAsn is essential. Accordingly, direct Lys₃→L-Lys₃ cross-links were not detected in the peptidoglycan of E. faecium M512 (Mainardi et al., 2000). To confirm the structure of the dimers obtained in vitro, the reaction was scaled up, and treated with ammonium hydroxide to cleave the ether link internal to MurNAc. This treatment produced lactoyl-peptides which are more amenable to sequencing by tandem mass spectrometry than disaccharide peptides (Arbeloa et al., 2004). This treatment was also found to convert iD-Asn into iD-Asp. The fragmentation patterns (FIGS. 10B and C) demonstrated the in vitro formation of L-Lys₃→D-iAsn-L-Lys₃ cross-links by Ldt_{fm}. Of note, dimer formation has not been obtained in the case of purified D,D-transpeptidase (PBPs), except in very special cases involving highly reactive artificial substrates (e.g. thioester) or atypical enzymes (e.g. the soluble R61 D,D-peptidase from Streptomyces spp.) (see Anderson et al., 2003, for a recent discussion). Thus, Ldt_{fm} differs from the PBPs in its capacity to function in a soluble acellular system, a feature that could be exploited to design screens for the identification of cross-linking inhibitors.

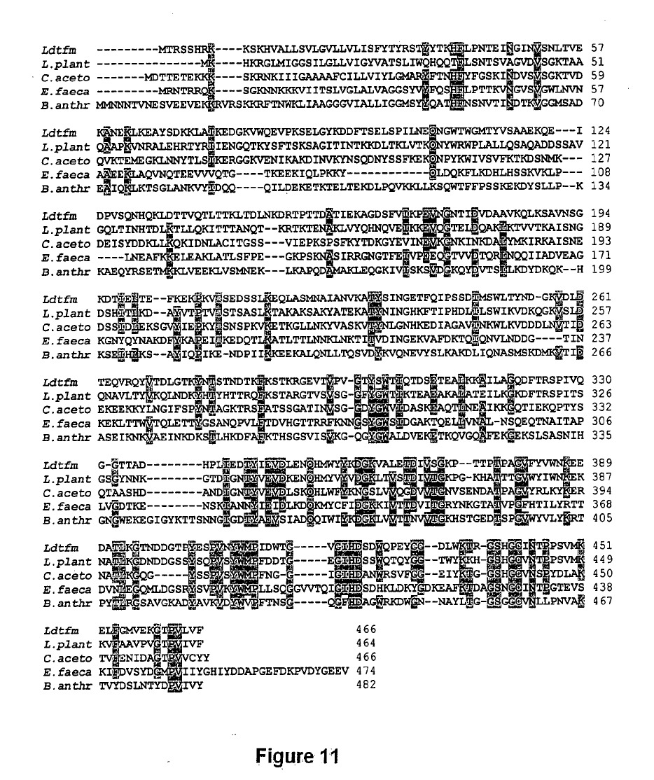

Sequence comparisons indicated that Ldt_{fm} is the first representative of a novel family of proteins which is sporadically distributed among taxonomically distant bacteria. Close homologs (FIG. 11) were detected in pathogenic Gram-positive bacteria including Bacillus anthracis and Enterococcus faecalis but not in Staphylococcus aureus and Streptococcus pneumoniae. Sequence similarity restricted to the C-terminus of Ldt_{fm} was also detected in proteins of unknown functions from other Gram-negative and Gram-positive bacteria (FIG. 9B), but the architecture and domain composition of the proteins were different. Highly conserved residues of the C-terminal domain included Ser and Cys, present at positions 439 and 442 of Ldt_{fm} (FIG. 9B), as potential catalytic residues. Site directed mutagenesis of Ldt_{fm} led to an inactive protein for the Cys442Ala substitution. The mutant protein with the Ser439Ala substitution retained 2% of the activity of the wild-type enzyme. These results suggest that Cys442 could be the catalytic residue of Ldt_{fm}. In contrast, the PBPs possess an active site Ser which is acylated by their substrate and by β-lactams. Accordingly, manual inspection of Ldt_{fm} did not reveal the presence of conserved motifs known to be essential for the activity of the D,D-transpeptidases belonging to the PBP family. Thus, Ldt_{fm} is the first characterized representative of a novel type of transpeptidase. The wide distribution of Ldt_{fm} homologs indicates that β-lactam-resistance by the L,D-transpeptidase bypass mechanism can potentially emerge in various pathogenic bacteria.

### Example 9

### Crystallisation of the L,D-Transpeptidase According to the Invention

1. Crystallization and Data Collection

EfLDT (119-466 of SEQ ID No 13) was crystallized using the sitting-drop vapour-diffusion method at 295 K. Rock-shaped crystals of SeMet-derivatised protein with approximate dimensions 200μ×200μ×200μ were obtained at a concentration of 10 mg/ml using 12.5% PEG 2000, 100 mM ammonium sulfate, 300 mM NaCl and 100 mM sodium acetate trihydrate pH 4.6. X-ray diffraction data (2.4 Å) were collected at the ESRF FIP-BM30A beamline, processed with the CCP4 program suite (MOSFLM and SCALA).

2. Structure Solution and Refinement

The structure of EfLDT was determined by single anomalous diffraction and the position of three ordered Se atoms (out of a possible 5) were found using the program CNS. After density modification using the CNS SAD phase, the model was manually built with one molecule per asymmetric unit. The final model consists of residues 217-398 and 400-466, one sulfate and one zinc ions and 295 water molecules. The 97 residues 119-216 could not be located in the map. Ramachandran analysis indicates that 83.3% of residues are in the most favored region, 15.3% are additionally allowed, and 1.4% are generously allowed.

3. Results

The results from the X-ray diffraction experiment of the crystallized L,D-transpeptidase consisting of the amino acid sequence 119-466 of SEQ ID No 13 are shown in Table 3 hereunder.

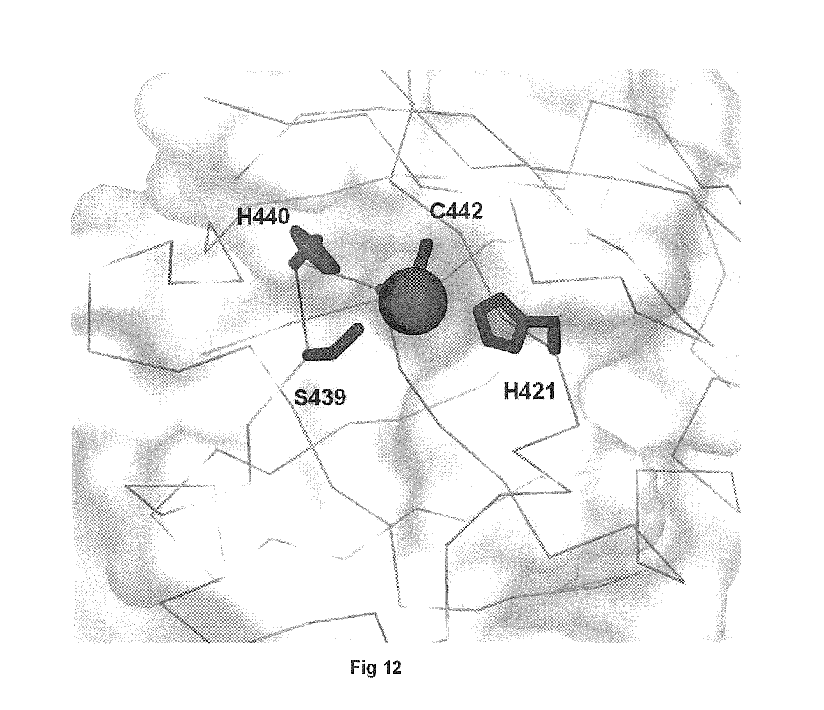

The three-dimensional structure of the crystallized L,D-transpeptidase consisting of the amino acid sequence 119-466 of SEQ ID No 13 is shown in FIG. 12.

The protein is constituted by 2 domains: the domain 1 is constituted by residues 217 to 338 (shown in light grey on top of FIG. 12A), and domain 2 by residues 339 to 466 (shown in dark grey at the bottom of FIG. 12A). The conserved cysteine and histidine are situated in domain 2, deep inside a hole accessible from the surface (see circle in FIG. 12A and FIG. 12B). The channel observed at the surface of the protein is compatible with the accommodation of the substrates, as it is shown in FIG. 12C.

TABLE 1
Molecular masses and composition of muropeptides from E. faecalis
JH2-2/pSJL2aslfm grown in presence of D-aspartate (50 mM)
Monomers (38.9%)*

Inferred structure^{†}

| Peak | (%) | Mass | Stem | Side chain |
|---|---|---|---|---|
| A | 6.6 | 532.2 | Tri | D-Asp |
| B | 4.1 | 603.3 | Tetra | D-Asp |
| C | 23.4 | 674.3 | Penta | D-Asp |
| 1 | 1.8 | 559.3 | Tri | L-Ala-L-Ala |
| 2 | 2.8 | 701.4 | Penta | L-Ala-L-Ala |

Multimers (61.1%)

| | | | Inferred structure | | |
|---|---|---|---|---|---|
| | | | Acceptor | | |
| Peak | (%) | Mass | Stem | Cross bridge^{‡} | Side chain^{§} |

Dimers (39.9%)

| | | | | | |
|---|---|---|---|---|---|
| D | 7.7 | 1117.6 | Tri | D-Asp | D-Asp |
| E | 2.2 | 1188.6 | Tetra | D-Asp | D-Asp |
| F | 18.6 | 1259.7 | Penta | D-Asp | D-Asp |
| G | 2.6 | 1144.6 | Tri | L-Ala-L-Ala | D-Asp |
| H | 0.7 | 1215.7 | Tetra | L-Ala-L-Ala | D-Asp |
| I | 3.1 | 1286.7 | Penta | L-Ala-L-Ala | D-Asp |
| 3 | 2.0 | 1171.7 | Tri | L-Ala-L-Ala | L-Ala-L-Ala |
| 4 | 3.0 | 1313.7 | Penta | L-Ala-L-Ala | L-Ala-L-Ala |

Trimers 17.4%

| | | | | | |
|---|---|---|---|---|---|
| J | 4.7 | 1702.9 | Tri | [D-Asp]x2 | D-Asp |
| K | 1 | 1773.9 | Tetra | [D-Asp]x2 | D-Asp |
| L | 6.8 | 1845.0 | Penta | [D-Asp]x2 | D-Asp |
| M | 1.1 | 1729.9 | Tri | L-Ala-L-Ala-D-Asp | D-Asp |
| N | 1.4 | 1872.0 | Penta | L-Ala-L-Ala-D-Asp | D-Asp |
| P | 0.5 | 1756.9 | Tri | [L-Ala-L-Ala]x2 | D-Asp |
| R | 1.8 | 1899.0 | Penta | [L-Ala-L-Ala]x2 | D-Asp |
| 5 | 1.9 | 1784.0 | Tri | [L-Ala-L-Ala]x2 | L-Ala-L-Ala |
| 6 | 1.8 | 1926.1 | Penta | [L-Ala-L-Ala]x2 | L-Ala-L-Ala |

Tetramers (3.9%)

| | | | | | |
|---|---|---|---|---|---|
| O | 0.6 | 2288.2 | Tri | [D-Asp]x3 | D-Asp |
| Q | 3.3 | 2430.2 | Penta | [D-Asp]x3 | D-Asp |

*The relative abundance (%) of the material in the 24 peaks was calculated by integration of the absorbance at 210 nm.
^{†}The structure was determined from the observed monoisotopic mass of lactoyl peptides and for monomers and dimers (indicated by star) directly determined by tandem mass spectrometry. Tri, tripeptide L-Ala₁-D-iGLN₂-L-Lys₃; Tetra, tetrapeptide; L-Ala₁-D-iGLN₂-L-Lys₃-D-Ala₄; penta, pentapeptide Ala₁-D-iGLN₂-L-Lys₃-D-Ala₄-D-Ala₅;
^{‡}amino acid(s) present in the cross-bridge between two stem peptides
^{§}amino acid(s) present in the free N-terminal side chain

TABLE 2
Exchange reaction catalyzed by Ldt_{fm} between
Ac₂-L-Lys-D-Ala and various acceptors*

| | Product | | |
|---|---|---|---|
| | Mono isotopic mass | | Relative intensity |
| Acceptor | Calculated | Observed | (%)^{†} |
| D-methionine | 361.17 | 361.17 | 50 |
| D-2-hydroxyhexanoic acid | 345.16 | 345.18 | 50 |
| D-lactic acid | 302.16 | 302.17 | 37 |
| D-asparagine | 344.17 | 344.18 | 20 |
| D-glutamine | 358.18 | 358.19 | 20 |
| D-serine | 317.17 | 317.17 | 18 |
| Glycine | 287.15 | 287.16 | 15 |
| D-glutamic acid | 359.17 | 359.17 | 10 |
| D-aspartic acid | 345.15 | 345.14 | 10 |
| D-malic acid | 346.12 | 346.12 | 10 |
| Glycolic acid | 288.13 | ND^{‡} | ND^{‡} |
| L-methionine | 361.17 | ND^{‡} | ND^{‡} |

*Ac₂-L-Lys-D-Ala (0.3 mM) was incubated with Ldt_{fm} (3 g) and various D-2-amino acids (0.3 mM) or D-2-hydroxyacids (0.3 mM) acceptors for 1 h at 37° C. Products were detected by mass spectrometry and the structure was confirmed by tandem mass spectrometry.
^{†}Ionic current intensity (product/product + substrate)
^{‡}ND, not detected

TABLE 3
Structural coordinates of the L, D transpeptidase of (119-466)
of SEQ ID N^{o} 13.

| | | |
|---|---|---|
| HEADER | TRANSFERASE | 07-APR-05 |
| 1ZAT | | |

| TITLE | | CRYSTAL STRUCTURE OF AN ENTEROCOCCUS FAECIUM PEPTIDOGLYCAN | |
|---|---|---|---|
| TITLE | 2 | BINDING PROTEIN AT 2.4 A RESOLUTION | |
| COMPND | | MOL_ID: 1; | |
| COMPND | 2 | MOLECULE: L, D-TRANSPEPTIDASE; | |
| COMPND | 3 | CHAIN: A; | |
| COMPND | 4 | ENGINEERED: YES | |
| SOURCE | | MOL_ID: 1; | |
| SOURCE | 2 | ORGANISM_SCIENTIFIC: ENTEROCOCCUS FAECIUM; | |
| SOURCE | 3 | ORGANISM_COMMON: BACTERIA; | |
| SOURCE | 4 | GENE: LDTFM; | |
| SOURCE | 5 | EXPRESSION_SYSTEM: ESCHERICHIA COLI; | |
| SOURCE | 6 | EXPRESSION_SYSTEM_COMMON: BACTERIA; | |
| SOURCE | 7 | EXPRESSION_SYSTEM_STRAIN: BL21 (DE3); | |
| SOURCE | 8 | EXPRESSION_SYSTEM_VECTOR_TYPE: PLASMID; | |
| SOURCE | 9 | EXPRESSION_SYSTEM_PLASMID: PET2818 | |
| KEYWDS | | L, D-TRANSPEPTIDATION, PEPTIDOGLYCAN, BETA-LACTAM | |
| KEYWDS | 2 | INSENSITIVE TRANSPEPTIDASE, ANTIBIOTIC RESISTANCE | |
| EXPDTA | | X-RAY DIFFRACTION | |
| AUTHOR | | S. BIARROTTE-SORIN, J.-E. HUGONNET, J.-L. MAINARDI, L. GUTMANN, | |
| AUTHOR | 2 | L. RICE, M. ARTHUR, C. MAYER | |
| JRNL | | AUTH | S. BIARROTTE-SORIN, J.-E. HUGONNET, J.-L. MAINARDI, |
| JRNL | | AUTH 2 | L. GUTMANN, L. RICE, M. ARTHUR, C. MAYER |
| JRNL | | TITL | CRYSTAL STRUCTURE OF AN ENTEROCOCCUS FAECIUM |
| JRNL | | TITL 2 | PEPTIDOGLYCAN BINDING PROTEIN |
| JRNL | | REF | TO BE PUBLISHED |
| REMARK | 1 | | |
| REMARK | 2 | | |

| REMARK | 2 | RESOLUTION. 2.40 ANGSTROMS. | |
|---|---|---|---|
| REMARK | 3 | | |
| REMARK | 3 | REFINEMENT. | |
| REMARK | 3 | PROGRAM | : CNS 1.1 |
| REMARK | 3 | AUTHORS | : BRUNGER, ADAMS, CLORE, DELANO, GROS, GROSSE- |
| REMARK | 3 | | : KUNSTLEVE, JIANG, KUSZEWSKI, NILGES, PANNU, |
| REMARK | 3 | | : READ, RICE, SIMONSON, WARREN |
| REMARK | 3 | | |

| REMARK | 3 | REFINEMENT TARGET: ENGH & HUBER | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | : 2.40 |
| REMARK | 3 | RESOLUTION RANGE LOW | (ANGSTROMS) | : 21.92 |
| REMARK | 3 | DATA CUTOFF | (SIGMA(F)) | : 0.000 |
| REMARK | 3 | DATA CUTOFF HIGH | (ABS(F)) | : 1323293.080 |
| REMARK | 3 | DATA CUTOFF LOW | (ABS(F)) | : 0.0000 |

| | | | | |
|---|---|---|---|---|
| REMARK | 3 | COMPLETENESS (WORKING + TEST) | (%) | : 99.2 |
| REMARK | 3 | NUMBER OF REFLECTIONS | | : 20838 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING SET) | : 0.220 |
| REMARK | 3 | FREE R VALUE | | : 0.257 |

| | | | | |
|---|---|---|---|---|
| REMARK | 3 | FREE R VALUE TEST SET SIZE | (%) | : 4.900 |

| REMARK | 3 | FREE R VALUE TEST SET COUNT | : 1012 | |
|---|---|---|---|---|
| REMARK | 3 | ESTIMATED ERROR OF FREE R VALUE | : 0.008 | |
| REMARK | 3 | | | |
| REMARK | 3 | FIT IN THE HIGHEST RESOLUTION BIN. | | |
| REMARK | 3 | TOTAL NUMBER OF BINS USED | | : 6 |
| REMARK | 3 | BIN RESOLUTION RANGE HIGH | (A) | : 2.40 |
| REMARK | 3 | BIN RESOLUTION RANGE LOW | (A) | : 2.55 |
| REMARK | 3 | BIN COMPLETENESS (WORKING + TEST) | (%) | : 99.50 |

| | | | | |
|---|---|---|---|---|
| REMARK | 3 | REFLECTIONS IN BIN | (WORKING SET) | : 3259 |
| REMARK | 3 | BIN R VALUE | (WORKING SET) | : 0.3630 |
| REMARK | 3 | BIN FREE R VALUE | | : 0.4260 |

| | | | | |
|---|---|---|---|---|
| REMARK | 3 | BIN FREE R VALUE TEST SET SIZE | (%) | : 5.00 |

| | | | |
|---|---|---|---|
| REMARK | 3 | BIN FREE R VALUE TEST SET COUNT | : 170 |
| REMARK | 3 | ESTIMATED ERROR OF BIN FREE R VALUE | : 0.033 |
| REMARK | 3 | | |

| REMARK | 3 | NUMBER OF NON-HYDROGEN ATOMS USED IN REFINEMENT. | |
|---|---|---|---|
| REMARK | 3 | PROTEIN ATOMS | : 1940 |
| REMARK | 3 | NUCLEIC ACID ATOMS | : 0 |
| REMARK | 3 | HETEROGEN ATOMS | : 4 |
| REMARK | 3 | SOLVENT ATOMS | : 295 |
| REMARK | 3 | | |
| REMARK | 3 | B VALUES. | |

| | | | | |
|---|---|---|---|---|
| REMARK | 3 | FROM WILSON PLOT | (A**2) | : 49.00 |
| REMARK | 3 | MEAN B VALUE | (OVERALL, A**2) | : 53.00 |

| REMARK | 3 | OVERALL ANISOTROPIC B VALUE. | | |
|---|---|---|---|---|
| REMARK | 3 | B11 (A**2): 7.26000 | | |
| REMARK | 3 | B22 (A**2) : 7.26000 | | |
| REMARK | 3 | B33 (A**2) : −14.52000 | | |
| REMARK | 3 | B12 (A**2) : 3.37000 | | |
| REMARK | 3 | B13 (A**2) : 0.00000 | | |
| REMARK | 3 | B23 (A**2) : 0.00000 | | |
| REMARK | 3 | | | |
| REMARK | 3 | ESTIMATED COORDINATE ERROR. | | |
| REMARK | 3 | ESD FROM LUZZATI PLOT | (A) | : 0.33 |
| REMARK | 3 | ESD FROM SIGMAA | (A) | : 0.42 |
| REMARK | 3 | LOW RESOLUTION CUTOFF | (A) | : 5.00 |
| REMARK | 3 | | | |
| REMARK | 3 | CROSS-VALIDATED ESTIMATED COORDINATE ERROR. | | |
| REMARK | 3 | ESD FROM C-V LUZZATI PLOT | (A) | : 0.41 |
| REMARK | 3 | ESD FROM C-V SIGMAA | (A) | : 0.49 |
| REMARK | 3 | | | |

| REMARK | 3 | RMS DEVIATIONS FROM IDEAL VALUES. | | |
|---|---|---|---|---|
| REMARK | 3 | BOND LENGTHS | (A) | : 0.008 |
| REMARK | 3 | BOND ANGLES | (DEGREES) | : 1.20 |
| REMARK | 3 | DIHEDRAL ANGLES | (DEGREES) | : 24.10 |
| REMARK | 3 | IMPROPER ANGLES | (DEGREES) | : 0.75 |
| REMARK | 3 | | | |

| REMARK | 3 | ISOTROPIC THERMAL MODEL: RESTRAINED | |
|---|---|---|---|
| REMARK | 3 | | |
| REMARK | 3 | ISOTROPIC THERMAL FACTOR RESTRAINTS. RMS SIGMA | |
| REMARK | 3 | MAIN-CHAIN BOND | (A**2) : 1.280; 1.500 |
| REMARK | 3 | MAIN-CHAIN ANGLE | (A**2) : 2.190; 2.000 |
| REMARK | 3 | SIDE-CHAIN BOND | (A**2) : 1.940; 2.000 |
| REMARK | 3 | SIDE-CHAIN ANGLE | (A**2) : 2.990; 2.500 |
| REMARK | 3 | | |
| REMARK | 3 | BULK SOLVENT MODELING. | |

| | | | |
|---|---|---|---|
| REMARK | 3 | METHOD USED | : FLAT MODEL |
| REMARK | 3 | KSOL | : 0.35 |
| REMARK | 3 | BSOL | : 55.71 |
| REMARK | 3 | | |

| | | | |
|---|---|---|---|
| REMARK | 3 | NCS MODEL: NULL | |
| REMARK | 3 | | |
| REMARK | 3 | NCS RESTRAINTS. | RMS SIGMA/WEIGHT |

| | | | | |
|---|---|---|---|---|
| REMARK | 3 | GROUP 1 POSITIONAL | (A) | : NULL; NULL |
| REMARK | 3 | GROUP 1 B-FACTOR | (A**2) | : NULL; NULL |
| REMARK | 3 | | | |

| | | | |
|---|---|---|---|
| REMARK | 3 | PARAMETER FILE 1 | : PROTEIN_REP.PARAM |
| REMARK | 3 | PARAMETER FILE 2 | : WATER_REP.PARAM |
| REMARK | 3 | PARAMETER FILE 3 | : ION.PARAM |
| REMARK | 3 | PARAMETER FILE 4 | : NULL |
| REMARK | 3 | TOPOLOGY FILE 1 | : PROTEIN.TOP |
| REMARK | 3 | TOPOLOGY FILE 2 | : WATER.TOP |
| REMARK | 3 | TOPOLOGY FILE 3 | : ION.TOP |
| REMARK | 3 | TOPOLOGY FILE 4 | : NULL |
| REMARK | 3 | | |

| REMARK | 3 | OTHER REFINEMENT REMARKS: NULL | | |
|---|---|---|---|---|
| REMARK | 4 | | | |
| REMARK | 4 | 1ZAT COMPLIES WITH FORMAT V. 2.3, 09-JULY-1998 | | |
| REMARK | 100 | | | |
| REMARK | 100 | THIS ENTRY HAS BEEN PROCESSED BY RCSB ON 12-APR-2005. | | |
| REMARK | 100 | THE RCSB ID CODE IS RCSB032508. | | |
| REMARK | 200 | | | |
| REMARK | 200 | EXPERIMENTAL DETAILS | | |
| REMARK | 200 | EXPERIMENT TYPE | | : X-RAY DIFFRACTION |
| REMARK | 200 | DATE OF DATA COLLECTION | | : 01-MAY-2004 |
| REMARK | 200 | TEMPERATURE | (KELVIN) | : 100.0 |
| REMARK | 200 | PH | | : 6.40 |

| | | | | |
|---|---|---|---|---|
| REMARK | 200 | NUMBER OF CRYSTALS USED | | : 1 |
| REMARK | 200 | | | |
| REMARK | 200 | SYNCHROTRON | (Y/N) | : Y |
| REMARK | 200 | RADIATION SOURCE | | : ESRF |
| REMARK | 200 | BEAMLINE | | : BM30A |
| REMARK | 200 | X-RAY GENERATOR MODEL | | : NULL |
| REMARK | 200 | MONOCHROMATIC OR LAUE | (M/L) | : M |
| REMARK | 200 | WAVELENGTH OR RANGE | (A) | : 0.979676 |

| REMARK | 200 | MONOCHROMATOR | | : SAGITALLY FOCUSED SI (111) |
|---|---|---|---|---|
| REMARK | 200 | OPTICS | | : MIRROR 1, DOUBLE CRYSTAL, |
| REMARK | 200 | | | MIRROR 2 |
| REMARK | 200 | | | |
| REMARK | 200 | DETECTOR TYPE | | : CCD |
| REMARK | 200 | DETECTOR MANUFACTURER | | : MARRESEARCH |
| REMARK | 200 | INTENSITY-INTEGRATION SOFTWARE | | : MOSFLM |
| REMARK | 200 | DATA SCALING SOFTWARE | | : SCALA |
| REMARK | 200 | | | |
| REMARK | 200 | NUMBER OF UNIQUE REFLECTIONS | | : 20893 |
| REMARK | 200 | RESOLUTION RANGE HIGH | (A) | : 2.400 |
| REMARK | 200 | RESOLUTION RANGE LOW | (A) | : 21.900 |

| | | | | |
|---|---|---|---|---|
| REMARK | 200 | REJECTION CRITERIA | (SIGMA(I)) | : 0.000 |
| REMARK | 200 | | | |
| REMARK | 200 | OVERALL. | | |

| | | | | |
|---|---|---|---|---|
| REMARK | 200 | COMPLETENESS FOR RANGE | (%) | : 99.5 |
| REMARK | 200 | DATA REDUNDANCY | | : 8.000 |
| REMARK | 200 | R MERGE | (I) | : NULL |
| REMARK | 200 | R SYM | (I) | : 0.09600 |

| | | | |
|---|---|---|---|
| REMARK | 200 | <I/SIGMA(I)> FOR THE DATA SET | : 6.4000 |
| REMARK | 200 | | |
| REMARK | 200 | IN THE HIGHEST RESOLUTION SHELL. | |

| | | | | |
|---|---|---|---|---|
| REMARK | 200 | HIGHEST RESOLUTION SHELL, RANGE HIGH | (A) | : 2.40 |
| REMARK | 200 | HIGHEST RESOLUTION SHELL, RANGE LOW | (A) | : 2.53 |

| | | | | |
|---|---|---|---|---|
| REMARK | 200 | COMPLETENESS FOR SHELL | (%) | : 99.5 |
| REMARK | 200 | DATA REDUNDANCY IN SHELL | | : 6.70 |
| REMARK | 200 | R MERGE FOR SHELL | (I) | : NULL |
| REMARK | 200 | R SYM FOR SHELL | (I) | : 0.46400 |
| REMARK | 200 | <I/SIGMA(I)> FOR SHELL | | : 1.500 |
| REMARK | 200 | | | |

| REMARK | 200 | DIFFRACTION PROTOCOL: SINGLE WAVELENGTH | | |
|---|---|---|---|---|
| REMARK | 200 | METHOD USED TO DETERMINE THE STRUCTURE: SAD | | |
| REMARK | 200 | SOFTWARE USED: CNS | | |
| REMARK | 200 | STARTING MODEL: NULL | | |
| REMARK | 200 | | | |
| REMARK | 200 | REMARK: NULL | | |
| REMARK | 280 | | | |
| REMARK | 280 | CRYSTAL | | |
| REMARK | 280 | SOLVENT CONTENT, VS (%): 70.68 | | |
| REMARK | 280 | MATTHEWS COEFFICIENT, VM (ANGSTROMS**3/DA): 4.23 | | |
| REMARK | 280 | | | |
| REMARK | 280 | CRYSTALLIZATION CONDITIONS: AMMONIUM SULFATE, SODIUM CHLORIDE, | | |
| REMARK | 280 | SODIUM ACETATE, PEG 2000, PH 6.4, VAPOR DIFFUSION, SITTING | | |
| REMARK | 280 | DROP, TEMPERATURE 100 K | | |
| REMARK | 290 | | | |
| REMARK | 290 | CRYSTALLOGRAPHIC SYMMETRY | | |
| REMARK | 290 | SYMMETRY OPERATORS FOR SPACE GROUP: P 31 2 1 | | |
| REMARK | 290 | | | |
| REMARK | 290 | | SYMOP | SYMMETRY |
| REMARK | 290 | | NNNMMM | OPERATOR |
| REMARK | 290 | | 1555 | X, Y, Z |
| REMARK | 290 | | 2555 | −Y, X − Y, ⅓ + Z |
| REMARK | 290 | | 3555 | −X + Y, −X, ⅔ + Z |
| REMARK | 290 | | 4555 | Y, X, −Z |
| REMARK | 290 | | 5555 | X − Y, −Y, ⅔ − Z |
| REMARK | 290 | | 6555 | −X, −X + Y, ⅓ − Z |
| REMARK | 290 | | | |

| REMARK | 290 | | WHERE | NNN -> OPERATOR NUMBER | | | | |
|---|---|---|---|---|---|---|---|---|
| REMARK | 290 | | | MMM -> TRANSLATION VECTOR | | | | |
| REMARK | 290 | | | | | | | |
| REMARK | 290 | CRYSTALLOGRAPHIC SYMMETRY TRANSFORMATIONS | | | | | | |
| REMARK | 290 | THE FOLLOWING TRANSFORMATIONS OPERATE ON THE ATOM/HETATM | | | | | | |
| REMARK | 290 | RECORDS IN THIS ENTRY TO PRODUCE CRYSTALLOGRAPHICALLY | | | | | | |
| REMARK | 290 | RELATED MOLECULES. | | | | | | |
| REMARK | 290 | | SMTRY1 | 1 | 1.000000 | 0.000000 | 0.000000 | 0.00000 |
| REMARK | 290 | | SMTRY2 | 1 | 0.000000 | 1.000000 | 0.000000 | 0.00000 |
| REMARK | 290 | | SMTRY3 | 1 | 0.000000 | 0.000000 | 1.000000 | 0.00000 |
| REMARK | 290 | | SMTRY1 | 2 | −0.500000 | −0.866025 | 0.000000 | 0.00000 |
| REMARK | 290 | | SMTRY2 | 2 | 0.866025 | −0.500000 | 0.000000 | 0.00000 |
| REMARK | 290 | | SMTRY3 | 2 | 0.000000 | 0.000000 | 1.000000 | 22.75833 |
| REMARK | 290 | | SMTRY1 | 3 | −0.500000 | 0.866025 | 0.000000 | 0.00000 |
| REMARK | 290 | | SMTRY2 | 3 | −0.866025 | −0.500000 | 0.000000 | 0.00000 |
| REMARK | 290 | | SMTRY3 | 3 | 0.000000 | 0.000000 | 1.000000 | 45.51667 |
| REMARK | 290 | | SMTRY1 | 4 | −0.500000 | 0.866025 | 0.000000 | 0.00000 |
| REMARK | 290 | | SMTRY2 | 4 | 0.866025 | 0.500000 | 0.000000 | 0.00000 |
| REMARK | 290 | | SMTRY3 | 4 | 0.000000 | 0.000000 | −1.000000 | 0.00000 |
| REMARK | 290 | | SMTRY1 | 5 | 1.000000 | 0.000000 | 0.000000 | 0.00000 |
| REMARK | 290 | | SMTRY2 | 5 | 0.000000 | −1.000000 | 0.000000 | 0.00000 |
| REMARK | 290 | | SMTRY3 | 5 | 0.000000 | 0.000000 | −1.000000 | 45.51667 |
| REMARK | 290 | | SMTRY1 | 6 | −0.500000 | −0.866025 | 0.000000 | 0.00000 |
| REMARK | 290 | | SMTRY2 | 6 | −0.866025 | 0.500000 | 0.000000 | 0.00000 |
| REMARK | 290 | | SMTRY3 | 6 | 0.000000 | 0.000000 | −1.000000 | 22.75833 |
| REMARK | 290 | | | | | | | |

| REMARK | 290 | REMARK: NULL | | | | | |
|---|---|---|---|---|---|---|---|
| REMARK | 300 | | | | | | |
| REMARK | 300 | BIOMOLECULE: 1 | | | | | |
| REMARK | 300 | THIS ENTRY CONTAINS THE CRYSTALLOGRAPHIC ASYMMETRIC | | | | | |
| | | UNIT | | | | | |
| REMARK | 300 | WHICH CONSISTS OF 1 CHAIN(S). SEE REMARK 350 FOR | | | | | |
| REMARK | 300 | INFORMATION ON GENERATING THE BIOLOGICAL MOLECULE(S). | | | | | |
| REMARK | 350 | | | | | | |
| REMARK | 350 | GENERATING THE BIOMOLECULE | | | | | |
| REMARK | 350 | COORDINATES FOR A COMPLETE MULTIMER REPRESENTING THE KNOWN | | | | | |
| REMARK | 350 | BIOLOGICALLY SIGNIFICANT OLIGOMERIZATION STATE OF THE | | | | | |
| REMARK | 350 | MOLECULE CAN BE GENERATED BY APPLYING BIOMT TRANSFORMATIONS | | | | | |
| REMARK | 350 | GIVEN BELOW. BOTH NON-CRYSTALLOGRAPHIC AND | | | | | |
| REMARK | 350 | CRYSTALLOGRAPHIC OPERATIONS ARE GIVEN. | | | | | |
| REMARK | 350 | | | | | | |
| REMARK | 350 | BIOMOLECULE: 1 | | | | | |
| REMARK | 350 | APPLY THE FOLLOWING TO CHAINS: A | | | | | |
| REMARK | 350 | BIOMT1 | 1 | 1.000000 | 0.000000 | 0.000000 | 0.00000 |
| REMARK | 350 | BIOMT2 | 1 | 0.000000 | 1.000000 | 0.000000 | 0.00000 |
| REMARK | 350 | BIOMT3 | 1 | 0.000000 | 0.000000 | 1.000000 | 0.00000 |
| REMARK | 375 | | | | | | |

| REMARK | 375 | SPECIAL POSITION | | | |
|---|---|---|---|---|---|
| REMARK | 375 | THE FOLLOWING ATOMS ARE FOUND TO BE WITHIN 0.15 ANGSTROMS | | | |
| REMARK | 375 | OF A SYMMETRY RELATED ATOM AND ARE ASSUMED TO BE ON SPECIAL | | | |
| REMARK | 375 | POSITIONS. | | | |
| REMARK | 375 | | | | |
| REMARK | 375 | ATOM | RES | CSSEQI | |
| REMARK | 375 | S | SO4 | 763 | LIES ON A SPECIAL POSITION. |
| REMARK | 465 | | | | |

| REMARK | 465 | MISSING RESIDUES | | | |
|---|---|---|---|---|---|
| REMARK | 465 | THE FOLLOWING RESIDUES WERE NOT LOCATED IN THE | | | |
| REMARK | 465 | EXPERIMENT. (M = MODEL NUMBER; RES = RESIDUE NAME; C = CHAIN | | | |
| REMARK | 465 | IDENTIFIER; SSSEQ = SEQUENCE NUMBER; I = INSERTION CODE.) | | | |
| REMARK | 465 | | | | |
| REMARK | 465 | M | RES | C | SSSEQI |
| REMARK | 465 | | ASP | A | 398 |
| REMARK | 465 | | ASP | A | 399 |
| REMARK | 470 | | | | |

| REMARK | 470 | MISSING ATOM | | | | | | | |
|---|---|---|---|---|---|---|---|---|---|
| REMARK | 470 | THE FOLLOWING RESIDUES HAVE MISSING ATOMS(M = MODEL NUMBER; | | | | | | | |
| REMARK | 470 | RES = RESIDUE NAME; C = CHAIN IDENTIFIER; SSEQ = SEQUENCE NUMBER; | | | | | | | |
| REMARK | 470 | I = INSERTION CODE): | | | | | | | |
| REMARK | 470 | M | RES | CSSEQI | ATOMS | | | | |
| REMARK | 470 | | GLU | A 404 | CB | CG | CD | OE1 | OE2 |
| REMARK | 470 | | GLU | A 428 | CG | CD | OE1 | OE2 | |
| REMARK | 500 | | | | | | | | |

| REMARK | 500 | GEOMETRY AND STEREOCHEMISTRY | | | | | | | | | |
|---|---|---|---|---|---|---|---|---|---|---|---|
| REMARK | 500 | SUBTOPIC: CLOSE CONTACTS | | | | | | | | | |
| REMARK | 500 | | | | | | | | | | |
| REMARK | 500 | THE FOLLOWING ATOMS THAT ARE RELATED BY CRYSTALLOGRAPHIC | | | | | | | | | |
| REMARK | 500 | SYMMETRY ARE IN CLOSE CONTACT. AN ATOM LOCATED WITHIN 0.15 | | | | | | | | | |
| REMARK | 500 | ANGSTROMS OF A SYMMETRY RELATED ATOM IS ASSUMED TO BE ON A | | | | | | | | | |
| REMARK | 500 | SPECIAL POSITION AND IS, THEREFORE, LISTED IN REMARK 375 | | | | | | | | | |
| REMARK | 500 | INSTEAD OF REMARK 500. ATOMS WITH NON-BLANK ALTERNATE | | | | | | | | | |
| REMARK | 500 | LOCATION INDICATORS ARE NOT INCLUDED IN THE CALCULATIONS. | | | | | | | | | |
| REMARK | 500 | | | | | | | | | | |
| REMARK | 500 | DISTANCE CUTOFF: | | | | | | | | | |
| REMARK | 500 | 2.2 ANGSTROMS FOR CONTACTS NOT INVOLVING HYDROGEN ATOMS | | | | | | | | | |
| REMARK | 500 | 1.6 ANGSTROMS FOR CONTACTS INVOLVING HYDROGEN ATOMS | | | | | | | | | |
| REMARK | 500 | | | | | | | | | | |
| REMARK | 500 | ATM1 | RES | C | SSEQI | ATM2 | RES | C | SSEQI | SSYMOP | DISTANCE |
| REMARK | 500 | S | SO4 | | 763 | O1 | SO4 | | 763 | 6555 | 1.51 |
| REMARK | 500 | S | SO4 | | 763 | O4 | SO4 | | 763 | 6555 | 1.71 |
| REMARK | 500 | | | | | | | | | | |

| REMARK | 500 | GEOMETRY AND STEREOCHEMISTRY | | | | | | | |
|---|---|---|---|---|---|---|---|---|---|
| REMARK | 500 | SUBTOPIC: COVALENT BOND LENGTHS | | | | | | | |
| REMARK | 500 | | | | | | | | |
| REMARK | 500 | THE STEREOCHEMICAL PARAMETERS OF THE FOLLOWING RESIDUES | | | | | | | |
| REMARK | 500 | HAVE VALUES WHICH DEVIATE FROM EXPECTED VALUES BY MORE | | | | | | | |
| REMARK | 500 | THAN 6*RMSD (M = MODEL NUMBER; RES = RESIDUE NAME; C = CHAIN | | | | | | | |
| REMARK | 500 | IDENTIFIER; SSEQ = SEQUENCE NUMBER; I = INSERTION CODE). | | | | | | | |
| REMARK | 500 | | | | | | | | |
| REMARK | 500 | STANDARD TABLE: | | | | | | | |
| REMARK | 500 | FORMAT: (10X, I3, 1X, 2(A3, 1X, A1, I4, A1, 1X, A4, 3X), F6.3) | | | | | | | |
| REMARK | 500 | | | | | | | | |
| REMARK | 500 | EXPECTED VALUES: ENGH AND HUBER, 1991 | | | | | | | |
| REMARK | 500 | | | | | | | | |
| REMARK | 500 | M | RES | CSSEQI | ATM1 | RES | CSSEQI | ATM2 | DEVIATION |
| REMARK | 500 | | LYS | A 217 | CD | LYS | A 217 | CE | 0.049 |
| REMARK | 500 | | LYS | A 257 | CE | LYS | A 257 | NZ | 0.055 |
| REMARK | 500 | | MET | A 411 | SD | MET | A 411 | CE | −0.067 |
| REMARK | 500 | | GLN | A 426 | CD | GLN | A 426 | NE2 | −0.081 |
| REMARK | 500 | | | | | | | | |

| REMARK | 500 | GEOMETRY AND STEREOCHEMISTRY | | | | | |
|---|---|---|---|---|---|---|---|
| REMARK | 500 | SUBTOPIC: COVALENT BOND ANGLES | | | | | |
| REMARK | 500 | | | | | | |
| REMARK | 500 | THE STEREOCHEMICAL PARAMETERS OF THE FOLLOWING RESIDUES | | | | | |
| REMARK | 500 | HAVE VALUES WHICH DEVIATE FROM EXPECTED VALUES BY MORE | | | | | |
| REMARK | 500 | THAN 6*RMSD (M = MODEL NUMBER; RES = RESIDUE NAME; C = CHAIN | | | | | |
| REMARK | 500 | IDENTIFIER; SSEQ = SEQUENCE NUMBER; I = INSERTION CODE). | | | | | |
| REMARK | 500 | | | | | | |
| REMARK | 500 | STANDARD TABLE: | | | | | |
| REMARK | 500 | FORMAT: (10X, I3, 1X, A3, 1X, A1, I4, A1, 3(1X, A4, 2X), 12X, F5.1) | | | | | |
| REMARK | 500 | | | | | | |
| REMARK | 500 | EXPECTED VALUES: ENGH AND HUBER, 1991 | | | | | |
| REMARK | 500 | | | | | | |
| REMARK | 500 | M | RES | CSSEQI | ATM1 | ATM2 | ATM3 |

| | | | | |
|---|---|---|---|---|
| REMARK | 500 | GLN | A 219 | N - CA - C ANGL. DEV. = −8.1 DEGREES |
| REMARK | 500 | ILE | A 242 | N - CA - C ANGL. DEV. = −7.5 DEGREES |
| REMARK | 500 | VAL | A 258 | N - CA - C ANGL. DEV. = −8.0 DEGREES |
| REMARK | 500 | TYR | A 276 | N - CA - C ANGL. DEV. = 8.2 DEGREES |
| REMARK | 500 | LYS | A 284 | N - CA - C ANGL. DEV. = −7.7 DEGREES |
| REMARK | 500 | THR | A 324 | N - CA - C ANGL. DEV. = −7.6 DEGREES |
| REMARK | 500 | SER | A 326 | N - CA - C ANGL. DEV. = −8.7 DEGREES |
| REMARK | 500 | THR | A 377 | N - CA - C ANGL. DEV. = −8.4 DEGREES |
| REMARK | 525 | | | |

| REMARK | 525 | SOLVENT | | |
|---|---|---|---|---|
| REMARK | 525 | THE FOLLOWING SOLVENT MOLECULES LIE FARTHER THAN EXPECTED | | |
| REMARK | 525 | FROM THE PROTEIN OR NUCLEIC ACID MOLECULE AND MAY BE | | |
| REMARK | 525 | ASSOCIATED WITH A SYMMETRY RELATED MOLECULE (M = MODEL | | |
| REMARK | 525 | NUMBER; RES = RESIDUE NAME; C = CHAIN IDENTIFIER; SSEQ = SEQUENCE | | |
| REMARK | 525 | NUMBER; I = INSERTION CODE): | | |
| REMARK | 525 | | | |
| REMARK | 525 | M | RES | CSSEQI |

| | | | | |
|---|---|---|---|---|
| REMARK | 525 | HOH | 515 | DISTANCE = 5.93 ANGSTROMS |
| REMARK | 525 | HOH | 516 | DISTANCE = 8.01 ANGSTROMS |
| REMARK | 525 | HOH | 593 | DISTANCE = 12.51 ANGSTROMS |
| REMARK | 525 | HOH | 633 | DISTANCE = 6.95 ANGSTROMS |
| REMARK | 525 | HOH | 661 | DISTANCE = 7.44 ANGSTROMS |
| REMARK | 525 | HOH | 662 | DISTANCE = 7.32 ANGSTROMS |
| REMARK | 525 | HOH | 663 | DISTANCE = 7.22 ANGSTROMS |
| REMARK | 525 | HOH | 671 | DISTANCE = 7.45 ANGSTROMS |
| REMARK | 525 | HOH | 672 | DISTANCE = 8.95 ANGSTROMS |
| REMARK | 525 | HOH | 676 | DISTANCE = 5.03 ANGSTROMS |
| REMARK | 525 | HOH | 693 | DISTANCE = 7.80 ANGSTROMS |
| REMARK | 525 | HOH | 725 | DISTANCE = 7.55 ANGSTROMS |
| REMARK | 525 | HOH | 747 | DISTANCE = 5.34 ANGSTROMS |
| REMARK | 525 | HOH | 752 | DISTANCE = 5.01 ANGSTROMS |
| REMARK | 525 | HOH | 762 | DISTANCE = 5.03 ANGSTROMS |

| | | | | | | |
|---|---|---|---|---|---|---|
| DBREF | 1ZAT | A | 217 | 466 | GB | 48825684 ZP_00286925 217 466 |

| | | | | | | | | | | | | | | | | |
|---|---|---|---|---|---|---|---|---|---|---|---|---|---|---|---|---|
| SEQRES | 1 | A | 250 | LYS | GLU | GLN | LEU | ALA | SER | MET | ASN | ALA | ILE | ALA | ASN | VAL |
| SEQRES | 2 | A | 250 | LYS | ALA | THR | TYR | SER | ILE | ASN | GLY | GLU | THR | PHE | GLN | ILE |
| SEQRES | 3 | A | 250 | PRO | SER | SER | ASP | ILE | MET | SER | TRP | LEU | THR | TYR | ASN | ASP |
| SEQRES | 4 | A | 250 | GLY | LYS | VAL | ASP | LEU | ASP | THR | GLU | GLN | VAL | ARG | GLN | TYR |
| SEQRES | 5 | A | 250 | VAL | THR | ASP | LEU | GLY | THR | LYS | TYR | ASN | THR | SER | THR | ASN |
| SEQRES | 6 | A | 250 | ASP | THR | LYS | PHE | LYS | SER | THR | LYS | ARG | GLY | GLU | VAL | THR |
| SEQRES | 7 | A | 250 | VAL | PRO | VAL | GLY | THR | TYR | SER | TRP | THR | ILE | GLN | THR | ASP |
| SEQRES | 8 | A | 250 | SER | GLU | THR | GLU | ALA | LEU | LYS | LYS | ALA | ILE | LEU | ALA | GLY |
| SEQRES | 9 | A | 250 | GLN | ASP | PHE | THR | ARG | SER | PRO | ILE | VAL | GLN | GLY | GLY | THR |
| SEQRES | 10 | A | 250 | THR | ALA | ASP | HIS | PRO | LEU | ILE | GLU | ASP | THR | TYR | ILE | GLU |
| SEQRES | 11 | A | 250 | VAL | ASP | LEU | GLU | ASN | GLN | HIS | MET | TRP | TYR | TYR | LYS | ASP |
| SEQRES | 12 | A | 250 | GLY | LYS | VAL | ALA | LEU | GLU | THR | ASP | ILE | VAL | SER | GLY | LYS |
| SEQRES | 13 | A | 250 | PRO | THR | THR | PRO | THR | PRO | ALA | GLY | VAL | PHE | TYR | VAL | TRP |
| SEQRES | 14 | A | 250 | ASN | LYS | GLU | GLU | ASP | ALA | THR | LEU | LYS | GLY | THR | ASN | ASP |
| SEQRES | 15 | A | 250 | ASP | GLY | THR | PRO | TYR | GLU | SER | PRO | VAL | ASN | TYR | TRP | MET |
| SEQRES | 16 | A | 250 | PRO | ILE | ASP | TRP | THR | GLY | VAL | GLY | ILE | HIS | ASP | SER | ASP |
| SEQRES | 17 | A | 250 | TRP | GLN | PRO | GLU | TYR | GLY | GLY | ASP | LEU | TRP | LYS | THR | ARG |
| SEQRES | 18 | A | 250 | GLY | SER | HIS | GLY | CYS | ILE | ASN | THR | PRO | PRO | SER | VAL | MET |
| SEQRES | 19 | A | 250 | LYS | GLU | LEU | PHE | GLY | MET | VAL | GLU | LYS | GLY | THR | PRO | VAL |
| SEQRES | 20 | A | 250 | LEU | VAL | PHE | | | | | | | | | | |

| | | | |
|---|---|---|---|
| HET | ZN | 467 | 1 |
| HET | SO4 | 763 | 3 |

| | | |
|---|---|---|
| HETNAM | ZN | ZINC ION |
| HETNAM | SO4 | SULFATE ION |

| | | | |
|---|---|---|---|
| FORMUL | 2 | ZN | ZN1 2+ |
| FORMUL | 3 | SO4 | O4 S1 2− |

| | | | |
|---|---|---|---|
| FORMUL | 4 | HOH | *295 (H2 O1) |

| | | | | | | | | | | |
|---|---|---|---|---|---|---|---|---|---|---|
| HELIX | 1 | 1 | GLU | A | 218 | VAL | A | 229 | 1 | 12 |
| HELIX | 2 | 2 | PRO | A | 243 | TRP | A | 250 | 1 | 8 |
| HELIX | 3 | 3 | ASP | A | 261 | ASN | A | 277 | 1 | 17 |
| HELIX | 4 | 4 | GLN | A | 305 | GLY | A | 320 | 1 | 16 |
| HELIX | 5 | 5 | ASP | A | 432 | GLY | A | 438 | 1 | 7 |
| HELIX | 6 | 6 | PRO | A | 446 | VAL | A | 457 | 1 | 12 |

| | | | | | | | | | | | | | | | | | | |
|---|---|---|---|---|---|---|---|---|---|---|---|---|---|---|---|---|---|---|
| SHEET | 1 | A | 3 | GLU | A | 238 | GLN | A | 241 | 0 | | | | | | | | |
| SHEET | 2 | A | 3 | ALA | A | 231 | ILE | A | 235 | −1 | N | ILE | A | 235 | O | GLU | A | 238 |
| SHEET | 3 | A | 3 | PHE | A | 323 | ARG | A | 325 | 1 | O | PHE | A | 323 | N | THR | A | 232 |
| SHEET | 1 | B | 2 | LEU | A | 251 | ASN | A | 254 | 0 | | | | | | | | |
| SHEET | 2 | B | 2 | LYS | A | 257 | LEU | A | 260 | −1 | O | ASP | A | 259 | N | THR | A | 252 |
| SHEET | 1 | C | 2 | THR | A | 283 | LYS | A | 286 | 0 | | | | | | | | |
| SHEET | 2 | C | 2 | GLU | A | 292 | VAL | A | 295 | −1 | O | VAL | A | 295 | N | THR | A | 283 |
| SHEET | 1 | D | 2 | TRP | A | 302 | ILE | A | 304 | 0 | | | | | | | | |
| SHEET | 2 | D | 2 | VAL | A | 329 | GLY | A | 331 | −1 | O | GLN | A | 330 | N | THR | A | 303 |
| SHEET | 1 | E | 5 | LYS | A | 361 | ASP | A | 367 | 0 | | | | | | | | |
| SHEET | 2 | E | 5 | HIS | A | 353 | LYS | A | 358 | −1 | N | TYR | A | 356 | O | ALA | A | 363 |
| SHEET | 3 | E | 5 | TYR | A | 344 | ASP | A | 348 | −1 | N | GLU | A | 346 | O | TRP | A | 355 |
| SHEET | 4 | E | 5 | PRO | A | 462 | PHE | A | 466 | 1 | O | LEU | A | 464 | N | VAL | A | 347 |
| SHEET | 5 | E | 5 | GLY | A | 380 | TYR | A | 383 | −1 | N | PHE | A | 382 | O | VAL | A | 463 |
| SHEET | 1 | F | 4 | GLU | A | 388 | THR | A | 392 | 0 | | | | | | | | |
| SHEET | 2 | F | 4 | PRO | A | 406 | PRO | A | 412 | −1 | O | TRP | A | 410 | N | GLU | A | 388 |
| SHEET | 3 | F | 4 | GLY | A | 419 | ASP | A | 422 | −1 | O | ILE | A | 420 | N | MET | A | 411 |
| SHEET | 4 | F | 4 | ILE | A | 443 | THR | A | 445 | 1 | O | ILE | A | 443 | N | GLY | A | 419 |

| | | | | | | | | | |
|---|---|---|---|---|---|---|---|---|---|
| CRYST1 | 115.976 | 115.976 | 68.275 | 90.00 | 90.00 | 120.00 | P | 31 | 2 1 6 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.008622 | 0.004978 | 0.000000 | 0.00000 |
| SCALE2 | 0.000000 | 0.009956 | 0.000000 | 0.00000 |
| SCALE3 | 0.000000 | 0.000000 | 0.014647 | 0.00000 |

| | | | | | | | | | | | |
|---|---|---|---|---|---|---|---|---|---|---|---|
| ATOM | 1 | N | LYS | A | 217 | −44.381 | 61.577 | 19.388 | 1.00 | 83.19 | N |
| ATOM | 2 | CA | LYS | A | 217 | −44.196 | 62.690 | 20.359 | 1.00 | 83.28 | C |
| ATOM | 3 | C | LYS | A | 217 | −43.445 | 63.837 | 19.716 | 1.00 | 83.58 | C |
| ATOM | 4 | O | LYS | A | 217 | −43.417 | 63.954 | 18.490 | 1.00 | 84.24 | O |
| ATOM | 5 | CB | LYS | A | 217 | −43.440 | 62.182 | 21.587 | 1.00 | 82.52 | C |
| ATOM | 6 | CG | LYS | A | 217 | −44.377 | 61.446 | 22.583 | 1.00 | 83.17 | C |
| ATOM | 7 | CD | LYS | A | 217 | −43.611 | 60.621 | 23.659 | 1.00 | 83.61 | C |
| ATOM | 8 | CE | LYS | A | 217 | −44.624 | 59.772 | 24.504 | 1.00 | 83.43 | C |
| ATOM | 9 | NZ | LYS | A | 217 | −43.959 | 58.869 | 25.546 | 1.00 | 82.62 | N |
| ATOM | 10 | N | GLU | A | 218 | −42.848 | 64.698 | 20.533 | 1.00 | 83.44 | N |
| ATOM | 11 | CA | GLU | A | 218 | −42.096 | 65.806 | 19.972 | 1.00 | 82.60 | C |
| ATOM | 12 | C | GLU | A | 218 | −40.602 | 65.572 | 19.957 | 1.00 | 81.16 | C |
| ATOM | 13 | O | GLU | A | 218 | −39.807 | 66.505 | 19.822 | 1.00 | 80.36 | O |
| ATOM | 14 | CB | GLU | A | 218 | −42.426 | 67.115 | 20.677 | 1.00 | 85.22 | C |
| ATOM | 15 | CG | GLU | A | 218 | −43.490 | 67.880 | 19.918 | 1.00 | 88.47 | C |
| ATOM | 16 | CD | GLU | A | 218 | −43.375 | 67.655 | 18.415 | 1.00 | 90.68 | C |
| ATOM | 17 | OE1 | GLU | A | 218 | −42.311 | 67.980 | 17.838 | 1.00 | 90.61 | O |
| ATOM | 18 | OE2 | GLU | A | 218 | −44.344 | 67.138 | 17.815 | 1.00 | 91.77 | O |
| ATOM | 19 | N | GLN | A | 219 | −40.220 | 64.307 | 20.084 | 1.00 | 79.04 | N |
| ATOM | 20 | CA | GLN | A | 219 | −38.820 | 63.959 | 20.019 | 1.00 | 75.74 | C |
| ATOM | 21 | C | GLN | A | 219 | −38.466 | 64.227 | 18.561 | 1.00 | 73.43 | C |
| ATOM | 22 | O | GLN | A | 219 | −37.313 | 64.122 | 18.153 | 1.00 | 73.75 | O |
| ATOM | 23 | CB | GLN | A | 219 | −38.624 | 62.493 | 20.406 | 1.00 | 76.45 | C |
| ATOM | 24 | CG | GLN | A | 219 | −39.008 | 62.235 | 21.862 | 1.00 | 77.70 | C |
| ATOM | 25 | CD | GLN | A | 219 | −38.674 | 60.831 | 22.340 | 1.00 | 79.40 | C |
| ATOM | 26 | OE1 | GLN | A | 219 | −39.127 | 59.839 | 21.767 | 1.00 | 80.46 | O |
| ATOM | 27 | NE2 | GLN | A | 219 | −37.886 | 60.744 | 23.405 | 1.00 | 79.06 | N |
| ATOM | 28 | N | LEU | A | 220 | −39.488 | 64.583 | 17.782 | 1.00 | 69.99 | N |
| ATOM | 29 | CA | LEU | A | 220 | −39.318 | 64.925 | 16.374 | 1.00 | 66.67 | C |
| ATOM | 30 | C | LEU | A | 220 | −38.622 | 66.272 | 16.345 | 1.00 | 65.57 | C |
| ATOM | 31 | O | LEU | A | 220 | −37.705 | 66.494 | 15.561 | 1.00 | 66.04 | O |
| ATOM | 32 | CB | LEU | A | 220 | −40.669 | 65.061 | 15.660 | 1.00 | 63.76 | C |
| ATOM | 33 | CG | LEU | A | 220 | −41.198 | 63.940 | 14.764 | 1.00 | 60.73 | C |
| ATOM | 34 | CD1 | LEU | A | 220 | −42.450 | 64.437 | 14.059 | 1.00 | 58.24 | C |
| ATOM | 35 | CD2 | LEU | A | 220 | −40.155 | 63.534 | 13.736 | 1.00 | 58.54 | C |
| ATOM | 36 | N | ALA | A | 221 | −39.084 | 67.181 | 17.197 | 1.00 | 65.14 | N |
| ATOM | 37 | CA | ALA | A | 221 | −38.486 | 68.508 | 17.284 | 1.00 | 64.05 | C |
| ATOM | 38 | C | ALA | A | 221 | −37.008 | 68.282 | 17.560 | 1.00 | 62.79 | C |
| ATOM | 39 | O | ALA | A | 221 | −36.142 | 68.992 | 17.050 | 1.00 | 61.55 | O |
| ATOM | 40 | CB | ALA | A | 221 | −39.117 | 69.292 | 18.421 | 1.00 | 63.08 | C |
| ATOM | 41 | N | SER | A | 222 | −36.740 | 67.264 | 18.370 | 1.00 | 62.30 | N |
| ATOM | 42 | CA | SER | A | 222 | −35.385 | 66.894 | 18.733 | 1.00 | 62.18 | C |
| ATOM | 43 | C | SER | A | 222 | −34.601 | 66.442 | 17.504 | 1.00 | 61.96 | C |
| ATOM | 44 | O | SER | A | 222 | −33.577 | 67.032 | 17.153 | 1.00 | 61.52 | O |
| ATOM | 45 | CB | SER | A | 222 | −35.423 | 65.771 | 19.765 | 1.00 | 62.39 | C |
| ATOM | 46 | OG | SER | A | 222 | −34.135 | 65.222 | 19.959 | 1.00 | 65.44 | O |
| ATOM | 47 | N | MET | A | 223 | −35.092 | 65.394 | 16.850 | 1.00 | 61.28 | N |
| ATOM | 48 | CA | MET | A | 223 | −34.439 | 64.859 | 15.665 | 1.00 | 60.63 | C |
| ATOM | 49 | C | MET | A | 223 | −34.252 | 65.933 | 14.579 | 1.00 | 59.30 | C |
| ATOM | 50 | O | MET | A | 223 | −33.278 | 65.900 | 13.825 | 1.00 | 59.97 | O |
| ATOM | 51 | CB | MET | A | 223 | −35.243 | 63.666 | 15.128 | 1.00 | 62.31 | C |
| ATOM | 52 | CG | MET | A | 223 | −35.368 | 62.486 | 16.111 | 1.00 | 66.06 | C |
| ATOM | 53 | SD | MET | A | 223 | −36.340 | 61.046 | 15.520 | 1.00 | 68.19 | S |
| ATOM | 54 | CE | MET | A | 223 | −35.044 | 60.037 | 14.725 | 1.00 | 68.35 | C |
| ATOM | 55 | N | ASN | A | 224 | −35.179 | 66.883 | 14.505 | 1.00 | 57.41 | N |
| ATOM | 56 | CA | ASN | A | 224 | −35.097 | 67.969 | 13.528 | 1.00 | 56.53 | C |
| ATOM | 57 | C | ASN | A | 224 | −34.009 | 68.944 | 13.944 | 1.00 | 55.81 | C |
| ATOM | 58 | O | ASN | A | 224 | −33.334 | 69.540 | 13.106 | 1.00 | 54.94 | O |
| ATOM | 59 | CB | ASN | A | 224 | −36.425 | 68.725 | 13.451 | 1.00 | 57.97 | C |
| ATOM | 60 | CG | ASN | A | 224 | −37.396 | 68.108 | 12.469 | 1.00 | 58.23 | C |
| ATOM | 61 | OD1 | ASN | A | 224 | −37.263 | 68.278 | 11.255 | 1.00 | 58.03 | O |
| ATOM | 62 | ND2 | ASN | A | 224 | −38.376 | 67.380 | 12.987 | 1.00 | 58.36 | N |
| ATOM | 63 | N | ALA | A | 225 | −33.859 | 69.108 | 15.252 | 1.00 | 54.94 | N |
| ATOM | 64 | CA | ALA | A | 225 | −32.865 | 70.012 | 15.803 | 1.00 | 54.50 | C |
| ATOM | 65 | C | ALA | A | 225 | −31.455 | 69.527 | 15.490 | 1.00 | 54.69 | C |
| ATOM | 66 | O | ALA | A | 225 | −30.617 | 70.297 | 15.014 | 1.00 | 54.27 | O |
| ATOM | 67 | CB | ALA | A | 225 | −33.056 | 70.134 | 17.309 | 1.00 | 53.69 | C |
| ATOM | 68 | N | ILE | A | 226 | −31.192 | 68.249 | 15.743 | 1.00 | 53.95 | N |
| ATOM | 69 | CA | ILE | A | 226 | −29.865 | 67.724 | 15.489 | 1.00 | 53.93 | C |
| ATOM | 70 | C | ILE | A | 226 | −29.584 | 67.584 | 14.002 | 1.00 | 54.23 | C |
| ATOM | 71 | O | ILE | A | 226 | −28.429 | 67.484 | 13.593 | 1.00 | 55.16 | O |
| ATOM | 72 | CB | ILE | A | 226 | −29.623 | 66.355 | 16.189 | 1.00 | 54.84 | C |
| ATOM | 73 | CG1 | ILE | A | 226 | −29.672 | 65.225 | 15.167 | 1.00 | 55.69 | C |
| ATOM | 74 | CG2 | ILE | A | 226 | −30.640 | 66.139 | 17.313 | 1.00 | 52.74 | C |
| ATOM | 75 | CD1 | ILE | A | 226 | −29.121 | 63.930 | 15.696 | 1.00 | 59.48 | C |
| ATOM | 76 | N | ALA | A | 227 | −30.632 | 67.575 | 13.187 | 1.00 | 54.60 | N |
| ATOM | 77 | CA | ALA | A | 227 | −30.443 | 67.456 | 11.745 | 1.00 | 53.40 | C |
| ATOM | 78 | C | ALA | A | 227 | −29.932 | 68.791 | 11.214 | 1.00 | 53.46 | C |
| ATOM | 79 | O | ALA | A | 227 | −29.261 | 68.848 | 10.182 | 1.00 | 54.51 | O |
| ATOM | 80 | CB | ALA | A | 227 | −31.760 | 67.084 | 11.066 | 1.00 | 53.97 | C |
| ATOM | 81 | N | ASN | A | 228 | −30.248 | 69.861 | 11.937 | 1.00 | 53.10 | N |
| ATOM | 82 | CA | ASN | A | 228 | −29.842 | 71.209 | 11.558 | 1.00 | 53.44 | C |
| ATOM | 83 | C | ASN | A | 228 | −28.613 | 71.697 | 12.312 | 1.00 | 53.04 | C |
| ATOM | 84 | O | ASN | A | 228 | −28.101 | 72.782 | 12.034 | 1.00 | 53.24 | O |
| ATOM | 85 | CB | ASN | A | 228 | −30.984 | 72.190 | 11.813 | 1.00 | 54.51 | C |
| ATOM | 86 | CG | ASN | A | 228 | −32.162 | 71.961 | 10.899 | 1.00 | 56.90 | C |
| ATOM | 87 | OD1 | ASN | A | 228 | −32.035 | 72.045 | 9.676 | 1.00 | 57.96 | O |
| ATOM | 88 | ND2 | ASN | A | 228 | −33.320 | 71.671 | 11.483 | 1.00 | 56.46 | N |
| ATOM | 89 | N | VAL | A | 229 | −28.143 | 70.907 | 13.269 | 1.00 | 51.83 | N |
| ATOM | 90 | CA | VAL | A | 229 | −26.986 | 71.304 | 14.057 | 1.00 | 50.89 | C |
| ATOM | 91 | C | VAL | A | 229 | −25.763 | 71.528 | 13.171 | 1.00 | 50.42 | C |
| ATOM | 92 | O | VAL | A | 229 | −25.451 | 70.714 | 12.302 | 1.00 | 51.08 | O |
| ATOM | 93 | CB | VAL | A | 229 | −26.645 | 70.236 | 15.121 | 1.00 | 50.12 | C |
| ATOM | 94 | CG1 | VAL | A | 229 | −26.019 | 69.029 | 14.463 | 1.00 | 50.82 | C |
| ATOM | 95 | CG2 | VAL | A | 229 | −25.710 | 70.809 | 16.157 | 1.00 | 51.17 | C |
| ATOM | 96 | N | LYS | A | 230 | −25.084 | 72.648 | 13.371 | 1.00 | 49.26 | N |
| ATOM | 97 | CA | LYS | A | 230 | −23.882 | 72.924 | 12.601 | 1.00 | 49.44 | C |
| ATOM | 98 | C | LYS | A | 230 | −22.720 | 72.455 | 13.472 | 1.00 | 46.91 | C |
| ATOM | 99 | O | LYS | A | 230 | −22.313 | 73.141 | 14.405 | 1.00 | 45.89 | O |
| ATOM | 100 | CB | LYS | A | 230 | −23.760 | 74.421 | 12.309 | 1.00 | 53.01 | C |
| ATOM | 101 | CG | LYS | A | 230 | −24.915 | 74.994 | 11.498 | 1.00 | 57.59 | C |
| ATOM | 102 | CD | LYS | A | 230 | −24.876 | 76.524 | 11.491 | 1.00 | 62.19 | C |
| ATOM | 103 | CE | LYS | A | 230 | −26.121 | 77.120 | 10.819 | 1.00 | 64.18 | C |
| ATOM | 104 | NZ | LYS | A | 230 | −26.145 | 78.612 | 10.897 | 1.00 | 63.70 | N |
| ATOM | 105 | N | ALA | A | 231 | −22.213 | 71.264 | 13.184 | 1.00 | 44.00 | N |
| ATOM | 106 | CA | ALA | A | 231 | −21.106 | 70.712 | 13.946 | 1.00 | 42.61 | C |
| ATOM | 107 | C | ALA | A | 231 | −19.799 | 71.103 | 13.265 | 1.00 | 41.24 | C |
| ATOM | 108 | O | ALA | A | 231 | −19.509 | 70.661 | 12.150 | 1.00 | 41.29 | O |
| ATOM | 109 | CB | ALA | A | 231 | −21.234 | 69.193 | 14.026 | 1.00 | 40.33 | C |
| ATOM | 110 | N | THR | A | 232 | −19.014 | 71.937 | 13.937 | 1.00 | 38.80 | N |
| ATOM | 111 | CA | THR | A | 232 | −17.747 | 72.385 | 13.379 | 1.00 | 38.06 | C |
| ATOM | 112 | C | THR | A | 232 | −16.526 | 71.800 | 14.080 | 1.00 | 36.79 | C |
| ATOM | 113 | O | THR | A | 232 | −16.407 | 71.867 | 15.304 | 1.00 | 37.42 | O |
| ATOM | 114 | CB | THR | A | 232 | −17.637 | 73.918 | 13.431 | 1.00 | 38.39 | C |
| ATOM | 115 | OG1 | THR | A | 232 | −18.659 | 74.497 | 12.608 | 1.00 | 41.17 | O |
| ATOM | 116 | CG2 | THR | A | 232 | −16.263 | 74.375 | 12.940 | 1.00 | 35.47 | C |
| ATOM | 117 | N | TYR | A | 233 | −15.624 | 71.225 | 13.292 | 1.00 | 35.83 | N |
| ATOM | 118 | CA | TYR | A | 233 | −14.384 | 70.660 | 13.809 | 1.00 | 36.14 | C |
| ATOM | 119 | C | TYR | A | 233 | −13.212 | 71.597 | 13.523 | 1.00 | 36.12 | C |
| ATOM | 120 | O | TYR | A | 233 | −13.135 | 72.207 | 12.455 | 1.00 | 35.82 | O |
| ATOM | 121 | CB | TYR | A | 233 | −14.074 | 69.322 | 13.144 | 1.00 | 34.69 | C |
| ATOM | 122 | CG | TYR | A | 233 | −14.400 | 68.122 | 13.983 | 1.00 | 37.00 | C |
| ATOM | 123 | CD1 | TYR | A | 233 | −13.748 | 67.894 | 15.193 | 1.00 | 36.01 | C |
| ATOM | 124 | CD2 | TYR | A | 233 | −15.361 | 67.198 | 13.563 | 1.00 | 35.56 | C |
| ATOM | 125 | CE1 | TYR | A | 233 | −14.050 | 66.766 | 15.971 | 1.00 | 35.79 | C |
| ATOM | 126 | CE2 | TYR | A | 233 | −15.665 | 66.077 | 14.325 | 1.00 | 35.96 | C |
| ATOM | 127 | CZ | TYR | A | 233 | −15.013 | 65.867 | 15.523 | 1.00 | 35.81 | C |
| ATOM | 128 | OH | TYR | A | 233 | −15.340 | 64.767 | 16.271 | 1.00 | 36.08 | O |
| ATOM | 129 | N | SER | A | 234 | −12.296 | 71.700 | 14.477 | 1.00 | 36.02 | N |
| ATOM | 130 | CA | SER | A | 234 | −11.108 | 72.519 | 14.299 | 1.00 | 34.94 | C |
| ATOM | 131 | C | SER | A | 234 | −9.950 | 71.551 | 14.519 | 1.00 | 34.26 | C |
| ATOM | 132 | O | SER | A | 234 | −9.672 | 71.167 | 15.648 | 1.00 | 34.99 | O |
| ATOM | 133 | CB | SER | A | 234 | −11.080 | 73.645 | 15.330 | 1.00 | 35.36 | C |
| ATOM | 134 | OG | SER | A | 234 | −9.923 | 74.444 | 15.174 | 1.00 | 38.43 | O |
| ATOM | 135 | N | ILE | A | 235 | −9.292 | 71.146 | 13.438 | 1.00 | 33.19 | N |
| ATOM | 136 | CA | ILE | A | 235 | −8.194 | 70.196 | 13.527 | 1.00 | 33.60 | C |
| ATOM | 137 | C | ILE | A | 235 | −6.962 | 70.677 | 12.785 | 1.00 | 34.63 | C |
| ATOM | 138 | O | ILE | A | 235 | −7.030 | 71.045 | 11.617 | 1.00 | 34.47 | O |
| ATOM | 139 | CB | ILE | A | 235 | −8.578 | 68.823 | 12.916 | 1.00 | 35.60 | C |
| ATOM | 140 | CG1 | ILE | A | 235 | −9.872 | 68.301 | 13.542 | 1.00 | 36.06 | C |
| ATOM | 141 | CG2 | ILE | A | 235 | −7.463 | 67.823 | 13.143 | 1.00 | 32.96 | C |
| ATOM | 142 | CD1 | ILE | A | 235 | −10.293 | 66.956 | 13.003 | 1.00 | 33.64 | C |
| ATOM | 143 | N | ASN | A | 236 | −5.832 | 70.643 | 13.480 | 1.00 | 36.26 | N |
| ATOM | 144 | CA | ASN | A | 236 | −4.547 | 71.046 | 12.931 | 1.00 | 35.55 | C |
| ATOM | 145 | C | ASN | A | 236 | −4.632 | 72.376 | 12.190 | 1.00 | 37.34 | C |
| ATOM | 146 | O | ASN | A | 236 | −3.981 | 72.574 | 11.159 | 1.00 | 35.11 | O |
| ATOM | 147 | CB | ASN | A | 236 | −4.010 | 69.951 | 12.004 | 1.00 | 35.32 | C |
| ATOM | 148 | CG | ASN | A | 236 | −2.504 | 69.964 | 11.915 | 1.00 | 35.32 | C |
| ATOM | 149 | OD1 | ASN | A | 236 | −1.845 | 70.542 | 12.764 | 1.00 | 38.06 | O |
| ATOM | 150 | ND2 | ASN | A | 236 | −1.951 | 69.320 | 10.897 | 1.00 | 38.61 | N |
| ATOM | 151 | N | GLY | A | 237 | −5.451 | 73.281 | 12.719 | 1.00 | 39.16 | N |
| ATOM | 152 | CA | GLY | A | 237 | −5.589 | 74.592 | 12.112 | 1.00 | 41.64 | C |
| ATOM | 153 | C | GLY | A | 237 | −6.636 | 74.712 | 11.021 | 1.00 | 43.21 | C |
| ATOM | 154 | O | GLY | A | 237 | −6.908 | 75.817 | 10.555 | 1.00 | 44.40 | O |
| ATOM | 155 | N | GLU | A | 238 | −7.216 | 73.591 | 10.602 | 1.00 | 43.89 | N |
| ATOM | 156 | CA | GLU | A | 238 | −8.239 | 73.607 | 9.559 | 1.00 | 45.26 | C |
| ATOM | 157 | C | GLU | A | 238 | −9.642 | 73.458 | 10.140 | 1.00 | 44.63 | C |
| ATOM | 158 | O | GLU | A | 238 | −9.884 | 72.652 | 11.044 | 1.00 | 45.76 | O |
| ATOM | 159 | CB | GLU | A | 238 | −7.991 | 72.492 | 8.535 | 1.00 | 47.99 | C |
| ATOM | 160 | CG | GLU | A | 238 | −6.828 | 72.749 | 7.581 | 1.00 | 54.04 | C |
| ATOM | 161 | CD | GLU | A | 238 | −6.988 | 74.046 | 6.788 | 1.00 | 59.58 | C |
| ATOM | 162 | OE1 | GLU | A | 238 | −7.991 | 74.174 | 6.044 | 1.00 | 61.15 | O |
| ATOM | 163 | OE2 | GLU | A | 238 | −6.113 | 74.939 | 6.908 | 1.00 | 60.19 | O |
| ATOM | 164 | N | THR | A | 239 | −10.566 | 74.245 | 9.611 | 1.00 | 42.88 | N |
| ATOM | 165 | CA | THR | A | 239 | −11.949 | 74.215 | 10.060 | 1.00 | 42.88 | C |
| ATOM | 166 | C | THR | A | 239 | −12.885 | 73.705 | 8.962 | 1.00 | 41.20 | C |
| ATOM | 167 | O | THR | A | 239 | −12.721 | 74.031 | 7.788 | 1.00 | 40.40 | O |
| ATOM | 168 | CB | THR | A | 239 | −12.391 | 75.625 | 10.513 | 1.00 | 43.23 | C |
| ATOM | 169 | OG1 | THR | A | 239 | −11.761 | 75.931 | 11.760 | 1.00 | 46.39 | O |
| ATOM | 170 | CG2 | THR | A | 239 | −13.893 | 75.705 | 10.689 | 1.00 | 45.31 | C |
| ATOM | 171 | N | PHE | A | 240 | −13.856 | 72.887 | 9.353 | 1.00 | 40.69 | N |
| ATOM | 172 | CA | PHE | A | 240 | −14.831 | 72.346 | 8.415 | 1.00 | 39.35 | C |
| ATOM | 173 | C | PHE | A | 240 | −16.073 | 71.918 | 9.183 | 1.00 | 40.46 | C |
| ATOM | 174 | O | PHE | A | 240 | −16.025 | 71.719 | 10.399 | 1.00 | 39.84 | O |
| ATOM | 175 | CB | PHE | A | 240 | −14.245 | 71.161 | 7.646 | 1.00 | 36.31 | C |
| ATOM | 176 | CG | PHE | A | 240 | −13.924 | 69.970 | 8.507 | 1.00 | 37.57 | C |
| ATOM | 177 | CD1 | PHE | A | 240 | −14.932 | 69.114 | 8.945 | 1.00 | 34.59 | C |
| ATOM | 178 | CD2 | PHE | A | 240 | −12.605 | 69.693 | 8.868 | 1.00 | 36.56 | C |
| ATOM | 179 | CE1 | PHE | A | 240 | −14.633 | 68.000 | 9.725 | 1.00 | 35.89 | C |
| ATOM | 180 | CE2 | PHE | A | 240 | −12.292 | 68.576 | 9.651 | 1.00 | 35.71 | C |
| ATOM | 181 | CZ | PHE | A | 240 | −13.306 | 67.728 | 10.080 | 1.00 | 34.95 | C |
| ATOM | 182 | N | GLN | A | 241 | −17.188 | 71.798 | 8.472 | 1.00 | 41.65 | N |
| ATOM | 183 | CA | GLN | A | 241 | −18.449 | 71.394 | 9.082 | 1.00 | 43.09 | C |
| ATOM | 184 | C | GLN | A | 241 | −18.780 | 69.953 | 8.720 | 1.00 | 41.59 | C |
| ATOM | 185 | O | GLN | A | 241 | −18.488 | 69.503 | 7.613 | 1.00 | 40.76 | O |
| ATOM | 186 | CB | GLN | A | 241 | −19.583 | 72.281 | 8.580 | 1.00 | 45.92 | C |
| ATOM | 187 | CG | GLN | A | 241 | −20.093 | 73.299 | 9.571 | 1.00 | 53.01 | C |
| ATOM | 188 | CD | GLN | A | 241 | −21.268 | 74.097 | 9.017 | 1.00 | 55.83 | C |
| ATOM | 189 | OE1 | GLN | A | 241 | −22.300 | 73.531 | 8.627 | 1.00 | 57.15 | O |
| ATOM | 190 | NE2 | GLN | A | 241 | −21.117 | 75.417 | 8.979 | 1.00 | 57.90 | N |
| ATOM | 191 | N | ILE | A | 242 | −19.375 | 69.226 | 9.657 | 1.00 | 41.04 | N |
| ATOM | 192 | CA | ILE | A | 242 | −19.785 | 67.861 | 9.374 | 1.00 | 40.35 | C |
| ATOM | 193 | C | ILE | A | 242 | −21.034 | 68.029 | 8.506 | 1.00 | 41.27 | C |
| ATOM | 194 | O | ILE | A | 242 | −21.995 | 68.685 | 8.911 | 1.00 | 41.46 | O |
| ATOM | 195 | CB | ILE | A | 242 | −20.160 | 67.090 | 10.658 | 1.00 | 39.32 | C |
| ATOM | 196 | CG1 | ILE | A | 242 | −18.928 | 66.923 | 11.551 | 1.00 | 38.14 | C |
| ATOM | 197 | CG2 | ILE | A | 242 | −20.729 | 65.722 | 10.292 | 1.00 | 38.76 | C |
| ATOM | 198 | CD1 | ILE | A | 242 | −19.195 | 66.151 | 12.839 | 1.00 | 36.01 | C |
| ATOM | 199 | N | PRO | A | 243 | −21.026 | 67.459 | 7.293 | 1.00 | 41.52 | N |
| ATOM | 200 | CA | PRO | A | 243 | −22.173 | 67.566 | 6.382 | 1.00 | 42.77 | C |
| ATOM | 201 | C | PRO | A | 243 | −23.476 | 67.116 | 7.033 | 1.00 | 43.70 | C |
| ATOM | 202 | O | PRO | A | 243 | −23.504 | 66.102 | 7.725 | 1.00 | 44.08 | O |
| ATOM | 203 | CB | PRO | A | 243 | −21.777 | 66.658 | 5.219 | 1.00 | 41.94 | C |
| ATOM | 204 | CG | PRO | A | 243 | −20.275 | 66.744 | 5.221 | 1.00 | 42.17 | C |
| ATOM | 205 | CD | PRO | A | 243 | −19.951 | 66.652 | 6.691 | 1.00 | 41.28 | C |
| ATOM | 206 | N | SER | A | 244 | −24.546 | 67.874 | 6.810 | 1.00 | 45.84 | N |
| ATOM | 207 | CA | SER | A | 244 | −25.858 | 67.542 | 7.362 | 1.00 | 47.86 | C |
| ATOM | 208 | C | SER | A | 244 | −26.273 | 66.138 | 6.931 | 1.00 | 48.51 | C |
| ATOM | 209 | O | SER | A | 244 | −26.875 | 65.389 | 7.699 | 1.00 | 46.87 | O |
| ATOM | 210 | CB | SER | A | 244 | −26.895 | 68.545 | 6.869 | 1.00 | 49.18 | C |
| ATOM | 211 | OG | SER | A | 244 | −26.435 | 69.863 | 7.096 | 1.00 | 54.30 | O |
| ATOM | 212 | N | SER | A | 245 | −25.937 | 65.787 | 5.695 | 1.00 | 50.26 | N |
| ATOM | 213 | CA | SER | A | 245 | −26.273 | 64.480 | 5.159 | 1.00 | 52.34 | C |
| ATOM | 214 | C | SER | A | 245 | −25.682 | 63.349 | 6.003 | 1.00 | 52.83 | C |
| ATOM | 215 | O | SER | A | 245 | −26.308 | 62.290 | 6.143 | 1.00 | 52.84 | O |
| ATOM | 216 | CB | SER | A | 245 | −25.797 | 64.363 | 3.706 | 1.00 | 53.94 | C |
| ATOM | 217 | OG | SER | A | 245 | −24.386 | 64.438 | 3.615 | 1.00 | 57.57 | O |
| ATOM | 218 | N | ASP | A | 246 | −24.486 | 63.556 | 6.557 | 1.00 | 52.04 | N |
| ATOM | 219 | CA | ASP | A | 246 | −23.877 | 62.523 | 7.395 | 1.00 | 52.85 | C |
| ATOM | 220 | C | ASP | A | 246 | −24.645 | 62.437 | 8.716 | 1.00 | 52.13 | C |
| ATOM | 221 | O | ASP | A | 246 | −24.908 | 61.347 | 9.231 | 1.00 | 49.39 | O |
| ATOM | 222 | CB | ASP | A | 246 | −22.403 | 62.824 | 7.689 | 1.00 | 54.28 | C |
| ATOM | 223 | CG | ASP | A | 246 | −21.521 | 62.690 | 6.467 | 1.00 | 58.27 | C |
| ATOM | 224 | OD1 | ASP | A | 246 | −21.869 | 61.912 | 5.551 | 1.00 | 60.24 | O |
| ATOM | 225 | OD2 | ASP | A | 246 | −20.464 | 63.354 | 6.433 | 1.00 | 59.13 | O |
| ATOM | 226 | N | ILE | A | 247 | −24.997 | 63.596 | 9.259 | 1.00 | 50.02 | N |
| ATOM | 227 | CA | ILE | A | 247 | −25.736 | 63.642 | 10.504 | 1.00 | 51.05 | C |
| ATOM | 228 | C | ILE | A | 247 | −27.089 | 62.946 | 10.339 | 1.00 | 52.45 | C |
| ATOM | 229 | O | ILE | A | 247 | −27.507 | 62.172 | 11.201 | 1.00 | 51.13 | O |
| ATOM | 230 | CB | ILE | A | 247 | −25.958 | 65.102 | 10.959 | 1.00 | 49.62 | C |
| ATOM | 231 | CG1 | ILE | A | 247 | −24.610 | 65.730 | 11.336 | 1.00 | 49.23 | C |
| ATOM | 232 | CG2 | ILE | A | 247 | −26.933 | 65.143 | 12.133 | 1.00 | 47.64 | C |
| ATOM | 233 | CD1 | ILE | A | 247 | −24.682 | 67.193 | 11.692 | 1.00 | 46.92 | C |
| ATOM | 234 | N | MET | A | 248 | −27.763 | 63.218 | 9.226 | 1.00 | 53.09 | N |
| ATOM | 235 | CA | MET | A | 248 | −29.060 | 62.615 | 8.968 | 1.00 | 54.82 | C |
| ATOM | 236 | C | MET | A | 248 | −28.943 | 61.097 | 8.811 | 1.00 | 54.48 | C |
| ATOM | 237 | O | MET | A | 248 | −29.843 | 60.358 | 9.210 | 1.00 | 54.01 | O |
| ATOM | 238 | CB | MET | A | 248 | −29.698 | 63.247 | 7.727 | 1.00 | 56.26 | C |
| ATOM | 239 | CG | MET | A | 248 | −29.818 | 64.766 | 7.835 | 1.00 | 60.90 | C |
| ATOM | 240 | SD | MET | A | 248 | −30.975 | 65.552 | 6.673 | 1.00 | 66.36 | S |
| ATOM | 241 | CE | MET | A | 248 | −29.875 | 65.940 | 5.290 | 1.00 | 65.53 | C |
| ATOM | 242 | N | SER | A | 249 | −27.830 | 60.634 | 8.251 | 1.00 | 53.21 | N |
| ATOM | 243 | CA | SER | A | 249 | −27.627 | 59.203 | 8.069 | 1.00 | 54.12 | C |
| ATOM | 244 | C | SER | A | 249 | −27.179 | 58.520 | 9.358 | 1.00 | 53.57 | C |
| ATOM | 245 | O | SER | A | 249 | −27.131 | 57.295 | 9.431 | 1.00 | 54.90 | O |
| ATOM | 246 | CB | SER | A | 249 | −26.611 | 58.936 | 6.951 | 1.00 | 54.82 | C |
| ATOM | 247 | OG | SER | A | 249 | −25.387 | 59.604 | 7.186 | 1.00 | 59.04 | O |
| ATOM | 248 | N | TRP | A | 250 | −26.846 | 59.312 | 10.371 | 1.00 | 52.21 | N |
| ATOM | 249 | CA | TRP | A | 250 | −26.428 | 58.760 | 11.655 | 1.00 | 51.88 | C |
| ATOM | 250 | C | TRP | A | 250 | −27.644 | 58.747 | 12.559 | 1.00 | 52.05 | C |
| ATOM | 251 | O | TRP | A | 250 | −27.731 | 57.962 | 13.508 | 1.00 | 48.90 | O |
| ATOM | 252 | CB | TRP | A | 250 | −25.360 | 59.629 | 12.315 | 1.00 | 51.69 | C |
| ATOM | 253 | CG | TRP | A | 250 | −24.086 | 59.741 | 11.567 | 1.00 | 52.58 | C |
| ATOM | 254 | CD1 | TRP | A | 250 | −23.652 | 58.939 | 10.552 | 1.00 | 53.06 | C |
| ATOM | 255 | CD2 | TRP | A | 250 | −23.041 | 60.688 | 11.810 | 1.00 | 52.72 | C |
| ATOM | 256 | NE1 | TRP | A | 250 | −22.397 | 59.329 | 10.147 | 1.00 | 53.59 | N |
| ATOM | 257 | CE2 | TRP | A | 250 | −21.998 | 60.400 | 10.904 | 1.00 | 53.78 | C |
| ATOM | 258 | CE3 | TRP | A | 250 | −22.885 | 61.751 | 12.710 | 1.00 | 52.08 | C |
| ATOM | 259 | CZ2 | TRP | A | 250 | −20.810 | 61.140 | 10.869 | 1.00 | 53.32 | C |
| ATOM | 260 | CZ3 | TRP | A | 250 | −21.702 | 62.486 | 12.676 | 1.00 | 53.95 | C |
| ATOM | 261 | CH2 | TRP | A | 250 | −20.681 | 62.176 | 11.760 | 1.00 | 53.18 | C |
| ATOM | 262 | N | LEU | A | 251 | −28.567 | 59.657 | 12.264 | 1.00 | 53.27 | N |
| ATOM | 263 | CA | LEU | A | 251 | −29.797 | 59.786 | 13.021 | 1.00 | 54.79 | C |
| ATOM | 264 | C | LEU | A | 251 | −30.514 | 58.451 | 13.079 | 1.00 | 56.13 | C |
| ATOM | 265 | O | LEU | A | 251 | −30.836 | 57.846 | 12.053 | 1.00 | 54.70 | O |
| ATOM | 266 | CB | LEU | A | 251 | −30.711 | 60.825 | 12.380 | 1.00 | 54.38 | C |
| ATOM | 267 | CG | LEU | A | 251 | −30.829 | 62.136 | 13.149 | 1.00 | 54.64 | C |
| ATOM | 268 | CD1 | LEU | A | 251 | −31.742 | 63.071 | 12.385 | 1.00 | 57.58 | C |
| ATOM | 269 | CD2 | LEU | A | 251 | −31.375 | 61.874 | 14.540 | 1.00 | 54.44 | C |
| ATOM | 270 | N | THR | A | 252 | −30.748 | 57.993 | 14.298 | 1.00 | 57.32 | N |
| ATOM | 271 | CA | THR | A | 252 | −31.428 | 56.736 | 14.515 | 1.00 | 58.41 | C |
| ATOM | 272 | C | THR | A | 252 | −32.483 | 56.963 | 15.577 | 1.00 | 59.55 | C |
| ATOM | 273 | O | THR | A | 252 | −32.652 | 58.072 | 16.080 | 1.00 | 59.22 | O |
| ATOM | 274 | CB | THR | A | 252 | −30.451 | 55.649 | 15.001 | 1.00 | 57.97 | C |
| ATOM | 275 | OG1 | THR | A | 252 | −31.137 | 54.395 | 15.086 | 1.00 | 59.52 | O |
| ATOM | 276 | CG2 | THR | A | 252 | −29.905 | 56.000 | 16.369 | 1.00 | 55.46 | C |
| ATOM | 277 | N | TYR | A | 253 | −33.191 | 55.902 | 15.917 | 1.00 | 61.60 | N |
| ATOM | 278 | CA | TYR | A | 253 | −34.220 | 55.993 | 16.922 | 1.00 | 62.61 | C |
| ATOM | 279 | C | TYR | A | 253 | −34.480 | 54.610 | 17.478 | 1.00 | 63.93 | C |
| ATOM | 280 | O | TYR | A | 253 | −34.840 | 53.694 | 16.744 | 1.00 | 64.25 | O |
| ATOM | 281 | CB | TYR | A | 253 | −35.491 | 56.577 | 16.310 | 1.00 | 63.09 | C |
| ATOM | 282 | CG | TYR | A | 253 | −36.612 | 56.724 | 17.302 | 1.00 | 65.11 | C |
| ATOM | 283 | CD1 | TYR | A | 253 | −37.320 | 55.608 | 17.751 | 1.00 | 66.49 | C |
| ATOM | 284 | CD2 | TYR | A | 253 | −36.936 | 57.967 | 17.833 | 1.00 | 64.95 | C |
| ATOM | 285 | CE1 | TYR | A | 253 | −38.316 | 55.725 | 18.703 | 1.00 | 66.68 | C |
| ATOM | 286 | CE2 | TYR | A | 253 | −37.931 | 58.096 | 18.787 | 1.00 | 66.45 | C |
| ATOM | 287 | CZ | TYR | A | 253 | −38.614 | 56.971 | 19.218 | 1.00 | 67.69 | C |
| ATOM | 288 | OH | TYR | A | 253 | −39.587 | 57.087 | 20.179 | 1.00 | 71.21 | O |
| ATOM | 289 | N | ASN | A | 254 | −34.260 | 54.453 | 18.775 | 1.00 | 66.00 | N |
| ATOM | 290 | CA | ASN | A | 254 | −34.502 | 53.183 | 19.443 | 1.00 | 68.88 | C |
| ATOM | 291 | C | ASN | A | 254 | −34.595 | 53.411 | 20.946 | 1.00 | 69.71 | C |
| ATOM | 292 | O | ASN | A | 254 | −34.009 | 54.356 | 21.481 | 1.00 | 69.16 | O |
| ATOM | 293 | CB | ASN | A | 254 | −33.418 | 52.155 | 19.088 | 1.00 | 70.38 | C |
| ATOM | 294 | CG | ASN | A | 254 | −32.027 | 52.681 | 19.292 | 1.00 | 72.63 | C |
| ATOM | 295 | OD1 | ASN | A | 254 | −31.632 | 53.672 | 18.681 | 1.00 | 74.69 | O |
| ATOM | 296 | ND2 | ASN | A | 254 | −31.265 | 52.019 | 20.154 | 1.00 | 72.90 | N |
| ATOM | 297 | N | ASP | A | 255 | −35.351 | 52.547 | 21.616 | 1.00 | 71.19 | N |
| ATOM | 298 | CA | ASP | A | 255 | −35.592 | 52.665 | 23.048 | 1.00 | 71.79 | C |
| ATOM | 299 | C | ASP | A | 255 | −36.333 | 53.970 | 23.305 | 1.00 | 71.07 | C |
| ATOM | 300 | O | ASP | A | 255 | −36.157 | 54.615 | 24.337 | 1.00 | 71.92 | O |
| ATOM | 301 | CB | ASP | A | 255 | −34.286 | 52.626 | 23.839 | 1.00 | 74.27 | C |
| ATOM | 302 | CG | ASP | A | 255 | −33.758 | 51.204 | 24.017 | 1.00 | 77.68 | C |
| ATOM | 303 | OD1 | ASP | A | 255 | −33.032 | 50.689 | 23.083 | 1.00 | 78.50 | O |
| ATOM | 304 | OD2 | ASP | A | 255 | −34.082 | 50.579 | 25.090 | 1.00 | 78.01 | O |
| ATOM | 305 | N | GLY | A | 256 | −37.164 | 54.347 | 22.341 | 1.00 | 71.07 | N |
| ATOM | 306 | CA | GLY | A | 256 | −37.948 | 55.562 | 22.452 | 1.00 | 71.30 | C |
| ATOM | 307 | C | GLY | A | 256 | −37.114 | 56.823 | 22.506 | 1.00 | 71.57 | C |
| ATOM | 308 | O | GLY | A | 256 | −37.644 | 57.914 | 22.729 | 1.00 | 71.59 | O |
| ATOM | 309 | N | LYS | A | 257 | −35.811 | 56.686 | 22.273 | 1.00 | 71.70 | N |
| ATOM | 310 | CA | LYS | A | 257 | −34.910 | 57.834 | 22.330 | 1.00 | 71.90 | C |
| ATOM | 311 | C | LYS | A | 257 | −34.285 | 58.199 | 20.990 | 1.00 | 69.53 | C |
| ATOM | 312 | O | LYS | A | 257 | −33.988 | 57.326 | 20.174 | 1.00 | 69.91 | O |
| ATOM | 313 | CB | LYS | A | 257 | −33.779 | 57.551 | 23.333 | 1.00 | 73.77 | C |
| ATOM | 314 | CG | LYS | A | 257 | −34.265 | 56.896 | 24.662 | 1.00 | 75.97 | C |
| ATOM | 315 | CD | LYS | A | 257 | −33.113 | 56.088 | 25.311 | 1.00 | 77.56 | C |
| ATOM | 316 | CE | LYS | A | 257 | −33.545 | 55.496 | 26.680 | 1.00 | 78.79 | C |
| ATOM | 317 | NZ | LYS | A | 257 | −32.343 | 54.836 | 27.390 | 1.00 | 78.47 | N |
| ATOM | 318 | N | VAL | A | 258 | −34.092 | 59.496 | 20.769 | 1.00 | 66.69 | N |
| ATOM | 319 | CA | VAL | A | 258 | −33.437 | 59.960 | 19.554 | 1.00 | 64.09 | C |
| ATOM | 320 | C | VAL | A | 258 | −31.967 | 59.618 | 19.798 | 1.00 | 61.80 | C |
| ATOM | 321 | O | VAL | A | 258 | −31.478 | 59.771 | 20.919 | 1.00 | 61.05 | O |
| ATOM | 322 | CB | VAL | A | 258 | −33.568 | 61.476 | 19.397 | 1.00 | 64.89 | C |
| ATOM | 323 | CG1 | VAL | A | 258 | −32.958 | 61.914 | 18.072 | 1.00 | 64.64 | C |
| ATOM | 324 | CG2 | VAL | A | 258 | −35.026 | 61.877 | 19.497 | 1.00 | 64.95 | C |
| ATOM | 325 | N | ASP | A | 259 | −31.261 | 59.148 | 18.776 | 1.00 | 59.42 | N |
| ATOM | 326 | CA | ASP | A | 259 | −29.860 | 58.775 | 18.971 | 1.00 | 56.66 | C |
| ATOM | 327 | C | ASP | A | 259 | −29.048 | 58.854 | 17.676 | 1.00 | 52.90 | C |
| ATOM | 328 | O | ASP | A | 259 | −29.531 | 59.332 | 16.649 | 1.00 | 52.53 | O |
| ATOM | 329 | CB | ASP | A | 259 | −29.792 | 57.346 | 19.533 | 1.00 | 58.54 | C |
| ATOM | 330 | CG | ASP | A | 259 | −28.554 | 57.101 | 20.385 | 1.00 | 61.39 | C |
| ATOM | 331 | OD1 | ASP | A | 259 | −27.477 | 57.652 | 20.068 | 1.00 | 63.40 | O |
| ATOM | 332 | OD2 | ASP | A | 259 | −28.657 | 56.339 | 21.370 | 1.00 | 62.27 | O |
| ATOM | 333 | N | LEU | A | 260 | −27.806 | 58.388 | 17.741 | 1.00 | 49.40 | N |
| ATOM | 334 | CA | LEU | A | 260 | −26.925 | 58.369 | 16.580 | 1.00 | 46.91 | C |
| ATOM | 335 | C | LEU | A | 260 | −26.315 | 56.983 | 16.462 | 1.00 | 46.30 | C |
| ATOM | 336 | O | LEU | A | 260 | −25.914 | 56.388 | 17.461 | 1.00 | 46.69 | O |
| ATOM | 337 | CB | LEU | A | 260 | −25.799 | 59.396 | 16.721 | 1.00 | 45.11 | C |
| ATOM | 338 | CG | LEU | A | 260 | −26.149 | 60.879 | 16.704 | 1.00 | 43.08 | C |
| ATOM | 339 | CD1 | LEU | A | 260 | −24.866 | 61.682 | 16.679 | 1.00 | 43.76 | C |
| ATOM | 340 | CD2 | LEU | A | 260 | −26.990 | 61.198 | 15.493 | 1.00 | 42.43 | C |
| ATOM | 341 | N | ASP | A | 261 | −26.258 | 56.465 | 15.241 | 1.00 | 46.92 | N |
| ATOM | 342 | CA | ASP | A | 261 | −25.675 | 55.155 | 15.012 | 1.00 | 47.47 | C |
| ATOM | 343 | C | ASP | A | 261 | −24.202 | 55.254 | 15.417 | 1.00 | 47.68 | C |
| ATOM | 344 | O | ASP | A | 261 | −23.352 | 55.696 | 14.643 | 1.00 | 46.88 | O |
| ATOM | 345 | CB | ASP | A | 261 | −25.799 | 54.774 | 13.536 | 1.00 | 47.57 | C |
| ATOM | 346 | CG | ASP | A | 261 | −25.269 | 53.383 | 13.245 | 1.00 | 50.74 | C |
| ATOM | 347 | OD1 | ASP | A | 261 | −24.666 | 52.766 | 14.153 | 1.00 | 51.47 | O |
| ATOM | 348 | OD2 | ASP | A | 261 | −25.453 | 52.910 | 12.100 | 1.00 | 52.80 | O |
| ATOM | 349 | N | THR | A | 262 | −23.916 | 54.849 | 16.646 | 1.00 | 47.65 | N |
| ATOM | 350 | CA | THR | A | 262 | −22.565 | 54.900 | 17.179 | 1.00 | 49.76 | C |
| ATOM | 351 | C | THR | A | 262 | −21.538 | 54.244 | 16.262 | 1.00 | 50.93 | C |
| ATOM | 352 | O | THR | A | 262 | −20.364 | 54.615 | 16.257 | 1.00 | 50.67 | O |
| ATOM | 353 | CB | THR | A | 262 | −22.503 | 54.228 | 18.559 | 1.00 | 48.62 | C |
| ATOM | 354 | OG1 | THR | A | 262 | −23.442 | 54.864 | 19.433 | 1.00 | 50.46 | O |
| ATOM | 355 | CG2 | THR | A | 262 | −21.103 | 54.353 | 19.157 | 1.00 | 48.56 | C |
| ATOM | 356 | N | GLU | A | 263 | −21.971 | 53.273 | 15.476 | 1.00 | 52.15 | N |
| ATOM | 357 | CA | GLU | A | 263 | −21.032 | 52.611 | 14.599 | 1.00 | 53.23 | C |
| ATOM | 358 | C | GLU | A | 263 | −20.587 | 53.569 | 13.511 | 1.00 | 50.93 | C |
| ATOM | 359 | O | GLU | A | 263 | −19.404 | 53.637 | 13.182 | 1.00 | 49.35 | O |
| ATOM | 360 | CB | GLU | A | 263 | −21.669 | 51.380 | 13.970 | 1.00 | 57.96 | C |
| ATOM | 361 | CG | GLU | A | 263 | −20.654 | 50.421 | 13.402 | 1.00 | 65.90 | C |
| ATOM | 362 | CD | GLU | A | 263 | −21.295 | 49.269 | 12.663 | 1.00 | 70.83 | C |
| ATOM | 363 | OE1 | GLU | A | 263 | −21.826 | 49.502 | 11.551 | 1.00 | 73.87 | O |
| ATOM | 364 | OE2 | GLU | A | 263 | −21.273 | 48.137 | 13.198 | 1.00 | 72.91 | O |
| ATOM | 365 | N | GLN | A | 264 | −21.541 | 54.313 | 12.958 | 1.00 | 48.84 | N |
| ATOM | 366 | CA | GLN | A | 264 | −21.247 | 55.257 | 11.889 | 1.00 | 47.72 | C |
| ATOM | 367 | C | GLN | A | 264 | −20.432 | 56.444 | 12.386 | 1.00 | 46.89 | C |
| ATOM | 368 | O | GLN | A | 264 | −19.515 | 56.912 | 11.707 | 1.00 | 47.36 | O |
| ATOM | 369 | CB | GLN | A | 264 | −22.543 | 55.745 | 11.235 | 1.00 | 48.20 | C |
| ATOM | 370 | CG | GLN | A | 264 | −23.278 | 54.667 | 10.457 | 1.00 | 49.05 | C |
| ATOM | 371 | CD | GLN | A | 264 | −24.523 | 55.191 | 9.758 | 1.00 | 52.09 | C |
| ATOM | 372 | OE1 | GLN | A | 264 | −24.441 | 56.042 | 8.867 | 1.00 | 51.23 | O |
| ATOM | 373 | NE2 | GLN | A | 264 | −25.687 | 54.682 | 10.162 | 1.00 | 52.53 | N |
| ATOM | 374 | N | VAL | A | 265 | −20.766 | 56.930 | 13.571 | 1.00 | 44.57 | N |
| ATOM | 375 | CA | VAL | A | 265 | −20.040 | 58.046 | 14.134 | 1.00 | 43.15 | C |
| ATOM | 376 | C | VAL | A | 265 | −18.593 | 57.608 | 14.390 | 1.00 | 43.97 | C |
| ATOM | 377 | O | VAL | A | 265 | −17.650 | 58.356 | 14.109 | 1.00 | 43.62 | O |
| ATOM | 378 | CB | VAL | A | 265 | −20.685 | 58.512 | 15.446 | 1.00 | 42.48 | C |
| ATOM | 379 | CG1 | VAL | A | 265 | −19.918 | 59.716 | 16.000 | 1.00 | 40.30 | C |
| ATOM | 380 | CG2 | VAL | A | 265 | −22.152 | 58.860 | 15.203 | 1.00 | 39.13 | C |
| ATOM | 381 | N | ARG | A | 266 | −18.419 | 56.391 | 14.903 | 1.00 | 41.76 | N |
| ATOM | 382 | CA | ARG | A | 266 | −17.085 | 55.879 | 15.180 | 1.00 | 42.08 | C |
| ATOM | 383 | C | ARG | A | 266 | −16.254 | 55.737 | 13.904 | 1.00 | 42.36 | C |
| ATOM | 384 | O | ARG | A | 266 | −15.037 | 55.914 | 13.925 | 1.00 | 42.58 | O |
| ATOM | 385 | CB | ARG | A | 266 | −17.152 | 54.529 | 15.900 | 1.00 | 41.44 | C |
| ATOM | 386 | CG | ARG | A | 266 | −15.778 | 54.011 | 16.322 | 1.00 | 41.57 | C |
| ATOM | 387 | CD | ARG | A | 266 | −15.860 | 52.833 | 17.281 | 1.00 | 42.89 | C |
| ATOM | 388 | NE | ARG | A | 266 | −14.584 | 52.588 | 17.962 | 1.00 | 45.83 | N |
| ATOM | 389 | CZ | ARG | A | 266 | −13.471 | 52.170 | 17.357 | 1.00 | 46.00 | C |
| ATOM | 390 | NH1 | ARG | A | 266 | −13.467 | 51.942 | 16.050 | 1.00 | 46.66 | N |
| ATOM | 391 | NH2 | ARG | A | 266 | −12.359 | 51.987 | 18.056 | 1.00 | 43.50 | N |
| ATOM | 392 | N | GLN | A | 267 | −16.904 | 55.418 | 12.793 | 1.00 | 42.21 | N |
| ATOM | 393 | CA | GLN | A | 267 | −16.180 | 55.269 | 11.540 | 1.00 | 42.69 | C |
| ATOM | 394 | C | GLN | A | 267 | −15.700 | 56.659 | 11.112 | 1.00 | 41.61 | C |
| ATOM | 395 | O | GLN | A | 267 | −14.574 | 56.832 | 10.642 | 1.00 | 41.10 | O |
| ATOM | 396 | CB | GLN | A | 267 | −17.093 | 54.662 | 10.463 | 1.00 | 44.02 | C |
| ATOM | 397 | CG | GLN | A | 267 | −16.412 | 54.415 | 9.125 | 1.00 | 46.36 | C |
| ATOM | 398 | CD | GLN | A | 267 | −15.185 | 53.535 | 9.261 | 1.00 | 52.27 | C |
| ATOM | 399 | OE1 | GLN | A | 267 | −15.276 | 52.389 | 9.707 | 1.00 | 55.17 | O |
| ATOM | 400 | NE2 | GLN | A | 267 | −14.027 | 54.066 | 8.883 | 1.00 | 53.18 | N |
| ATOM | 401 | N | TYR | A | 268 | −16.564 | 57.651 | 11.293 | 1.00 | 39.04 | N |
| ATOM | 402 | CA | TYR | A | 268 | −16.230 | 59.019 | 10.931 | 1.00 | 38.54 | C |
| ATOM | 403 | C | TYR | A | 268 | −15.012 | 59.500 | 11.731 | 1.00 | 38.27 | C |
| ATOM | 404 | O | TYR | A | 268 | −14.102 | 60.110 | 11.177 | 1.00 | 37.18 | O |
| ATOM | 405 | CB | TYR | A | 268 | −17.425 | 59.932 | 11.202 | 1.00 | 37.58 | C |
| ATOM | 406 | CG | TYR | A | 268 | −17.234 | 61.353 | 10.747 | 1.00 | 37.59 | C |
| ATOM | 407 | CD1 | TYR | A | 268 | −17.442 | 61.716 | 9.418 | 1.00 | 37.41 | C |
| ATOM | 408 | CD2 | TYR | A | 268 | −16.842 | 62.338 | 11.648 | 1.00 | 37.76 | C |
| ATOM | 409 | CE1 | TYR | A | 268 | −17.265 | 63.031 | 9.001 | 1.00 | 39.46 | C |
| ATOM | 410 | CE2 | TYR | A | 268 | −16.660 | 63.656 | 11.242 | 1.00 | 38.31 | C |
| ATOM | 411 | CZ | TYR | A | 268 | −16.872 | 63.999 | 9.924 | 1.00 | 39.99 | C |
| ATOM | 412 | OH | TYR | A | 268 | −16.694 | 65.309 | 9.536 | 1.00 | 41.68 | O |
| ATOM | 413 | N | VAL | A | 269 | −14.994 | 59.226 | 13.032 | 1.00 | 37.22 | N |
| ATOM | 414 | CA | VAL | A | 269 | −13.873 | 59.651 | 13.852 | 1.00 | 37.15 | C |
| ATOM | 415 | C | VAL | A | 269 | −12.634 | 58.836 | 13.499 | 1.00 | 38.97 | C |
| ATOM | 416 | O | VAL | A | 269 | −11.514 | 59.344 | 13.558 | 1.00 | 37.81 | O |
| ATOM | 417 | CB | VAL | A | 269 | −14.192 | 59.511 | 15.350 | 1.00 | 35.58 | C |
| ATOM | 418 | CG1 | VAL | A | 269 | −12.960 | 59.819 | 16.177 | 1.00 | 34.19 | C |
| ATOM | 419 | CG2 | VAL | A | 269 | −15.317 | 60.466 | 15.723 | 1.00 | 32.94 | C |
| ATOM | 420 | N | THR | A | 270 | −12.833 | 57.573 | 13.127 | 1.00 | 38.57 | N |
| ATOM | 421 | CA | THR | A | 270 | −11.711 | 56.733 | 12.741 | 1.00 | 37.98 | C |
| ATOM | 422 | C | THR | A | 270 | −11.124 | 57.309 | 11.458 | 1.00 | 38.57 | C |
| ATOM | 423 | O | THR | A | 270 | −9.927 | 57.204 | 11.206 | 1.00 | 39.06 | O |
| ATOM | 424 | CB | THR | A | 270 | −12.147 | 55.273 | 12.503 | 1.00 | 37.78 | C |
| ATOM | 425 | OG1 | THR | A | 270 | −12.314 | 54.619 | 13.768 | 1.00 | 40.02 | O |
| ATOM | 426 | CG2 | THR | A | 270 | −11.107 | 54.520 | 11.683 | 1.00 | 33.38 | C |
| ATOM | 427 | N | ASP | A | 271 | −11.971 | 57.931 | 10.649 | 1.00 | 38.54 | N |
| ATOM | 428 | CA | ASP | A | 271 | −11.501 | 58.531 | 9.409 | 1.00 | 39.33 | C |
| ATOM | 429 | C | ASP | A | 271 | −10.787 | 59.859 | 9.681 | 1.00 | 39.67 | C |
| ATOM | 430 | O | ASP | A | 271 | −9.823 | 60.204 | 8.983 | 1.00 | 40.02 | O |
| ATOM | 431 | CB | ASP | A | 271 | −12.662 | 58.735 | 8.438 | 1.00 | 39.60 | C |
| ATOM | 432 | CG | ASP | A | 271 | −13.212 | 57.414 | 7.898 | 1.00 | 43.63 | C |
| ATOM | 433 | OD1 | ASP | A | 271 | −12.512 | 56.372 | 8.029 | 1.00 | 44.09 | O |
| ATOM | 434 | OD2 | ASP | A | 271 | −14.334 | 57.422 | 7.329 | 1.00 | 42.12 | O |
| ATOM | 435 | N | LEU | A | 272 | −11.250 | 60.601 | 10.686 | 1.00 | 36.50 | N |
| ATOM | 436 | CA | LEU | A | 272 | −10.599 | 61.857 | 11.030 | 1.00 | 36.28 | C |
| ATOM | 437 | C | LEU | A | 272 | −9.181 | 61.496 | 11.447 | 1.00 | 34.58 | C |
| ATOM | 438 | O | LEU | A | 272 | −8.213 | 62.142 | 11.055 | 1.00 | 33.56 | O |
| ATOM | 439 | CB | LEU | A | 272 | −11.313 | 62.541 | 12.194 | 1.00 | 35.12 | C |
| ATOM | 440 | CG | LEU | A | 272 | −12.683 | 63.117 | 11.850 | 1.00 | 36.69 | C |
| ATOM | 441 | CD1 | LEU | A | 272 | −13.339 | 63.676 | 13.108 | 1.00 | 36.86 | C |
| ATOM | 442 | CD2 | LEU | A | 272 | −12.522 | 64.191 | 10.771 | 1.00 | 33.96 | C |
| ATOM | 443 | N | GLY | A | 273 | −9.076 | 60.433 | 12.232 | 1.00 | 32.74 | N |
| ATOM | 444 | CA | GLY | A | 273 | −7.785 | 59.993 | 12.703 | 1.00 | 32.74 | C |
| ATOM | 445 | C | GLY | A | 273 | −6.838 | 59.662 | 11.572 | 1.00 | 34.58 | C |
| ATOM | 446 | O | GLY | A | 273 | −5.694 | 60.130 | 11.550 | 1.00 | 34.16 | O |
| ATOM | 447 | N | THR | A | 274 | −7.305 | 58.869 | 10.615 | 1.00 | 33.91 | N |
| ATOM | 448 | CA | THR | A | 274 | −6.440 | 58.482 | 9.513 | 1.00 | 36.65 | C |
| ATOM | 449 | C | THR | A | 274 | −6.076 | 59.641 | 8.602 | 1.00 | 35.93 | C |
| ATOM | 450 | O | THR | A | 274 | −4.994 | 59.658 | 8.017 | 1.00 | 34.64 | O |
| ATOM | 451 | CB | THR | A | 274 | −7.068 | 57.367 | 8.657 | 1.00 | 38.75 | C |
| ATOM | 452 | OG1 | THR | A | 274 | −6.149 | 56.998 | 7.622 | 1.00 | 41.32 | O |
| ATOM | 453 | CG2 | THR | A | 274 | −8.359 | 57.840 | 8.022 | 1.00 | 39.78 | C |
| ATOM | 454 | N | LYS | A | 275 | −6.972 | 60.615 | 8.492 | 1.00 | 36.31 | N |
| ATOM | 455 | CA | LYS | A | 275 | −6.721 | 61.756 | 7.626 | 1.00 | 37.64 | C |
| ATOM | 456 | C | LYS | A | 275 | −5.888 | 62.871 | 8.255 | 1.00 | 38.01 | C |
| ATOM | 457 | O | LYS | A | 275 | −5.052 | 63.472 | 7.579 | 1.00 | 37.62 | O |
| ATOM | 458 | CB | LYS | A | 275 | −8.052 | 62.346 | 7.119 | 1.00 | 38.24 | C |
| ATOM | 459 | CG | LYS | A | 275 | −8.858 | 61.393 | 6.241 | 1.00 | 40.86 | C |
| ATOM | 460 | CD | LYS | A | 275 | −9.794 | 62.113 | 5.262 | 1.00 | 41.62 | C |
| ATOM | 461 | CE | LYS | A | 275 | −11.015 | 62.714 | 5.937 | 1.00 | 43.29 | C |
| ATOM | 462 | NZ | LYS | A | 275 | −11.958 | 63.288 | 4.926 | 1.00 | 46.29 | N |
| ATOM | 463 | N | TYR | A | 276 | −6.088 | 63.121 | 9.548 | 1.00 | 37.70 | N |
| ATOM | 464 | CA | TYR | A | 276 | −5.401 | 64.219 | 10.209 | 1.00 | 36.58 | C |
| ATOM | 465 | C | TYR | A | 276 | −4.364 | 63.917 | 11.268 | 1.00 | 36.81 | C |
| ATOM | 466 | O | TYR | A | 276 | −3.512 | 64.766 | 11.545 | 1.00 | 37.07 | O |
| ATOM | 467 | CB | TYR | A | 276 | −6.438 | 65.167 | 10.789 | 1.00 | 38.53 | C |
| ATOM | 468 | CG | TYR | A | 276 | −7.472 | 65.569 | 9.776 | 1.00 | 45.01 | C |
| ATOM | 469 | CD1 | TYR | A | 276 | −7.094 | 66.118 | 8.552 | 1.00 | 48.77 | C |
| ATOM | 470 | CD2 | TYR | A | 276 | −8.831 | 65.375 | 10.021 | 1.00 | 47.38 | C |
| ATOM | 471 | CE1 | TYR | A | 276 | −8.044 | 66.460 | 7.591 | 1.00 | 51.85 | C |
| ATOM | 472 | CE2 | TYR | A | 276 | −9.790 | 65.715 | 9.073 | 1.00 | 48.88 | C |
| ATOM | 473 | CZ | TYR | A | 276 | −9.394 | 66.253 | 7.860 | 1.00 | 52.19 | C |
| ATOM | 474 | OH | TYR | A | 276 | −10.339 | 66.550 | 6.898 | 1.00 | 55.56 | O |
| ATOM | 475 | N | ASN | A | 277 | −4.424 | 62.738 | 11.879 | 1.00 | 35.37 | N |
| ATOM | 476 | CA | ASN | A | 277 | −3.440 | 62.410 | 12.898 | 1.00 | 34.11 | C |
| ATOM | 477 | C | ASN | A | 277 | −2.037 | 62.760 | 12.423 | 1.00 | 35.89 | C |
| ATOM | 478 | O | ASN | A | 277 | −1.552 | 62.225 | 11.425 | 1.00 | 35.26 | O |
| ATOM | 479 | CB | ASN | A | 277 | −3.469 | 60.929 | 13.248 | 1.00 | 34.33 | C |
| ATOM | 480 | CG | ASN | A | 277 | −4.620 | 60.566 | 14.137 | 1.00 | 35.92 | C |
| ATOM | 481 | OD1 | ASN | A | 277 | −5.202 | 61.424 | 14.796 | 1.00 | 35.31 | O |
| ATOM | 482 | ND2 | ASN | A | 277 | −4.948 | 59.277 | 14.182 | 1.00 | 36.97 | N |
| ATOM | 483 | N | THR | A | 278 | −1.393 | 63.668 | 13.143 | 1.00 | 35.98 | N |
| ATOM | 484 | CA | THR | A | 278 | −0.041 | 64.068 | 12.816 | 1.00 | 35.85 | C |
| ATOM | 485 | C | THR | A | 278 | 0.909 | 62.933 | 13.184 | 1.00 | 37.11 | C |
| ATOM | 486 | O | THR | A | 278 | 2.119 | 63.026 | 12.970 | 1.00 | 38.93 | O |
| ATOM | 487 | CB | THR | A | 278 | 0.351 | 65.325 | 13.584 | 1.00 | 35.26 | C |
| ATOM | 488 | OG1 | THR | A | 278 | 0.038 | 65.152 | 14.978 | 1.00 | 36.23 | O |
| ATOM | 489 | CG2 | THR | A | 278 | −0.397 | 66.533 | 13.026 | 1.00 | 33.45 | C |
| ATOM | 490 | N | SER | A | 279 | 0.357 | 61.858 | 13.742 | 1.00 | 36.44 | N |
| ATOM | 491 | CA | SER | A | 279 | 1.176 | 60.715 | 14.107 | 1.00 | 36.47 | C |
| ATOM | 492 | C | SER | A | 279 | 1.287 | 59.762 | 12.915 | 1.00 | 35.78 | C |
| ATOM | 493 | O | SER | A | 279 | 2.156 | 58.898 | 12.888 | 1.00 | 35.80 | O |
| ATOM | 494 | CB | SER | A | 279 | 0.592 | 59.984 | 15.328 | 1.00 | 36.36 | C |
| ATOM | 495 | OG | SER | A | 279 | −0.644 | 59.342 | 15.040 | 1.00 | 36.51 | O |
| ATOM | 496 | N | THR | A | 280 | 0.409 | 59.920 | 11.929 | 1.00 | 35.46 | N |
| ATOM | 497 | CA | THR | A | 280 | 0.457 | 59.061 | 10.747 | 1.00 | 36.83 | C |
| ATOM | 498 | C | THR | A | 280 | 0.364 | 59.845 | 9.428 | 1.00 | 36.87 | C |
| ATOM | 499 | O | THR | A | 280 | 0.193 | 59.267 | 8.350 | 1.00 | 38.00 | O |
| ATOM | 500 | CB | THR | A | 280 | −0.654 | 57.987 | 10.782 | 1.00 | 37.60 | C |
| ATOM | 501 | OG1 | THR | A | 280 | −1.934 | 58.621 | 10.871 | 1.00 | 37.48 | O |
| ATOM | 502 | CG2 | THR | A | 280 | −0.464 | 57.064 | 11.981 | 1.00 | 35.29 | C |
| ATOM | 503 | N | ASN | A | 281 | 0.478 | 61.165 | 9.523 | 1.00 | 35.48 | N |
| ATOM | 504 | CA | ASN | A | 281 | 0.447 | 62.015 | 8.348 | 1.00 | 34.63 | C |
| ATOM | 505 | C | ASN | A | 281 | 1.590 | 63.006 | 8.448 | 1.00 | 35.79 | C |
| ATOM | 506 | O | ASN | A | 281 | 1.769 | 63.666 | 9.472 | 1.00 | 37.32 | O |
| ATOM | 507 | CB | ASN | A | 281 | −0.888 | 62.744 | 8.237 | 1.00 | 35.26 | C |
| ATOM | 508 | CG | ASN | A | 281 | −2.044 | 61.792 | 7.987 | 1.00 | 38.05 | C |
| ATOM | 509 | OD1 | ASN | A | 281 | −2.759 | 61.402 | 8.915 | 1.00 | 37.03 | O |
| ATOM | 510 | ND2 | ASN | A | 281 | −2.216 | 61.390 | 6.727 | 1.00 | 35.98 | N |
| ATOM | 511 | N | ASP | A | 282 | 2.383 | 63.088 | 7.389 | 1.00 | 36.01 | N |
| ATOM | 512 | CA | ASP | A | 282 | 3.516 | 63.993 | 7.377 | 1.00 | 37.35 | C |
| ATOM | 513 | C | ASP | A | 282 | 3.057 | 65.439 | 7.462 | 1.00 | 36.87 | C |
| ATOM | 514 | O | ASP | A | 282 | 1.876 | 65.742 | 7.282 | 1.00 | 35.90 | O |
| ATOM | 515 | CB | ASP | A | 282 | 4.356 | 63.774 | 6.125 | 1.00 | 38.33 | C |
| ATOM | 516 | CG | ASP | A | 282 | 4.869 | 62.358 | 6.021 | 1.00 | 42.51 | C |
| ATOM | 517 | OD1 | ASP | A | 282 | 5.360 | 61.831 | 7.046 | 1.00 | 45.12 | O |
| ATOM | 518 | OD2 | ASP | A | 282 | 4.785 | 61.774 | 4.920 | 1.00 | 45.72 | O |
| ATOM | 519 | N | THR | A | 283 | 4.003 | 66.327 | 7.740 | 1.00 | 35.56 | N |
| ATOM | 520 | CA | THR | A | 283 | 3.700 | 67.736 | 7.877 | 1.00 | 36.22 | C |
| ATOM | 521 | C | THR | A | 283 | 4.270 | 68.584 | 6.751 | 1.00 | 38.33 | C |
| ATOM | 522 | O | THR | A | 283 | 5.435 | 68.424 | 6.368 | 1.00 | 38.70 | O |
| ATOM | 523 | CB | THR | A | 283 | 4.253 | 68.260 | 9.207 | 1.00 | 35.27 | C |
| ATOM | 524 | OG1 | THR | A | 283 | 3.517 | 67.671 | 10.287 | 1.00 | 35.45 | O |
| ATOM | 525 | CG2 | THR | A | 283 | 4.156 | 69.783 | 9.274 | 1.00 | 35.45 | C |
| ATOM | 526 | N | LYS | A | 284 | 3.439 | 69.480 | 6.218 | 1.00 | 37.15 | N |
| ATOM | 527 | CA | LYS | A | 284 | 3.880 | 70.394 | 5.172 | 1.00 | 35.82 | C |
| ATOM | 528 | C | LYS | A | 284 | 4.559 | 71.515 | 5.944 | 1.00 | 36.08 | C |
| ATOM | 529 | O | LYS | A | 284 | 3.960 | 72.103 | 6.841 | 1.00 | 37.27 | O |
| ATOM | 530 | CB | LYS | A | 284 | 2.692 | 70.933 | 4.383 | 1.00 | 34.42 | C |
| ATOM | 531 | CG | LYS | A | 284 | 1.817 | 69.837 | 3.811 | 1.00 | 36.44 | C |
| ATOM | 532 | CD | LYS | A | 284 | 1.047 | 70.292 | 2.575 | 1.00 | 39.36 | C |
| ATOM | 533 | CE | LYS | A | 284 | −0.128 | 69.365 | 2.301 | 1.00 | 40.60 | C |
| ATOM | 534 | NZ | LYS | A | 284 | 0.208 | 67.928 | 2.495 | 1.00 | 41.02 | N |
| ATOM | 535 | N | PHE | A | 285 | 5.815 | 71.793 | 5.604 | 1.00 | 35.44 | N |
| ATOM | 536 | CA | PHE | A | 285 | 6.608 | 72.806 | 6.296 | 1.00 | 33.13 | C |
| ATOM | 537 | C | PHE | A | 285 | 7.179 | 73.865 | 5.348 | 1.00 | 33.92 | C |
| ATOM | 538 | O | PHE | A | 285 | 7.717 | 73.546 | 4.282 | 1.00 | 33.31 | O |
| ATOM | 539 | CB | PHE | A | 285 | 7.752 | 72.104 | 7.046 | 1.00 | 32.15 | C |
| ATOM | 540 | CG | PHE | A | 285 | 8.729 | 73.041 | 7.700 | 1.00 | 32.22 | C |
| ATOM | 541 | CD1 | PHE | A | 285 | 8.327 | 73.886 | 8.727 | 1.00 | 31.93 | C |
| ATOM | 542 | CD2 | PHE | A | 285 | 10.058 | 73.070 | 7.294 | 1.00 | 32.18 | C |
| ATOM | 543 | CE1 | PHE | A | 285 | 9.241 | 74.751 | 9.344 | 1.00 | 33.63 | C |
| ATOM | 544 | CE2 | PHE | A | 285 | 10.980 | 73.931 | 7.906 | 1.00 | 32.50 | C |
| ATOM | 545 | CZ | PHE | A | 285 | 10.571 | 74.771 | 8.931 | 1.00 | 31.98 | C |
| ATOM | 546 | N | LYS | A | 286 | 7.069 | 75.128 | 5.741 | 1.00 | 34.77 | N |
| ATOM | 547 | CA | LYS | A | 286 | 7.601 | 76.210 | 4.921 | 1.00 | 35.63 | C |
| ATOM | 548 | C | LYS | A | 286 | 9.069 | 76.413 | 5.305 | 1.00 | 35.74 | C |
| ATOM | 549 | O | LYS | A | 286 | 9.388 | 77.163 | 6.235 | 1.00 | 35.14 | O |
| ATOM | 550 | CB | LYS | A | 286 | 6.792 | 77.476 | 5.163 | 1.00 | 35.38 | C |
| ATOM | 551 | CG | LYS | A | 286 | 5.352 | 77.332 | 4.731 | 1.00 | 37.94 | C |
| ATOM | 552 | CD | LYS | A | 286 | 4.484 | 78.344 | 5.425 | 1.00 | 41.93 | C |
| ATOM | 553 | CE | LYS | A | 286 | 3.044 | 78.184 | 5.018 | 1.00 | 44.44 | C |
| ATOM | 554 | NZ | LYS | A | 286 | 2.175 | 79.065 | 5.842 | 1.00 | 49.68 | N |
| ATOM | 555 | N | SER | A | 287 | 9.955 | 75.721 | 4.597 | 1.00 | 34.41 | N |
| ATOM | 556 | CA | SER | A | 287 | 11.377 | 75.813 | 4.877 | 1.00 | 38.48 | C |
| ATOM | 557 | C | SER | A | 287 | 11.912 | 77.203 | 4.569 | 1.00 | 39.97 | C |
| ATOM | 558 | O | SER | A | 287 | 11.244 | 78.027 | 3.938 | 1.00 | 42.65 | O |
| ATOM | 559 | CB | SER | A | 287 | 12.153 | 74.788 | 4.053 | 1.00 | 37.86 | C |
| ATOM | 560 | OG | SER | A | 287 | 12.139 | 75.156 | 2.686 | 1.00 | 41.23 | O |
| ATOM | 561 | N | THR | A | 288 | 13.132 | 77.454 | 5.013 | 1.00 | 40.79 | N |
| ATOM | 562 | CA | THR | A | 288 | 13.767 | 78.738 | 4.787 | 1.00 | 41.69 | C |
| ATOM | 563 | C | THR | A | 288 | 14.334 | 78.864 | 3.369 | 1.00 | 41.57 | C |
| ATOM | 564 | O | THR | A | 288 | 14.185 | 79.905 | 2.736 | 1.00 | 41.30 | O |
| ATOM | 565 | CB | THR | A | 288 | 14.898 | 78.963 | 5.822 | 1.00 | 41.56 | C |
| ATOM | 566 | OG1 | THR | A | 288 | 14.329 | 79.034 | 7.136 | 1.00 | 42.23 | O |
| ATOM | 567 | CG2 | THR | A | 288 | 15.651 | 80.259 | 5.542 | 1.00 | 41.64 | C |
| ATOM | 568 | N | LYS | A | 289 | 14.940 | 77.793 | 2.860 | 1.00 | 42.02 | N |
| ATOM | 569 | CA | LYS | A | 289 | 15.582 | 77.821 | 1.543 | 1.00 | 43.70 | C |
| ATOM | 570 | C | LYS | A | 289 | 14.953 | 76.975 | 0.437 | 1.00 | 43.78 | C |
| ATOM | 571 | O | LYS | A | 289 | 15.597 | 76.736 | −0.585 | 1.00 | 45.44 | O |
| ATOM | 572 | CB | LYS | A | 289 | 17.040 | 77.359 | 1.682 | 1.00 | 44.54 | C |
| ATOM | 573 | CG | LYS | A | 289 | 17.804 | 77.945 | 2.857 | 1.00 | 48.49 | C |
| ATOM | 574 | CD | LYS | A | 289 | 18.170 | 79.404 | 2.636 | 1.00 | 51.86 | C |
| ATOM | 575 | CE | LYS | A | 289 | 19.160 | 79.553 | 1.495 | 1.00 | 53.84 | C |
| ATOM | 576 | NZ | LYS | A | 289 | 19.585 | 80.972 | 1.321 | 1.00 | 58.05 | N |
| ATOM | 577 | N | ARG | A | 290 | 13.714 | 76.532 | 0.595 | 1.00 | 43.68 | N |
| ATOM | 578 | CA | ARG | A | 290 | 13.150 | 75.657 | −0.426 | 1.00 | 42.00 | C |
| ATOM | 579 | C | ARG | A | 290 | 11.633 | 75.750 | −0.563 | 1.00 | 39.99 | C |
| ATOM | 580 | O | ARG | A | 290 | 11.020 | 74.988 | −1.313 | 1.00 | 38.44 | O |
| ATOM | 581 | CB | ARG | A | 290 | 13.551 | 74.232 | −0.064 | 1.00 | 44.72 | C |
| ATOM | 582 | CG | ARG | A | 290 | 13.816 | 73.280 | −1.193 | 1.00 | 51.28 | C |
| ATOM | 583 | CD | ARG | A | 290 | 14.496 | 72.053 | −0.605 | 1.00 | 55.59 | C |
| ATOM | 584 | NE | ARG | A | 290 | 15.729 | 72.453 | 0.070 | 1.00 | 62.30 | N |
| ATOM | 585 | CZ | ARG | A | 290 | 16.253 | 71.842 | 1.130 | 1.00 | 63.49 | C |
| ATOM | 586 | NH1 | ARG | A | 290 | 15.656 | 70.781 | 1.662 | 1.00 | 64.14 | N |
| ATOM | 587 | NH2 | ARG | A | 290 | 17.376 | 72.305 | 1.663 | 1.00 | 64.45 | N |
| ATOM | 588 | N | GLY | A | 291 | 11.020 | 76.684 | 0.155 | 1.00 | 39.57 | N |
| ATOM | 589 | CA | GLY | A | 291 | 9.576 | 76.799 | 0.080 | 1.00 | 39.27 | C |
| ATOM | 590 | C | GLY | A | 291 | 8.949 | 75.681 | 0.896 | 1.00 | 38.96 | C |
| ATOM | 591 | O | GLY | A | 291 | 9.574 | 75.166 | 1.830 | 1.00 | 38.45 | O |
| ATOM | 592 | N | GLU | A | 292 | 7.730 | 75.286 | 0.545 | 1.00 | 37.63 | N |
| ATOM | 593 | CA | GLU | A | 292 | 7.048 | 74.242 | 1.288 | 1.00 | 36.78 | C |
| ATOM | 594 | C | GLU | A | 292 | 7.496 | 72.836 | 0.914 | 1.00 | 36.58 | C |
| ATOM | 595 | O | GLU | A | 292 | 7.448 | 72.435 | −0.247 | 1.00 | 35.88 | O |
| ATOM | 596 | CB | GLU | A | 292 | 5.541 | 74.366 | 1.114 | 1.00 | 37.62 | C |
| ATOM | 597 | CG | GLU | A | 292 | 4.789 | 73.485 | 2.087 | 1.00 | 41.78 | C |
| ATOM | 598 | CD | GLU | A | 292 | 3.314 | 73.770 | 2.112 | 1.00 | 42.36 | C |
| ATOM | 599 | OE1 | GLU | A | 292 | 2.660 | 73.586 | 1.066 | 1.00 | 44.63 | O |
| ATOM | 600 | OE2 | GLU | A | 292 | 2.813 | 74.178 | 3.180 | 1.00 | 45.12 | O |
| ATOM | 601 | N | VAL | A | 293 | 7.935 | 72.091 | 1.916 | 1.00 | 35.95 | N |
| ATOM | 602 | CA | VAL | A | 293 | 8.401 | 70.729 | 1.708 | 1.00 | 38.23 | C |
| ATOM | 603 | C | VAL | A | 293 | 7.649 | 69.815 | 2.658 | 1.00 | 37.87 | C |
| ATOM | 604 | O | VAL | A | 293 | 6.891 | 70.286 | 3.507 | 1.00 | 38.20 | O |
| ATOM | 605 | CB | VAL | A | 293 | 9.920 | 70.607 | 1.995 | 1.00 | 39.26 | C |
| ATOM | 606 | CG1 | VAL | A | 293 | 10.695 | 71.639 | 1.168 | 1.00 | 39.50 | C |
| ATOM | 607 | CG2 | VAL | A | 293 | 10.191 | 70.801 | 3.483 | 1.00 | 37.49 | C |
| ATOM | 608 | N | THR | A | 294 | 7.855 | 68.512 | 2.518 | 1.00 | 37.84 | N |
| ATOM | 609 | CA | THR | A | 294 | 7.186 | 67.563 | 3.390 | 1.00 | 38.58 | C |
| ATOM | 610 | C | THR | A | 294 | 8.155 | 66.985 | 4.412 | 1.00 | 39.01 | C |
| ATOM | 611 | O | THR | A | 294 | 9.163 | 66.389 | 4.053 | 1.00 | 39.87 | O |
| ATOM | 612 | CB | THR | A | 294 | 6.559 | 66.401 | 2.585 | 1.00 | 40.43 | C |
| ATOM | 613 | OG1 | THR | A | 294 | 5.511 | 66.905 | 1.747 | 1.00 | 40.32 | O |
| ATOM | 614 | CG2 | THR | A | 294 | 5.969 | 65.355 | 3.523 | 1.00 | 40.59 | C |
| ATOM | 615 | N | VAL | A | 295 | 7.848 | 67.182 | 5.690 | 1.00 | 39.85 | N |
| ATOM | 616 | CA | VAL | A | 295 | 8.671 | 66.656 | 6.772 | 1.00 | 38.53 | C |
| ATOM | 617 | C | VAL | A | 295 | 7.952 | 65.417 | 7.312 | 1.00 | 39.28 | C |
| ATOM | 618 | O | VAL | A | 295 | 6.795 | 65.490 | 7.733 | 1.00 | 38.97 | O |
| ATOM | 619 | CB | VAL | A | 295 | 8.824 | 67.674 | 7.924 | 1.00 | 39.12 | C |
| ATOM | 620 | CG1 | VAL | A | 295 | 9.712 | 67.087 | 9.020 | 1.00 | 38.50 | C |
| ATOM | 621 | CG2 | VAL | A | 295 | 9.407 | 68.982 | 7.403 | 1.00 | 38.83 | C |
| ATOM | 622 | N | PRO | A | 296 | 8.635 | 64.266 | 7.318 | 1.00 | 39.65 | N |
| ATOM | 623 | CA | PRO | A | 296 | 8.066 | 63.002 | 7.804 | 1.00 | 39.09 | C |
| ATOM | 624 | C | PRO | A | 296 | 7.633 | 63.037 | 9.273 | 1.00 | 38.67 | C |
| ATOM | 625 | O | PRO | A | 296 | 8.110 | 63.867 | 10.070 | 1.00 | 37.65 | O |
| ATOM | 626 | CB | PRO | A | 296 | 9.206 | 61.996 | 7.600 | 1.00 | 39.33 | C |
| ATOM | 627 | CG | PRO | A | 296 | 10.098 | 62.642 | 6.590 | 1.00 | 40.93 | C |
| ATOM | 628 | CD | PRO | A | 296 | 10.055 | 64.095 | 6.974 | 1.00 | 40.49 | C |
| ATOM | 629 | N | VAL | A | 297 | 6.732 | 62.119 | 9.619 | 1.00 | 37.32 | N |
| ATOM | 630 | CA | VAL | A | 297 | 6.243 | 61.988 | 10.987 | 1.00 | 35.34 | C |
| ATOM | 631 | C | VAL | A | 297 | 7.453 | 61.819 | 11.903 | 1.00 | 34.97 | C |
| ATOM | 632 | O | VAL | A | 297 | 8.374 | 61.065 | 11.590 | 1.00 | 34.55 | O |
| ATOM | 633 | CB | VAL | A | 297 | 5.339 | 60.736 | 11.145 | 1.00 | 34.00 | C |
| ATOM | 634 | CG1 | VAL | A | 297 | 5.054 | 60.480 | 12.625 | 1.00 | 32.22 | C |
| ATOM | 635 | CG2 | VAL | A | 297 | 4.033 | 60.926 | 10.364 | 1.00 | 32.72 | C |
| ATOM | 636 | N | GLY | A | 298 | 7.449 | 62.536 | 13.021 | 1.00 | 34.31 | N |
| ATOM | 637 | CA | GLY | A | 298 | 8.535 | 62.435 | 13.973 | 1.00 | 33.78 | C |
| ATOM | 638 | C | GLY | A | 298 | 7.986 | 62.065 | 15.342 | 1.00 | 36.81 | C |
| ATOM | 639 | O | GLY | A | 298 | 6.997 | 61.339 | 15.454 | 1.00 | 37.60 | O |
| ATOM | 640 | N | THR | A | 299 | 8.619 | 62.575 | 16.390 | 1.00 | 36.18 | N |
| ATOM | 641 | CA | THR | A | 299 | 8.184 | 62.291 | 17.748 | 1.00 | 35.69 | C |
| ATOM | 642 | C | THR | A | 299 | 7.178 | 63.320 | 18.277 | 1.00 | 36.44 | C |
| ATOM | 643 | O | THR | A | 299 | 6.507 | 63.078 | 19.284 | 1.00 | 36.53 | O |
| ATOM | 644 | CB | THR | A | 299 | 9.388 | 62.259 | 18.691 | 1.00 | 36.46 | C |
| ATOM | 645 | OG1 | THR | A | 299 | 10.026 | 63.545 | 18.686 | 1.00 | 33.17 | O |
| ATOM | 646 | CG2 | THR | A | 299 | 10.385 | 61.192 | 18.235 | 1.00 | 34.49 | C |
| ATOM | 647 | N | TYR | A | 300 | 7.085 | 64.471 | 17.613 | 1.00 | 35.39 | N |
| ATOM | 648 | CA | TYR | A | 300 | 6.152 | 65.519 | 18.033 | 1.00 | 34.86 | C |
| ATOM | 649 | C | TYR | A | 300 | 4.853 | 65.347 | 17.246 | 1.00 | 35.10 | C |
| ATOM | 650 | O | TYR | A | 300 | 4.771 | 65.738 | 16.085 | 1.00 | 35.35 | O |
| ATOM | 651 | CB | TYR | A | 300 | 6.747 | 66.901 | 17.760 | 1.00 | 33.21 | C |
| ATOM | 652 | CG | TYR | A | 300 | 5.944 | 68.031 | 18.364 | 1.00 | 33.68 | C |
| ATOM | 653 | CD1 | TYR | A | 300 | 5.714 | 68.086 | 19.742 | 1.00 | 33.67 | C |
| ATOM | 654 | CD2 | TYR | A | 300 | 5.407 | 69.043 | 17.564 | 1.00 | 32.51 | C |
| ATOM | 655 | CE1 | TYR | A | 300 | 4.967 | 69.118 | 20.311 | 1.00 | 33.26 | C |
| ATOM | 656 | CE2 | TYR | A | 300 | 4.660 | 70.081 | 18.124 | 1.00 | 33.69 | C |
| ATOM | 657 | CZ | TYR | A | 300 | 4.444 | 70.109 | 19.496 | 1.00 | 33.62 | C |
| ATOM | 658 | OH | TYR | A | 300 | 3.696 | 71.117 | 20.055 | 1.00 | 35.60 | O |
| ATOM | 659 | N | SER | A | 301 | 3.839 | 64.772 | 17.882 | 1.00 | 34.35 | N |
| ATOM | 660 | CA | SER | A | 301 | 2.578 | 64.526 | 17.198 | 1.00 | 34.42 | C |
| ATOM | 661 | C | SER | A | 301 | 1.436 | 64.204 | 18.155 | 1.00 | 34.73 | C |
| ATOM | 662 | O | SER | A | 301 | 1.613 | 64.184 | 19.371 | 1.00 | 36.16 | O |
| ATOM | 663 | CB | SER | A | 301 | 2.763 | 63.350 | 16.245 | 1.00 | 35.03 | C |
| ATOM | 664 | OG | SER | A | 301 | 3.127 | 62.190 | 16.976 | 1.00 | 35.81 | O |
| ATOM | 665 | N | TRP | A | 302 | 0.258 | 63.953 | 17.592 | 1.00 | 34.21 | N |
| ATOM | 666 | CA | TRP | A | 302 | −0.912 | 63.595 | 18.391 | 1.00 | 33.23 | C |
| ATOM | 667 | C | TRP | A | 302 | −1.769 | 62.580 | 17.655 | 1.00 | 33.16 | C |
| ATOM | 668 | O | TRP | A | 302 | −1.680 | 62.448 | 16.433 | 1.00 | 33.62 | O |
| ATOM | 669 | CB | TRP | A | 302 | −1.764 | 64.824 | 18.758 | 1.00 | 29.86 | C |
| ATOM | 670 | CG | TRP | A | 302 | −1.982 | 65.828 | 17.663 | 1.00 | 31.09 | C |
| ATOM | 671 | CD1 | TRP | A | 302 | −1.292 | 66.994 | 17.474 | 1.00 | 32.32 | C |
| ATOM | 672 | CD2 | TRP | A | 302 | −2.959 | 65.769 | 16.616 | 1.00 | 30.38 | C |
| ATOM | 673 | NE1 | TRP | A | 302 | −1.779 | 67.664 | 16.375 | 1.00 | 31.04 | N |
| ATOM | 674 | CE2 | TRP | A | 302 | −2.801 | 66.936 | 15.829 | 1.00 | 30.60 | C |
| ATOM | 675 | CE3 | TRP | A | 302 | −3.953 | 64.843 | 16.264 | 1.00 | 31.40 | C |
| ATOM | 676 | CZ2 | TRP | A | 302 | −3.600 | 67.206 | 14.712 | 1.00 | 30.87 | C |
| ATOM | 677 | CZ3 | TRP | A | 302 | −4.753 | 65.110 | 15.147 | 1.00 | 31.32 | C |
| ATOM | 678 | CH2 | TRP | A | 302 | −4.568 | 66.285 | 14.386 | 1.00 | 32.60 | C |
| ATOM | 679 | N | THR | A | 303 | −2.595 | 61.865 | 18.414 | 1.00 | 32.21 | N |
| ATOM | 680 | CA | THR | A | 303 | −3.472 | 60.848 | 17.864 | 1.00 | 31.99 | C |
| ATOM | 681 | C | THR | A | 303 | −4.863 | 60.986 | 18.468 | 1.00 | 32.11 | C |
| ATOM | 682 | O | THR | A | 303 | −5.026 | 61.027 | 19.696 | 1.00 | 32.94 | O |
| ATOM | 683 | CB | THR | A | 303 | −2.936 | 59.441 | 18.182 | 1.00 | 33.00 | C |
| ATOM | 684 | OG1 | THR | A | 303 | −1.601 | 59.306 | 17.669 | 1.00 | 33.83 | O |
| ATOM | 685 | CG2 | THR | A | 303 | −3.834 | 58.379 | 17.565 | 1.00 | 31.67 | C |
| ATOM | 686 | N | ILE | A | 304 | −5.871 | 61.071 | 17.611 | 1.00 | 30.83 | N |
| ATOM | 687 | CA | ILE | A | 304 | −7.238 | 61.186 | 18.095 | 1.00 | 31.62 | C |
| ATOM | 688 | C | ILE | A | 304 | −7.620 | 59.889 | 18.841 | 1.00 | 32.38 | C |
| ATOM | 689 | O | ILE | A | 304 | −7.410 | 58.786 | 18.336 | 1.00 | 30.55 | O |
| ATOM | 690 | CB | ILE | A | 304 | −8.223 | 61.429 | 16.910 | 1.00 | 30.23 | C |
| ATOM | 691 | CG1 | ILE | A | 304 | −7.957 | 62.805 | 16.285 | 1.00 | 28.56 | C |
| ATOM | 692 | CG2 | ILE | A | 304 | −9.670 | 61.323 | 17.388 | 1.00 | 29.87 | C |
| ATOM | 693 | CD1 | ILE | A | 304 | −8.834 | 63.129 | 15.078 | 1.00 | 25.59 | C |
| ATOM | 694 | N | GLN | A | 305 | −8.136 | 60.017 | 20.058 | 1.00 | 33.35 | N |
| ATOM | 695 | CA | GLN | A | 305 | −8.558 | 58.839 | 20.812 | 1.00 | 35.31 | C |
| ATOM | 696 | C | GLN | A | 305 | −9.957 | 58.534 | 20.291 | 1.00 | 36.56 | C |
| ATOM | 697 | O | GLN | A | 305 | −10.927 | 59.180 | 20.679 | 1.00 | 38.27 | O |
| ATOM | 698 | CB | GLN | A | 305 | −8.606 | 59.146 | 22.305 | 1.00 | 33.96 | C |
| ATOM | 699 | CG | GLN | A | 305 | −7.260 | 59.534 | 22.896 | 1.00 | 38.10 | C |
| ATOM | 700 | CD | GLN | A | 305 | −6.164 | 58.535 | 22.570 | 1.00 | 39.69 | C |
| ATOM | 701 | OE1 | GLN | A | 305 | −5.478 | 58.654 | 21.551 | 1.00 | 40.47 | O |
| ATOM | 702 | NE2 | GLN | A | 305 | −6.001 | 57.536 | 23.431 | 1.00 | 41.14 | N |
| ATOM | 703 | N | THR | A | 306 | −10.054 | 57.555 | 19.400 | 1.00 | 37.13 | N |
| ATOM | 704 | CA | THR | A | 306 | −11.322 | 57.209 | 18.780 | 1.00 | 39.50 | C |
| ATOM | 705 | C | THR | A | 306 | −12.557 | 57.090 | 19.672 | 1.00 | 40.06 | C |
| ATOM | 706 | O | THR | A | 306 | −13.545 | 57.792 | 19.443 | 1.00 | 39.85 | O |
| ATOM | 707 | CB | THR | A | 306 | −11.175 | 55.932 | 17.972 | 1.00 | 40.79 | C |
| ATOM | 708 | OG1 | THR | A | 306 | −10.103 | 56.102 | 17.036 | 1.00 | 42.58 | O |
| ATOM | 709 | CG2 | THR | A | 306 | −12.459 | 55.634 | 17.217 | 1.00 | 39.85 | C |
| ATOM | 710 | N | ASP | A | 307 | −12.509 | 56.224 | 20.684 | 1.00 | 39.78 | N |
| ATOM | 711 | CA | ASP | A | 307 | −13.663 | 56.037 | 21.564 | 1.00 | 40.01 | C |
| ATOM | 712 | C | ASP | A | 307 | −14.042 | 57.294 | 22.339 | 1.00 | 39.48 | C |
| ATOM | 713 | O | ASP | A | 307 | −15.228 | 57.619 | 22.448 | 1.00 | 39.18 | O |
| ATOM | 714 | CB | ASP | A | 307 | −13.432 | 54.866 | 22.531 | 1.00 | 41.46 | C |
| ATOM | 715 | CG | ASP | A | 307 | −13.282 | 53.527 | 21.807 | 1.00 | 44.89 | C |
| ATOM | 716 | OD1 | ASP | A | 307 | −13.984 | 53.303 | 20.793 | 1.00 | 45.59 | O |
| ATOM | 717 | OD2 | ASP | A | 307 | −12.467 | 52.690 | 22.257 | 1.00 | 50.25 | O |
| ATOM | 718 | N | SER | A | 308 | −13.055 | 58.007 | 22.875 | 1.00 | 38.09 | N |
| ATOM | 719 | CA | SER | A | 308 | −13.359 | 59.239 | 23.599 | 1.00 | 38.68 | C |
| ATOM | 720 | C | SER | A | 308 | −14.008 | 60.257 | 22.665 | 1.00 | 38.63 | C |
| ATOM | 721 | O | SER | A | 308 | −15.015 | 60.863 | 23.021 | 1.00 | 39.33 | O |
| ATOM | 722 | CB | SER | A | 308 | −12.097 | 59.856 | 24.196 | 1.00 | 38.40 | C |
| ATOM | 723 | OG | SER | A | 308 | −11.527 | 58.990 | 25.148 | 1.00 | 42.21 | O |
| ATOM | 724 | N | GLU | A | 309 | −13.439 | 60.441 | 21.472 | 1.00 | 38.07 | N |
| ATOM | 725 | CA | GLU | A | 309 | −13.982 | 61.406 | 20.518 | 1.00 | 40.43 | C |
| ATOM | 726 | C | GLU | A | 309 | −15.369 | 61.037 | 20.044 | 1.00 | 40.38 | C |
| ATOM | 727 | O | GLU | A | 309 | −16.251 | 61.892 | 19.989 | 1.00 | 40.62 | O |
| ATOM | 728 | CB | GLU | A | 309 | −13.077 | 61.564 | 19.293 | 1.00 | 40.41 | C |
| ATOM | 729 | CG | GLU | A | 309 | −12.374 | 62.903 | 19.225 | 1.00 | 43.72 | C |
| ATOM | 730 | CD | GLU | A | 309 | −13.315 | 64.106 | 19.327 | 1.00 | 44.28 | C |
| ATOM | 731 | OE1 | GLU | A | 309 | −13.990 | 64.450 | 18.335 | 1.00 | 44.81 | O |
| ATOM | 732 | OE2 | GLU | A | 309 | −13.373 | 64.716 | 20.411 | 1.00 | 43.52 | O |
| ATOM | 733 | N | THR | A | 310 | −15.558 | 59.771 | 19.679 | 1.00 | 40.65 | N |
| ATOM | 734 | CA | THR | A | 310 | −16.864 | 59.310 | 19.213 | 1.00 | 40.88 | C |
| ATOM | 735 | C | THR | A | 310 | −17.947 | 59.645 | 20.239 | 1.00 | 40.74 | C |
| ATOM | 736 | O | THR | A | 310 | −18.998 | 60.179 | 19.882 | 1.00 | 39.81 | O |
| ATOM | 737 | CB | THR | A | 310 | −16.877 | 57.787 | 18.967 | 1.00 | 41.99 | C |
| ATOM | 738 | OG1 | THR | A | 310 | −15.941 | 57.457 | 17.931 | 1.00 | 39.66 | O |
| ATOM | 739 | CG2 | THR | A | 310 | −18.273 | 57.332 | 18.547 | 1.00 | 40.56 | C |
| ATOM | 740 | N | GLU | A | 311 | −17.687 | 59.337 | 21.508 | 1.00 | 41.77 | N |
| ATOM | 741 | CA | GLU | A | 311 | −18.652 | 59.626 | 22.571 | 1.00 | 44.39 | C |
| ATOM | 742 | C | GLU | A | 311 | −18.904 | 61.116 | 22.714 | 1.00 | 43.62 | C |
| ATOM | 743 | O | GLU | A | 311 | −20.062 | 61.554 | 22.775 | 1.00 | 42.88 | O |
| ATOM | 744 | CB | GLU | A | 311 | −18.171 | 59.063 | 23.906 | 1.00 | 47.96 | C |
| ATOM | 745 | CG | GLU | A | 311 | −18.588 | 57.625 | 24.130 | 1.00 | 57.86 | C |
| ATOM | 746 | CD | GLU | A | 311 | −17.808 | 56.962 | 25.244 | 1.00 | 64.00 | C |
| ATOM | 747 | OE1 | GLU | A | 311 | −17.817 | 57.495 | 26.383 | 1.00 | 66.48 | O |
| ATOM | 748 | OE2 | GLU | A | 311 | −17.188 | 55.905 | 24.974 | 1.00 | 66.89 | O |
| ATOM | 749 | N | ALA | A | 312 | −17.820 | 61.890 | 22.767 | 1.00 | 40.71 | N |
| ATOM | 750 | CA | ALA | A | 312 | −17.926 | 63.337 | 22.893 | 1.00 | 40.18 | C |
| ATOM | 751 | C | ALA | A | 312 | −18.675 | 63.914 | 21.681 | 1.00 | 39.34 | C |
| ATOM | 752 | O | ALA | A | 312 | −19.525 | 64.787 | 21.835 | 1.00 | 39.82 | O |
| ATOM | 753 | CB | ALA | A | 312 | −16.529 | 63.964 | 23.014 | 1.00 | 37.68 | C |
| ATOM | 754 | N | LEU | A | 313 | −18.370 | 63.415 | 20.485 | 1.00 | 37.86 | N |
| ATOM | 755 | CA | LEU | A | 313 | −19.032 | 63.890 | 19.267 | 1.00 | 38.91 | C |
| ATOM | 756 | C | LEU | A | 313 | −20.546 | 63.659 | 19.348 | 1.00 | 39.34 | C |
| ATOM | 757 | O | LEU | A | 313 | −21.332 | 64.588 | 19.135 | 1.00 | 36.86 | O |
| ATOM | 758 | CB | LEU | A | 313 | −18.490 | 63.162 | 18.038 | 1.00 | 36.09 | C |
| ATOM | 759 | CG | LEU | A | 313 | −18.299 | 63.979 | 16.759 | 1.00 | 36.70 | C |
| ATOM | 760 | CD1 | LEU | A | 313 | −18.157 | 63.019 | 15.586 | 1.00 | 33.85 | C |
| ATOM | 761 | CD2 | LEU | A | 313 | −19.454 | 64.927 | 16.534 | 1.00 | 35.83 | C |
| ATOM | 762 | N | LYS | A | 314 | −20.939 | 62.415 | 19.646 | 1.00 | 40.53 | N |
| ATOM | 763 | CA | LYS | A | 314 | −22.350 | 62.048 | 19.774 | 1.00 | 43.03 | C |
| ATOM | 764 | C | LYS | A | 314 | −23.070 | 62.959 | 20.751 | 1.00 | 43.70 | C |
| ATOM | 765 | O | LYS | A | 314 | −24.128 | 63.505 | 20.442 | 1.00 | 44.27 | O |
| ATOM | 766 | CB | LYS | A | 314 | −22.507 | 60.620 | 20.285 | 1.00 | 44.99 | C |
| ATOM | 767 | CG | LYS | A | 314 | −22.202 | 59.539 | 19.291 | 1.00 | 50.51 | C |
| ATOM | 768 | CD | LYS | A | 314 | −22.626 | 58.177 | 19.849 | 1.00 | 55.21 | C |
| ATOM | 769 | CE | LYS | A | 314 | −24.128 | 58.139 | 20.141 | 1.00 | 57.96 | C |
| ATOM | 770 | NZ | LYS | A | 314 | −24.597 | 56.798 | 20.602 | 1.00 | 59.37 | N |
| ATOM | 771 | N | LYS | A | 315 | −22.501 | 63.106 | 21.943 | 1.00 | 44.12 | N |
| ATOM | 772 | CA | LYS | A | 315 | −23.116 | 63.948 | 22.952 | 1.00 | 45.56 | C |
| ATOM | 773 | C | LYS | A | 315 | −23.320 | 65.345 | 22.386 | 1.00 | 45.09 | C |
| ATOM | 774 | O | LYS | A | 315 | −24.396 | 65.927 | 22.517 | 1.00 | 46.69 | O |
| ATOM | 775 | CB | LYS | A | 315 | −22.245 | 64.011 | 24.209 | 1.00 | 47.36 | C |
| ATOM | 776 | CG | LYS | A | 315 | −22.959 | 64.625 | 25.406 | 1.00 | 50.42 | C |
| ATOM | 777 | CD | LYS | A | 315 | −22.056 | 64.723 | 26.624 | 1.00 | 54.17 | C |
| ATOM | 778 | CE | LYS | A | 315 | −22.809 | 65.285 | 27.830 | 1.00 | 56.18 | C |
| ATOM | 779 | NZ | LYS | A | 315 | −21.944 | 65.394 | 29.042 | 1.00 | 57.51 | N |
| ATOM | 780 | N | ALA | A | 316 | −22.293 | 65.878 | 21.736 | 1.00 | 43.01 | N |
| ATOM | 781 | CA | ALA | A | 316 | −22.388 | 67.211 | 21.167 | 1.00 | 41.94 | C |
| ATOM | 782 | C | ALA | A | 316 | −23.506 | 67.300 | 20.134 | 1.00 | 41.80 | C |
| ATOM | 783 | O | ALA | A | 316 | −24.340 | 68.202 | 20.185 | 1.00 | 43.19 | O |
| ATOM | 784 | CB | ALA | A | 316 | −21.065 | 67.605 | 20.534 | 1.00 | 40.62 | C |
| ATOM | 785 | N | ILE | A | 317 | −23.519 | 66.367 | 19.192 | 1.00 | 40.21 | N |
| ATOM | 786 | CA | ILE | A | 317 | −24.536 | 66.372 | 18.155 | 1.00 | 40.12 | C |
| ATOM | 787 | C | ILE | A | 317 | −25.942 | 66.185 | 18.721 | 1.00 | 41.67 | C |
| ATOM | 788 | O | ILE | A | 317 | −26.853 | 66.939 | 18.379 | 1.00 | 41.65 | O |
| ATOM | 789 | CB | ILE | A | 317 | −24.245 | 65.284 | 17.100 | 1.00 | 38.95 | C |
| ATOM | 790 | CG1 | ILE | A | 317 | −22.982 | 65.666 | 16.320 | 1.00 | 36.98 | C |
| ATOM | 791 | CG2 | ILE | A | 317 | −25.433 | 65.130 | 16.151 | 1.00 | 39.22 | C |
| ATOM | 792 | CD1 | ILE | A | 317 | −22.533 | 64.639 | 15.318 | 1.00 | 34.08 | C |
| ATOM | 793 | N | LEU | A | 318 | −26.116 | 65.193 | 19.592 | 1.00 | 42.83 | N |
| ATOM | 794 | CA | LEU | A | 318 | −27.420 | 64.924 | 20.183 | 1.00 | 44.42 | C |
| ATOM | 795 | C | LEU | A | 318 | −27.985 | 66.099 | 20.967 | 1.00 | 46.51 | C |
| ATOM | 796 | O | LEU | A | 318 | −29.199 | 66.234 | 21.080 | 1.00 | 47.51 | O |
| ATOM | 797 | CB | LEU | A | 318 | −27.351 | 63.695 | 21.082 | 1.00 | 44.27 | C |
| ATOM | 798 | CG | LEU | A | 318 | −27.242 | 62.363 | 20.347 | 1.00 | 44.79 | C |
| ATOM | 799 | CD1 | LEU | A | 318 | −27.152 | 61.247 | 21.360 | 1.00 | 45.44 | C |
| ATOM | 800 | CD2 | LEU | A | 318 | −28.452 | 62.164 | 19.447 | 1.00 | 45.18 | C |
| ATOM | 801 | N | ALA | A | 319 | −27.113 | 66.947 | 21.511 | 1.00 | 49.23 | N |
| ATOM | 802 | CA | ALA | A | 319 | −27.556 | 68.119 | 22.269 | 1.00 | 49.43 | C |
| ATOM | 803 | C | ALA | A | 319 | −28.328 | 69.055 | 21.346 | 1.00 | 50.99 | C |
| ATOM | 804 | O | ALA | A | 319 | −29.147 | 69.861 | 21.797 | 1.00 | 52.12 | O |
| ATOM | 805 | CB | ALA | A | 319 | −26.362 | 68.845 | 22.867 | 1.00 | 50.35 | C |
| ATOM | 806 | N | GLY | A | 320 | −28.043 | 68.952 | 20.051 | 1.00 | 50.36 | N |
| ATOM | 807 | CA | GLY | A | 320 | −28.735 | 69.759 | 19.065 | 1.00 | 51.25 | C |
| ATOM | 808 | C | GLY | A | 320 | −28.373 | 71.225 | 18.970 | 1.00 | 52.05 | C |
| ATOM | 809 | O | GLY | A | 320 | −29.143 | 72.010 | 18.423 | 1.00 | 52.88 | O |
| ATOM | 810 | N | GLN | A | 321 | −27.211 | 71.606 | 19.486 | 1.00 | 53.24 | N |
| ATOM | 811 | CA | GLN | A | 321 | −26.791 | 73.006 | 19.424 | 1.00 | 53.75 | C |
| ATOM | 812 | C | GLN | A | 321 | −25.468 | 73.171 | 18.686 | 1.00 | 51.57 | C |
| ATOM | 813 | O | GLN | A | 321 | −24.545 | 72.375 | 18.860 | 1.00 | 49.91 | O |
| ATOM | 814 | CB | GLN | A | 321 | −26.679 | 73.582 | 20.836 | 1.00 | 56.27 | C |
| ATOM | 815 | CG | GLN | A | 321 | −27.991 | 73.527 | 21.606 | 1.00 | 64.49 | C |
| ATOM | 816 | CD | GLN | A | 321 | −27.847 | 73.980 | 23.045 | 1.00 | 68.32 | C |
| ATOM | 817 | OE1 | GLN | A | 321 | −27.414 | 75.105 | 23.311 | 1.00 | 72.34 | O |
| ATOM | 818 | NE2 | GLN | A | 321 | −28.212 | 73.106 | 23.985 | 1.00 | 69.61 | N |
| ATOM | 819 | N | ASP | A | 322 | −25.382 | 74.202 | 17.854 | 1.00 | 50.10 | N |
| ATOM | 820 | CA | ASP | A | 322 | −24.163 | 74.447 | 17.101 | 1.00 | 49.44 | C |
| ATOM | 821 | C | ASP | A | 322 | −22.965 | 74.467 | 18.037 | 1.00 | 47.83 | C |
| ATOM | 822 | O | ASP | A | 322 | −23.021 | 75.037 | 19.125 | 1.00 | 46.47 | O |
| ATOM | 823 | CB | ASP | A | 322 | −24.250 | 75.770 | 16.338 | 1.00 | 50.56 | C |
| ATOM | 824 | CG | ASP | A | 322 | −25.403 | 75.797 | 15.354 | 1.00 | 52.77 | C |
| ATOM | 825 | OD1 | ASP | A | 322 | −25.821 | 74.714 | 14.882 | 1.00 | 53.32 | O |
| ATOM | 826 | OD2 | ASP | A | 322 | −25.883 | 76.902 | 15.042 | 1.00 | 53.39 | O |
| ATOM | 827 | N | PHE | A | 323 | −21.881 | 73.832 | 17.608 | 1.00 | 45.19 | N |
| ATOM | 828 | CA | PHE | A | 323 | −20.682 | 73.771 | 18.413 | 1.00 | 44.28 | C |
| ATOM | 829 | C | PHE | A | 323 | −19.439 | 73.654 | 17.548 | 1.00 | 43.95 | C |
| ATOM | 830 | O | PHE | A | 323 | −19.500 | 73.237 | 16.391 | 1.00 | 44.15 | O |
| ATOM | 831 | CB | PHE | A | 323 | −20.768 | 72.578 | 19.372 | 1.00 | 43.87 | C |
| ATOM | 832 | CG | PHE | A | 323 | −20.765 | 71.244 | 18.683 | 1.00 | 45.16 | C |
| ATOM | 833 | CD1 | PHE | A | 323 | −19.565 | 70.607 | 18.373 | 1.00 | 44.89 | C |
| ATOM | 834 | CD2 | PHE | A | 323 | −21.960 | 70.631 | 18.321 | 1.00 | 44.36 | C |
| ATOM | 835 | CE1 | PHE | A | 323 | −19.558 | 69.380 | 17.713 | 1.00 | 45.93 | C |
| ATOM | 836 | CE2 | PHE | A | 323 | −21.963 | 69.404 | 17.659 | 1.00 | 44.15 | C |
| ATOM | 837 | CZ | PHE | A | 323 | −20.762 | 68.777 | 17.355 | 1.00 | 45.48 | C |
| ATOM | 838 | N | THR | A | 324 | −18.311 | 74.055 | 18.117 | 1.00 | 43.72 | N |
| ATOM | 839 | CA | THR | A | 324 | −17.028 | 73.967 | 17.439 | 1.00 | 43.44 | C |
| ATOM | 840 | C | THR | A | 324 | −16.164 | 73.171 | 18.408 | 1.00 | 42.73 | C |
| ATOM | 841 | O | THR | A | 324 | −16.255 | 73.355 | 19.626 | 1.00 | 44.48 | O |
| ATOM | 842 | CB | THR | A | 324 | −16.402 | 75.360 | 17.230 | 1.00 | 43.93 | C |
| ATOM | 843 | OG1 | THR | A | 324 | −17.299 | 76.171 | 16.462 | 1.00 | 45.37 | O |
| ATOM | 844 | CG2 | THR | A | 324 | −15.060 | 75.247 | 16.500 | 1.00 | 43.46 | C |
| ATOM | 845 | N | ARG | A | 325 | −15.345 | 72.265 | 17.899 | 1.00 | 39.87 | N |
| ATOM | 846 | CA | ARG | A | 325 | −14.514 | 71.506 | 18.812 | 1.00 | 36.33 | C |
| ATOM | 847 | C | ARG | A | 325 | −13.263 | 70.976 | 18.170 | 1.00 | 34.62 | C |
| ATOM | 848 | O | ARG | A | 325 | −13.152 | 70.903 | 16.947 | 1.00 | 35.68 | O |
| ATOM | 849 | CB | ARG | A | 325 | −15.302 | 70.330 | 19.416 | 1.00 | 35.19 | C |
| ATOM | 850 | CG | ARG | A | 325 | −15.706 | 69.255 | 18.416 | 1.00 | 33.29 | C |
| ATOM | 851 | CD | ARG | A | 325 | −16.439 | 68.081 | 19.091 | 1.00 | 30.61 | C |
| ATOM | 852 | NE | ARG | A | 325 | −15.548 | 67.203 | 19.850 | 1.00 | 31.33 | N |
| ATOM | 853 | CZ | ARG | A | 325 | −15.419 | 67.207 | 21.176 | 1.00 | 32.99 | C |
| ATOM | 854 | NH1 | ARG | A | 325 | −16.128 | 68.042 | 21.924 | 1.00 | 32.26 | N |
| ATOM | 855 | NH2 | ARG | A | 325 | −14.564 | 66.377 | 21.763 | 1.00 | 32.67 | N |
| ATOM | 856 | N | SER | A | 326 | −12.306 | 70.642 | 19.019 | 1.00 | 32.04 | N |
| ATOM | 857 | CA | SER | A | 326 | −11.072 | 70.045 | 18.575 | 1.00 | 32.88 | C |
| ATOM | 858 | C | SER | A | 326 | −11.111 | 68.678 | 19.237 | 1.00 | 34.05 | C |
| ATOM | 859 | O | SER | A | 326 | −11.525 | 68.544 | 20.399 | 1.00 | 34.85 | O |
| ATOM | 860 | CB | SER | A | 326 | −9.882 | 70.869 | 19.032 | 1.00 | 32.79 | C |
| ATOM | 861 | OG | SER | A | 326 | −9.771 | 72.021 | 18.212 | 1.00 | 33.86 | O |
| ATOM | 862 | N | PRO | A | 327 | −10.704 | 67.638 | 18.505 | 1.00 | 33.77 | N |
| ATOM | 863 | CA | PRO | A | 327 | −10.708 | 66.265 | 19.014 | 1.00 | 33.48 | C |
| ATOM | 864 | C | PRO | A | 327 | −9.868 | 65.968 | 20.238 | 1.00 | 35.48 | C |
| ATOM | 865 | O | PRO | A | 327 | −8.835 | 66.606 | 20.481 | 1.00 | 35.13 | O |
| ATOM | 866 | CB | PRO | A | 327 | −10.259 | 65.452 | 17.807 | 1.00 | 34.01 | C |
| ATOM | 867 | CG | PRO | A | 327 | −9.300 | 66.403 | 17.122 | 1.00 | 33.50 | C |
| ATOM | 868 | CD | PRO | A | 327 | −10.073 | 67.706 | 17.174 | 1.00 | 33.70 | C |
| ATOM | 869 | N | ILE | A | 328 | −10.334 | 64.986 | 21.008 | 1.00 | 34.40 | N |
| ATOM | 870 | CA | ILE | A | 328 | −9.624 | 64.530 | 22.190 | 1.00 | 33.40 | C |
| ATOM | 871 | C | ILE | A | 328 | −8.424 | 63.759 | 21.636 | 1.00 | 33.96 | C |
| ATOM | 872 | O | ILE | A | 328 | −8.589 | 62.837 | 20.829 | 1.00 | 32.48 | O |
| ATOM | 873 | CB | ILE | A | 328 | −10.504 | 63.580 | 23.021 | 1.00 | 32.50 | C |
| ATOM | 874 | CG1 | ILE | A | 328 | −11.635 | 64.369 | 23.676 | 1.00 | 32.65 | C |
| ATOM | 875 | CG2 | ILE | A | 328 | −9.662 | 62.853 | 24.056 | 1.00 | 30.39 | C |
| ATOM | 876 | CD1 | ILE | A | 328 | −12.664 | 63.494 | 24.346 | 1.00 | 32.58 | C |
| ATOM | 877 | N | VAL | A | 329 | −7.221 | 64.133 | 22.054 | 1.00 | 33.19 | N |
| ATOM | 878 | CA | VAL | A | 329 | −6.038 | 63.465 | 21.548 | 1.00 | 33.26 | C |
| ATOM | 879 | C | VAL | A | 329 | −5.008 | 63.156 | 22.612 | 1.00 | 34.78 | C |
| ATOM | 880 | O | VAL | A | 329 | −5.109 | 63.585 | 23.761 | 1.00 | 35.98 | O |
| ATOM | 881 | CB | VAL | A | 329 | −5.333 | 64.310 | 20.458 | 1.00 | 32.77 | C |
| ATOM | 882 | CG1 | VAL | A | 329 | −6.320 | 64.674 | 19.364 | 1.00 | 32.84 | C |
| ATOM | 883 | CG2 | VAL | A | 329 | −4.728 | 65.568 | 21.080 | 1.00 | 31.49 | C |
| ATOM | 884 | N | GLN | A | 330 | −4.004 | 62.405 | 22.197 | 1.00 | 36.55 | N |
| ATOM | 885 | CA | GLN | A | 330 | −2.909 | 62.027 | 23.060 | 1.00 | 38.67 | C |
| ATOM | 886 | C | GLN | A | 330 | −1.654 | 62.315 | 22.264 | 1.00 | 37.32 | C |
| ATOM | 887 | O | GLN | A | 330 | −1.561 | 61.952 | 21.089 | 1.00 | 38.02 | O |
| ATOM | 888 | CB | GLN | A | 330 | −2.992 | 60.542 | 23.405 | 1.00 | 40.05 | C |
| ATOM | 889 | CG | GLN | A | 330 | −3.292 | 60.284 | 24.861 | 1.00 | 46.72 | C |
| ATOM | 890 | CD | GLN | A | 330 | −3.614 | 58.832 | 25.129 | 1.00 | 50.91 | C |
| ATOM | 891 | OE1 | GLN | A | 330 | −2.871 | 57.934 | 24.716 | 1.00 | 53.09 | O |
| ATOM | 892 | NE2 | GLN | A | 330 | −4.727 | 58.587 | 25.825 | 1.00 | 52.66 | N |
| ATOM | 893 | N | GLY | A | 331 | −0.699 | 62.984 | 22.898 | 1.00 | 37.23 | N |
| ATOM | 894 | CA | GLY | A | 331 | 0.541 | 63.307 | 22.217 | 1.00 | 36.18 | C |
| ATOM | 895 | C | GLY | A | 331 | 1.243 | 64.488 | 22.846 | 1.00 | 34.56 | C |
| ATOM | 896 | O | GLY | A | 331 | 1.022 | 64.787 | 24.022 | 1.00 | 34.05 | O |
| ATOM | 897 | N | GLY | A | 332 | 2.071 | 65.165 | 22.056 | 1.00 | 34.37 | N |
| ATOM | 898 | CA | GLY | A | 332 | 2.831 | 66.298 | 22.551 | 1.00 | 36.06 | C |
| ATOM | 899 | C | GLY | A | 332 | 2.089 | 67.619 | 22.587 | 1.00 | 38.62 | C |
| ATOM | 900 | O | GLY | A | 332 | 2.635 | 68.618 | 23.059 | 1.00 | 41.41 | O |
| ATOM | 901 | N | THR | A | 333 | 0.853 | 67.637 | 22.095 | 1.00 | 36.73 | N |
| ATOM | 902 | CA | THR | A | 333 | 0.068 | 68.861 | 22.086 | 1.00 | 35.15 | C |
| ATOM | 903 | C | THR | A | 333 | −1.344 | 68.554 | 21.595 | 1.00 | 36.05 | C |
| ATOM | 904 | O | THR | A | 333 | −1.592 | 67.472 | 21.062 | 1.00 | 37.04 | O |
| ATOM | 905 | CB | THR | A | 333 | 0.746 | 69.936 | 21.181 | 1.00 | 35.03 | C |
| ATOM | 906 | OG1 | THR | A | 333 | 0.032 | 71.171 | 21.287 | 1.00 | 37.24 | O |
| ATOM | 907 | CG2 | THR | A | 333 | 0.777 | 69.499 | 19.740 | 1.00 | 30.06 | C |
| ATOM | 908 | N | THR | A | 334 | −2.273 | 69.485 | 21.793 | 1.00 | 36.07 | N |
| ATOM | 909 | CA | THR | A | 334 | −3.654 | 69.280 | 21.354 | 1.00 | 36.17 | C |
| ATOM | 910 | C | THR | A | 334 | −3.806 | 69.633 | 19.880 | 1.00 | 37.44 | C |
| ATOM | 911 | O | THR | A | 334 | −2.876 | 70.158 | 19.265 | 1.00 | 38.70 | O |
| ATOM | 912 | CB | THR | A | 334 | −4.641 | 70.124 | 22.183 | 1.00 | 36.07 | C |
| ATOM | 913 | OG1 | THR | A | 334 | −4.095 | 71.432 | 22.389 | 1.00 | 34.21 | O |
| ATOM | 914 | CG2 | THR | A | 334 | −4.914 | 69.460 | 23.524 | 1.00 | 33.73 | C |
| ATOM | 915 | N | ALA | A | 335 | −4.977 | 69.353 | 19.312 | 1.00 | 37.55 | N |
| ATOM | 916 | CA | ALA | A | 335 | −5.211 | 69.610 | 17.894 | 1.00 | 36.41 | C |
| ATOM | 917 | C | ALA | A | 335 | −5.990 | 70.885 | 17.601 | 1.00 | 37.28 | C |
| ATOM | 918 | O | ALA | A | 335 | −6.411 | 71.117 | 16.466 | 1.00 | 34.11 | O |
| ATOM | 919 | CB | ALA | A | 335 | −5.917 | 68.413 | 17.265 | 1.00 | 35.84 | C |
| ATOM | 920 | N | ASP | A | 336 | −6.170 | 71.716 | 18.621 | 1.00 | 38.47 | N |
| ATOM | 921 | CA | ASP | A | 336 | −6.901 | 72.971 | 18.464 | 1.00 | 38.23 | C |
| ATOM | 922 | C | ASP | A | 336 | −6.052 | 74.069 | 17.815 | 1.00 | 37.04 | C |
| ATOM | 923 | O | ASP | A | 336 | −6.430 | 75.236 | 17.825 | 1.00 | 39.04 | O |
| ATOM | 924 | CB | ASP | A | 336 | −7.397 | 73.448 | 19.827 | 1.00 | 40.61 | C |
| ATOM | 925 | CG | ASP | A | 336 | −6.263 | 73.684 | 20.804 | 1.00 | 44.95 | C |
| ATOM | 926 | OD1 | ASP | A | 336 | −5.347 | 72.832 | 20.870 | 1.00 | 46.62 | O |
| ATOM | 927 | OD2 | ASP | A | 336 | −6.287 | 74.716 | 21.506 | 1.00 | 46.35 | O |
| ATOM | 928 | N | HIS | A | 337 | −4.906 | 73.696 | 17.258 | 1.00 | 34.14 | N |
| ATOM | 929 | CA | HIS | A | 337 | −4.030 | 74.656 | 16.600 | 1.00 | 33.84 | C |
| ATOM | 930 | C | HIS | A | 337 | −3.108 | 73.870 | 15.678 | 1.00 | 32.85 | C |
| ATOM | 931 | O | HIS | A | 337 | −2.965 | 72.661 | 15.831 | 1.00 | 32.74 | O |
| ATOM | 932 | CB | HIS | A | 337 | −3.176 | 75.421 | 17.624 | 1.00 | 35.55 | C |
| ATOM | 933 | CG | HIS | A | 337 | −2.131 | 74.574 | 18.282 | 1.00 | 38.21 | C |
| ATOM | 934 | ND1 | HIS | A | 337 | −2.343 | 73.925 | 19.480 | 1.00 | 40.97 | N |
| ATOM | 935 | CD2 | HIS | A | 337 | −0.911 | 74.174 | 17.851 | 1.00 | 38.52 | C |
| ATOM | 936 | CE1 | HIS | A | 337 | −1.304 | 73.155 | 19.752 | 1.00 | 40.23 | C |
| ATOM | 937 | NE2 | HIS | A | 337 | −0.421 | 73.286 | 18.778 | 1.00 | 40.26 | N |
| ATOM | 938 | N | PRO | A | 338 | −2.451 | 74.550 | 14.726 | 1.00 | 31.84 | N |
| ATOM | 939 | CA | PRO | A | 338 | −1.539 | 73.871 | 13.795 | 1.00 | 32.74 | C |
| ATOM | 940 | C | PRO | A | 338 | −0.344 | 73.259 | 14.551 | 1.00 | 32.81 | C |
| ATOM | 941 | O | PRO | A | 338 | 0.147 | 73.850 | 15.507 | 1.00 | 32.49 | O |
| ATOM | 942 | CB | PRO | A | 338 | −1.085 | 75.000 | 12.852 | 1.00 | 30.72 | C |
| ATOM | 943 | CG | PRO | A | 338 | −2.085 | 76.131 | 13.082 | 1.00 | 30.03 | C |
| ATOM | 944 | CD | PRO | A | 338 | −2.406 | 76.013 | 14.540 | 1.00 | 31.43 | C |
| ATOM | 945 | N | LEU | A | 339 | 0.131 | 72.095 | 14.117 | 1.00 | 32.48 | N |
| ATOM | 946 | CA | LEU | A | 339 | 1.273 | 71.459 | 14.772 | 1.00 | 34.28 | C |
| ATOM | 947 | C | LEU | A | 339 | 2.470 | 72.428 | 14.830 | 1.00 | 35.08 | C |
| ATOM | 948 | O | LEU | A | 339 | 3.115 | 72.570 | 15.865 | 1.00 | 35.86 | O |
| ATOM | 949 | CB | LEU | A | 339 | 1.678 | 70.185 | 14.026 | 1.00 | 31.75 | C |
| ATOM | 950 | CG | LEU | A | 339 | 2.696 | 69.305 | 14.758 | 1.00 | 34.02 | C |
| ATOM | 951 | CD1 | LEU | A | 339 | 2.066 | 68.784 | 16.059 | 1.00 | 32.23 | C |
| ATOM | 952 | CD2 | LEU | A | 339 | 3.125 | 68.131 | 13.866 | 1.00 | 31.57 | C |
| ATOM | 953 | N | ILE | A | 340 | 2.767 | 73.092 | 13.719 | 1.00 | 35.80 | N |
| ATOM | 954 | CA | ILE | A | 340 | 3.876 | 74.044 | 13.691 | 1.00 | 37.04 | C |
| ATOM | 955 | C | ILE | A | 340 | 3.366 | 75.483 | 13.578 | 1.00 | 37.40 | C |
| ATOM | 956 | O | ILE | A | 340 | 2.675 | 75.828 | 12.623 | 1.00 | 36.16 | O |
| ATOM | 957 | CB | ILE | A | 340 | 4.825 | 73.775 | 12.506 | 1.00 | 36.93 | C |
| ATOM | 958 | CG1 | ILE | A | 340 | 5.192 | 72.282 | 12.449 | 1.00 | 34.97 | C |
| ATOM | 959 | CG2 | ILE | A | 340 | 6.080 | 74.634 | 12.652 | 1.00 | 36.68 | C |
| ATOM | 960 | CD1 | ILE | A | 340 | 5.812 | 71.732 | 13.728 | 1.00 | 32.70 | C |
| ATOM | 961 | N | GLU | A | 341 | 3.693 | 76.307 | 14.567 | 1.00 | 39.07 | N |
| ATOM | 962 | CA | GLU | A | 341 | 3.283 | 77.706 | 14.574 | 1.00 | 41.74 | C |
| ATOM | 963 | C | GLU | A | 341 | 4.424 | 78.523 | 13.964 | 1.00 | 42.95 | C |
| ATOM | 964 | O | GLU | A | 341 | 4.898 | 78.231 | 12.861 | 1.00 | 41.64 | O |
| ATOM | 965 | CB | GLU | A | 341 | 3.041 | 78.194 | 16.003 | 1.00 | 45.85 | C |
| ATOM | 966 | CG | GLU | A | 341 | 2.226 | 77.262 | 16.875 | 1.00 | 51.43 | C |
| ATOM | 967 | CD | GLU | A | 341 | 0.745 | 77.376 | 16.622 | 1.00 | 54.45 | C |
| ATOM | 968 | OE1 | GLU | A | 341 | 0.353 | 77.360 | 15.437 | 1.00 | 57.80 | O |
| ATOM | 969 | OE2 | GLU | A | 341 | −0.024 | 77.471 | 17.607 | 1.00 | 55.31 | O |
| ATOM | 970 | N | ASP | A | 342 | 4.870 | 79.545 | 14.686 | 1.00 | 43.37 | N |
| ATOM | 971 | CA | ASP | A | 342 | 5.953 | 80.372 | 14.187 | 1.00 | 47.02 | C |
| ATOM | 972 | C | ASP | A | 342 | 7.165 | 80.348 | 15.122 | 1.00 | 47.79 | C |
| ATOM | 973 | O | ASP | A | 342 | 8.039 | 81.217 | 15.058 | 1.00 | 48.27 | O |
| ATOM | 974 | CB | ASP | A | 342 | 5.467 | 81.809 | 13.965 | 1.00 | 49.63 | C |
| ATOM | 975 | CG | ASP | A | 342 | 4.825 | 82.408 | 15.196 | 1.00 | 54.32 | C |
| ATOM | 976 | OD1 | ASP | A | 342 | 5.139 | 81.948 | 16.318 | 1.00 | 57.47 | O |
| ATOM | 977 | OD2 | ASP | A | 342 | 4.015 | 83.353 | 15.042 | 1.00 | 58.06 | O |
| ATOM | 978 | N | THR | A | 343 | 7.210 | 79.348 | 15.996 | 1.00 | 46.70 | N |
| ATOM | 979 | CA | THR | A | 343 | 8.328 | 79.193 | 16.918 | 1.00 | 44.88 | C |
| ATOM | 980 | C | THR | A | 343 | 8.781 | 77.755 | 16.806 | 1.00 | 42.51 | C |
| ATOM | 981 | O | THR | A | 343 | 8.049 | 76.835 | 17.150 | 1.00 | 42.39 | O |
| ATOM | 982 | CB | THR | A | 343 | 7.921 | 79.494 | 18.369 | 1.00 | 44.94 | C |
| ATOM | 983 | OG1 | THR | A | 343 | 7.507 | 80.863 | 18.470 | 1.00 | 49.06 | O |
| ATOM | 984 | CG2 | THR | A | 343 | 9.094 | 79.251 | 19.314 | 1.00 | 42.70 | C |
| ATOM | 985 | N | TYR | A | 344 | 9.996 | 77.559 | 16.320 | 1.00 | 40.58 | N |
| ATOM | 986 | CA | TYR | A | 344 | 10.494 | 76.210 | 16.145 | 1.00 | 40.17 | C |
| ATOM | 987 | C | TYR | A | 344 | 11.941 | 76.254 | 15.714 | 1.00 | 40.73 | C |
| ATOM | 988 | O | TYR | A | 344 | 12.464 | 77.313 | 15.384 | 1.00 | 40.98 | O |
| ATOM | 989 | CB | TYR | A | 344 | 9.663 | 75.513 | 15.069 | 1.00 | 37.79 | C |
| ATOM | 990 | CG | TYR | A | 344 | 9.563 | 76.335 | 13.801 | 1.00 | 36.88 | C |
| ATOM | 991 | CD1 | TYR | A | 344 | 10.589 | 76.324 | 12.861 | 1.00 | 37.23 | C |
| ATOM | 992 | CD2 | TYR | A | 344 | 8.478 | 77.193 | 13.583 | 1.00 | 38.58 | C |
| ATOM | 993 | CE1 | TYR | A | 344 | 10.547 | 77.151 | 11.732 | 1.00 | 37.53 | C |
| ATOM | 994 | CE2 | TYR | A | 344 | 8.429 | 78.031 | 12.457 | 1.00 | 37.32 | C |
| ATOM | 995 | CZ | TYR | A | 344 | 9.469 | 78.001 | 11.542 | 1.00 | 35.53 | C |
| ATOM | 996 | OH | TYR | A | 344 | 9.456 | 78.836 | 10.453 | 1.00 | 34.84 | O |
| ATOM | 997 | N | ILE | A | 345 | 12.581 | 75.093 | 15.717 | 1.00 | 41.08 | N |
| ATOM | 998 | CA | ILE | A | 345 | 13.963 | 74.986 | 15.294 | 1.00 | 40.62 | C |
| ATOM | 999 | C | ILE | A | 345 | 13.941 | 74.231 | 13.971 | 1.00 | 42.63 | C |
| ATOM | 1000 | O | ILE | A | 345 | 13.374 | 73.142 | 13.881 | 1.00 | 42.67 | O |
| ATOM | 1001 | CB | ILE | A | 345 | 14.801 | 74.218 | 16.328 | 1.00 | 39.22 | C |
| ATOM | 1002 | CG1 | ILE | A | 345 | 14.920 | 75.055 | 17.602 | 1.00 | 39.09 | C |
| ATOM | 1003 | CG2 | ILE | A | 345 | 16.168 | 73.883 | 15.750 | 1.00 | 37.28 | C |
| ATOM | 1004 | CD1 | ILE | A | 345 | 15.705 | 74.398 | 18.706 | 1.00 | 39.50 | C |
| ATOM | 1005 | N | GLU | A | 346 | 14.535 | 74.828 | 12.943 | 1.00 | 43.58 | N |
| ATOM | 1006 | CA | GLU | A | 346 | 14.579 | 74.218 | 11.618 | 1.00 | 43.37 | C |
| ATOM | 1007 | C | GLU | A | 346 | 15.984 | 73.720 | 11.334 | 1.00 | 43.15 | C |
| ATOM | 1008 | O | GLU | A | 346 | 16.952 | 74.466 | 11.468 | 1.00 | 44.37 | O |
| ATOM | 1009 | CB | GLU | A | 346 | 14.162 | 75.244 | 10.554 | 1.00 | 41.97 | C |
| ATOM | 1010 | CG | GLU | A | 346 | 14.454 | 74.828 | 9.115 | 1.00 | 39.53 | C |
| ATOM | 1011 | CD | GLU | A | 346 | 14.009 | 75.881 | 8.111 | 1.00 | 38.64 | C |
| ATOM | 1012 | OE1 | GLU | A | 346 | 13.641 | 76.990 | 8.557 | 1.00 | 37.41 | O |
| ATOM | 1013 | OE2 | GLU | A | 346 | 14.032 | 75.604 | 6.888 | 1.00 | 35.04 | O |
| ATOM | 1014 | N | VAL | A | 347 | 16.090 | 72.458 | 10.942 | 1.00 | 42.11 | N |
| ATOM | 1015 | CA | VAL | A | 347 | 17.380 | 71.857 | 10.652 | 1.00 | 43.31 | C |
| ATOM | 1016 | C | VAL | A | 347 | 17.419 | 71.348 | 9.217 | 1.00 | 44.81 | C |
| ATOM | 1017 | O | VAL | A | 347 | 16.941 | 70.249 | 8.913 | 1.00 | 47.34 | O |
| ATOM | 1018 | CB | VAL | A | 347 | 17.671 | 70.697 | 11.627 | 1.00 | 41.82 | C |
| ATOM | 1019 | CG1 | VAL | A | 347 | 19.028 | 70.098 | 11.334 | 1.00 | 42.29 | C |
| ATOM | 1020 | CG2 | VAL | A | 347 | 17.610 | 71.205 | 13.060 | 1.00 | 40.74 | C |
| ATOM | 1021 | N | ASP | A | 348 | 17.990 | 72.165 | 8.338 | 1.00 | 45.54 | N |
| ATOM | 1022 | CA | ASP | A | 348 | 18.101 | 71.836 | 6.927 | 1.00 | 45.47 | C |
| ATOM | 1023 | C | ASP | A | 348 | 19.295 | 70.919 | 6.711 | 1.00 | 46.39 | C |
| ATOM | 1024 | O | ASP | A | 348 | 20.435 | 71.381 | 6.679 | 1.00 | 47.29 | O |
| ATOM | 1025 | CB | ASP | A | 348 | 18.298 | 73.111 | 6.113 | 1.00 | 44.76 | C |
| ATOM | 1026 | CG | ASP | A | 348 | 18.122 | 72.885 | 4.630 | 1.00 | 45.99 | C |
| ATOM | 1027 | OD1 | ASP | A | 348 | 18.379 | 71.751 | 4.161 | 1.00 | 44.36 | O |
| ATOM | 1028 | OD2 | ASP | A | 348 | 17.735 | 73.847 | 3.932 | 1.00 | 47.60 | O |
| ATOM | 1029 | N | LEU | A | 349 | 19.040 | 69.626 | 6.555 | 1.00 | 46.04 | N |
| ATOM | 1030 | CA | LEU | A | 349 | 20.132 | 68.688 | 6.358 | 1.00 | 48.20 | C |
| ATOM | 1031 | C | LEU | A | 349 | 20.922 | 68.976 | 5.086 | 1.00 | 50.55 | C |
| ATOM | 1032 | O | LEU | A | 349 | 22.152 | 69.001 | 5.111 | 1.00 | 50.59 | O |
| ATOM | 1033 | CB | LEU | A | 349 | 19.610 | 67.248 | 6.348 | 1.00 | 45.72 | C |
| ATOM | 1034 | CG | LEU | A | 349 | 19.060 | 66.764 | 7.692 | 1.00 | 45.26 | C |
| ATOM | 1035 | CD1 | LEU | A | 349 | 18.675 | 65.298 | 7.597 | 1.00 | 43.83 | C |
| ATOM | 1036 | CD2 | LEU | A | 349 | 20.111 | 66.973 | 8.777 | 1.00 | 42.75 | C |
| ATOM | 1037 | N | GLU | A | 350 | 20.220 | 69.207 | 3.982 | 1.00 | 52.87 | N |
| ATOM | 1038 | CA | GLU | A | 350 | 20.872 | 69.494 | 2.704 | 1.00 | 55.20 | C |
| ATOM | 1039 | C | GLU | A | 350 | 21.876 | 70.649 | 2.815 | 1.00 | 55.41 | C |
| ATOM | 1040 | O | GLU | A | 350 | 22.977 | 70.580 | 2.265 | 1.00 | 55.52 | O |
| ATOM | 1041 | CB | GLU | A | 350 | 19.813 | 69.827 | 1.653 | 1.00 | 57.91 | C |
| ATOM | 1042 | CG | GLU | A | 350 | 20.341 | 70.012 | 0.247 | 1.00 | 63.38 | C |
| ATOM | 1043 | CD | GLU | A | 350 | 19.215 | 70.175 | −0.763 | 1.00 | 67.80 | C |
| ATOM | 1044 | OE1 | GLU | A | 350 | 18.316 | 69.301 | −0.791 | 1.00 | 69.57 | O |
| ATOM | 1045 | OE2 | GLU | A | 350 | 19.227 | 71.167 | −1.527 | 1.00 | 69.49 | O |
| ATOM | 1046 | N | ASN | A | 351 | 21.496 | 71.704 | 3.531 | 1.00 | 54.53 | N |
| ATOM | 1047 | CA | ASN | A | 351 | 22.367 | 72.863 | 3.706 | 1.00 | 54.52 | C |
| ATOM | 1048 | C | ASN | A | 351 | 23.182 | 72.827 | 5.003 | 1.00 | 55.08 | C |
| ATOM | 1049 | O | ASN | A | 351 | 23.928 | 73.767 | 5.302 | 1.00 | 53.99 | O |
| ATOM | 1050 | CB | ASN | A | 351 | 21.549 | 74.157 | 3.637 | 1.00 | 53.99 | C |
| ATOM | 1051 | CG | ASN | A | 351 | 21.046 | 74.448 | 2.238 | 1.00 | 55.37 | C |
| ATOM | 1052 | OD1 | ASN | A | 351 | 21.834 | 74.577 | 1.300 | 1.00 | 55.35 | O |
| ATOM | 1053 | ND2 | ASN | A | 351 | 19.732 | 74.551 | 2.088 | 1.00 | 55.25 | N |
| ATOM | 1054 | N | GLN | A | 352 | 23.029 | 71.752 | 5.773 | 1.00 | 54.22 | N |
| ATOM | 1055 | CA | GLN | A | 352 | 23.777 | 71.588 | 7.013 | 1.00 | 53.98 | C |
| ATOM | 1056 | C | GLN | A | 352 | 23.699 | 72.870 | 7.833 | 1.00 | 53.32 | C |
| ATOM | 1057 | O | GLN | A | 352 | 24.680 | 73.283 | 8.446 | 1.00 | 53.45 | O |
| ATOM | 1058 | CB | GLN | A | 352 | 25.237 | 71.274 | 6.674 | 1.00 | 54.55 | C |
| ATOM | 1059 | CG | GLN | A | 352 | 25.894 | 70.245 | 7.564 | 1.00 | 58.31 | C |
| ATOM | 1060 | CD | GLN | A | 352 | 25.181 | 68.909 | 7.534 | 1.00 | 59.65 | C |
| ATOM | 1061 | OE1 | GLN | A | 352 | 24.981 | 68.320 | 6.475 | 1.00 | 61.09 | O |
| ATOM | 1062 | NE2 | GLN | A | 352 | 24.798 | 68.422 | 8.703 | 1.00 | 62.74 | N |
| ATOM | 1063 | N | HIS | A | 353 | 22.521 | 73.484 | 7.851 | 1.00 | 53.06 | N |
| ATOM | 1064 | CA | HIS | A | 353 | 22.315 | 74.745 | 8.559 | 1.00 | 52.52 | C |
| ATOM | 1065 | C | HIS | A | 353 | 21.110 | 74.653 | 9.505 | 1.00 | 51.55 | C |
| ATOM | 1066 | O | HIS | A | 353 | 20.157 | 73.921 | 9.238 | 1.00 | 51.86 | O |
| ATOM | 1067 | CB | HIS | A | 353 | 22.107 | 75.846 | 7.510 | 1.00 | 55.13 | C |
| ATOM | 1068 | CG | HIS | A | 353 | 22.344 | 77.234 | 8.013 | 1.00 | 56.74 | C |
| ATOM | 1069 | ND1 | HIS | A | 353 | 21.336 | 78.025 | 8.518 | 1.00 | 59.44 | N |
| ATOM | 1070 | CD2 | HIS | A | 353 | 23.472 | 77.982 | 8.066 | 1.00 | 57.39 | C |
| ATOM | 1071 | CE1 | HIS | A | 353 | 21.831 | 79.202 | 8.859 | 1.00 | 60.25 | C |
| ATOM | 1072 | NE2 | HIS | A | 353 | 23.126 | 79.202 | 8.595 | 1.00 | 59.21 | N |
| ATOM | 1073 | N | MET | A | 354 | 21.152 | 75.397 | 10.608 | 1.00 | 49.47 | N |
| ATOM | 1074 | CA | MET | A | 354 | 20.065 | 75.378 | 11.581 | 1.00 | 47.67 | C |
| ATOM | 1075 | C | MET | A | 354 | 19.546 | 76.783 | 11.867 | 1.00 | 48.47 | C |
| ATOM | 1076 | O | MET | A | 354 | 20.318 | 77.730 | 12.001 | 1.00 | 50.30 | O |
| ATOM | 1077 | CB | MET | A | 354 | 20.537 | 74.701 | 12.882 | 1.00 | 44.47 | C |
| ATOM | 1078 | CG | MET | A | 354 | 19.521 | 74.668 | 14.030 | 1.00 | 43.09 | C |
| ATOM | 1079 | SD | MET | A | 354 | 19.988 | 73.516 | 15.384 | 1.00 | 37.07 | S |
| ATOM | 1080 | CE | MET | A | 354 | 21.165 | 74.499 | 16.282 | 1.00 | 42.56 | C |
| ATOM | 1081 | N | TRP | A | 355 | 18.225 | 76.906 | 11.945 | 1.00 | 48.87 | N |
| ATOM | 1082 | CA | TRP | A | 355 | 17.564 | 78.180 | 12.222 | 1.00 | 48.14 | C |
| ATOM | 1083 | C | TRP | A | 355 | 16.668 | 77.999 | 13.432 | 1.00 | 47.55 | C |
| ATOM | 1084 | O | TRP | A | 355 | 16.125 | 76.920 | 13.656 | 1.00 | 48.98 | O |
| ATOM | 1085 | CB | TRP | A | 355 | 16.650 | 78.601 | 11.059 | 1.00 | 47.79 | C |
| ATOM | 1086 | CG | TRP | A | 355 | 17.317 | 79.041 | 9.795 | 1.00 | 47.72 | C |
| ATOM | 1087 | CD1 | TRP | A | 355 | 17.617 | 80.326 | 9.424 | 1.00 | 48.14 | C |
| ATOM | 1088 | CD2 | TRP | A | 355 | 17.738 | 78.202 | 8.712 | 1.00 | 47.23 | C |
| ATOM | 1089 | NE1 | TRP | A | 355 | 18.194 | 80.334 | 8.175 | 1.00 | 47.59 | N |
| ATOM | 1090 | CE2 | TRP | A | 355 | 18.283 | 79.046 | 7.717 | 1.00 | 46.30 | C |
| ATOM | 1091 | CE3 | TRP | A | 355 | 17.708 | 76.819 | 8.486 | 1.00 | 45.06 | C |
| ATOM | 1092 | CZ2 | TRP | A | 355 | 18.796 | 78.550 | 6.517 | 1.00 | 46.70 | C |
| ATOM | 1093 | CZ3 | TRP | A | 355 | 18.218 | 76.327 | 7.291 | 1.00 | 45.12 | C |
| ATOM | 1094 | CH2 | TRP | A | 355 | 18.755 | 77.192 | 6.322 | 1.00 | 45.85 | C |
| ATOM | 1095 | N | TYR | A | 356 | 16.513 | 79.057 | 14.211 | 1.00 | 47.01 | N |
| ATOM | 1096 | CA | TYR | A | 356 | 15.615 | 79.016 | 15.343 | 1.00 | 46.69 | C |
| ATOM | 1097 | C | TYR | A | 356 | 14.694 | 80.207 | 15.187 | 1.00 | 48.27 | C |
| ATOM | 1098 | O | TYR | A | 356 | 15.129 | 81.358 | 15.267 | 1.00 | 49.57 | O |
| ATOM | 1099 | CB | TYR | A | 356 | 16.344 | 79.125 | 16.672 | 1.00 | 45.53 | C |
| ATOM | 1100 | CG | TYR | A | 356 | 15.384 | 79.389 | 17.809 | 1.00 | 45.50 | C |
| ATOM | 1101 | CD1 | TYR | A | 356 | 14.260 | 78.579 | 17.998 | 1.00 | 44.50 | C |
| ATOM | 1102 | CD2 | TYR | A | 356 | 15.578 | 80.465 | 18.680 | 1.00 | 45.68 | C |
| ATOM | 1103 | CE1 | TYR | A | 356 | 13.350 | 78.831 | 19.023 | 1.00 | 42.93 | C |
| ATOM | 1104 | CE2 | TYR | A | 356 | 14.673 | 80.729 | 19.711 | 1.00 | 44.77 | C |
| ATOM | 1105 | CZ | TYR | A | 356 | 13.560 | 79.905 | 19.875 | 1.00 | 44.84 | C |
| ATOM | 1106 | OH | TYR | A | 356 | 12.669 | 80.157 | 20.892 | 1.00 | 43.12 | O |
| ATOM | 1107 | N | TYR | A | 357 | 13.421 | 79.937 | 14.943 | 1.00 | 47.03 | N |
| ATOM | 1108 | CA | TYR | A | 357 | 12.468 | 81.013 | 14.781 | 1.00 | 47.20 | C |
| ATOM | 1109 | C | TYR | A | 357 | 11.694 | 81.228 | 16.066 | 1.00 | 48.30 | C |
| ATOM | 1110 | O | TYR | A | 357 | 11.309 | 80.275 | 16.748 | 1.00 | 48.70 | O |
| ATOM | 1111 | CB | TYR | A | 357 | 11.491 | 80.713 | 13.635 | 1.00 | 46.87 | C |
| ATOM | 1112 | CG | TYR | A | 357 | 12.072 | 80.841 | 12.242 | 1.00 | 43.65 | C |
| ATOM | 1113 | CD1 | TYR | A | 357 | 12.875 | 79.837 | 11.699 | 1.00 | 42.63 | C |
| ATOM | 1114 | CD2 | TYR | A | 357 | 11.805 | 81.967 | 11.460 | 1.00 | 42.31 | C |
| ATOM | 1115 | CE1 | TYR | A | 357 | 13.397 | 79.953 | 10.402 | 1.00 | 43.36 | C |
| ATOM | 1116 | CE2 | TYR | A | 357 | 12.320 | 82.094 | 10.171 | 1.00 | 41.63 | C |
| ATOM | 1117 | CZ | TYR | A | 357 | 13.113 | 81.087 | 9.646 | 1.00 | 42.76 | C |
| ATOM | 1118 | OH | TYR | A | 357 | 13.613 | 81.212 | 8.369 | 1.00 | 43.50 | O |
| ATOM | 1119 | N | LYS | A | 358 | 11.475 | 82.494 | 16.392 | 1.00 | 50.47 | N |
| ATOM | 1120 | CA | LYS | A | 358 | 10.729 | 82.872 | 17.580 | 1.00 | 52.43 | C |
| ATOM | 1121 | C | LYS | A | 358 | 9.698 | 83.876 | 17.087 | 1.00 | 53.16 | C |
| ATOM | 1122 | O | LYS | A | 358 | 10.043 | 84.975 | 16.658 | 1.00 | 54.51 | O |
| ATOM | 1123 | CB | LYS | A | 358 | 11.655 | 83.523 | 18.606 | 1.00 | 53.77 | C |
| ATOM | 1124 | CG | LYS | A | 358 | 11.383 | 83.122 | 20.045 | 1.00 | 56.04 | C |
| ATOM | 1125 | CD | LYS | A | 358 | 9.983 | 83.499 | 20.497 | 1.00 | 56.64 | C |
| ATOM | 1126 | CE | LYS | A | 358 | 9.720 | 82.989 | 21.907 | 1.00 | 57.66 | C |
| ATOM | 1127 | NZ | LYS | A | 358 | 8.330 | 83.266 | 22.360 | 1.00 | 59.37 | N |
| ATOM | 1128 | N | ASP | A | 359 | 8.434 | 83.480 | 17.122 | 1.00 | 54.05 | N |
| ATOM | 1129 | CA | ASP | A | 359 | 7.348 | 84.336 | 16.662 | 1.00 | 55.06 | C |
| ATOM | 1130 | C | ASP | A | 359 | 7.487 | 84.825 | 15.219 | 1.00 | 54.06 | C |
| ATOM | 1131 | O | ASP | A | 359 | 7.145 | 85.965 | 14.907 | 1.00 | 54.73 | O |
| ATOM | 1132 | CB | ASP | A | 359 | 7.174 | 85.530 | 17.604 | 1.00 | 56.59 | C |
| ATOM | 1133 | CG | ASP | A | 359 | 6.748 | 85.107 | 18.997 | 1.00 | 59.86 | C |
| ATOM | 1134 | OD1 | ASP | A | 359 | 5.842 | 84.248 | 19.107 | 1.00 | 60.48 | O |
| ATOM | 1135 | OD2 | ASP | A | 359 | 7.310 | 85.634 | 19.981 | 1.00 | 62.46 | O |
| ATOM | 1136 | N | GLY | A | 360 | 7.988 | 83.958 | 14.344 | 1.00 | 52.99 | N |
| ATOM | 1137 | CA | GLY | A | 360 | 8.109 | 84.309 | 12.942 | 1.00 | 52.11 | C |
| ATOM | 1138 | C | GLY | A | 360 | 9.409 | 84.925 | 12.478 | 1.00 | 52.46 | C |
| ATOM | 1139 | O | GLY | A | 360 | 9.611 | 85.096 | 11.276 | 1.00 | 51.80 | O |
| ATOM | 1140 | N | LYS | A | 361 | 10.293 | 85.262 | 13.410 | 1.00 | 53.79 | N |
| ATOM | 1141 | CA | LYS | A | 361 | 11.565 | 85.870 | 13.037 | 1.00 | 54.73 | C |
| ATOM | 1142 | C | LYS | A | 361 | 12.753 | 85.057 | 13.516 | 1.00 | 54.19 | C |
| ATOM | 1143 | O | LYS | A | 361 | 12.720 | 84.474 | 14.601 | 1.00 | 53.39 | O |
| ATOM | 1144 | CB | LYS | A | 361 | 11.652 | 87.292 | 13.592 | 1.00 | 56.26 | C |
| ATOM | 1145 | CG | LYS | A | 361 | 10.658 | 88.245 | 12.959 | 1.00 | 60.78 | C |
| ATOM | 1146 | CD | LYS | A | 361 | 10.702 | 89.626 | 13.588 | 1.00 | 64.85 | C |
| ATOM | 1147 | CE | LYS | A | 361 | 9.652 | 90.536 | 12.952 | 1.00 | 68.32 | C |
| ATOM | 1148 | NZ | LYS | A | 361 | 9.588 | 91.883 | 13.596 | 1.00 | 70.53 | N |
| ATOM | 1149 | N | VAL | A | 362 | 13.798 | 85.008 | 12.695 | 1.00 | 53.64 | N |
| ATOM | 1150 | CA | VAL | A | 362 | 15.003 | 84.272 | 13.048 | 1.00 | 53.46 | C |
| ATOM | 1151 | C | VAL | A | 362 | 15.608 | 84.916 | 14.289 | 1.00 | 54.43 | C |
| ATOM | 1152 | O | VAL | A | 362 | 15.882 | 86.113 | 14.298 | 1.00 | 55.93 | O |
| ATOM | 1153 | CB | VAL | A | 362 | 16.035 | 84.318 | 11.917 | 1.00 | 52.06 | C |
| ATOM | 1154 | CG1 | VAL | A | 362 | 17.258 | 83.503 | 12.296 | 1.00 | 51.66 | C |
| ATOM | 1155 | CG2 | VAL | A | 362 | 15.422 | 83.787 | 10.638 | 1.00 | 52.48 | C |
| ATOM | 1156 | N | ALA | A | 363 | 15.793 | 84.127 | 15.343 | 1.00 | 54.64 | N |
| ATOM | 1157 | CA | ALA | A | 363 | 16.365 | 84.632 | 16.585 | 1.00 | 54.00 | C |
| ATOM | 1158 | C | ALA | A | 363 | 17.814 | 84.183 | 16.684 | 1.00 | 54.46 | C |
| ATOM | 1159 | O | ALA | A | 363 | 18.566 | 84.663 | 17.531 | 1.00 | 53.89 | O |
| ATOM | 1160 | CB | ALA | A | 363 | 15.574 | 84.112 | 17.774 | 1.00 | 54.00 | C |
| ATOM | 1161 | N | LEU | A | 364 | 18.187 | 83.257 | 15.805 | 1.00 | 54.48 | N |
| ATOM | 1162 | CA | LEU | A | 364 | 19.536 | 82.711 | 15.748 | 1.00 | 55.15 | C |
| ATOM | 1163 | C | LEU | A | 364 | 19.613 | 81.645 | 14.670 | 1.00 | 55.62 | C |
| ATOM | 1164 | O | LEU | A | 364 | 18.653 | 80.918 | 14.435 | 1.00 | 56.11 | O |
| ATOM | 1165 | CB | LEU | A | 364 | 19.921 | 82.100 | 17.101 | 1.00 | 56.86 | C |
| ATOM | 1166 | CG | LEU | A | 364 | 21.140 | 81.166 | 17.188 | 1.00 | 58.14 | C |
| ATOM | 1167 | CD1 | LEU | A | 364 | 21.549 | 80.994 | 18.643 | 1.00 | 57.33 | C |
| ATOM | 1168 | CD2 | LEU | A | 364 | 20.814 | 79.806 | 16.566 | 1.00 | 57.79 | C |
| ATOM | 1169 | N | GLU | A | 365 | 20.762 | 81.558 | 14.015 | 1.00 | 55.48 | N |
| ATOM | 1170 | CA | GLU | A | 365 | 20.980 | 80.559 | 12.984 | 1.00 | 55.94 | C |
| ATOM | 1171 | C | GLU | A | 365 | 22.463 | 80.223 | 12.990 | 1.00 | 56.15 | C |
| ATOM | 1172 | O | GLU | A | 365 | 23.265 | 80.962 | 13.560 | 1.00 | 57.53 | O |
| ATOM | 1173 | CB | GLU | A | 365 | 20.549 | 81.081 | 11.612 | 1.00 | 57.87 | C |
| ATOM | 1174 | CG | GLU | A | 365 | 21.355 | 82.254 | 11.081 | 1.00 | 60.57 | C |
| ATOM | 1175 | CD | GLU | A | 365 | 20.954 | 82.640 | 9.661 | 1.00 | 62.27 | C |
| ATOM | 1176 | OE1 | GLU | A | 365 | 21.213 | 81.852 | 8.722 | 1.00 | 61.78 | O |
| ATOM | 1177 | OE2 | GLU | A | 365 | 20.373 | 83.733 | 9.483 | 1.00 | 63.59 | O |
| ATOM | 1178 | N | THR | A | 366 | 22.830 | 79.113 | 12.361 | 1.00 | 54.79 | N |
| ATOM | 1179 | CA | THR | A | 366 | 24.224 | 78.689 | 12.343 | 1.00 | 54.31 | C |
| ATOM | 1180 | C | THR | A | 366 | 24.418 | 77.410 | 11.562 | 1.00 | 53.81 | C |
| ATOM | 1181 | O | THR | A | 366 | 23.504 | 76.597 | 11.446 | 1.00 | 53.23 | O |
| ATOM | 1182 | CB | THR | A | 366 | 24.740 | 78.403 | 13.768 | 1.00 | 55.61 | C |
| ATOM | 1183 | OG1 | THR | A | 366 | 25.986 | 77.705 | 13.690 | 1.00 | 54.67 | O |
| ATOM | 1184 | CG2 | THR | A | 366 | 23.748 | 77.523 | 14.533 | 1.00 | 55.89 | C |
| ATOM | 1185 | N | ASP | A | 367 | 25.617 | 77.229 | 11.024 | 1.00 | 53.59 | N |
| ATOM | 1186 | CA | ASP | A | 367 | 25.912 | 76.002 | 10.307 | 1.00 | 54.18 | C |
| ATOM | 1187 | C | ASP | A | 367 | 25.987 | 74.938 | 11.388 | 1.00 | 52.60 | C |
| ATOM | 1188 | O | ASP | A | 367 | 26.208 | 75.244 | 12.560 | 1.00 | 51.11 | O |
| ATOM | 1189 | CB | ASP | A | 367 | 27.257 | 76.087 | 9.590 | 1.00 | 57.67 | C |
| ATOM | 1190 | CG | ASP | A | 367 | 27.228 | 77.033 | 8.414 | 1.00 | 61.75 | C |
| ATOM | 1191 | OD1 | ASP | A | 367 | 26.468 | 76.768 | 7.454 | 1.00 | 62.98 | O |
| ATOM | 1192 | OD2 | ASP | A | 367 | 27.967 | 78.041 | 8.452 | 1.00 | 64.57 | O |
| ATOM | 1193 | N | ILE | A | 368 | 25.804 | 73.690 | 10.993 | 1.00 | 50.46 | N |
| ATOM | 1194 | CA | ILE | A | 368 | 25.850 | 72.594 | 11.941 | 1.00 | 49.93 | C |
| ATOM | 1195 | C | ILE | A | 368 | 26.451 | 71.404 | 11.223 | 1.00 | 49.24 | C |
| ATOM | 1196 | O | ILE | A | 368 | 26.661 | 71.452 | 10.017 | 1.00 | 49.09 | O |
| ATOM | 1197 | CB | ILE | A | 368 | 24.410 | 72.229 | 12.449 | 1.00 | 49.07 | C |
| ATOM | 1198 | CG1 | ILE | A | 368 | 23.485 | 71.901 | 11.267 | 1.00 | 46.25 | C |
| ATOM | 1199 | CG2 | ILE | A | 368 | 23.816 | 73.395 | 13.239 | 1.00 | 47.52 | C |
| ATOM | 1200 | CD1 | ILE | A | 368 | 23.676 | 70.522 | 10.679 | 1.00 | 43.46 | C |
| ATOM | 1201 | N | VAL | A | 369 | 26.742 | 70.345 | 11.963 | 1.00 | 49.66 | N |
| ATOM | 1202 | CA | VAL | A | 369 | 27.279 | 69.139 | 11.356 | 1.00 | 50.05 | C |
| ATOM | 1203 | C | VAL | A | 369 | 26.391 | 68.005 | 11.851 | 1.00 | 51.77 | C |
| ATOM | 1204 | O | VAL | A | 369 | 26.365 | 67.693 | 13.047 | 1.00 | 52.29 | O |
| ATOM | 1205 | CB | VAL | A | 369 | 28.753 | 68.883 | 11.769 | 1.00 | 49.26 | C |
| ATOM | 1206 | CG1 | VAL | A | 369 | 29.286 | 67.655 | 11.042 | 1.00 | 47.99 | C |
| ATOM | 1207 | CG2 | VAL | A | 369 | 29.609 | 70.097 | 11.437 | 1.00 | 47.45 | C |
| ATOM | 1208 | N | SER | A | 370 | 25.642 | 67.411 | 10.931 | 1.00 | 52.36 | N |
| ATOM | 1209 | CA | SER | A | 370 | 24.740 | 66.329 | 11.279 | 1.00 | 53.67 | C |
| ATOM | 1210 | C | SER | A | 370 | 25.473 | 65.001 | 11.260 | 1.00 | 55.44 | C |
| ATOM | 1211 | O | SER | A | 370 | 26.679 | 64.952 | 11.020 | 1.00 | 56.07 | O |
| ATOM | 1212 | CB | SER | A | 370 | 23.563 | 66.284 | 10.303 | 1.00 | 53.83 | C |
| ATOM | 1213 | OG | SER | A | 370 | 23.984 | 65.930 | 8.997 | 1.00 | 52.99 | O |
| ATOM | 1214 | N | GLY | A | 371 | 24.735 | 63.924 | 11.500 | 1.00 | 56.76 | N |
| ATOM | 1215 | CA | GLY | A | 371 | 25.333 | 62.604 | 11.524 | 1.00 | 59.24 | C |
| ATOM | 1216 | C | GLY | A | 371 | 25.962 | 62.164 | 10.219 | 1.00 | 60.73 | C |
| ATOM | 1217 | O | GLY | A | 371 | 25.493 | 62.528 | 9.139 | 1.00 | 61.36 | O |
| ATOM | 1218 | N | LYS | A | 372 | 27.030 | 61.377 | 10.333 | 1.00 | 61.62 | N |
| ATOM | 1219 | CA | LYS | A | 372 | 27.751 | 60.849 | 9.177 | 1.00 | 62.55 | C |
| ATOM | 1220 | C | LYS | A | 372 | 26.844 | 59.894 | 8.390 | 1.00 | 62.24 | C |
| ATOM | 1221 | O | LYS | A | 372 | 25.867 | 59.376 | 8.924 | 1.00 | 61.94 | O |
| ATOM | 1222 | CB | LYS | A | 372 | 29.017 | 60.116 | 9.641 | 1.00 | 63.61 | C |
| ATOM | 1223 | CG | LYS | A | 372 | 28.761 | 58.956 | 10.600 | 1.00 | 64.81 | C |
| ATOM | 1224 | CD | LYS | A | 372 | 30.053 | 58.216 | 10.943 | 1.00 | 64.56 | C |
| ATOM | 1225 | CE | LYS | A | 372 | 29.797 | 57.054 | 11.893 | 1.00 | 63.55 | C |
| ATOM | 1226 | NZ | LYS | A | 372 | 29.250 | 57.511 | 13.201 | 1.00 | 62.89 | N |
| ATOM | 1227 | N | PRO | A | 373 | 27.164 | 59.647 | 7.109 | 1.00 | 62.18 | N |
| ATOM | 1228 | CA | PRO | A | 373 | 26.385 | 58.762 | 6.235 | 1.00 | 62.47 | C |
| ATOM | 1229 | C | PRO | A | 373 | 25.941 | 57.421 | 6.823 | 1.00 | 63.14 | C |
| ATOM | 1230 | O | PRO | A | 373 | 24.872 | 56.912 | 6.482 | 1.00 | 63.86 | O |
| ATOM | 1231 | CB | PRO | A | 373 | 27.296 | 58.597 | 5.024 | 1.00 | 61.82 | C |
| ATOM | 1232 | CG | PRO | A | 373 | 27.934 | 59.945 | 4.932 | 1.00 | 62.09 | C |
| ATOM | 1233 | CD | PRO | A | 373 | 28.307 | 60.222 | 6.377 | 1.00 | 61.57 | C |
| ATOM | 1234 | N | THR | A | 374 | 26.756 | 56.842 | 7.696 | 1.00 | 64.11 | N |
| ATOM | 1235 | CA | THR | A | 374 | 26.409 | 55.559 | 8.304 | 1.00 | 63.84 | C |
| ATOM | 1236 | C | THR | A | 374 | 25.383 | 55.718 | 9.427 | 1.00 | 62.73 | C |
| ATOM | 1237 | O | THR | A | 374 | 24.512 | 54.866 | 9.607 | 1.00 | 63.63 | O |
| ATOM | 1238 | CB | THR | A | 374 | 27.666 | 54.849 | 8.849 | 1.00 | 64.69 | C |
| ATOM | 1239 | OG1 | THR | A | 374 | 28.412 | 55.750 | 9.681 | 1.00 | 65.43 | O |
| ATOM | 1240 | CG2 | THR | A | 374 | 28.540 | 54.376 | 7.693 | 1.00 | 63.91 | C |
| ATOM | 1241 | N | THR | A | 375 | 25.499 | 56.803 | 10.185 | 1.00 | 60.28 | N |
| ATOM | 1242 | CA | THR | A | 375 | 24.567 | 57.093 | 11.272 | 1.00 | 57.84 | C |
| ATOM | 1243 | C | THR | A | 375 | 23.979 | 58.472 | 10.977 | 1.00 | 55.46 | C |
| ATOM | 1244 | O | THR | A | 375 | 24.299 | 59.463 | 11.639 | 1.00 | 54.48 | O |
| ATOM | 1245 | CB | THR | A | 375 | 25.280 | 57.098 | 12.655 | 1.00 | 58.75 | C |
| ATOM | 1246 | OG1 | THR | A | 375 | 26.284 | 58.125 | 12.694 | 1.00 | 57.05 | O |
| ATOM | 1247 | CG2 | THR | A | 375 | 25.925 | 55.746 | 12.910 | 1.00 | 57.33 | C |
| ATOM | 1248 | N | PRO | A | 376 | 23.100 | 58.544 | 9.964 | 1.00 | 53.99 | N |
| ATOM | 1249 | CA | PRO | A | 376 | 22.449 | 59.788 | 9.537 | 1.00 | 51.67 | C |
| ATOM | 1250 | C | PRO | A | 376 | 21.445 | 60.340 | 10.536 | 1.00 | 49.03 | C |
| ATOM | 1251 | O | PRO | A | 376 | 20.776 | 59.580 | 11.239 | 1.00 | 48.29 | O |
| ATOM | 1252 | CB | PRO | A | 376 | 21.747 | 59.396 | 8.228 | 1.00 | 52.06 | C |
| ATOM | 1253 | CG | PRO | A | 376 | 22.290 | 58.026 | 7.877 | 1.00 | 53.15 | C |
| ATOM | 1254 | CD | PRO | A | 376 | 22.573 | 57.399 | 9.204 | 1.00 | 53.16 | C |
| ATOM | 1255 | N | THR | A | 377 | 21.354 | 61.666 | 10.598 | 1.00 | 47.36 | N |
| ATOM | 1256 | CA | THR | A | 377 | 20.378 | 62.319 | 11.461 | 1.00 | 46.31 | C |
| ATOM | 1257 | C | THR | A | 377 | 19.056 | 62.032 | 10.753 | 1.00 | 45.09 | C |
| ATOM | 1258 | O | THR | A | 377 | 18.883 | 62.412 | 9.597 | 1.00 | 44.92 | O |
| ATOM | 1259 | CB | THR | A | 377 | 20.581 | 63.834 | 11.490 | 1.00 | 47.57 | C |
| ATOM | 1260 | OG1 | THR | A | 377 | 21.881 | 64.132 | 12.007 | 1.00 | 48.02 | O |
| ATOM | 1261 | CG2 | THR | A | 377 | 19.516 | 64.502 | 12.363 | 1.00 | 48.81 | C |
| ATOM | 1262 | N | PRO | A | 378 | 18.116 | 61.344 | 11.425 | 1.00 | 43.96 | N |
| ATOM | 1263 | CA | PRO | A | 378 | 16.823 | 61.028 | 10.803 | 1.00 | 42.22 | C |
| ATOM | 1264 | C | PRO | A | 378 | 15.938 | 62.261 | 10.587 | 1.00 | 40.68 | C |
| ATOM | 1265 | O | PRO | A | 378 | 15.914 | 63.171 | 11.410 | 1.00 | 39.22 | O |
| ATOM | 1266 | CB | PRO | A | 378 | 16.212 | 60.040 | 11.787 | 1.00 | 42.20 | C |
| ATOM | 1267 | CG | PRO | A | 378 | 16.675 | 60.585 | 13.101 | 1.00 | 42.62 | C |
| ATOM | 1268 | CD | PRO | A | 378 | 18.141 | 60.896 | 12.831 | 1.00 | 43.73 | C |
| ATOM | 1269 | N | ALA | A | 379 | 15.229 | 62.298 | 9.466 | 1.00 | 40.31 | N |
| ATOM | 1270 | CA | ALA | A | 379 | 14.346 | 63.421 | 9.182 | 1.00 | 40.43 | C |
| ATOM | 1271 | C | ALA | A | 379 | 13.036 | 63.190 | 9.922 | 1.00 | 40.66 | C |
| ATOM | 1272 | O | ALA | A | 379 | 12.679 | 62.040 | 10.226 | 1.00 | 39.82 | O |
| ATOM | 1273 | CB | ALA | A | 379 | 14.084 | 63.523 | 7.689 | 1.00 | 40.14 | C |
| ATOM | 1274 | N | GLY | A | 380 | 12.329 | 64.276 | 10.223 | 1.00 | 38.46 | N |
| ATOM | 1275 | CA | GLY | A | 380 | 11.058 | 64.145 | 10.910 | 1.00 | 39.04 | C |
| ATOM | 1276 | C | GLY | A | 380 | 10.672 | 65.343 | 11.748 | 1.00 | 38.30 | C |
| ATOM | 1277 | O | GLY | A | 380 | 11.478 | 66.257 | 11.949 | 1.00 | 40.09 | O |
| ATOM | 1278 | N | VAL | A | 381 | 9.429 | 65.345 | 12.224 | 1.00 | 36.75 | N |
| ATOM | 1279 | CA | VAL | A | 381 | 8.940 | 66.426 | 13.068 | 1.00 | 35.33 | C |
| ATOM | 1280 | C | VAL | A | 381 | 9.211 | 66.044 | 14.520 | 1.00 | 37.01 | C |
| ATOM | 1281 | O | VAL | A | 381 | 8.487 | 65.243 | 15.112 | 1.00 | 38.55 | O |
| ATOM | 1282 | CB | VAL | A | 381 | 7.426 | 66.650 | 12.886 | 1.00 | 34.53 | C |
| ATOM | 1283 | CG1 | VAL | A | 381 | 6.968 | 67.794 | 13.782 | 1.00 | 32.17 | C |
| ATOM | 1284 | CG2 | VAL | A | 381 | 7.112 | 66.956 | 11.433 | 1.00 | 31.90 | C |
| ATOM | 1285 | N | PHE | A | 382 | 10.257 | 66.623 | 15.093 | 1.00 | 36.52 | N |
| ATOM | 1286 | CA | PHE | A | 382 | 10.629 | 66.322 | 16.463 | 1.00 | 35.98 | C |
| ATOM | 1287 | C | PHE | A | 382 | 10.355 | 67.518 | 17.362 | 1.00 | 37.45 | C |
| ATOM | 1288 | O | PHE | A | 382 | 9.729 | 68.494 | 16.946 | 1.00 | 35.95 | O |
| ATOM | 1289 | CB | PHE | A | 382 | 12.119 | 65.958 | 16.514 | 1.00 | 37.04 | C |
| ATOM | 1290 | CG | PHE | A | 382 | 12.494 | 64.796 | 15.626 | 1.00 | 37.57 | C |
| ATOM | 1291 | CD1 | PHE | A | 382 | 12.130 | 63.494 | 15.963 | 1.00 | 38.37 | C |
| ATOM | 1292 | CD2 | PHE | A | 382 | 13.206 | 65.005 | 14.450 | 1.00 | 36.96 | C |
| ATOM | 1293 | CE1 | PHE | A | 382 | 12.472 | 62.412 | 15.138 | 1.00 | 38.25 | C |
| ATOM | 1294 | CE2 | PHE | A | 382 | 13.554 | 63.933 | 13.618 | 1.00 | 38.00 | C |
| ATOM | 1295 | CZ | PHE | A | 382 | 13.185 | 62.635 | 13.965 | 1.00 | 38.39 | C |
| ATOM | 1296 | N | TYR | A | 383 | 10.818 | 67.433 | 18.604 | 1.00 | 36.70 | N |
| ATOM | 1297 | CA | TYR | A | 383 | 10.645 | 68.526 | 19.537 | 1.00 | 38.04 | C |
| ATOM | 1298 | C | TYR | A | 383 | 11.583 | 68.349 | 20.713 | 1.00 | 39.35 | C |
| ATOM | 1299 | O | TYR | A | 383 | 11.986 | 67.229 | 21.034 | 1.00 | 38.84 | O |
| ATOM | 1300 | CB | TYR | A | 383 | 9.202 | 68.594 | 20.027 | 1.00 | 38.47 | C |
| ATOM | 1301 | CG | TYR | A | 383 | 8.804 | 67.522 | 21.022 | 1.00 | 37.92 | C |
| ATOM | 1302 | CD1 | TYR | A | 383 | 8.774 | 66.177 | 20.662 | 1.00 | 37.72 | C |
| ATOM | 1303 | CD2 | TYR | A | 383 | 8.386 | 67.869 | 22.311 | 1.00 | 37.00 | C |
| ATOM | 1304 | CE1 | TYR | A | 383 | 8.326 | 65.204 | 21.562 | 1.00 | 37.15 | C |
| ATOM | 1305 | CE2 | TYR | A | 383 | 7.940 | 66.909 | 23.211 | 1.00 | 34.98 | C |
| ATOM | 1306 | CZ | TYR | A | 383 | 7.909 | 65.584 | 22.832 | 1.00 | 36.27 | C |
| ATOM | 1307 | OH | TYR | A | 383 | 7.437 | 64.645 | 23.720 | 1.00 | 34.98 | O |
| ATOM | 1308 | N | VAL | A | 384 | 11.941 | 69.463 | 21.342 | 1.00 | 40.95 | N |
| ATOM | 1309 | CA | VAL | A | 384 | 12.833 | 69.439 | 22.495 | 1.00 | 43.40 | C |
| ATOM | 1310 | C | VAL | A | 384 | 12.036 | 68.986 | 23.714 | 1.00 | 44.55 | C |
| ATOM | 1311 | O | VAL | A | 384 | 11.306 | 69.776 | 24.325 | 1.00 | 44.53 | O |
| ATOM | 1312 | CB | VAL | A | 384 | 13.432 | 70.842 | 22.750 | 1.00 | 44.85 | C |
| ATOM | 1313 | CG1 | VAL | A | 384 | 14.127 | 70.883 | 24.109 | 1.00 | 46.80 | C |
| ATOM | 1314 | CG2 | VAL | A | 384 | 14.417 | 71.196 | 21.629 | 1.00 | 44.56 | C |
| ATOM | 1315 | N | TRP | A | 385 | 12.162 | 67.709 | 24.060 | 1.00 | 44.44 | N |
| ATOM | 1316 | CA | TRP | A | 385 | 11.426 | 67.183 | 25.196 | 1.00 | 46.86 | C |
| ATOM | 1317 | C | TRP | A | 385 | 12.146 | 67.312 | 26.533 | 1.00 | 48.40 | C |
| ATOM | 1318 | O | TRP | A | 385 | 11.555 | 67.076 | 27.591 | 1.00 | 47.70 | O |
| ATOM | 1319 | CB | TRP | A | 385 | 11.032 | 65.724 | 24.954 | 1.00 | 46.96 | C |
| ATOM | 1320 | CG | TRP | A | 385 | 12.148 | 64.803 | 24.577 | 1.00 | 46.20 | C |
| ATOM | 1321 | CD1 | TRP | A | 385 | 12.606 | 64.541 | 23.320 | 1.00 | 44.89 | C |
| ATOM | 1322 | CD2 | TRP | A | 385 | 12.873 | 63.938 | 25.457 | 1.00 | 45.68 | C |
| ATOM | 1323 | NE1 | TRP | A | 385 | 13.560 | 63.556 | 23.359 | 1.00 | 45.36 | N |
| ATOM | 1324 | CE2 | TRP | A | 385 | 13.746 | 63.168 | 24.659 | 1.00 | 45.24 | C |
| ATOM | 1325 | CE3 | TRP | A | 385 | 12.865 | 63.734 | 26.842 | 1.00 | 46.32 | C |
| ATOM | 1326 | CZ2 | TRP | A | 385 | 14.604 | 62.208 | 25.197 | 1.00 | 45.60 | C |
| ATOM | 1327 | CZ3 | TRP | A | 385 | 13.721 | 62.774 | 27.381 | 1.00 | 47.28 | C |
| ATOM | 1328 | CH2 | TRP | A | 385 | 14.578 | 62.025 | 26.555 | 1.00 | 46.14 | C |
| ATOM | 1329 | N | ASN | A | 386 | 13.415 | 67.701 | 26.487 | 1.00 | 50.25 | N |
| ATOM | 1330 | CA | ASN | A | 386 | 14.191 | 67.874 | 27.704 | 1.00 | 51.87 | C |
| ATOM | 1331 | C | ASN | A | 386 | 15.414 | 68.740 | 27.455 | 1.00 | 53.45 | C |
| ATOM | 1332 | O | ASN | A | 386 | 15.878 | 68.864 | 26.326 | 1.00 | 53.55 | O |
| ATOM | 1333 | CB | ASN | A | 386 | 14.610 | 66.508 | 28.258 | 1.00 | 50.25 | C |
| ATOM | 1334 | CG | ASN | A | 386 | 15.299 | 66.609 | 29.610 | 1.00 | 50.70 | C |
| ATOM | 1335 | OD1 | ASN | A | 386 | 14.985 | 67.481 | 30.422 | 1.00 | 46.79 | O |
| ATOM | 1336 | ND2 | ASN | A | 386 | 16.231 | 65.698 | 29.864 | 1.00 | 51.63 | N |
| ATOM | 1337 | N | LYS | A | 387 | 15.913 | 69.356 | 28.517 | 1.00 | 56.23 | N |
| ATOM | 1338 | CA | LYS | A | 387 | 17.101 | 70.201 | 28.442 | 1.00 | 59.11 | C |
| ATOM | 1339 | C | LYS | A | 387 | 18.016 | 69.831 | 29.594 | 1.00 | 61.72 | C |
| ATOM | 1340 | O | LYS | A | 387 | 17.603 | 69.836 | 30.755 | 1.00 | 63.29 | O |
| ATOM | 1341 | CB | LYS | A | 387 | 16.729 | 71.683 | 28.534 | 1.00 | 56.20 | C |
| ATOM | 1342 | CG | LYS | A | 387 | 16.008 | 72.208 | 27.310 | 1.00 | 54.45 | C |
| ATOM | 1343 | CD | LYS | A | 387 | 15.608 | 73.669 | 27.478 | 1.00 | 52.91 | C |
| ATOM | 1344 | CE | LYS | A | 387 | 16.816 | 74.578 | 27.581 | 1.00 | 50.26 | C |
| ATOM | 1345 | NZ | LYS | A | 387 | 16.384 | 75.983 | 27.786 | 1.00 | 51.99 | N |
| ATOM | 1346 | N | GLU | A | 388 | 19.255 | 69.494 | 29.264 | 1.00 | 64.65 | N |
| ATOM | 1347 | CA | GLU | A | 388 | 20.238 | 69.113 | 30.267 | 1.00 | 67.92 | C |
| ATOM | 1348 | C | GLU | A | 388 | 21.532 | 69.876 | 30.071 | 1.00 | 69.46 | C |
| ATOM | 1349 | O | GLU | A | 388 | 21.935 | 70.148 | 28.940 | 1.00 | 70.26 | O |
| ATOM | 1350 | CB | GLU | A | 388 | 20.540 | 67.614 | 30.186 | 1.00 | 68.19 | C |
| ATOM | 1351 | CG | GLU | A | 388 | 19.553 | 66.725 | 30.918 | 1.00 | 70.95 | C |
| ATOM | 1352 | CD | GLU | A | 388 | 19.978 | 65.265 | 30.917 | 1.00 | 72.03 | C |
| ATOM | 1353 | OE1 | GLU | A | 388 | 21.195 | 65.007 | 31.055 | 1.00 | 72.26 | O |
| ATOM | 1354 | OE2 | GLU | A | 388 | 19.098 | 64.382 | 30.795 | 1.00 | 71.01 | O |
| ATOM | 1355 | N | GLU | A | 389 | 22.179 | 70.221 | 31.177 | 1.00 | 70.67 | N |
| ATOM | 1356 | CA | GLU | A | 389 | 23.452 | 70.923 | 31.123 | 1.00 | 71.12 | C |
| ATOM | 1357 | C | GLU | A | 389 | 24.530 | 69.947 | 31.579 | 1.00 | 71.23 | C |
| ATOM | 1358 | O | GLU | A | 389 | 24.265 | 69.056 | 32.386 | 1.00 | 71.31 | O |
| ATOM | 1359 | CB | GLU | A | 389 | 23.422 | 72.156 | 32.026 | 1.00 | 71.38 | C |
| ATOM | 1360 | CG | GLU | A | 389 | 22.396 | 73.188 | 31.595 | 1.00 | 73.84 | C |
| ATOM | 1361 | CD | GLU | A | 389 | 22.534 | 74.500 | 32.338 | 1.00 | 75.24 | C |
| ATOM | 1362 | OE1 | GLU | A | 389 | 23.632 | 75.094 | 32.294 | 1.00 | 76.60 | O |
| ATOM | 1363 | OE2 | GLU | A | 389 | 21.546 | 74.940 | 32.961 | 1.00 | 76.68 | O |
| ATOM | 1364 | N | ASP | A | 390 | 25.736 | 70.107 | 31.046 | 1.00 | 71.32 | N |
| ATOM | 1365 | CA | ASP | A | 390 | 26.853 | 69.235 | 31.393 | 1.00 | 71.43 | C |
| ATOM | 1366 | C | ASP | A | 390 | 26.405 | 67.774 | 31.429 | 1.00 | 70.25 | C |
| ATOM | 1367 | O | ASP | A | 390 | 26.494 | 67.103 | 32.455 | 1.00 | 70.43 | O |
| ATOM | 1368 | CB | ASP | A | 390 | 27.450 | 69.640 | 32.751 | 1.00 | 72.64 | C |
| ATOM | 1369 | CG | ASP | A | 390 | 27.908 | 71.097 | 32.785 | 1.00 | 75.29 | C |
| ATOM | 1370 | OD1 | ASP | A | 390 | 28.668 | 71.517 | 31.882 | 1.00 | 76.78 | O |
| ATOM | 1371 | OD2 | ASP | A | 390 | 27.512 | 71.826 | 33.724 | 1.00 | 75.97 | O |
| ATOM | 1372 | N | ALA | A | 391 | 25.906 | 67.290 | 30.300 | 1.00 | 69.10 | N |
| ATOM | 1373 | CA | ALA | A | 391 | 25.462 | 65.908 | 30.198 | 1.00 | 68.84 | C |
| ATOM | 1374 | C | ALA | A | 391 | 26.604 | 65.091 | 29.604 | 1.00 | 68.45 | C |
| ATOM | 1375 | O | ALA | A | 391 | 27.677 | 65.626 | 29.335 | 1.00 | 68.07 | O |
| ATOM | 1376 | CB | ALA | A | 391 | 24.229 | 65.817 | 29.310 | 1.00 | 67.78 | C |
| ATOM | 1377 | N | THR | A | 392 | 26.374 | 63.800 | 29.398 | 1.00 | 69.12 | N |
| ATOM | 1378 | CA | THR | A | 392 | 27.401 | 62.931 | 28.839 | 1.00 | 71.39 | C |
| ATOM | 1379 | C | THR | A | 392 | 26.779 | 61.852 | 27.953 | 1.00 | 72.93 | C |
| ATOM | 1380 | O | THR | A | 392 | 26.385 | 60.788 | 28.436 | 1.00 | 73.64 | O |
| ATOM | 1381 | CB | THR | A | 392 | 28.218 | 62.251 | 29.962 | 1.00 | 71.83 | C |
| ATOM | 1382 | OG1 | THR | A | 392 | 28.699 | 63.246 | 30.877 | 1.00 | 71.90 | O |
| ATOM | 1383 | CG2 | THR | A | 392 | 29.403 | 61.495 | 29.375 | 1.00 | 71.82 | C |
| ATOM | 1384 | N | LEU | A | 393 | 26.696 | 62.130 | 26.656 | 1.00 | 74.82 | N |
| ATOM | 1385 | CA | LEU | A | 393 | 26.112 | 61.189 | 25.701 | 1.00 | 76.70 | C |
| ATOM | 1386 | C | LEU | A | 393 | 26.875 | 59.866 | 25.693 | 1.00 | 77.18 | C |
| ATOM | 1387 | O | LEU | A | 393 | 28.043 | 59.821 | 26.057 | 1.00 | 78.36 | O |
| ATOM | 1388 | CB | LEU | A | 393 | 26.115 | 61.796 | 24.289 | 1.00 | 77.37 | C |
| ATOM | 1389 | CG | LEU | A | 393 | 25.294 | 63.059 | 23.971 | 1.00 | 78.09 | C |
| ATOM | 1390 | CD1 | LEU | A | 393 | 23.829 | 62.782 | 24.224 | 1.00 | 79.22 | C |
| ATOM | 1391 | CD2 | LEU | A | 393 | 25.760 | 64.240 | 24.809 | 1.00 | 78.23 | C |
| ATOM | 1392 | N | LYS | A | 394 | 26.208 | 58.792 | 25.283 | 1.00 | 78.84 | N |
| ATOM | 1393 | CA | LYS | A | 394 | 26.837 | 57.474 | 25.221 | 1.00 | 79.99 | C |
| ATOM | 1394 | C | LYS | A | 394 | 26.287 | 56.630 | 24.071 | 1.00 | 80.34 | C |
| ATOM | 1395 | O | LYS | A | 394 | 25.073 | 56.537 | 23.881 | 1.00 | 80.63 | O |
| ATOM | 1396 | CB | LYS | A | 394 | 26.637 | 56.718 | 26.539 | 1.00 | 81.74 | C |
| ATOM | 1397 | CG | LYS | A | 394 | 27.451 | 57.238 | 27.718 | 1.00 | 83.69 | C |
| ATOM | 1398 | CD | LYS | A | 394 | 27.401 | 56.250 | 28.882 | 1.00 | 84.91 | C |
| ATOM | 1399 | CE | LYS | A | 394 | 28.189 | 56.743 | 30.087 | 1.00 | 85.89 | C |
| ATOM | 1400 | NZ | LYS | A | 394 | 27.606 | 57.986 | 30.670 | 1.00 | 87.01 | N |
| ATOM | 1401 | N | GLY | A | 395 | 27.185 | 56.010 | 23.313 | 1.00 | 80.54 | N |
| ATOM | 1402 | CA | GLY | A | 395 | 26.763 | 55.183 | 22.199 | 1.00 | 82.12 | C |
| ATOM | 1403 | C | GLY | A | 395 | 27.845 | 54.226 | 21.739 | 1.00 | 83.36 | C |
| ATOM | 1404 | O | GLY | A | 395 | 28.788 | 53.952 | 22.479 | 1.00 | 83.58 | O |
| ATOM | 1405 | N | THR | A | 396 | 27.712 | 53.723 | 20.515 | 1.00 | 84.50 | N |
| ATOM | 1406 | CA | THR | A | 396 | 28.676 | 52.785 | 19.950 | 1.00 | 85.71 | C |
| ATOM | 1407 | C | THR | A | 396 | 29.231 | 53.336 | 18.638 | 1.00 | 87.07 | C |
| ATOM | 1408 | O | THR | A | 396 | 28.933 | 54.469 | 18.266 | 1.00 | 88.19 | O |
| ATOM | 1409 | CB | THR | A | 396 | 28.014 | 51.423 | 19.679 | 1.00 | 85.51 | C |
| ATOM | 1410 | OG1 | THR | A | 396 | 27.183 | 51.068 | 20.792 | 1.00 | 84.76 | O |
| ATOM | 1411 | CG2 | THR | A | 396 | 29.075 | 50.344 | 19.491 | 1.00 | 85.71 | C |
| ATOM | 1412 | N | ASN | A | 397 | 30.039 | 52.538 | 17.942 | 1.00 | 88.17 | N |
| ATOM | 1413 | CA | ASN | A | 397 | 30.628 | 52.956 | 16.671 | 1.00 | 89.40 | C |
| ATOM | 1414 | C | ASN | A | 397 | 30.551 | 51.854 | 15.617 | 1.00 | 89.73 | C |
| ATOM | 1415 | O | ASN | A | 397 | 31.597 | 51.541 | 15.008 | 1.00 | 90.32 | O |
| ATOM | 1416 | CB | ASN | A | 397 | 32.092 | 53.365 | 16.871 | 1.00 | 90.42 | C |
| ATOM | 1417 | CG | ASN | A | 397 | 32.239 | 54.648 | 17.668 | 1.00 | 91.41 | C |
| ATOM | 1418 | OD1 | ASN | A | 397 | 31.761 | 54.751 | 18.797 | 1.00 | 91.21 | O |
| ATOM | 1419 | ND2 | ASN | A | 397 | 32.907 | 55.635 | 17.080 | 1.00 | 92.32 | N |
| ATOM | 1420 | N | GLY | A | 400 | 32.241 | 47.872 | 16.726 | 1.00 | 89.87 | N |
| ATOM | 1421 | CA | GLY | A | 400 | 31.687 | 48.470 | 17.976 | 1.00 | 90.16 | C |
| ATOM | 1422 | C | GLY | A | 400 | 32.631 | 49.490 | 18.584 | 1.00 | 90.67 | C |
| ATOM | 1423 | O | GLY | A | 400 | 33.332 | 50.196 | 17.851 | 1.00 | 90.79 | O |
| ATOM | 1424 | N | THR | A | 401 | 32.643 | 49.554 | 19.918 | 1.00 | 90.47 | N |
| ATOM | 1425 | CA | THR | A | 401 | 33.485 | 50.470 | 20.699 | 1.00 | 90.77 | C |
| ATOM | 1426 | C | THR | A | 401 | 32.707 | 51.700 | 21.148 | 1.00 | 91.21 | C |
| ATOM | 1427 | O | THR | A | 401 | 32.570 | 52.664 | 20.397 | 1.00 | 91.80 | O |
| ATOM | 1428 | CB | THR | A | 401 | 34.721 | 50.966 | 19.912 | 1.00 | 91.15 | C |
| ATOM | 1429 | OG1 | THR | A | 401 | 35.458 | 49.842 | 19.416 | 1.00 | 91.54 | O |
| ATOM | 1430 | CG2 | THR | A | 401 | 35.626 | 51.809 | 20.812 | 1.00 | 90.31 | C |
| ATOM | 1431 | N | PRO | A | 402 | 32.189 | 51.683 | 22.386 | 1.00 | 91.40 | N |
| ATOM | 1432 | CA | PRO | A | 402 | 31.416 | 52.797 | 22.954 | 1.00 | 90.67 | C |
| ATOM | 1433 | C | PRO | A | 402 | 32.167 | 54.133 | 22.983 | 1.00 | 89.18 | C |
| ATOM | 1434 | O | PRO | A | 402 | 33.396 | 54.166 | 22.893 | 1.00 | 89.21 | O |
| ATOM | 1435 | CB | PRO | A | 402 | 31.078 | 52.297 | 24.359 | 1.00 | 91.36 | C |
| ATOM | 1436 | CG | PRO | A | 402 | 30.944 | 50.811 | 24.152 | 1.00 | 92.53 | C |
| ATOM | 1437 | CD | PRO | A | 402 | 32.150 | 50.511 | 23.281 | 1.00 | 92.05 | C |
| ATOM | 1438 | N | TYR | A | 403 | 31.416 | 55.228 | 23.103 | 1.00 | 87.20 | N |
| ATOM | 1439 | CA | TYR | A | 403 | 32.000 | 56.567 | 23.163 | 1.00 | 85.37 | C |
| ATOM | 1440 | C | TYR | A | 403 | 31.356 | 57.361 | 24.296 | 1.00 | 84.31 | C |
| ATOM | 1441 | O | TYR | A | 403 | 30.273 | 57.014 | 24.767 | 1.00 | 84.01 | O |
| ATOM | 1442 | CB | TYR | A | 403 | 31.818 | 57.304 | 21.823 | 1.00 | 85.14 | C |
| ATOM | 1443 | CG | TYR | A | 403 | 30.380 | 57.597 | 21.430 | 1.00 | 84.69 | C |
| ATOM | 1444 | CD1 | TYR | A | 403 | 29.662 | 58.632 | 22.036 | 1.00 | 84.91 | C |
| ATOM | 1445 | CD2 | TYR | A | 403 | 29.735 | 56.834 | 20.457 | 1.00 | 84.19 | C |
| ATOM | 1446 | CE1 | TYR | A | 403 | 28.340 | 58.899 | 21.681 | 1.00 | 84.14 | C |
| ATOM | 1447 | CE2 | TYR | A | 403 | 28.415 | 57.091 | 20.098 | 1.00 | 84.30 | C |
| ATOM | 1448 | CZ | TYR | A | 403 | 27.724 | 58.125 | 20.713 | 1.00 | 84.49 | C |
| ATOM | 1449 | OH | TYR | A | 403 | 26.422 | 58.383 | 20.353 | 1.00 | 84.28 | O |
| ATOM | 1450 | N | GLU | A | 404 | 32.030 | 58.416 | 24.739 | 1.00 | 83.12 | N |
| ATOM | 1451 | CA | GLU | A | 404 | 31.498 | 59.230 | 25.815 | 1.00 | 82.62 | C |
| ATOM | 1452 | C | GLU | A | 404 | 31.217 | 60.644 | 25.347 | 1.00 | 82.81 | C |
| ATOM | 1453 | O | GLU | A | 404 | 30.064 | 61.036 | 25.176 | 1.00 | 83.82 | O |
| ATOM | 1454 | N | SER | A | 405 | 32.279 | 61.412 | 25.136 | 1.00 | 82.20 | N |
| ATOM | 1455 | CA | SER | A | 405 | 32.158 | 62.795 | 24.681 | 1.00 | 81.05 | C |
| ATOM | 1456 | C | SER | A | 405 | 31.154 | 63.613 | 25.500 | 1.00 | 79.37 | C |
| ATOM | 1457 | O | SER | A | 405 | 29.952 | 63.622 | 25.217 | 1.00 | 77.99 | O |
| ATOM | 1458 | CB | SER | A | 405 | 31.770 | 62.835 | 23.195 | 1.00 | 81.52 | C |
| ATOM | 1459 | OG | SER | A | 405 | 30.512 | 62.220 | 22.970 | 1.00 | 80.61 | O |
| ATOM | 1460 | N | PRO | A | 406 | 31.641 | 64.294 | 26.548 | 1.00 | 77.66 | N |
| ATOM | 1461 | CA | PRO | A | 406 | 30.779 | 65.118 | 27.398 | 1.00 | 76.05 | C |
| ATOM | 1462 | C | PRO | A | 406 | 30.267 | 66.329 | 26.618 | 1.00 | 73.42 | C |
| ATOM | 1463 | O | PRO | A | 406 | 30.895 | 66.773 | 25.657 | 1.00 | 72.02 | O |
| ATOM | 1464 | CB | PRO | A | 406 | 31.708 | 65.514 | 28.545 | 1.00 | 76.51 | C |
| ATOM | 1465 | CG | PRO | A | 406 | 32.616 | 64.333 | 28.652 | 1.00 | 77.02 | C |
| ATOM | 1466 | CD | PRO | A | 406 | 32.943 | 64.069 | 27.199 | 1.00 | 77.47 | C |
| ATOM | 1467 | N | VAL | A | 407 | 29.130 | 66.862 | 27.045 | 1.00 | 70.91 | N |
| ATOM | 1468 | CA | VAL | A | 407 | 28.525 | 68.003 | 26.374 | 1.00 | 68.84 | C |
| ATOM | 1469 | C | VAL | A | 407 | 28.024 | 69.033 | 27.376 | 1.00 | 66.70 | C |
| ATOM | 1470 | O | VAL | A | 407 | 27.486 | 68.675 | 28.421 | 1.00 | 66.63 | O |
| ATOM | 1471 | CB | VAL | A | 407 | 27.347 | 67.529 | 25.484 | 1.00 | 69.61 | C |
| ATOM | 1472 | CG1 | VAL | A | 407 | 26.447 | 68.689 | 25.136 | 1.00 | 69.41 | C |
| ATOM | 1473 | CG2 | VAL | A | 407 | 27.887 | 66.877 | 24.214 | 1.00 | 69.44 | C |
| ATOM | 1474 | N | ASN | A | 408 | 28.206 | 70.312 | 27.058 | 1.00 | 65.51 | N |
| ATOM | 1475 | CA | ASN | A | 408 | 27.752 | 71.388 | 27.937 | 1.00 | 65.56 | C |
| ATOM | 1476 | C | ASN | A | 408 | 26.241 | 71.589 | 27.887 | 1.00 | 64.39 | C |
| ATOM | 1477 | O | ASN | A | 408 | 25.616 | 71.923 | 28.899 | 1.00 | 64.25 | O |
| ATOM | 1478 | CB | ASN | A | 408 | 28.439 | 72.706 | 27.580 | 1.00 | 67.10 | C |
| ATOM | 1479 | CG | ASN | A | 408 | 29.812 | 72.832 | 28.202 | 1.00 | 69.91 | C |
| ATOM | 1480 | OD1 | ASN | A | 408 | 29.961 | 72.765 | 29.427 | 1.00 | 70.97 | O |
| ATOM | 1481 | ND2 | ASN | A | 408 | 30.826 | 73.019 | 27.364 | 1.00 | 70.61 | N |
| ATOM | 1482 | N | TYR | A | 409 | 25.655 | 71.400 | 26.708 | 1.00 | 62.20 | N |
| ATOM | 1483 | CA | TYR | A | 409 | 24.216 | 71.560 | 26.553 | 1.00 | 59.63 | C |
| ATOM | 1484 | C | TYR | A | 409 | 23.614 | 70.442 | 25.709 | 1.00 | 57.30 | C |
| ATOM | 1485 | O | TYR | A | 409 | 24.058 | 70.177 | 24.595 | 1.00 | 57.48 | O |
| ATOM | 1486 | CB | TYR | A | 409 | 23.897 | 72.924 | 25.943 | 1.00 | 59.90 | C |
| ATOM | 1487 | CG | TYR | A | 409 | 24.450 | 74.079 | 26.747 | 1.00 | 61.15 | C |
| ATOM | 1488 | CD1 | TYR | A | 409 | 25.721 | 74.596 | 26.483 | 1.00 | 61.08 | C |
| ATOM | 1489 | CD2 | TYR | A | 409 | 23.712 | 74.643 | 27.788 | 1.00 | 62.60 | C |
| ATOM | 1490 | CE1 | TYR | A | 409 | 26.244 | 75.650 | 27.235 | 1.00 | 60.67 | C |
| ATOM | 1491 | CE2 | TYR | A | 409 | 24.222 | 75.696 | 28.546 | 1.00 | 62.79 | C |
| ATOM | 1492 | CZ | TYR | A | 409 | 25.490 | 76.194 | 28.263 | 1.00 | 62.38 | C |
| ATOM | 1493 | OH | TYR | A | 409 | 25.995 | 77.231 | 29.014 | 1.00 | 61.98 | O |
| ATOM | 1494 | N | TRP | A | 410 | 22.596 | 69.794 | 26.264 | 1.00 | 54.45 | N |
| ATOM | 1495 | CA | TRP | A | 410 | 21.916 | 68.686 | 25.610 | 1.00 | 52.22 | C |
| ATOM | 1496 | C | TRP | A | 410 | 20.433 | 68.993 | 25.435 | 1.00 | 50.64 | C |
| ATOM | 1497 | O | TRP | A | 410 | 19.766 | 69.438 | 26.374 | 1.00 | 50.65 | O |
| ATOM | 1498 | CB | TRP | A | 410 | 22.063 | 67.425 | 26.468 | 1.00 | 50.75 | C |
| ATOM | 1499 | CG | TRP | A | 410 | 21.413 | 66.187 | 25.922 | 1.00 | 49.07 | C |
| ATOM | 1500 | CD1 | TRP | A | 410 | 20.734 | 65.238 | 26.639 | 1.00 | 48.69 | C |
| ATOM | 1501 | CD2 | TRP | A | 410 | 21.470 | 65.705 | 24.574 | 1.00 | 48.27 | C |
| ATOM | 1502 | NE1 | TRP | A | 410 | 20.373 | 64.192 | 25.822 | 1.00 | 49.42 | N |
| ATOM | 1503 | CE2 | TRP | A | 410 | 20.812 | 64.452 | 24.550 | 1.00 | 48.57 | C |
| ATOM | 1504 | CE3 | TRP | A | 410 | 22.018 | 66.204 | 23.386 | 1.00 | 47.52 | C |
| ATOM | 1505 | CZ2 | TRP | A | 410 | 20.689 | 63.692 | 23.382 | 1.00 | 48.02 | C |
| ATOM | 1506 | CZ3 | TRP | A | 410 | 21.895 | 65.450 | 22.226 | 1.00 | 47.07 | C |
| ATOM | 1507 | CH2 | TRP | A | 410 | 21.236 | 64.205 | 22.233 | 1.00 | 47.32 | C |
| ATOM | 1508 | N | MET | A | 411 | 19.929 | 68.765 | 24.227 | 1.00 | 48.41 | N |
| ATOM | 1509 | CA | MET | A | 411 | 18.518 | 68.965 | 23.928 | 1.00 | 44.89 | C |
| ATOM | 1510 | C | MET | A | 411 | 18.060 | 67.760 | 23.116 | 1.00 | 43.99 | C |
| ATOM | 1511 | O | MET | A | 411 | 18.209 | 67.732 | 21.898 | 1.00 | 43.33 | O |
| ATOM | 1512 | CB | MET | A | 411 | 18.287 | 70.258 | 23.132 | 1.00 | 43.96 | C |
| ATOM | 1513 | CG | MET | A | 411 | 18.591 | 71.548 | 23.905 | 1.00 | 43.65 | C |
| ATOM | 1514 | SD | MET | A | 411 | 17.971 | 73.074 | 23.113 | 1.00 | 43.96 | S |
| ATOM | 1515 | CE | MET | A | 411 | 17.175 | 73.811 | 24.453 | 1.00 | 44.14 | C |
| ATOM | 1516 | N | PRO | A | 412 | 17.532 | 66.725 | 23.791 | 1.00 | 44.39 | N |
| ATOM | 1517 | CA | PRO | A | 412 | 17.061 | 65.529 | 23.083 | 1.00 | 43.40 | C |
| ATOM | 1518 | C | PRO | A | 412 | 15.799 | 65.934 | 22.333 | 1.00 | 42.43 | C |
| ATOM | 1519 | O | PRO | A | 412 | 14.981 | 66.688 | 22.864 | 1.00 | 41.97 | O |
| ATOM | 1520 | CB | PRO | A | 412 | 16.733 | 64.545 | 24.213 | 1.00 | 41.69 | C |
| ATOM | 1521 | CG | PRO | A | 412 | 17.433 | 65.094 | 25.408 | 1.00 | 43.29 | C |
| ATOM | 1522 | CD | PRO | A | 412 | 17.330 | 66.579 | 25.241 | 1.00 | 43.54 | C |
| ATOM | 1523 | N | ILE | A | 413 | 15.633 | 65.439 | 21.115 | 1.00 | 41.57 | N |
| ATOM | 1524 | CA | ILE | A | 413 | 14.453 | 65.782 | 20.328 | 1.00 | 42.23 | C |
| ATOM | 1525 | C | ILE | A | 413 | 13.858 | 64.487 | 19.817 | 1.00 | 43.19 | C |
| ATOM | 1526 | O | ILE | A | 413 | 12.820 | 64.471 | 19.156 | 1.00 | 43.14 | O |
| ATOM | 1527 | CB | ILE | A | 413 | 14.833 | 66.645 | 19.107 | 1.00 | 39.38 | C |
| ATOM | 1528 | CG1 | ILE | A | 413 | 15.620 | 65.801 | 18.103 | 1.00 | 38.35 | C |
| ATOM | 1529 | CG2 | ILE | A | 413 | 15.677 | 67.828 | 19.545 | 1.00 | 39.96 | C |
| ATOM | 1530 | CD1 | ILE | A | 413 | 15.975 | 66.535 | 16.824 | 1.00 | 37.51 | C |
| ATOM | 1531 | N | ASP | A | 414 | 14.522 | 63.401 | 20.177 | 1.00 | 44.65 | N |
| ATOM | 1532 | CA | ASP | A | 414 | 14.178 | 62.073 | 19.710 | 1.00 | 45.84 | C |
| ATOM | 1533 | C | ASP | A | 414 | 14.044 | 61.085 | 20.874 | 1.00 | 46.06 | C |
| ATOM | 1534 | O | ASP | A | 414 | 14.311 | 61.434 | 22.025 | 1.00 | 44.81 | O |
| ATOM | 1535 | CB | ASP | A | 414 | 15.311 | 61.667 | 18.774 | 1.00 | 49.05 | C |
| ATOM | 1536 | CG | ASP | A | 414 | 15.029 | 60.424 | 18.012 | 1.00 | 53.03 | C |
| ATOM | 1537 | OD1 | ASP | A | 414 | 13.909 | 60.300 | 17.476 | 1.00 | 58.68 | O |
| ATOM | 1538 | OD2 | ASP | A | 414 | 15.942 | 59.583 | 17.934 | 1.00 | 54.11 | O |
| ATOM | 1539 | N | TRP | A | 415 | 13.618 | 59.860 | 20.573 | 1.00 | 45.81 | N |
| ATOM | 1540 | CA | TRP | A | 415 | 13.483 | 58.819 | 21.592 | 1.00 | 47.08 | C |
| ATOM | 1541 | C | TRP | A | 415 | 14.607 | 57.802 | 21.380 | 1.00 | 49.91 | C |
| ATOM | 1542 | O | TRP | A | 415 | 14.642 | 56.760 | 22.036 | 1.00 | 50.98 | O |
| ATOM | 1543 | CB | TRP | A | 415 | 12.145 | 58.074 | 21.463 | 1.00 | 45.24 | C |
| ATOM | 1544 | CG | TRP | A | 415 | 10.910 | 58.915 | 21.598 | 1.00 | 42.89 | C |
| ATOM | 1545 | CD1 | TRP | A | 415 | 9.792 | 58.852 | 20.814 | 1.00 | 40.72 | C |
| ATOM | 1546 | CD2 | TRP | A | 415 | 10.659 | 59.936 | 22.570 | 1.00 | 41.72 | C |
| ATOM | 1547 | NE1 | TRP | A | 415 | 8.866 | 59.773 | 21.232 | 1.00 | 40.48 | N |
| ATOM | 1548 | CE2 | TRP | A | 415 | 9.368 | 60.452 | 22.309 | 1.00 | 41.24 | C |
| ATOM | 1549 | CE3 | TRP | A | 415 | 11.398 | 60.465 | 23.637 | 1.00 | 40.89 | C |
| ATOM | 1550 | CZ2 | TRP | A | 415 | 8.798 | 61.476 | 23.074 | 1.00 | 42.02 | C |
| ATOM | 1551 | CZ3 | TRP | A | 415 | 10.834 | 61.482 | 24.398 | 1.00 | 43.29 | C |
| ATOM | 1552 | CH2 | TRP | A | 415 | 9.541 | 61.978 | 24.111 | 1.00 | 41.91 | C |
| ATOM | 1553 | N | THR | A | 416 | 15.512 | 58.095 | 20.448 | 1.00 | 51.18 | N |
| ATOM | 1554 | CA | THR | A | 416 | 16.609 | 57.178 | 20.155 | 1.00 | 51.96 | C |
| ATOM | 1555 | C | THR | A | 416 | 18.005 | 57.774 | 20.352 | 1.00 | 51.78 | C |
| ATOM | 1556 | O | THR | A | 416 | 18.981 | 57.248 | 19.815 | 1.00 | 53.72 | O |
| ATOM | 1557 | CB | THR | A | 416 | 16.511 | 56.620 | 18.701 | 1.00 | 51.67 | C |
| ATOM | 1558 | OG1 | THR | A | 416 | 17.058 | 57.565 | 17.771 | 1.00 | 52.23 | O |
| ATOM | 1559 | CG2 | THR | A | 416 | 15.064 | 56.352 | 18.336 | 1.00 | 51.10 | C |
| ATOM | 1560 | N | GLY | A | 417 | 18.104 | 58.861 | 21.115 | 1.00 | 51.13 | N |
| ATOM | 1561 | CA | GLY | A | 417 | 19.403 | 59.471 | 21.360 | 1.00 | 50.03 | C |
| ATOM | 1562 | C | GLY | A | 417 | 19.729 | 60.726 | 20.561 | 1.00 | 50.48 | C |
| ATOM | 1563 | O | GLY | A | 417 | 20.609 | 61.500 | 20.951 | 1.00 | 51.42 | O |
| ATOM | 1564 | N | VAL | A | 418 | 19.025 | 60.933 | 19.448 | 1.00 | 48.73 | N |
| ATOM | 1565 | CA | VAL | A | 418 | 19.243 | 62.101 | 18.599 | 1.00 | 44.99 | C |
| ATOM | 1566 | C | VAL | A | 418 | 18.854 | 63.392 | 19.315 | 1.00 | 45.27 | C |
| ATOM | 1567 | O | VAL | A | 418 | 17.859 | 63.438 | 20.045 | 1.00 | 44.33 | O |
| ATOM | 1568 | CB | VAL | A | 418 | 18.419 | 62.000 | 17.298 | 1.00 | 44.74 | C |
| ATOM | 1569 | CG1 | VAL | A | 418 | 18.733 | 63.182 | 16.391 | 1.00 | 42.93 | C |
| ATOM | 1570 | CG2 | VAL | A | 418 | 18.700 | 60.678 | 16.600 | 1.00 | 43.01 | C |
| ATOM | 1571 | N | GLY | A | 419 | 19.638 | 64.442 | 19.094 | 1.00 | 45.22 | N |
| ATOM | 1572 | CA | GLY | A | 419 | 19.358 | 65.719 | 19.724 | 1.00 | 43.80 | C |
| ATOM | 1573 | C | GLY | A | 419 | 20.321 | 66.796 | 19.264 | 1.00 | 45.22 | C |
| ATOM | 1574 | O | GLY | A | 419 | 21.141 | 66.578 | 18.373 | 1.00 | 43.48 | O |
| ATOM | 1575 | N | ILE | A | 420 | 20.218 | 67.966 | 19.880 | 1.00 | 47.08 | N |
| ATOM | 1576 | CA | ILE | A | 420 | 21.073 | 69.101 | 19.559 | 1.00 | 48.66 | C |
| ATOM | 1577 | C | ILE | A | 420 | 22.024 | 69.342 | 20.735 | 1.00 | 51.49 | C |
| ATOM | 1578 | O | ILE | A | 420 | 21.610 | 69.269 | 21.895 | 1.00 | 50.60 | O |
| ATOM | 1579 | CB | ILE | A | 420 | 20.217 | 70.371 | 19.329 | 1.00 | 47.83 | C |
| ATOM | 1580 | CG1 | ILE | A | 420 | 19.201 | 70.117 | 18.210 | 1.00 | 47.02 | C |
| ATOM | 1581 | CG2 | ILE | A | 420 | 21.103 | 71.555 | 18.983 | 1.00 | 46.71 | C |
| ATOM | 1582 | CD1 | ILE | A | 420 | 18.172 | 71.220 | 18.061 | 1.00 | 45.85 | C |
| ATOM | 1583 | N | HIS | A | 421 | 23.296 | 69.609 | 20.438 | 1.00 | 54.64 | N |
| ATOM | 1584 | CA | HIS | A | 421 | 24.290 | 69.873 | 21.481 | 1.00 | 57.06 | C |
| ATOM | 1585 | C | HIS | A | 421 | 25.569 | 70.480 | 20.922 | 1.00 | 59.03 | C |
| ATOM | 1586 | O | HIS | A | 421 | 25.859 | 70.341 | 19.737 | 1.00 | 58.74 | O |
| ATOM | 1587 | CB | HIS | A | 421 | 24.634 | 68.584 | 22.240 | 1.00 | 56.41 | C |
| ATOM | 1588 | CG | HIS | A | 421 | 25.359 | 67.562 | 21.420 | 1.00 | 56.78 | C |
| ATOM | 1589 | ND1 | HIS | A | 421 | 26.594 | 67.798 | 20.853 | 1.00 | 57.01 | N |
| ATOM | 1590 | CD2 | HIS | A | 421 | 25.039 | 66.285 | 21.103 | 1.00 | 56.80 | C |
| ATOM | 1591 | CE1 | HIS | A | 421 | 27.002 | 66.710 | 20.225 | 1.00 | 56.73 | C |
| ATOM | 1592 | NE2 | HIS | A | 421 | 26.076 | 65.777 | 20.361 | 1.00 | 55.68 | N |
| ATOM | 1593 | N | ASP | A | 422 | 26.331 | 71.154 | 21.781 | 1.00 | 62.73 | N |
| ATOM | 1594 | CA | ASP | A | 422 | 27.592 | 71.765 | 21.366 | 1.00 | 65.68 | C |
| ATOM | 1595 | C | ASP | A | 422 | 28.607 | 70.706 | 20.953 | 1.00 | 66.65 | C |
| ATOM | 1596 | O | ASP | A | 422 | 28.783 | 69.688 | 21.625 | 1.00 | 65.10 | O |
| ATOM | 1597 | CB | ASP | A | 422 | 28.181 | 72.639 | 22.479 | 1.00 | 67.56 | C |
| ATOM | 1598 | CG | ASP | A | 422 | 28.175 | 71.954 | 23.828 | 1.00 | 69.25 | C |
| ATOM | 1599 | OD1 | ASP | A | 422 | 28.896 | 72.420 | 24.736 | 1.00 | 70.04 | O |
| ATOM | 1600 | OD2 | ASP | A | 422 | 27.444 | 70.958 | 23.987 | 1.00 | 70.88 | O |
| ATOM | 1601 | N | SER | A | 423 | 29.264 | 70.964 | 19.829 | 1.00 | 69.62 | N |
| ATOM | 1602 | CA | SER | A | 423 | 30.254 | 70.056 | 19.277 | 1.00 | 72.96 | C |
| ATOM | 1603 | C | SER | A | 423 | 31.647 | 70.649 | 19.445 | 1.00 | 74.78 | C |
| ATOM | 1604 | O | SER | A | 423 | 32.346 | 70.926 | 18.471 | 1.00 | 74.16 | O |
| ATOM | 1605 | CB | SER | A | 423 | 29.966 | 69.823 | 17.799 | 1.00 | 73.31 | C |
| ATOM | 1606 | OG | SER | A | 423 | 30.545 | 68.612 | 17.360 | 1.00 | 76.94 | O |
| ATOM | 1607 | N | ASP | A | 424 | 32.025 | 70.843 | 20.701 | 1.00 | 76.90 | N |
| ATOM | 1608 | CA | ASP | A | 424 | 33.316 | 71.394 | 21.082 | 1.00 | 79.50 | C |
| ATOM | 1609 | C | ASP | A | 424 | 34.489 | 70.864 | 20.245 | 1.00 | 80.26 | C |
| ATOM | 1610 | O | ASP | A | 424 | 35.358 | 71.626 | 19.819 | 1.00 | 79.99 | O |
| ATOM | 1611 | CB | ASP | A | 424 | 33.544 | 71.081 | 22.559 | 1.00 | 81.09 | C |
| ATOM | 1612 | CG | ASP | A | 424 | 34.842 | 71.617 | 23.073 | 1.00 | 82.13 | C |
| ATOM | 1613 | OD1 | ASP | A | 424 | 35.074 | 72.834 | 22.927 | 1.00 | 83.89 | O |
| ATOM | 1614 | OD2 | ASP | A | 424 | 35.624 | 70.818 | 23.629 | 1.00 | 82.66 | O |
| ATOM | 1615 | N | TRP | A | 425 | 34.491 | 69.554 | 20.012 | 1.00 | 81.36 | N |
| ATOM | 1616 | CA | TRP | A | 425 | 35.539 | 68.870 | 19.255 | 1.00 | 82.02 | C |
| ATOM | 1617 | C | TRP | A | 425 | 35.469 | 69.039 | 17.739 | 1.00 | 82.77 | C |
| ATOM | 1618 | O | TRP | A | 425 | 36.140 | 68.315 | 17.005 | 1.00 | 83.09 | O |
| ATOM | 1619 | CB | TRP | A | 425 | 35.509 | 67.379 | 19.594 | 1.00 | 82.45 | C |
| ATOM | 1620 | CG | TRP | A | 425 | 34.174 | 66.750 | 19.348 | 1.00 | 83.22 | C |
| ATOM | 1621 | CD1 | TRP | A | 425 | 33.781 | 66.060 | 18.236 | 1.00 | 83.73 | C |
| ATOM | 1622 | CD2 | TRP | A | 425 | 33.034 | 66.798 | 20.214 | 1.00 | 83.59 | C |
| ATOM | 1623 | NE1 | TRP | A | 425 | 32.466 | 65.677 | 18.355 | 1.00 | 83.76 | N |
| ATOM | 1624 | CE2 | TRP | A | 425 | 31.983 | 66.117 | 19.560 | 1.00 | 84.05 | C |
| ATOM | 1625 | CE3 | TRP | A | 425 | 32.798 | 67.355 | 21.480 | 1.00 | 83.31 | C |
| ATOM | 1626 | CZ2 | TRP | A | 425 | 30.711 | 65.976 | 20.130 | 1.00 | 83.54 | C |
| ATOM | 1627 | CZ3 | TRP | A | 425 | 31.534 | 67.216 | 22.047 | 1.00 | 83.18 | C |
| ATOM | 1628 | CH2 | TRP | A | 425 | 30.507 | 66.531 | 21.370 | 1.00 | 83.40 | C |
| ATOM | 1629 | N | GLN | A | 426 | 34.657 | 69.984 | 17.272 | 1.00 | 83.91 | N |
| ATOM | 1630 | CA | GLN | A | 426 | 34.518 | 70.242 | 15.837 | 1.00 | 84.05 | C |
| ATOM | 1631 | C | GLN | A | 426 | 35.098 | 71.611 | 15.482 | 1.00 | 84.24 | C |
| ATOM | 1632 | O | GLN | A | 426 | 34.624 | 72.642 | 15.957 | 1.00 | 83.84 | O |
| ATOM | 1633 | CB | GLN | A | 426 | 33.041 | 70.171 | 15.422 | 1.00 | 83.96 | C |
| ATOM | 1634 | CG | GLN | A | 426 | 32.470 | 68.757 | 15.417 | 1.00 | 83.28 | C |
| ATOM | 1635 | CD | GLN | A | 426 | 32.615 | 68.057 | 14.079 | 1.00 | 83.21 | C |
| ATOM | 1636 | OE1 | GLN | A | 426 | 33.623 | 68.292 | 13.379 | 1.00 | 84.79 | O |
| ATOM | 1637 | NE2 | GLN | A | 426 | 31.722 | 67.259 | 13.730 | 1.00 | 82.29 | N |
| ATOM | 1638 | N | PRO | A | 427 | 36.138 | 71.633 | 14.636 | 1.00 | 84.58 | N |
| ATOM | 1639 | CA | PRO | A | 427 | 36.786 | 72.878 | 14.218 | 1.00 | 84.51 | C |
| ATOM | 1640 | C | PRO | A | 427 | 35.984 | 73.634 | 13.162 | 1.00 | 84.48 | C |
| ATOM | 1641 | O | PRO | A | 427 | 36.008 | 74.865 | 13.118 | 1.00 | 84.02 | O |
| ATOM | 1642 | CB | PRO | A | 427 | 38.121 | 72.391 | 13.678 | 1.00 | 84.70 | C |
| ATOM | 1643 | CG | PRO | A | 427 | 37.722 | 71.112 | 12.997 | 1.00 | 84.92 | C |
| ATOM | 1644 | CD | PRO | A | 427 | 36.804 | 70.467 | 14.024 | 1.00 | 84.90 | C |
| ATOM | 1645 | N | GLU | A | 428 | 35.278 | 72.889 | 12.314 | 1.00 | 84.68 | N |
| ATOM | 1646 | CA | GLU | A | 428 | 34.479 | 73.490 | 11.248 | 1.00 | 84.41 | C |
| ATOM | 1647 | C | GLU | A | 428 | 33.079 | 72.877 | 11.116 | 1.00 | 83.76 | C |
| ATOM | 1648 | O | GLU | A | 428 | 32.896 | 71.663 | 11.270 | 1.00 | 83.56 | O |
| ATOM | 1649 | CB | GLU | A | 428 | 35.227 | 73.378 | 9.918 | 1.00 | 83.91 | C |
| ATOM | 1650 | N | TYR | A | 429 | 32.102 | 73.735 | 10.823 | 1.00 | 82.13 | N |
| ATOM | 1651 | CA | TYR | A | 429 | 30.714 | 73.321 | 10.653 | 1.00 | 80.89 | C |
| ATOM | 1652 | C | TYR | A | 429 | 30.201 | 73.717 | 9.271 | 1.00 | 80.26 | C |
| ATOM | 1653 | O | TYR | A | 429 | 30.792 | 74.561 | 8.600 | 1.00 | 80.24 | O |
| ATOM | 1654 | CB | TYR | A | 429 | 29.820 | 73.982 | 11.702 | 1.00 | 80.64 | C |
| ATOM | 1655 | CG | TYR | A | 429 | 30.174 | 73.688 | 13.141 | 1.00 | 81.34 | C |
| ATOM | 1656 | CD1 | TYR | A | 429 | 31.301 | 74.254 | 13.737 | 1.00 | 81.31 | C |
| ATOM | 1657 | CD2 | TYR | A | 429 | 29.355 | 72.870 | 13.921 | 1.00 | 81.53 | C |
| ATOM | 1658 | CE1 | TYR | A | 429 | 31.601 | 74.017 | 15.080 | 1.00 | 82.04 | C |
| ATOM | 1659 | CE2 | TYR | A | 429 | 29.644 | 72.625 | 15.261 | 1.00 | 81.97 | C |
| ATOM | 1660 | CZ | TYR | A | 429 | 30.765 | 73.202 | 15.835 | 1.00 | 82.91 | C |
| ATOM | 1661 | OH | TYR | A | 429 | 31.032 | 72.976 | 17.168 | 1.00 | 83.51 | O |
| ATOM | 1662 | N | GLY | A | 430 | 29.088 | 73.113 | 8.864 | 1.00 | 79.59 | N |
| ATOM | 1663 | CA | GLY | A | 430 | 28.497 | 73.421 | 7.573 | 1.00 | 78.98 | C |
| ATOM | 1664 | C | GLY | A | 430 | 29.039 | 72.578 | 6.434 | 1.00 | 79.06 | C |
| ATOM | 1665 | O | GLY | A | 430 | 29.715 | 71.572 | 6.659 | 1.00 | 78.55 | O |
| ATOM | 1666 | N | GLY | A | 431 | 28.720 | 72.985 | 5.207 | 1.00 | 77.99 | N |
| ATOM | 1667 | CA | GLY | A | 431 | 29.191 | 72.282 | 4.027 | 1.00 | 77.61 | C |
| ATOM | 1668 | C | GLY | A | 431 | 28.998 | 70.778 | 3.988 | 1.00 | 77.99 | C |
| ATOM | 1669 | O | GLY | A | 431 | 27.931 | 70.270 | 4.333 | 1.00 | 79.00 | O |
| ATOM | 1670 | N | ASP | A | 432 | 30.042 | 70.072 | 3.555 | 1.00 | 78.13 | N |
| ATOM | 1671 | CA | ASP | A | 432 | 30.029 | 68.612 | 3.432 | 1.00 | 77.54 | C |
| ATOM | 1672 | C | ASP | A | 432 | 30.703 | 67.910 | 4.617 | 1.00 | 75.91 | C |
| ATOM | 1673 | O | ASP | A | 432 | 30.887 | 66.691 | 4.596 | 1.00 | 75.66 | O |
| ATOM | 1674 | CB | ASP | A | 432 | 30.746 | 68.186 | 2.138 | 1.00 | 80.08 | C |
| ATOM | 1675 | CG | ASP | A | 432 | 29.972 | 68.553 | 0.870 | 1.00 | 83.04 | C |
| ATOM | 1676 | OD1 | ASP | A | 432 | 29.576 | 69.730 | 0.707 | 1.00 | 84.31 | O |
| ATOM | 1677 | OD2 | ASP | A | 432 | 29.774 | 67.655 | 0.021 | 1.00 | 84.14 | O |
| ATOM | 1678 | N | LEU | A | 433 | 31.068 | 68.673 | 5.644 | 1.00 | 74.30 | N |
| ATOM | 1679 | CA | LEU | A | 433 | 31.737 | 68.106 | 6.812 | 1.00 | 73.86 | C |
| ATOM | 1680 | C | LEU | A | 433 | 31.154 | 66.780 | 7.309 | 1.00 | 73.37 | C |
| ATOM | 1681 | O | LEU | A | 433 | 31.898 | 65.832 | 7.566 | 1.00 | 73.73 | O |
| ATOM | 1682 | CB | LEU | A | 433 | 31.757 | 69.115 | 7.970 | 1.00 | 74.42 | C |
| ATOM | 1683 | CG | LEU | A | 433 | 32.808 | 70.238 | 8.000 | 1.00 | 76.24 | C |
| ATOM | 1684 | CD1 | LEU | A | 433 | 34.202 | 69.642 | 7.933 | 1.00 | 77.25 | C |
| ATOM | 1685 | CD2 | LEU | A | 433 | 32.603 | 71.185 | 6.843 | 1.00 | 77.36 | C |
| ATOM | 1686 | N | TRP | A | 434 | 29.832 | 66.707 | 7.435 | 1.00 | 72.27 | N |
| ATOM | 1687 | CA | TRP | A | 434 | 29.178 | 65.494 | 7.928 | 1.00 | 71.44 | C |
| ATOM | 1688 | C | TRP | A | 434 | 29.603 | 64.178 | 7.268 | 1.00 | 72.42 | C |
| ATOM | 1689 | O | TRP | A | 434 | 29.588 | 63.133 | 7.920 | 1.00 | 72.54 | O |
| ATOM | 1690 | CB | TRP | A | 434 | 27.657 | 65.625 | 7.819 | 1.00 | 68.39 | C |
| ATOM | 1691 | CG | TRP | A | 434 | 27.176 | 65.790 | 6.421 | 1.00 | 65.05 | C |
| ATOM | 1692 | CD1 | TRP | A | 434 | 27.252 | 66.918 | 5.655 | 1.00 | 65.22 | C |
| ATOM | 1693 | CD2 | TRP | A | 434 | 26.557 | 64.790 | 5.605 | 1.00 | 64.08 | C |
| ATOM | 1694 | NE1 | TRP | A | 434 | 26.716 | 66.684 | 4.412 | 1.00 | 64.45 | N |
| ATOM | 1695 | CE2 | TRP | A | 434 | 26.282 | 65.385 | 4.353 | 1.00 | 63.90 | C |
| ATOM | 1696 | CE3 | TRP | A | 434 | 26.209 | 63.448 | 5.809 | 1.00 | 63.52 | C |
| ATOM | 1697 | CZ2 | TRP | A | 434 | 25.674 | 64.683 | 3.307 | 1.00 | 63.30 | C |
| ATOM | 1698 | CZ3 | TRP | A | 434 | 25.604 | 62.748 | 4.769 | 1.00 | 62.95 | C |
| ATOM | 1699 | CH2 | TRP | A | 434 | 25.344 | 63.369 | 3.533 | 1.00 | 63.46 | C |
| ATOM | 1700 | N | LYS | A | 435 | 29.964 | 64.216 | 5.986 | 1.00 | 73.52 | N |
| ATOM | 1701 | CA | LYS | A | 435 | 30.373 | 62.996 | 5.287 | 1.00 | 74.81 | C |
| ATOM | 1702 | C | LYS | A | 435 | 31.749 | 62.524 | 5.742 | 1.00 | 75.61 | C |
| ATOM | 1703 | O | LYS | A | 435 | 32.086 | 61.345 | 5.622 | 1.00 | 74.96 | O |
| ATOM | 1704 | CB | LYS | A | 435 | 30.376 | 63.220 | 3.773 | 1.00 | 74.99 | C |
| ATOM | 1705 | CG | LYS | A | 435 | 28.988 | 63.400 | 3.183 | 1.00 | 76.65 | C |
| ATOM | 1706 | CD | LYS | A | 435 | 29.002 | 63.389 | 1.662 | 1.00 | 78.11 | C |
| ATOM | 1707 | CE | LYS | A | 435 | 29.796 | 64.554 | 1.090 | 1.00 | 79.68 | C |
| ATOM | 1708 | NZ | LYS | A | 435 | 29.786 | 64.544 | −0.405 | 1.00 | 79.62 | N |
| ATOM | 1709 | N | THR | A | 436 | 32.531 | 63.460 | 6.272 | 1.00 | 77.01 | N |
| ATOM | 1710 | CA | THR | A | 436 | 33.880 | 63.188 | 6.763 | 1.00 | 77.96 | C |
| ATOM | 1711 | C | THR | A | 436 | 33.851 | 62.971 | 8.269 | 1.00 | 77.81 | C |
| ATOM | 1712 | O | THR | A | 436 | 33.990 | 61.841 | 8.744 | 1.00 | 78.03 | O |
| ATOM | 1713 | CB | THR | A | 436 | 34.820 | 64.366 | 6.456 | 1.00 | 78.70 | C |
| ATOM | 1714 | OG1 | THR | A | 436 | 34.940 | 64.512 | 5.036 | 1.00 | 79.39 | O |
| ATOM | 1715 | CG2 | THR | A | 436 | 36.197 | 64.136 | 7.073 | 1.00 | 80.17 | C |
| ATOM | 1716 | N | ARG | A | 437 | 33.678 | 64.058 | 9.018 | 1.00 | 77.01 | N |
| ATOM | 1717 | CA | ARG | A | 437 | 33.614 | 63.966 | 10.469 | 1.00 | 77.08 | C |
| ATOM | 1718 | C | ARG | A | 437 | 32.192 | 64.147 | 10.985 | 1.00 | 75.59 | C |
| ATOM | 1719 | O | ARG | A | 437 | 31.940 | 64.942 | 11.892 | 1.00 | 73.97 | O |
| ATOM | 1720 | CB | ARG | A | 437 | 34.554 | 64.987 | 11.118 | 1.00 | 79.41 | C |
| ATOM | 1721 | CG | ARG | A | 437 | 34.639 | 66.338 | 10.434 | 1.00 | 81.35 | C |
| ATOM | 1722 | CD | ARG | A | 437 | 35.499 | 67.269 | 11.280 | 1.00 | 84.12 | C |
| ATOM | 1723 | NE | ARG | A | 437 | 35.917 | 68.475 | 10.570 | 1.00 | 86.73 | N |
| ATOM | 1724 | CZ | ARG | A | 437 | 36.746 | 68.481 | 9.529 | 1.00 | 87.20 | C |
| ATOM | 1725 | NH1 | ARG | A | 437 | 37.251 | 67.342 | 9.068 | 1.00 | 87.20 | N |
| ATOM | 1726 | NH2 | ARG | A | 437 | 37.076 | 69.631 | 8.954 | 1.00 | 87.39 | N |
| ATOM | 1727 | N | GLY | A | 438 | 31.271 | 63.385 | 10.400 | 1.00 | 74.86 | N |
| ATOM | 1728 | CA | GLY | A | 438 | 29.875 | 63.448 | 10.788 | 1.00 | 73.59 | C |
| ATOM | 1729 | C | GLY | A | 438 | 29.612 | 62.883 | 12.170 | 1.00 | 73.40 | C |
| ATOM | 1730 | O | GLY | A | 438 | 30.393 | 62.082 | 12.689 | 1.00 | 73.95 | O |
| ATOM | 1731 | N | SER | A | 439 | 28.495 | 63.301 | 12.758 | 1.00 | 72.24 | N |
| ATOM | 1732 | CA | SER | A | 439 | 28.088 | 62.877 | 14.094 | 1.00 | 70.31 | C |
| ATOM | 1733 | C | SER | A | 439 | 27.507 | 61.466 | 14.111 | 1.00 | 68.70 | C |
| ATOM | 1734 | O | SER | A | 439 | 27.483 | 60.783 | 13.086 | 1.00 | 67.74 | O |
| ATOM | 1735 | CB | SER | A | 439 | 27.048 | 63.861 | 14.636 | 1.00 | 70.72 | C |
| ATOM | 1736 | OG | SER | A | 439 | 26.725 | 63.579 | 15.982 | 1.00 | 74.02 | O |
| ATOM | 1737 | N | HIS | A | 440 | 27.056 | 61.030 | 15.286 | 1.00 | 67.64 | N |
| ATOM | 1738 | CA | HIS | A | 440 | 26.446 | 59.709 | 15.433 | 1.00 | 67.21 | C |
| ATOM | 1739 | C | HIS | A | 440 | 24.940 | 59.838 | 15.211 | 1.00 | 64.75 | C |
| ATOM | 1740 | O | HIS | A | 440 | 24.182 | 58.887 | 15.402 | 1.00 | 64.70 | O |
| ATOM | 1741 | CB | HIS | A | 440 | 26.711 | 59.126 | 16.830 | 1.00 | 69.59 | C |
| ATOM | 1742 | CG | HIS | A | 440 | 28.105 | 58.612 | 17.022 | 1.00 | 71.67 | C |
| ATOM | 1743 | ND1 | HIS | A | 440 | 29.158 | 59.425 | 17.388 | 1.00 | 72.98 | N |
| ATOM | 1744 | CD2 | HIS | A | 440 | 28.624 | 57.369 | 16.872 | 1.00 | 72.37 | C |
| ATOM | 1745 | CE1 | HIS | A | 440 | 30.265 | 58.705 | 17.456 | 1.00 | 72.92 | C |
| ATOM | 1746 | NE2 | HIS | A | 440 | 29.968 | 57.454 | 17.148 | 1.00 | 72.82 | N |
| ATOM | 1747 | N | GLY | A | 441 | 24.520 | 61.029 | 14.801 | 1.00 | 61.59 | N |
| ATOM | 1748 | CA | GLY | A | 441 | 23.117 | 61.278 | 14.554 | 1.00 | 58.21 | C |
| ATOM | 1749 | C | GLY | A | 441 | 22.722 | 62.652 | 15.051 | 1.00 | 56.27 | C |
| ATOM | 1750 | O | GLY | A | 441 | 21.888 | 63.318 | 14.449 | 1.00 | 55.40 | O |
| ATOM | 1751 | N | CYS | A | 442 | 23.332 | 63.083 | 16.148 | 1.00 | 54.26 | N |
| ATOM | 1752 | CA | CYS | A | 442 | 23.022 | 64.383 | 16.717 | 1.00 | 54.06 | C |
| ATOM | 1753 | C | CYS | A | 442 | 23.406 | 65.536 | 15.814 | 1.00 | 53.40 | C |
| ATOM | 1754 | O | CYS | A | 442 | 24.158 | 65.377 | 14.853 | 1.00 | 53.14 | O |
| ATOM | 1755 | CB | CYS | A | 442 | 23.722 | 64.558 | 18.064 | 1.00 | 55.28 | C |
| ATOM | 1756 | SG | CYS | A | 442 | 23.061 | 63.519 | 19.375 | 1.00 | 59.64 | S |
| ATOM | 1757 | N | ILE | A | 443 | 22.865 | 66.703 | 16.134 | 1.00 | 53.05 | N |
| ATOM | 1758 | CA | ILE | A | 443 | 23.152 | 67.910 | 15.390 | 1.00 | 53.10 | C |
| ATOM | 1759 | C | ILE | A | 443 | 24.216 | 68.658 | 16.174 | 1.00 | 53.44 | C |
| ATOM | 1760 | O | ILE | A | 443 | 23.933 | 69.265 | 17.211 | 1.00 | 51.86 | O |
| ATOM | 1761 | CB | ILE | A | 443 | 21.890 | 68.800 | 15.231 | 1.00 | 53.53 | C |
| ATOM | 1762 | CG1 | ILE | A | 443 | 20.998 | 68.246 | 14.118 | 1.00 | 52.96 | C |
| ATOM | 1763 | CG2 | ILE | A | 443 | 22.284 | 70.238 | 14.891 | 1.00 | 53.77 | C |
| ATOM | 1764 | CD1 | ILE | A | 443 | 20.348 | 66.936 | 14.440 | 1.00 | 55.23 | C |
| ATOM | 1765 | N | ASN | A | 444 | 25.451 | 68.581 | 15.683 | 1.00 | 54.47 | N |
| ATOM | 1766 | CA | ASN | A | 444 | 26.573 | 69.253 | 16.320 | 1.00 | 54.28 | C |
| ATOM | 1767 | C | ASN | A | 444 | 26.457 | 70.753 | 16.080 | 1.00 | 53.16 | C |
| ATOM | 1768 | O | ASN | A | 444 | 26.266 | 71.196 | 14.953 | 1.00 | 52.11 | O |
| ATOM | 1769 | CB | ASN | A | 444 | 27.887 | 68.697 | 15.768 | 1.00 | 57.26 | C |
| ATOM | 1770 | CG | ASN | A | 444 | 28.093 | 67.224 | 16.128 | 1.00 | 59.12 | C |
| ATOM | 1771 | OD1 | ASN | A | 444 | 27.834 | 66.806 | 17.259 | 1.00 | 59.78 | O |
| ATOM | 1772 | ND2 | ASN | A | 444 | 28.568 | 66.439 | 15.169 | 1.00 | 60.27 | N |
| ATOM | 1773 | N | THR | A | 445 | 26.574 | 71.525 | 17.153 | 1.00 | 54.13 | N |
| ATOM | 1774 | CA | THR | A | 445 | 26.435 | 72.978 | 17.092 | 1.00 | 55.75 | C |
| ATOM | 1775 | C | THR | A | 445 | 27.626 | 73.745 | 17.680 | 1.00 | 57.28 | C |
| ATOM | 1776 | O | THR | A | 445 | 28.184 | 73.356 | 18.707 | 1.00 | 57.44 | O |
| ATOM | 1777 | CB | THR | A | 445 | 25.160 | 73.405 | 17.846 | 1.00 | 55.08 | C |
| ATOM | 1778 | OG1 | THR | A | 445 | 24.034 | 72.728 | 17.276 | 1.00 | 56.22 | O |
| ATOM | 1779 | CG2 | THR | A | 445 | 24.953 | 74.910 | 17.764 | 1.00 | 54.30 | C |
| ATOM | 1780 | N | PRO | A | 446 | 28.029 | 74.852 | 17.034 | 1.00 | 58.05 | N |
| ATOM | 1781 | CA | PRO | A | 446 | 29.157 | 75.629 | 17.552 | 1.00 | 58.97 | C |
| ATOM | 1782 | C | PRO | A | 446 | 28.909 | 75.972 | 19.017 | 1.00 | 60.54 | C |
| ATOM | 1783 | O | PRO | A | 446 | 27.882 | 76.571 | 19.356 | 1.00 | 61.39 | O |
| ATOM | 1784 | CB | PRO | A | 446 | 29.157 | 76.864 | 16.659 | 1.00 | 59.15 | C |
| ATOM | 1785 | CG | PRO | A | 446 | 28.675 | 76.316 | 15.350 | 1.00 | 58.73 | C |
| ATOM | 1786 | CD | PRO | A | 446 | 27.526 | 75.432 | 15.776 | 1.00 | 58.36 | C |
| ATOM | 1787 | N | PRO | A | 447 | 29.841 | 75.586 | 19.906 | 1.00 | 60.39 | N |
| ATOM | 1788 | CA | PRO | A | 447 | 29.753 | 75.835 | 21.347 | 1.00 | 60.05 | C |
| ATOM | 1789 | C | PRO | A | 447 | 29.175 | 77.187 | 21.772 | 1.00 | 60.19 | C |
| ATOM | 1790 | O | PRO | A | 447 | 28.329 | 77.248 | 22.662 | 1.00 | 60.48 | O |
| ATOM | 1791 | CB | PRO | A | 447 | 31.193 | 75.638 | 21.804 | 1.00 | 59.76 | C |
| ATOM | 1792 | CG | PRO | A | 447 | 31.618 | 74.477 | 20.956 | 1.00 | 59.75 | C |
| ATOM | 1793 | CD | PRO | A | 447 | 31.082 | 74.855 | 19.582 | 1.00 | 59.96 | C |
| ATOM | 1794 | N | SER | A | 448 | 29.622 | 78.271 | 21.149 | 1.00 | 61.01 | N |
| ATOM | 1795 | CA | SER | A | 448 | 29.116 | 79.594 | 21.522 | 1.00 | 61.99 | C |
| ATOM | 1796 | C | SER | A | 448 | 27.644 | 79.777 | 21.151 | 1.00 | 61.09 | C |
| ATOM | 1797 | O | SER | A | 448 | 26.869 | 80.344 | 21.920 | 1.00 | 60.46 | O |
| ATOM | 1798 | CB | SER | A | 448 | 29.965 | 80.698 | 20.871 | 1.00 | 62.82 | C |
| ATOM | 1799 | OG | SER | A | 448 | 30.093 | 80.502 | 19.470 | 1.00 | 65.62 | O |
| ATOM | 1800 | N | VAL | A | 449 | 27.267 | 79.292 | 19.973 | 1.00 | 61.06 | N |
| ATOM | 1801 | CA | VAL | A | 449 | 25.891 | 79.397 | 19.504 | 1.00 | 61.02 | C |
| ATOM | 1802 | C | VAL | A | 449 | 24.979 | 78.544 | 20.378 | 1.00 | 60.19 | C |
| ATOM | 1803 | O | VAL | A | 449 | 23.959 | 79.019 | 20.878 | 1.00 | 58.87 | O |
| ATOM | 1804 | CB | VAL | A | 449 | 25.761 | 78.921 | 18.032 | 1.00 | 61.95 | C |
| ATOM | 1805 | CG1 | VAL | A | 449 | 24.313 | 78.998 | 17.587 | 1.00 | 62.45 | C |
| ATOM | 1806 | CG2 | VAL | A | 449 | 26.632 | 79.777 | 17.124 | 1.00 | 61.93 | C |
| ATOM | 1807 | N | MET | A | 450 | 25.363 | 77.285 | 20.564 | 1.00 | 59.93 | N |
| ATOM | 1808 | CA | MET | A | 450 | 24.584 | 76.349 | 21.365 | 1.00 | 60.40 | C |
| ATOM | 1809 | C | MET | A | 450 | 24.202 | 76.936 | 22.715 | 1.00 | 62.48 | C |
| ATOM | 1810 | O | MET | A | 450 | 23.090 | 76.715 | 23.197 | 1.00 | 62.55 | O |
| ATOM | 1811 | CB | MET | A | 450 | 25.369 | 75.058 | 21.583 | 1.00 | 56.88 | C |
| ATOM | 1812 | CG | MET | A | 450 | 24.586 | 73.958 | 22.288 | 1.00 | 54.25 | C |
| ATOM | 1813 | SD | MET | A | 450 | 23.278 | 73.199 | 21.285 | 1.00 | 47.37 | S |
| ATOM | 1814 | CE | MET | A | 450 | 21.874 | 73.407 | 22.381 | 1.00 | 49.68 | C |
| ATOM | 1815 | N | LYS | A | 451 | 25.122 | 77.682 | 23.323 | 1.00 | 64.01 | N |
| ATOM | 1816 | CA | LYS | A | 451 | 24.876 | 78.294 | 24.630 | 1.00 | 65.57 | C |
| ATOM | 1817 | C | LYS | A | 451 | 23.758 | 79.321 | 24.573 | 1.00 | 65.90 | C |
| ATOM | 1818 | O | LYS | A | 451 | 22.921 | 79.400 | 25.475 | 1.00 | 65.90 | O |
| ATOM | 1819 | CB | LYS | A | 451 | 26.146 | 78.970 | 25.155 | 1.00 | 66.58 | C |
| ATOM | 1820 | CG | LYS | A | 451 | 25.952 | 79.749 | 26.454 | 1.00 | 66.59 | C |
| ATOM | 1821 | CD | LYS | A | 451 | 27.290 | 80.229 | 27.006 | 1.00 | 68.80 | C |
| ATOM | 1822 | CE | LYS | A | 451 | 27.128 | 81.002 | 28.304 | 1.00 | 69.61 | C |
| ATOM | 1823 | NZ | LYS | A | 451 | 26.386 | 82.280 | 28.099 | 1.00 | 71.96 | N |
| ATOM | 1824 | N | GLU | A | 452 | 23.765 | 80.111 | 23.505 | 1.00 | 66.01 | N |
| ATOM | 1825 | CA | GLU | A | 452 | 22.771 | 81.153 | 23.297 | 1.00 | 66.26 | C |
| ATOM | 1826 | C | GLU | A | 452 | 21.435 | 80.516 | 22.900 | 1.00 | 64.92 | C |
| ATOM | 1827 | O | GLU | A | 452 | 20.363 | 81.009 | 23.254 | 1.00 | 64.19 | O |
| ATOM | 1828 | CB | GLU | A | 452 | 23.273 | 82.089 | 22.202 | 1.00 | 68.59 | C |
| ATOM | 1829 | CG | GLU | A | 452 | 22.599 | 83.434 | 22.142 | 1.00 | 72.17 | C |
| ATOM | 1830 | CD | GLU | A | 452 | 23.332 | 84.370 | 21.210 | 1.00 | 75.06 | C |
| ATOM | 1831 | OE1 | GLU | A | 452 | 24.529 | 84.638 | 21.468 | 1.00 | 76.84 | O |
| ATOM | 1832 | OE2 | GLU | A | 452 | 22.724 | 84.830 | 20.220 | 1.00 | 76.34 | O |
| ATOM | 1833 | N | LEU | A | 453 | 21.520 | 79.413 | 22.162 | 1.00 | 63.01 | N |
| ATOM | 1834 | CA | LEU | A | 453 | 20.344 | 78.676 | 21.719 | 1.00 | 61.06 | C |
| ATOM | 1835 | C | LEU | A | 453 | 19.644 | 78.135 | 22.962 | 1.00 | 60.25 | C |
| ATOM | 1836 | O | LEU | A | 453 | 18.504 | 78.498 | 23.263 | 1.00 | 59.14 | O |
| ATOM | 1837 | CB | LEU | A | 453 | 20.779 | 77.512 | 20.820 | 1.00 | 59.64 | C |
| ATOM | 1838 | CG | LEU | A | 453 | 19.714 | 76.663 | 20.124 | 1.00 | 58.35 | C |
| ATOM | 1839 | CD1 | LEU | A | 453 | 18.933 | 77.521 | 19.141 | 1.00 | 56.69 | C |
| ATOM | 1840 | CD2 | LEU | A | 453 | 20.387 | 75.510 | 19.397 | 1.00 | 56.49 | C |
| ATOM | 1841 | N | PHE | A | 454 | 20.359 | 77.276 | 23.684 | 1.00 | 59.23 | N |
| ATOM | 1842 | CA | PHE | A | 454 | 19.870 | 76.643 | 24.903 | 1.00 | 57.66 | C |
| ATOM | 1843 | C | PHE | A | 454 | 19.217 | 77.635 | 25.854 | 1.00 | 57.07 | C |
| ATOM | 1844 | O | PHE | A | 454 | 18.199 | 77.337 | 26.470 | 1.00 | 57.50 | O |
| ATOM | 1845 | CB | PHE | A | 454 | 21.031 | 75.930 | 25.601 | 1.00 | 56.99 | C |
| ATOM | 1846 | CG | PHE | A | 454 | 20.624 | 75.102 | 26.788 | 1.00 | 56.17 | C |
| ATOM | 1847 | CD1 | PHE | A | 454 | 20.299 | 75.705 | 28.001 | 1.00 | 54.50 | C |
| ATOM | 1848 | CD2 | PHE | A | 454 | 20.612 | 73.709 | 26.704 | 1.00 | 56.04 | C |
| ATOM | 1849 | CE1 | PHE | A | 454 | 19.977 | 74.936 | 29.112 | 1.00 | 54.55 | C |
| ATOM | 1850 | CE2 | PHE | A | 454 | 20.291 | 72.928 | 27.810 | 1.00 | 54.65 | C |
| ATOM | 1851 | CZ | PHE | A | 454 | 19.974 | 73.541 | 29.017 | 1.00 | 54.72 | C |
| ATOM | 1852 | N | GLY | A | 455 | 19.803 | 78.817 | 25.968 | 1.00 | 57.05 | N |
| ATOM | 1853 | CA | GLY | A | 455 | 19.249 | 79.817 | 26.857 | 1.00 | 57.11 | C |
| ATOM | 1854 | C | GLY | A | 455 | 17.906 | 80.361 | 26.415 | 1.00 | 57.55 | C |
| ATOM | 1855 | O | GLY | A | 455 | 17.090 | 80.763 | 27.250 | 1.00 | 57.59 | O |
| ATOM | 1856 | N | MET | A | 456 | 17.659 | 80.369 | 25.107 | 1.00 | 58.09 | N |
| ATOM | 1857 | CA | MET | A | 456 | 16.398 | 80.894 | 24.592 | 1.00 | 58.46 | C |
| ATOM | 1858 | C | MET | A | 456 | 15.381 | 79.865 | 24.086 | 1.00 | 58.38 | C |
| ATOM | 1859 | O | MET | A | 456 | 14.217 | 80.203 | 23.882 | 1.00 | 58.19 | O |
| ATOM | 1860 | CB | MET | A | 456 | 16.670 | 81.940 | 23.500 | 1.00 | 57.18 | C |
| ATOM | 1861 | CG | MET | A | 456 | 17.514 | 81.462 | 22.335 | 1.00 | 56.29 | C |
| ATOM | 1862 | SD | MET | A | 456 | 17.915 | 82.817 | 21.183 | 1.00 | 56.59 | S |
| ATOM | 1863 | CE | MET | A | 456 | 18.862 | 81.928 | 20.005 | 1.00 | 55.70 | C |
| ATOM | 1864 | N | VAL | A | 457 | 15.803 | 78.619 | 23.885 | 1.00 | 58.39 | N |
| ATOM | 1865 | CA | VAL | A | 457 | 14.875 | 77.595 | 23.411 | 1.00 | 58.13 | C |
| ATOM | 1866 | C | VAL | A | 457 | 14.093 | 76.992 | 24.568 | 1.00 | 58.35 | C |
| ATOM | 1867 | O | VAL | A | 457 | 14.654 | 76.317 | 25.429 | 1.00 | 60.17 | O |
| ATOM | 1868 | CB | VAL | A | 457 | 15.602 | 76.454 | 22.643 | 1.00 | 57.84 | C |
| ATOM | 1869 | CG1 | VAL | A | 457 | 14.665 | 75.258 | 22.459 | 1.00 | 55.16 | C |
| ATOM | 1870 | CG2 | VAL | A | 457 | 16.059 | 76.959 | 21.277 | 1.00 | 57.06 | C |
| ATOM | 1871 | N | GLU | A | 458 | 12.791 | 77.247 | 24.580 | 1.00 | 57.85 | N |
| ATOM | 1872 | CA | GLU | A | 458 | 11.910 | 76.732 | 25.620 | 1.00 | 58.00 | C |
| ATOM | 1873 | C | GLU | A | 458 | 11.658 | 75.237 | 25.376 | 1.00 | 55.77 | C |
| ATOM | 1874 | O | GLU | A | 458 | 11.790 | 74.750 | 24.257 | 1.00 | 55.18 | O |
| ATOM | 1875 | CB | GLU | A | 458 | 10.596 | 77.515 | 25.591 | 1.00 | 59.46 | C |
| ATOM | 1876 | CG | GLU | A | 458 | 9.700 | 77.333 | 26.798 | 1.00 | 65.90 | C |
| ATOM | 1877 | CD | GLU | A | 458 | 8.499 | 78.273 | 26.772 | 1.00 | 69.27 | C |
| ATOM | 1878 | OE1 | GLU | A | 458 | 8.708 | 79.508 | 26.780 | 1.00 | 70.55 | O |
| ATOM | 1879 | OE2 | GLU | A | 458 | 7.348 | 77.776 | 26.743 | 1.00 | 70.99 | O |
| ATOM | 1880 | N | LYS | A | 459 | 11.319 | 74.505 | 26.427 | 1.00 | 54.68 | N |
| ATOM | 1881 | CA | LYS | A | 459 | 11.048 | 73.078 | 26.296 | 1.00 | 53.40 | C |
| ATOM | 1882 | C | LYS | A | 459 | 9.731 | 72.915 | 25.540 | 1.00 | 51.51 | C |
| ATOM | 1883 | O | LYS | A | 459 | 8.833 | 73.748 | 25.665 | 1.00 | 51.82 | O |
| ATOM | 1884 | CB | LYS | A | 459 | 10.944 | 72.443 | 27.681 | 1.00 | 55.22 | C |
| ATOM | 1885 | CG | LYS | A | 459 | 10.913 | 70.929 | 27.686 | 1.00 | 57.22 | C |
| ATOM | 1886 | CD | LYS | A | 459 | 10.702 | 70.416 | 29.102 | 1.00 | 60.73 | C |
| ATOM | 1887 | CE | LYS | A | 459 | 9.315 | 70.784 | 29.619 | 1.00 | 62.32 | C |
| ATOM | 1888 | NZ | LYS | A | 459 | 8.262 | 69.883 | 29.055 | 1.00 | 64.25 | N |
| ATOM | 1889 | N | GLY | A | 460 | 9.616 | 71.850 | 24.755 | 1.00 | 49.99 | N |
| ATOM | 1890 | CA | GLY | A | 460 | 8.403 | 71.631 | 23.983 | 1.00 | 47.36 | C |
| ATOM | 1891 | C | GLY | A | 460 | 8.488 | 72.223 | 22.579 | 1.00 | 45.43 | C |
| ATOM | 1892 | O | GLY | A | 460 | 7.601 | 72.015 | 21.751 | 1.00 | 46.08 | O |
| ATOM | 1893 | N | THR | A | 461 | 9.559 | 72.962 | 22.306 | 1.00 | 42.75 | N |
| ATOM | 1894 | CA | THR | A | 461 | 9.766 | 73.586 | 20.998 | 1.00 | 41.12 | C |
| ATOM | 1895 | C | THR | A | 461 | 9.944 | 72.567 | 19.865 | 1.00 | 39.78 | C |
| ATOM | 1896 | O | THR | A | 461 | 10.815 | 71.698 | 19.912 | 1.00 | 38.77 | O |
| ATOM | 1897 | CB | THR | A | 461 | 11.009 | 74.507 | 21.017 | 1.00 | 41.19 | C |
| ATOM | 1898 | OG1 | THR | A | 461 | 10.823 | 75.534 | 21.993 | 1.00 | 42.37 | O |
| ATOM | 1899 | CG2 | THR | A | 461 | 11.220 | 75.155 | 19.659 | 1.00 | 40.93 | C |
| ATOM | 1900 | N | PRO | A | 462 | 9.117 | 72.674 | 18.820 | 1.00 | 38.72 | N |
| ATOM | 1901 | CA | PRO | A | 462 | 9.211 | 71.747 | 17.687 | 1.00 | 37.84 | C |
| ATOM | 1902 | C | PRO | A | 462 | 10.558 | 71.870 | 16.991 | 1.00 | 37.04 | C |
| ATOM | 1903 | O | PRO | A | 462 | 11.146 | 72.953 | 16.937 | 1.00 | 37.47 | O |
| ATOM | 1904 | CB | PRO | A | 462 | 8.068 | 72.191 | 16.775 | 1.00 | 36.39 | C |
| ATOM | 1905 | CG | PRO | A | 462 | 7.080 | 72.806 | 17.737 | 1.00 | 37.83 | C |
| ATOM | 1906 | CD | PRO | A | 462 | 7.973 | 73.588 | 18.664 | 1.00 | 37.41 | C |
| ATOM | 1907 | N | VAL | A | 463 | 11.046 | 70.761 | 16.457 | 1.00 | 35.68 | N |
| ATOM | 1908 | CA | VAL | A | 463 | 12.309 | 70.769 | 15.743 | 1.00 | 37.59 | C |
| ATOM | 1909 | C | VAL | A | 463 | 12.099 | 69.974 | 14.467 | 1.00 | 38.39 | C |
| ATOM | 1910 | O | VAL | A | 463 | 11.921 | 68.758 | 14.507 | 1.00 | 40.64 | O |
| ATOM | 1911 | CB | VAL | A | 463 | 13.436 | 70.109 | 16.567 | 1.00 | 38.12 | C |
| ATOM | 1912 | CG1 | VAL | A | 463 | 14.748 | 70.162 | 15.796 | 1.00 | 37.89 | C |
| ATOM | 1913 | CG2 | VAL | A | 463 | 13.581 | 70.810 | 17.900 | 1.00 | 37.66 | C |
| ATOM | 1914 | N | LEU | A | 464 | 12.104 | 70.661 | 13.332 | 1.00 | 38.58 | N |
| ATOM | 1915 | CA | LEU | A | 464 | 11.911 | 69.985 | 12.061 | 1.00 | 39.44 | C |
| ATOM | 1916 | C | LEU | A | 464 | 13.237 | 69.675 | 11.414 | 1.00 | 39.39 | C |
| ATOM | 1917 | O | LEU | A | 464 | 14.082 | 70.545 | 11.269 | 1.00 | 40.89 | O |
| ATOM | 1918 | CB | LEU | A | 464 | 11.063 | 70.841 | 11.123 | 1.00 | 39.47 | C |
| ATOM | 1919 | CG | LEU | A | 464 | 9.720 | 71.176 | 11.765 | 1.00 | 41.40 | C |
| ATOM | 1920 | CD1 | LEU | A | 464 | 9.739 | 72.614 | 12.246 | 1.00 | 40.22 | C |
| ATOM | 1921 | CD2 | LEU | A | 464 | 8.603 | 70.942 | 10.772 | 1.00 | 43.14 | C |
| ATOM | 1922 | N | VAL | A | 465 | 13.417 | 68.417 | 11.040 | 1.00 | 39.92 | N |
| ATOM | 1923 | CA | VAL | A | 465 | 14.638 | 67.980 | 10.398 | 1.00 | 39.01 | C |
| ATOM | 1924 | C | VAL | A | 465 | 14.241 | 67.361 | 9.064 | 1.00 | 41.20 | C |
| ATOM | 1925 | O | VAL | A | 465 | 13.465 | 66.406 | 9.018 | 1.00 | 42.40 | O |
| ATOM | 1926 | CB | VAL | A | 465 | 15.370 | 66.935 | 11.278 | 1.00 | 39.18 | C |
| ATOM | 1927 | CG1 | VAL | A | 465 | 16.633 | 66.440 | 10.578 | 1.00 | 38.35 | C |
| ATOM | 1928 | CG2 | VAL | A | 465 | 15.715 | 67.552 | 12.630 | 1.00 | 34.80 | C |
| ATOM | 1929 | N | PHE | A | 466 | 14.759 | 67.917 | 7.974 | 1.00 | 41.85 | N |
| ATOM | 1930 | CA | PHE | A | 466 | 14.440 | 67.407 | 6.646 | 1.00 | 41.45 | C |
| ATOM | 1931 | C | PHE | A | 466 | 15.652 | 67.522 | 5.728 | 1.00 | 42.14 | C |
| ATOM | 1932 | O | PHE | A | 466 | 16.573 | 68.287 | 6.084 | 1.00 | 43.81 | O |
| ATOM | 1933 | CB | PHE | A | 466 | 13.268 | 68.196 | 6.054 | 1.00 | 40.09 | C |
| ATOM | 1934 | CG | PHE | A | 466 | 13.477 | 69.682 | 6.068 | 1.00 | 37.93 | C |
| ATOM | 1935 | CD1 | PHE | A | 466 | 13.268 | 70.414 | 7.229 | 1.00 | 39.56 | C |
| ATOM | 1936 | CD2 | PHE | A | 466 | 13.930 | 70.342 | 4.937 | 1.00 | 38.83 | C |
| ATOM | 1937 | CE1 | PHE | A | 466 | 13.510 | 71.782 | 7.263 | 1.00 | 41.25 | C |
| ATOM | 1938 | CE2 | PHE | A | 466 | 14.177 | 71.713 | 4.961 | 1.00 | 41.28 | C |
| ATOM | 1939 | CZ | PHE | A | 466 | 13.967 | 72.432 | 6.127 | 1.00 | 40.51 | C |
| ATOM | 1940 | OXT | PHE | A | 466 | 15.659 | 66.867 | 4.663 | 1.00 | 42.58 | O |
| TER | 1941 | | PHE | A | 466 | | | | | | |
| HETATM | 1942 | ZN | ZN | | 467 | 27.755 | 63.139 | 18.832 | 1.00 | 103.49 | ZN |
| HETATM | 1943 | S | SO4 | | 763 | −36.350 | 62.961 | 11.333 | 1.00 | 69.23 | S |
| HETATM | 1944 | O1 | SO4 | | 763 | −35.152 | 62.880 | 10.506 | 1.00 | 68.42 | O |
| HETATM | 1945 | O4 | SO4 | | 763 | −37.529 | 63.326 | 10.241 | 1.00 | 70.45 | O |
| HETATM | 1946 | O | HOH | | 468 | −33.656 | 53.189 | 14.378 | 1.00 | 40.93 | O |
| HETATM | 1947 | O | HOH | | 469 | −28.676 | 52.376 | 11.972 | 1.00 | 55.65 | O |
| HETATM | 1948 | O | HOH | | 470 | −24.406 | 50.714 | 11.227 | 1.00 | 58.54 | O |
| HETATM | 1949 | O | HOH | | 471 | −19.811 | 54.095 | 7.787 | 1.00 | 54.24 | O |
| HETATM | 1950 | O | HOH | | 472 | −17.751 | 54.957 | 5.586 | 1.00 | 65.38 | O |
| HETATM | 1951 | O | HOH | | 473 | −18.441 | 60.105 | 6.342 | 1.00 | 49.29 | O |
| HETATM | 1952 | O | HOH | | 474 | −18.270 | 62.456 | 5.110 | 1.00 | 38.56 | O |
| HETATM | 1953 | O | HOH | | 475 | −17.176 | 65.688 | 7.072 | 1.00 | 48.35 | O |
| HETATM | 1954 | O | HOH | | 476 | −16.465 | 67.649 | 5.086 | 1.00 | 57.48 | O |
| HETATM | 1955 | O | HOH | | 477 | −13.160 | 66.590 | 5.458 | 1.00 | 37.00 | O |
| HETATM | 1956 | O | HOH | | 478 | −13.667 | 64.814 | 7.045 | 1.00 | 38.73 | O |
| HETATM | 1957 | O | HOH | | 479 | −13.608 | 62.609 | 7.888 | 1.00 | 42.07 | O |
| HETATM | 1958 | O | HOH | | 480 | −14.195 | 60.804 | 6.138 | 1.00 | 40.34 | O |
| HETATM | 1959 | O | HOH | | 481 | −13.911 | 60.623 | 3.697 | 1.00 | 40.20 | O |
| HETATM | 1960 | O | HOH | | 482 | −14.195 | 62.848 | 2.351 | 1.00 | 51.75 | O |
| HETATM | 1961 | O | HOH | | 483 | −11.493 | 64.899 | 2.292 | 1.00 | 42.08 | O |
| HETATM | 1962 | O | HOH | | 484 | −8.908 | 64.882 | 3.039 | 1.00 | 55.31 | O |
| HETATM | 1963 | O | HOH | | 485 | −9.115 | 67.857 | 3.855 | 1.00 | 74.27 | O |
| HETATM | 1964 | O | HOH | | 486 | −9.348 | 69.769 | 6.165 | 1.00 | 46.03 | O |
| HETATM | 1965 | O | HOH | | 487 | −6.951 | 69.043 | 8.431 | 1.00 | 64.21 | O |
| HETATM | 1966 | O | HOH | | 488 | −6.109 | 69.372 | 5.875 | 1.00 | 71.31 | O |
| HETATM | 1967 | O | HOH | | 489 | −3.922 | 68.672 | 7.493 | 1.00 | 53.17 | O |
| HETATM | 1968 | O | HOH | | 490 | −3.773 | 71.131 | 6.782 | 1.00 | 68.17 | O |
| HETATM | 1969 | O | HOH | | 491 | −1.335 | 72.291 | 4.839 | 1.00 | 58.79 | O |
| HETATM | 1970 | O | HOH | | 492 | 0.899 | 69.681 | 7.567 | 1.00 | 39.91 | O |
| HETATM | 1971 | O | HOH | | 493 | −0.814 | 67.970 | 6.535 | 1.00 | 59.58 | O |
| HETATM | 1972 | O | HOH | | 494 | −0.351 | 66.722 | 8.774 | 1.00 | 47.11 | O |
| HETATM | 1973 | O | HOH | | 495 | −3.087 | 66.709 | 9.754 | 1.00 | 50.46 | O |
| HETATM | 1974 | O | HOH | | 496 | −3.259 | 66.082 | 7.133 | 1.00 | 52.39 | O |
| HETATM | 1975 | O | HOH | | 497 | −4.775 | 63.739 | 4.849 | 1.00 | 55.12 | O |
| HETATM | 1976 | O | HOH | | 498 | −1.400 | 65.259 | 3.961 | 1.00 | 65.80 | O |
| HETATM | 1977 | O | HOH | | 499 | −1.393 | 62.848 | 4.149 | 1.00 | 54.88 | O |
| HETATM | 1978 | O | HOH | | 500 | 1.816 | 61.509 | 5.150 | 1.00 | 42.75 | O |
| HETATM | 1979 | O | HOH | | 501 | 3.711 | 59.301 | 4.739 | 1.00 | 67.48 | O |
| HETATM | 1980 | O | HOH | | 502 | 3.271 | 56.327 | 5.249 | 1.00 | 54.81 | O |
| HETATM | 1981 | O | HOH | | 503 | 2.689 | 57.125 | 8.955 | 1.00 | 72.05 | O |
| HETATM | 1982 | O | HOH | | 504 | 6.666 | 59.157 | 7.169 | 1.00 | 46.90 | O |
| HETATM | 1983 | O | HOH | | 505 | 8.593 | 59.118 | 5.012 | 1.00 | 60.35 | O |
| HETATM | 1984 | O | HOH | | 506 | 6.538 | 57.372 | 4.477 | 1.00 | 71.45 | O |
| HETATM | 1985 | O | HOH | | 507 | 7.170 | 61.314 | 3.411 | 1.00 | 68.63 | O |
| HETATM | 1986 | O | HOH | | 508 | 12.464 | 61.862 | 3.959 | 1.00 | 53.71 | O |
| HETATM | 1987 | O | HOH | | 509 | 13.277 | 64.868 | 4.314 | 1.00 | 48.69 | O |
| HETATM | 1988 | O | HOH | | 510 | 16.603 | 62.695 | 4.827 | 1.00 | 76.94 | O |
| HETATM | 1989 | O | HOH | | 511 | 17.447 | 65.218 | 3.726 | 1.00 | 59.34 | O |
| HETATM | 1990 | O | HOH | | 512 | 16.697 | 67.035 | 2.393 | 1.00 | 60.28 | O |
| HETATM | 1991 | O | HOH | | 513 | 16.598 | 67.172 | −0.263 | 1.00 | 62.61 | O |
| HETATM | 1992 | O | HOH | | 514 | 19.404 | 66.531 | 0.015 | 1.00 | 75.76 | O |
| HETATM | 1993 | O | HOH | | 515 | 20.876 | 64.256 | −2.559 | 1.00 | 57.85 | O |
| HETATM | 1994 | O | HOH | | 516 | 20.969 | 61.824 | 0.979 | 1.00 | 63.03 | O |
| HETATM | 1995 | O | HOH | | 517 | 22.674 | 65.354 | 2.236 | 1.00 | 46.47 | O |
| HETATM | 1996 | O | HOH | | 518 | 21.754 | 63.114 | 5.970 | 1.00 | 56.75 | O |
| HETATM | 1997 | O | HOH | | 519 | 22.600 | 63.167 | 8.639 | 1.00 | 43.42 | O |
| HETATM | 1998 | O | HOH | | 520 | 19.016 | 61.650 | 7.151 | 1.00 | 63.60 | O |
| HETATM | 1999 | O | HOH | | 521 | 15.782 | 60.297 | 7.747 | 1.00 | 51.49 | O |
| HETATM | 2000 | O | HOH | | 522 | 17.768 | 57.885 | 8.380 | 1.00 | 67.65 | O |
| HETATM | 2001 | O | HOH | | 523 | 18.957 | 54.855 | 8.355 | 1.00 | 61.94 | O |
| HETATM | 2002 | O | HOH | | 524 | 17.374 | 56.767 | 13.097 | 1.00 | 70.61 | O |
| HETATM | 2003 | O | HOH | | 525 | 14.592 | 57.320 | 13.296 | 1.00 | 53.68 | O |
| HETATM | 2004 | O | HOH | | 526 | 13.828 | 58.758 | 15.416 | 1.00 | 48.40 | O |
| HETATM | 2005 | O | HOH | | 527 | 11.652 | 59.044 | 13.867 | 1.00 | 45.62 | O |
| HETATM | 2006 | O | HOH | | 528 | 10.992 | 60.601 | 11.825 | 1.00 | 44.54 | O |
| HETATM | 2007 | O | HOH | | 529 | 13.010 | 59.703 | 9.219 | 1.00 | 48.46 | O |
| HETATM | 2008 | O | HOH | | 530 | 8.014 | 59.337 | 9.796 | 1.00 | 47.91 | O |
| HETATM | 2009 | O | HOH | | 531 | 6.830 | 57.231 | 14.038 | 1.00 | 59.33 | O |
| HETATM | 2010 | O | HOH | | 532 | 4.142 | 57.919 | 14.892 | 1.00 | 38.25 | O |
| HETATM | 2011 | O | HOH | | 533 | 4.727 | 60.014 | 16.207 | 1.00 | 33.33 | O |
| HETATM | 2012 | O | HOH | | 534 | 2.652 | 57.543 | 18.689 | 1.00 | 65.55 | O |
| HETATM | 2013 | O | HOH | | 535 | −0.455 | 56.669 | 18.701 | 1.00 | 73.68 | O |
| HETATM | 2014 | O | HOH | | 536 | −2.994 | 56.678 | 21.148 | 1.00 | 48.98 | O |
| HETATM | 2015 | O | HOH | | 537 | −5.152 | 55.158 | 20.274 | 1.00 | 63.11 | O |
| HETATM | 2016 | O | HOH | | 538 | −5.207 | 54.811 | 18.011 | 1.00 | 66.37 | O |
| HETATM | 2017 | O | HOH | | 539 | −5.733 | 55.727 | 15.667 | 1.00 | 45.43 | O |
| HETATM | 2018 | O | HOH | | 540 | −6.841 | 58.216 | 15.974 | 1.00 | 38.56 | O |
| HETATM | 2019 | O | HOH | | 541 | −9.563 | 58.137 | 15.321 | 1.00 | 37.63 | O |
| HETATM | 2020 | O | HOH | | 542 | −7.625 | 55.580 | 19.338 | 1.00 | 48.91 | O |
| HETATM | 2021 | O | HOH | | 543 | −9.882 | 54.690 | 21.633 | 1.00 | 45.68 | O |
| HETATM | 2022 | O | HOH | | 544 | −10.390 | 56.442 | 23.562 | 1.00 | 42.14 | O |
| HETATM | 2023 | O | HOH | | 545 | −9.170 | 59.832 | 26.205 | 1.00 | 43.92 | O |
| HETATM | 2024 | O | HOH | | 546 | −6.817 | 60.744 | 26.878 | 1.00 | 59.12 | O |
| HETATM | 2025 | O | HOH | | 547 | −6.456 | 63.453 | 26.007 | 1.00 | 36.99 | O |
| HETATM | 2026 | O | HOH | | 548 | −1.340 | 62.845 | 26.011 | 1.00 | 33.90 | O |
| HETATM | 2027 | O | HOH | | 549 | 1.549 | 62.082 | 25.928 | 1.00 | 58.27 | O |
| HETATM | 2028 | O | HOH | | 550 | 3.264 | 64.224 | 25.861 | 1.00 | 64.09 | O |
| HETATM | 2029 | O | HOH | | 551 | 7.423 | 65.057 | 26.391 | 1.00 | 46.09 | O |
| HETATM | 2030 | O | HOH | | 552 | 8.714 | 66.752 | 27.762 | 1.00 | 49.98 | O |
| HETATM | 2031 | O | HOH | | 553 | 6.435 | 69.503 | 26.053 | 1.00 | 50.03 | O |
| HETATM | 2032 | O | HOH | | 554 | 6.198 | 71.900 | 27.690 | 1.00 | 62.59 | O |
| HETATM | 2033 | O | HOH | | 555 | 5.077 | 72.024 | 22.112 | 1.00 | 40.40 | O |
| HETATM | 2034 | O | HOH | | 556 | 1.071 | 73.567 | 23.198 | 1.00 | 56.49 | O |
| HETATM | 2035 | O | HOH | | 557 | −1.521 | 74.718 | 22.532 | 1.00 | 52.73 | O |
| HETATM | 2036 | O | HOH | | 558 | −4.343 | 75.122 | 23.356 | 1.00 | 41.75 | O |
| HETATM | 2037 | O | HOH | | 559 | −3.410 | 78.399 | 20.262 | 1.00 | 59.45 | O |
| HETATM | 2038 | O | HOH | | 560 | 13.630 | 69.081 | 1.249 | 1.00 | 32.54 | O |
| HETATM | 2039 | O | HOH | | 561 | −11.103 | 74.018 | 19.288 | 1.00 | 46.09 | O |
| HETATM | 2040 | O | HOH | | 562 | −12.223 | 76.586 | 18.142 | 1.00 | 46.73 | O |
| HETATM | 2041 | O | HOH | | 563 | −14.816 | 77.973 | 19.117 | 1.00 | 70.51 | O |
| HETATM | 2042 | O | HOH | | 564 | −15.828 | 78.048 | 15.219 | 1.00 | 64.29 | O |
| HETATM | 2043 | O | HOH | | 565 | −18.095 | 77.980 | 12.220 | 1.00 | 71.15 | O |
| HETATM | 2044 | O | HOH | | 566 | −21.615 | 77.612 | 12.690 | 1.00 | 55.98 | O |
| HETATM | 2045 | O | HOH | | 567 | −23.598 | 78.884 | 14.601 | 1.00 | 52.22 | O |
| HETATM | 2046 | O | HOH | | 568 | −20.281 | 76.888 | 16.968 | 1.00 | 36.27 | O |
| HETATM | 2047 | O | HOH | | 569 | −20.516 | 75.066 | 14.212 | 1.00 | 48.90 | O |
| HETATM | 2048 | O | HOH | | 570 | −15.954 | 75.739 | 8.629 | 1.00 | 51.32 | O |
| HETATM | 2049 | O | HOH | | 571 | −17.069 | 72.902 | 5.821 | 1.00 | 55.96 | O |
| HETATM | 2050 | O | HOH | | 572 | −15.015 | 69.081 | 2.370 | 1.00 | 74.26 | O |
| HETATM | 2051 | O | HOH | | 573 | −11.372 | 72.139 | 5.256 | 1.00 | 58.12 | O |
| HETATM | 2052 | O | HOH | | 574 | −9.890 | 76.072 | 7.248 | 1.00 | 53.73 | O |
| HETATM | 2053 | O | HOH | | 575 | −10.292 | 78.083 | 3.947 | 1.00 | 67.09 | O |
| HETATM | 2054 | O | HOH | | 576 | −3.566 | 76.534 | 6.469 | 1.00 | 55.48 | O |
| HETATM | 2055 | O | HOH | | 577 | −1.301 | 72.659 | 9.950 | 1.00 | 41.40 | O |
| HETATM | 2056 | O | HOH | | 578 | 1.373 | 72.105 | 11.069 | 1.00 | 35.50 | O |
| HETATM | 2057 | O | HOH | | 579 | 0.994 | 69.015 | 10.395 | 1.00 | 26.14 | O |
| HETATM | 2058 | O | HOH | | 580 | 3.870 | 65.190 | 11.227 | 1.00 | 31.68 | O |
| HETATM | 2059 | O | HOH | | 581 | 4.890 | 64.422 | 13.387 | 1.00 | 36.51 | O |
| HETATM | 2060 | O | HOH | | 582 | 9.112 | 59.124 | 15.061 | 1.00 | 63.70 | O |
| HETATM | 2061 | O | HOH | | 583 | 12.450 | 58.392 | 18.276 | 1.00 | 32.08 | O |
| HETATM | 2062 | O | HOH | | 584 | 11.611 | 55.585 | 17.941 | 1.00 | 58.81 | O |
| HETATM | 2063 | O | HOH | | 585 | 6.372 | 60.468 | 20.316 | 1.00 | 40.35 | O |
| HETATM | 2064 | O | HOH | | 586 | 4.421 | 64.003 | 20.808 | 1.00 | 32.35 | O |
| HETATM | 2065 | O | HOH | | 587 | 4.896 | 63.058 | 23.239 | 1.00 | 49.79 | O |
| HETATM | 2066 | O | HOH | | 588 | 0.437 | 60.417 | 19.259 | 1.00 | 39.50 | O |
| HETATM | 2067 | O | HOH | | 589 | −2.628 | 55.833 | 15.176 | 1.00 | 56.97 | O |
| HETATM | 2068 | O | HOH | | 590 | −2.941 | 53.469 | 13.029 | 1.00 | 58.36 | O |
| HETATM | 2069 | O | HOH | | 591 | −4.209 | 56.921 | 12.259 | 1.00 | 37.80 | O |
| HETATM | 2070 | O | HOH | | 592 | −3.056 | 57.950 | 8.545 | 1.00 | 40.74 | O |
| HETATM | 2071 | O | HOH | | 593 | −2.447 | 45.966 | 13.363 | 1.00 | 72.71 | O |
| HETATM | 2072 | O | HOH | | 594 | −13.608 | 51.340 | 11.454 | 1.00 | 59.73 | O |
| HETATM | 2073 | O | HOH | | 595 | −14.461 | 52.497 | 13.712 | 1.00 | 40.43 | O |
| HETATM | 2074 | O | HOH | | 596 | −17.361 | 51.770 | 13.271 | 1.00 | 50.00 | O |
| HETATM | 2075 | O | HOH | | 597 | −17.871 | 51.109 | 10.590 | 1.00 | 64.38 | O |
| HETATM | 2076 | O | HOH | | 598 | −14.875 | 49.048 | 17.257 | 1.00 | 62.30 | O |
| HETATM | 2077 | O | HOH | | 599 | −15.856 | 49.579 | 19.958 | 1.00 | 85.79 | O |
| HETATM | 2078 | O | HOH | | 600 | −13.318 | 49.462 | 20.138 | 1.00 | 64.03 | O |
| HETATM | 2079 | O | HOH | | 601 | −18.662 | 51.511 | 18.197 | 1.00 | 61.02 | O |
| HETATM | 2080 | O | HOH | | 602 | −20.289 | 55.723 | 22.461 | 1.00 | 62.40 | O |
| HETATM | 2081 | O | HOH | | 603 | −21.842 | 57.470 | 23.576 | 1.00 | 69.23 | O |
| HETATM | 2082 | O | HOH | | 604 | −22.004 | 59.982 | 24.148 | 1.00 | 49.28 | O |
| HETATM | 2083 | O | HOH | | 605 | −22.020 | 61.081 | 26.706 | 1.00 | 54.17 | O |
| HETATM | 2084 | O | HOH | | 606 | −24.860 | 61.581 | 27.783 | 1.00 | 70.71 | O |
| HETATM | 2085 | O | HOH | | 607 | −26.083 | 62.576 | 24.881 | 1.00 | 56.16 | O |
| HETATM | 2086 | O | HOH | | 608 | −26.276 | 65.042 | 24.393 | 1.00 | 51.72 | O |
| HETATM | 2087 | O | HOH | | 609 | −24.942 | 66.995 | 26.795 | 1.00 | 70.26 | O |
| HETATM | 2088 | O | HOH | | 610 | −24.195 | 69.464 | 26.160 | 1.00 | 70.50 | O |
| HETATM | 2089 | O | HOH | | 611 | −23.483 | 71.478 | 23.444 | 1.00 | 55.94 | O |
| HETATM | 2090 | O | HOH | | 612 | −24.247 | 70.721 | 20.903 | 1.00 | 43.20 | O |
| HETATM | 2091 | O | HOH | | 613 | −22.181 | 74.612 | 21.806 | 1.00 | 56.57 | O |
| HETATM | 2092 | O | HOH | | 614 | −24.542 | 77.207 | 20.091 | 1.00 | 44.50 | O |
| HETATM | 2093 | O | HOH | | 615 | 18.387 | 82.576 | 3.202 | 1.00 | 66.83 | O |
| HETATM | 2094 | O | HOH | | 616 | −21.584 | 68.621 | 24.076 | 1.00 | 50.88 | O |
| HETATM | 2095 | O | HOH | | 617 | −19.490 | 66.383 | 23.995 | 1.00 | 40.44 | O |
| HETATM | 2096 | O | HOH | | 618 | −17.964 | 65.105 | 26.024 | 1.00 | 39.91 | O |
| HETATM | 2097 | O | HOH | | 619 | −15.579 | 65.138 | 26.803 | 1.00 | 48.08 | O |
| HETATM | 2098 | O | HOH | | 620 | −14.197 | 67.134 | 24.980 | 1.00 | 24.89 | O |
| HETATM | 2099 | O | HOH | | 621 | −15.590 | 68.492 | 26.282 | 1.00 | 54.87 | O |
| HETATM | 2100 | O | HOH | | 622 | −18.239 | 69.600 | 21.480 | 1.00 | 39.73 | O |
| HETATM | 2101 | O | HOH | | 623 | −17.654 | 72.539 | 21.749 | 1.00 | 67.53 | O |
| HETATM | 2102 | O | HOH | | 624 | −18.469 | 62.582 | 26.532 | 1.00 | 48.07 | O |
| HETATM | 2103 | O | HOH | | 625 | −15.776 | 61.282 | 25.964 | 1.00 | 45.14 | O |
| HETATM | 2104 | O | HOH | | 626 | −14.096 | 62.640 | 27.828 | 1.00 | 58.72 | O |
| HETATM | 2105 | O | HOH | | 627 | −16.022 | 64.187 | 29.567 | 1.00 | 82.76 | O |
| HETATM | 2106 | O | HOH | | 628 | −11.325 | 60.544 | 27.641 | 1.00 | 56.51 | O |
| HETATM | 2107 | O | HOH | | 629 | −13.233 | 58.623 | 28.272 | 1.00 | 55.61 | O |
| HETATM | 2108 | O | HOH | | 630 | −15.747 | 58.734 | 26.927 | 1.00 | 58.22 | O |
| HETATM | 2109 | O | HOH | | 631 | −17.862 | 57.251 | 29.668 | 1.00 | 65.41 | O |
| HETATM | 2110 | O | HOH | | 632 | −21.717 | 62.831 | 29.564 | 1.00 | 85.47 | O |
| HETATM | 2111 | O | HOH | | 633 | −26.295 | 58.260 | 27.618 | 1.00 | 60.97 | O |
| HETATM | 2112 | O | HOH | | 634 | −25.445 | 57.414 | 23.608 | 1.00 | 72.88 | O |
| HETATM | 2113 | O | HOH | | 635 | −30.175 | 61.383 | 23.145 | 1.00 | 65.26 | O |
| HETATM | 2114 | O | HOH | | 636 | −29.432 | 64.628 | 24.998 | 1.00 | 64.48 | O |
| HETATM | 2115 | O | HOH | | 637 | −29.054 | 70.191 | 24.812 | 1.00 | 67.06 | O |
| HETATM | 2116 | O | HOH | | 638 | −32.436 | 68.973 | 21.237 | 1.00 | 69.37 | O |
| HETATM | 2117 | O | HOH | | 639 | −30.181 | 72.774 | 15.576 | 1.00 | 53.02 | O |
| HETATM | 2118 | O | HOH | | 640 | −29.782 | 74.395 | 8.598 | 1.00 | 60.73 | O |
| HETATM | 2119 | O | HOH | | 641 | −28.470 | 76.921 | 8.282 | 1.00 | 75.84 | O |
| HETATM | 2120 | O | HOH | | 642 | −27.190 | 72.678 | 8.724 | 1.00 | 72.31 | O |
| HETATM | 2121 | O | HOH | | 643 | −27.282 | 71.733 | 5.689 | 1.00 | 52.88 | O |
| HETATM | 2122 | O | HOH | | 644 | −24.128 | 70.475 | 5.794 | 1.00 | 58.51 | O |
| HETATM | 2123 | O | HOH | | 645 | −24.940 | 67.423 | 3.516 | 1.00 | 60.33 | O |
| HETATM | 2124 | O | HOH | | 646 | −22.480 | 69.300 | 2.638 | 1.00 | 67.37 | O |
| HETATM | 2125 | O | HOH | | 647 | −21.152 | 63.968 | 2.625 | 1.00 | 72.94 | O |
| HETATM | 2126 | O | HOH | | 648 | −23.445 | 61.936 | 2.809 | 1.00 | 69.21 | O |
| HETATM | 2127 | O | HOH | | 649 | −41.302 | 55.452 | 22.313 | 1.00 | 50.18 | O |
| HETATM | 2128 | O | HOH | | 650 | −43.630 | 58.640 | 20.148 | 1.00 | 74.74 | O |
| HETATM | 2129 | O | HOH | | 651 | −30.816 | 69.577 | 6.910 | 1.00 | 69.37 | O |
| HETATM | 2130 | O | HOH | | 652 | −32.068 | 64.171 | 8.936 | 1.00 | 39.83 | O |
| HETATM | 2131 | O | HOH | | 653 | −30.430 | 57.760 | 9.618 | 1.00 | 39.04 | O |
| HETATM | 2132 | O | HOH | | 654 | −28.347 | 53.609 | 17.321 | 1.00 | 54.10 | O |
| HETATM | 2133 | O | HOH | | 655 | −26.649 | 53.470 | 19.506 | 1.00 | 68.04 | O |
| HETATM | 2134 | O | HOH | | 656 | −36.228 | 50.752 | 18.389 | 1.00 | 63.46 | O |
| HETATM | 2135 | O | HOH | | 657 | −25.808 | 61.624 | 1.134 | 1.00 | 58.21 | O |
| HETATM | 2136 | O | HOH | | 658 | −39.296 | 53.448 | 25.834 | 1.00 | 74.80 | O |
| HETATM | 2137 | O | HOH | | 659 | −43.811 | 56.417 | 25.826 | 1.00 | 69.74 | O |
| HETATM | 2138 | O | HOH | | 660 | −44.827 | 66.932 | 14.145 | 1.00 | 63.92 | O |
| HETATM | 2139 | O | HOH | | 661 | −42.055 | 74.741 | 20.921 | 1.00 | 63.79 | O |
| HETATM | 2140 | O | HOH | | 662 | −38.677 | 76.618 | 10.842 | 1.00 | 58.76 | O |
| HETATM | 2141 | O | HOH | | 663 | −19.746 | 65.538 | −1.810 | 1.00 | 60.23 | O |
| HETATM | 2142 | O | HOH | | 664 | 13.544 | 53.997 | 22.981 | 1.00 | 61.06 | O |
| HETATM | 2143 | O | HOH | | 665 | −21.512 | 58.500 | 7.701 | 1.00 | 66.41 | O |
| HETATM | 2144 | O | HOH | | 666 | −12.897 | 66.296 | 12.599 | 1.00 | 102.12 | O |
| HETATM | 2145 | O | HOH | | 667 | −7.246 | 68.905 | 20.411 | 1.00 | 33.79 | O |
| HETATM | 2146 | O | HOH | | 668 | −7.442 | 73.387 | 15.028 | 1.00 | 40.46 | O |
| HETATM | 2147 | O | HOH | | 669 | −9.267 | 78.013 | 14.462 | 1.00 | 65.70 | O |
| HETATM | 2148 | O | HOH | | 670 | −11.604 | 77.610 | 15.410 | 1.00 | 59.10 | O |
| HETATM | 2149 | O | HOH | | 671 | −18.617 | 83.152 | 14.210 | 1.00 | 68.87 | O |
| HETATM | 2150 | O | HOH | | 672 | 3.704 | 90.473 | 26.584 | 1.00 | 67.77 | O |
| HETATM | 2151 | O | HOH | | 673 | 3.430 | 88.060 | 16.800 | 1.00 | 62.32 | O |
| HETATM | 2152 | O | HOH | | 674 | 3.446 | 86.911 | 11.669 | 1.00 | 62.75 | O |
| HETATM | 2153 | O | HOH | | 675 | 1.369 | 83.892 | 13.951 | 1.00 | 67.39 | O |
| HETATM | 2154 | O | HOH | | 676 | 1.312 | 81.464 | 10.173 | 1.00 | 67.07 | O |
| HETATM | 2155 | O | HOH | | 677 | 7.455 | 84.196 | 6.958 | 1.00 | 71.13 | O |
| HETATM | 2156 | O | HOH | | 678 | 9.086 | 83.246 | 8.770 | 1.00 | 72.81 | O |
| HETATM | 2157 | O | HOH | | 679 | 8.574 | 81.425 | 10.695 | 1.00 | 52.44 | O |
| HETATM | 2158 | O | HOH | | 680 | 10.287 | 81.190 | 6.947 | 1.00 | 49.87 | O |
| HETATM | 2159 | O | HOH | | 681 | 7.745 | 80.478 | 7.047 | 1.00 | 63.06 | O |
| HETATM | 2160 | O | HOH | | 682 | 11.136 | 78.184 | 8.288 | 1.00 | 43.48 | O |
| HETATM | 2161 | O | HOH | | 683 | 5.050 | 77.863 | 9.976 | 1.00 | 60.26 | O |
| HETATM | 2162 | O | HOH | | 684 | 5.256 | 75.725 | 8.406 | 1.00 | 40.48 | O |
| HETATM | 2163 | O | HOH | | 685 | 4.166 | 73.318 | 9.138 | 1.00 | 52.81 | O |
| HETATM | 2164 | O | HOH | | 686 | 5.248 | 76.001 | 16.876 | 1.00 | 42.43 | O |
| HETATM | 2165 | O | HOH | | 687 | 3.763 | 75.662 | 19.193 | 1.00 | 44.46 | O |
| HETATM | 2166 | O | HOH | | 688 | 2.645 | 73.025 | 18.461 | 1.00 | 38.56 | O |
| HETATM | 2167 | O | HOH | | 689 | −1.615 | 70.578 | 16.565 | 1.00 | 36.76 | O |
| HETATM | 2168 | O | HOH | | 690 | 3.057 | 66.251 | 2.509 | 1.00 | 46.50 | O |
| HETATM | 2169 | O | HOH | | 691 | −3.362 | 73.790 | 6.547 | 1.00 | 71.52 | O |
| HETATM | 2170 | O | HOH | | 692 | −1.709 | 54.980 | 23.259 | 1.00 | 52.42 | O |
| HETATM | 2171 | O | HOH | | 693 | 11.677 | 54.178 | 10.005 | 1.00 | 70.54 | O |
| HETATM | 2172 | O | HOH | | 694 | 21.521 | 57.119 | 12.926 | 1.00 | 61.28 | O |
| HETATM | 2173 | O | HOH | | 695 | 22.623 | 59.226 | 18.427 | 1.00 | 61.56 | O |
| HETATM | 2174 | O | HOH | | 696 | 22.935 | 60.139 | 22.338 | 1.00 | 56.73 | O |
| HETATM | 2175 | O | HOH | | 697 | 20.705 | 61.025 | 28.058 | 1.00 | 83.44 | O |
| HETATM | 2176 | O | HOH | | 698 | 21.639 | 62.920 | 29.646 | 1.00 | 62.23 | O |
| HETATM | 2177 | O | HOH | | 699 | 18.439 | 62.095 | 26.971 | 1.00 | 59.70 | O |
| HETATM | 2178 | O | HOH | | 700 | 16.982 | 63.304 | 28.939 | 1.00 | 69.50 | O |
| HETATM | 2179 | O | HOH | | 701 | 17.426 | 59.750 | 28.282 | 1.00 | 55.46 | O |
| HETATM | 2180 | O | HOH | | 702 | 17.178 | 66.943 | 33.610 | 1.00 | 73.30 | O |
| HETATM | 2181 | O | HOH | | 703 | 20.130 | 70.303 | 33.456 | 1.00 | 61.69 | O |
| HETATM | 2182 | O | HOH | | 704 | 17.093 | 72.668 | 32.161 | 1.00 | 81.18 | O |
| HETATM | 2183 | O | HOH | | 705 | −3.412 | 50.191 | 13.243 | 1.00 | 73.13 | O |
| HETATM | 2184 | O | HOH | | 706 | −0.271 | 53.405 | 12.494 | 1.00 | 64.17 | O |
| HETATM | 2185 | O | HOH | | 707 | −2.907 | 55.639 | 9.723 | 1.00 | 66.83 | O |
| HETATM | 2186 | O | HOH | | 708 | 11.992 | 75.784 | 29.125 | 1.00 | 53.51 | O |
| HETATM | 2187 | O | HOH | | 709 | 14.483 | 77.919 | 28.487 | 1.00 | 62.22 | O |
| HETATM | 2188 | O | HOH | | 710 | 12.161 | 79.052 | 28.800 | 1.00 | 68.58 | O |
| HETATM | 2189 | O | HOH | | 711 | 13.447 | 80.922 | 27.112 | 1.00 | 65.37 | O |
| HETATM | 2190 | O | HOH | | 712 | 11.263 | 78.005 | 22.378 | 1.00 | 44.18 | O |
| HETATM | 2191 | O | HOH | | 713 | 14.353 | 84.695 | 21.464 | 1.00 | 60.35 | O |
| HETATM | 2192 | O | HOH | | 714 | 22.565 | 84.204 | 14.853 | 1.00 | 47.13 | O |
| HETATM | 2193 | O | HOH | | 715 | 25.074 | 83.351 | 16.453 | 1.00 | 72.26 | O |
| HETATM | 2194 | O | HOH | | 716 | 26.028 | 81.816 | 14.278 | 1.00 | 61.62 | O |
| HETATM | 2195 | O | HOH | | 717 | 27.766 | 85.002 | 15.241 | 1.00 | 71.89 | O |
| HETATM | 2196 | O | HOH | | 718 | 25.267 | 82.443 | 10.235 | 1.00 | 65.31 | O |
| HETATM | 2197 | O | HOH | | 719 | 27.391 | 79.686 | 11.631 | 1.00 | 52.47 | O |
| HETATM | 2198 | O | HOH | | 720 | 23.176 | 84.456 | 7.901 | 1.00 | 79.57 | O |
| HETATM | 2199 | O | HOH | | 721 | 20.089 | 82.285 | 6.248 | 1.00 | 66.45 | O |
| HETATM | 2200 | O | HOH | | 722 | 18.298 | 84.042 | 7.724 | 1.00 | 61.48 | O |
| HETATM | 2201 | O | HOH | | 723 | 18.821 | 86.960 | 10.709 | 1.00 | 61.87 | O |
| HETATM | 2202 | O | HOH | | 724 | 13.421 | 86.582 | 10.509 | 1.00 | 66.32 | O |
| HETATM | 2203 | O | HOH | | 725 | 10.729 | 93.123 | 6.237 | 1.00 | 59.03 | O |
| HETATM | 2204 | O | HOH | | 726 | 13.043 | 82.317 | 3.143 | 1.00 | 60.19 | O |
| HETATM | 2205 | O | HOH | | 727 | 15.892 | 82.962 | 2.541 | 1.00 | 58.66 | O |
| HETATM | 2206 | O | HOH | | 728 | 15.763 | 81.183 | 0.696 | 1.00 | 44.04 | O |
| HETATM | 2207 | O | HOH | | 729 | 11.793 | 79.433 | 1.119 | 1.00 | 59.65 | O |
| HETATM | 2208 | O | HOH | | 730 | 10.263 | 81.362 | 2.146 | 1.00 | 49.52 | O |
| HETATM | 2209 | O | HOH | | 731 | 16.597 | 79.171 | −2.168 | 1.00 | 54.99 | O |
| HETATM | 2210 | O | HOH | | 732 | 19.469 | 75.622 | −3.425 | 1.00 | 65.73 | O |
| HETATM | 2211 | O | HOH | | 733 | 18.236 | 74.536 | −0.875 | 1.00 | 54.83 | O |
| HETATM | 2212 | O | HOH | | 734 | 22.784 | 74.568 | −3.264 | 1.00 | 81.48 | O |
| HETATM | 2213 | O | HOH | | 735 | 23.154 | 77.551 | −2.312 | 1.00 | 73.50 | O |
| HETATM | 2214 | O | HOH | | 736 | 24.449 | 75.919 | 2.327 | 1.00 | 61.99 | O |
| HETATM | 2215 | O | HOH | | 737 | 21.685 | 78.594 | 4.389 | 1.00 | 62.89 | O |
| HETATM | 2216 | O | HOH | | 738 | 15.411 | 75.408 | 4.602 | 1.00 | 36.84 | O |
| HETATM | 2217 | O | HOH | | 739 | −8.699 | 76.433 | 21.810 | 1.00 | 39.31 | O |
| HETATM | 2218 | O | HOH | | 740 | 27.366 | 67.231 | −0.637 | 1.00 | 72.03 | O |
| HETATM | 2219 | O | HOH | | 741 | 31.549 | 73.635 | 1.024 | 1.00 | 76.00 | O |
| HETATM | 2220 | O | HOH | | 742 | 40.965 | 67.185 | 6.028 | 1.00 | 52.82 | O |
| HETATM | 2221 | O | HOH | | 743 | 36.191 | 59.524 | 9.724 | 1.00 | 74.34 | O |
| HETATM | 2222 | O | HOH | | 744 | 33.049 | 59.397 | 9.603 | 1.00 | 66.08 | O |
| HETATM | 2223 | O | HOH | | 745 | 30.384 | 59.620 | 14.421 | 1.00 | 54.94 | O |
| HETATM | 2224 | O | HOH | | 746 | 30.239 | 53.959 | 11.925 | 1.00 | 64.60 | O |
| HETATM | 2225 | O | HOH | | 747 | 33.994 | 50.098 | 10.459 | 1.00 | 61.90 | O |
| HETATM | 2226 | O | HOH | | 748 | 30.109 | 59.464 | 1.853 | 1.00 | 74.31 | O |
| HETATM | 2227 | O | HOH | | 749 | 39.718 | 67.881 | 16.059 | 1.00 | 69.40 | O |
| HETATM | 2228 | O | HOH | | 750 | 33.895 | 74.270 | 17.489 | 1.00 | 49.98 | O |
| HETATM | 2229 | O | HOH | | 751 | 31.807 | 78.282 | 19.062 | 1.00 | 61.18 | O |
| HETATM | 2230 | O | HOH | | 752 | 34.763 | 77.751 | 22.032 | 1.00 | 64.88 | O |
| HETATM | 2231 | O | HOH | | 753 | 30.927 | 71.309 | 23.558 | 1.00 | 58.13 | O |
| HETATM | 2232 | O | HOH | | 754 | 31.127 | 72.209 | 34.605 | 1.00 | 70.51 | O |
| HETATM | 2233 | O | HOH | | 755 | 26.727 | 75.342 | 34.607 | 1.00 | 75.15 | O |
| HETATM | 2234 | O | HOH | | 756 | 22.784 | 75.066 | 37.365 | 1.00 | 74.95 | O |
| HETATM | 2235 | O | HOH | | 757 | 21.196 | 79.013 | 29.923 | 1.00 | 58.45 | O |
| HETATM | 2236 | O | HOH | | 758 | 23.818 | 80.895 | 31.141 | 1.00 | 61.72 | O |
| HETATM | 2237 | O | HOH | | 759 | 25.137 | 82.966 | 25.564 | 1.00 | 56.13 | O |
| HETATM | 2238 | O | HOH | | 760 | 32.676 | 60.389 | 30.626 | 1.00 | 68.80 | O |
| HETATM | 2239 | O | HOH | | 761 | −35.004 | 60.164 | 24.083 | 1.00 | 54.66 | O |
| HETATM | 2240 | O | HOH | | 762 | −35.688 | 63.599 | 26.908 | 1.00 | 71.05 | O |

| | | | |
|---|---|---|---|
| CONECT | 1943 | 1944 | 1945 |
| CONECT | 1944 | 1943 | |
| CONECT | 1945 | 1943 | |

| | | | | | | | | | | | |
|---|---|---|---|---|---|---|---|---|---|---|---|
| MASTER | 332 | 0 | 2 | 6 | 18 | 0 | 0 | 6 2239 | 1 | 3 | 20 |
| END | | | | | | | | | | | |

### REFERENCES

Amrein et al., Proc. Natl. Acad. Sci. U.S.A. 92, 1048 (1995).
Anderson et al., Biochem. J. 373, 949 (2003).
Arbeloa, A., Hugonnet, J. E., Sentilhes, A. C., Josseaume, N., Dubost, L., Monsempes, C., Blanot, D., Brouard, J. P. & Arthur, M. (2004) J Biol Chem 279, 41546-56.
Arbeloa, A., Segal, H., Hugonnet, J. E., Josseaume, N., Dubost, L., Brouard, J. P., Gutmann, L., Mengin-Lecreuix, D. & Arthur, M. (2004b) J Bacteriol 186, 1221-8.
Beddell, 1985, Chem. Soc. Reviews, 279. Billot-Klein, D., Shlaes, D., Bryant, D., Bell, D., Legrand, R., Gutmann, L. & van Heijenoort, J. (1997) J Bacteriol 179, 4684-8.
Billot-Klein, L. Gutmann, E. Collatz, J. van Heijenoort, Antimicrob. Agents Chemother. 36, 1487 (1992).
Blundell et al., 1987, Nature, Vol. 326: 347.
Bouhss, A., Josseaume, N., Severin, A., Tabei, K., Hugonnet, J. E., Shlaes, D., Mengin-Lecreuix, D., Van Heijenoort, J. & Arthur, M. (2002) J Biol Chem 277, 45935-41.
Brunger, A. T., Adams, P. D., Clore, G. M., Delano, W. L., Gros, P., Grosse-Kunstleve, R W., Jiang, J.-S., Kuszewski, J., Nilges, M., Pannu, N. S., Read, R. J., Rice, L. M., Simonson, T., and Warren, G. L. (1998). Crystallography & NMR system: a new software for macromolecular structure determination. Acta Crystallogr. D54, 905-921.
Chastanet, A., Fert, J. & Msadek, T. (2003) Mol Microbiol 47, 1061-73.
Coyette, H. R. Perkins, I. Polacheck, G. D. Shockman, J. M. Ghuysen, Eur. J. Biochem. 44, 459 (1974).
Galperin, M. Y. & Koonin, E. V. (1997) Protein Sci 6, 2639-43.
Goodford, 1984, J. Med. Chem., Vol. 27: 557.
Grohs, L. Gutmann, R. Legrand, B. Schoot, J. L. Mainardi, J. Bacteriol. 182, 6228 (2000).
Hol, 1986, Angew. Chem., Vol. 25: 767.
Jones, T. A., Zou, J. Y., Cowan, S. W., and Kjeldgaard, M. (1991). Improved methods for building protein models in electron density maps and the location of errors in theses models. Acta Cryst. A47, 110-119
Kleywegt, G. J., and Jones, T. A. (1994). Detection, delineation, measurement and display of cavities in macromolecular structures. Acta Crystallogr. D50, 178-185.
Mainardi et al., J. Biol. Chem. 275, 16490 (2000).
Mainardi et al., J. Biol. Chem. 277, 35801 (2002).
Mainardi, J. L., Billot-Klein, D., Coutrot, A., Legrand, R., Schoot, B. & Gutmann, L. (1998) Microbiology 144(Pt 10), 2679-85.
Mc Ree, D. E. 1993. Practical protein crystallography. Academic Press.
Mengin-Lecreuix et al., J. Bacteriol. 181, 5909 (1999).
Messer, M. Griffiths, P. D. Rismiller, D.C. Shaw, Comp. Biochem. Physiol. Biochem. Mol. Biol. 118B, 403 (1997).
Navaza, J. (1994). AMoRe: an automated package for molecular replacement. Acta Crystallogr. A50, 157-163.
Pisabarro, M. A De Pedro, D. Vazquez, J. Bacteriol. 161, 238 (1985).
Sheridan and Venkataraghavan, 1987, Acc. Chem. Res., Vol. 20: 322
Templin, A. Ursinus, J. V. Höltje, EMBO J. 18, 4108 (1999).
Verlinde C. L. M. J. & Hol, W. G. J., 1994, Structure, Vol. 2: 577.
Williamson, R., le Bouguenec, C., Gutmann, L. & Horaud, T. (1985) J Gen Microbiol 131, 1933-40.

Table of the sequences

| SEQ ID N° | Type | Description |
|---|---|---|
| 1 | protein | D-aspartate ligase from E. faecium |
| 2 | protein | D-aspartate ligase from L. lactis |
| 3 | protein | D-aspartate ligase from L. cremoris |
| 4 | protein | D-aspartate ligase from L. gasseri |
| 5 | protein | D-aspartate ligase from L. johnsonii |
| 6 | protein | D-aspartate ligase from L. Delbruckei subsp. |
| | | Bulgaricus |
| 7 | protein | D-aspartate ligase from L. casei |
| 8 | protein | D-aspartate ligase from L. acidophilus |
| 9 | protein | D-aspartate ligase from L. brevis |
| 10 | protein | D-aspartate ligase from Pediococcus |
| | | pentosaceus |
| 11 | protein | (340-466) C-terminal portion of the |
| | | L,D-transpeptidase from E. faecium |
| 12 | protein | (216-466) C-terminal portion of the |
| | | L,D-transpeptidase from E. faecium |
| 13 | protein | D-aspartate ligase from E. faecium |
| 14 | DNA | primer for D-aspartate ligase coding |
| | | sequence |
| 15 | DNA | primer for D-aspartate ligase coding |
| | | sequence |
| 16 | DNA | primer for D-aspartate ligase coding |
| | | sequence |
| 17 | DNA | primer for D-aspartate ligase coding |
| | | sequence |
| 18 | DNA | primer for L,D-transpeptidase coding |
| | | sequence |
| 19 | DNA | primer for L,D-transpeptidase coding |
| | | sequence |
| 20 | Protein | N-terminal amino acid sequence |
| 21 | DNA | transcriptional initiation site |
| 22-31 | DNA | Nucleic acids encoding amino acid |
| | | sequences of SEQ ID N° 1 to 10 |
| 32 | DNA | Nucleic acid encoding the amino acid |
| | | sequence of SEQ ID N° 13 |
| 33 | Protein | (119-466) portion of the |
| | | L,D-transpeptidase from E. faecium |

1-48. (canceled)

49. A method of screening antibacterial substances comprising determining the ability of a candidate substance to inhibit the activity of a purified enzyme further defined as:
a D-aspartate ligase comprising a polypeptide having an amino acid sequence possessing at least 50% amino acid identity with an amino acid sequence of any of SEQ ID NOs: 1 to 10, or a biologically active fragment thereof; or
a L,D-transpeptidase comprising a polypeptide having an amino acid sequence possessing at least 50% amino acid identity with the amino acid sequence of SEQ ID NO: 11, or a biologically active fragment thereof.

50. The method of claim 49, further defined as comprising:
providing a composition comprising said purified enzyme and a substrate thereof;
adding the candidate substance to be tested to the composition to make a test composition;
comparing activity of said enzyme in said test composition with activity of the same enzyme in the absence of said candidate substance; and
selecting positively a candidate substance that inhibits the catalytic activity of said enzyme.

51. The method of claim 49, wherein said enzyme is a D-aspartate ligase comprising a polypeptide having an amino acid sequence possessing at least 60% amino acid identity with an amino acid sequence selected from the group consisting of SEQ ID NO: 1 to SEQ ID NO: 10, or a biologically active fragment thereof.

52. The method of claim 51, wherein said enzyme is a D-aspartate ligase comprising a polypeptide having an amino acid sequence possessing at least 90% amino acid identity with the amino acid sequence of SEQ ID NO: 1, or a biologically active fragment thereof.

53. The method of claim 51, wherein said enzyme is a D-aspartate ligase comprising a polypeptide having the amino acid sequence of SEQ ID NO: 1, or a biologically active fragment thereof.

54. The method of claim 51, wherein the D-aspartate ligase activity is assessed using, as substrates, D-aspartate and UDP-MurNac pentapeptide or UDP-MurNac tetrapeptide.

55. The method of claim 54, wherein the D-aspartate ligase activity is assessed by quantifying UDP-MurNac pentapeptide-Asp or UDP-MurNac tetrapeptide-Asp that is produced.

56. The method of claim 49, wherein said enzyme is a L,D-transpeptidase comprising a polypeptide having an amino acid sequence possessing at least 60% amino acid identity with the amino acid sequence of SEQ ID NO: 11, or a biologically active fragment thereof.

57. The method of claim 56, wherein said enzyme is a L,D-transpeptidase comprising a polypeptide having an amino acid sequence possessing at least 90% amino acid identity with the amino acid sequence of SEQ ID NO: 11, or a biologically active fragment thereof.

58. The method of claim 56, wherein said enzyme is a L,D-transpeptidase comprising a polypeptide having the amino acid sequence of SEQ ID NO: 11, or a biologically active fragment thereof.

59. The method of claim 56, wherein said enzyme is a L,D-transpeptidase comprising a polypeptide having the amino acid sequence possessing at least 90% amino acid identity with the amino acid sequence of SEQ ID NO: 12, or a biologically active fragment thereof.

60. The method of claim 56, wherein said enzyme is a L,D-transpeptidase comprising a polypeptide having the amino acid sequence of SEQ ID NO: 12, or a biologically active fragment thereof

61. The method of claim 56, wherein said enzyme is a L,D-transpeptidase having 90% amino acid identity with the amino acid sequence of SEQ ID NO: 13, or a biologically active peptide fragment thereof.

62. The method of claim 56, wherein said enzyme is a L,D-transpeptidase comprising a polypeptide consisting of the amino acid sequence of SEQ ID NO: 13, or a biologically active peptide fragment thereof.

63. The method of claim 56, wherein the L,D-transpeptidase activity is assessed using, as substrates, (i) a donor compound consisting of a tetrapeptide and (ii) an acceptor compound further defined as a D-amino acid or a D-hydroxy acid.

64. The method of claim 63, wherein the tetrapeptide is L-Ala-D-Glu-L-Lys-D-Ala, Ac₂-L-Lys-D-Ala or disaccharide-tetrapeptide(iAsn).

65. The method of claim 63, wherein said D-amino acid is D-methionine, D-asparagine or D-serine.

66. The method of claim 63, wherein said D-hydroxy acid is D-2-hydroxyhexanoic acid or D-lactic acid.

67. A method for the screening of antibacterial substances comprising:
providing a candidate substance;
assaying said candidate substance for its ability to bind to:
a D-aspartate ligase comprising a polypeptide having an amino acid sequence possessing at least 50% amino acid identity with an amino acid sequence of any of SEQ ID NOs: 1 to 10, or a biologically active fragment thereof; or
a L,D-transpeptidase comprising a polypeptide having an amino acid sequence possessing at least 50% amino acid identity with the amino acid sequence of SEQ ID NO: 11, or a biologically active fragment thereof.

68. The method of claim 67, further comprising determining the ability of said candidate substance to inhibit the activity of a purified:
D-aspartate ligase comprising a polypeptide having an amino acid sequence possessing at least 50% amino acid identity with an amino acid sequence of any of SEQ ID NOs: 1 to 10, or a biologically active fragment thereof; or
L,D-transpeptidase comprising a polypeptide having an amino acid sequence possessing at least 50% amino acid identity with the amino acid sequence of SEQ ID NO: 11, or a biologically active fragment thereof.

69. A crystallized L,D-transpeptidase having the amino acid sequence starting at the amino acid located in position 119 and ending at the amino acid located in position 466 of the amino acid sequence of SEQ ID NO: 13.

70. A method for selecting a compound that interacts with a catalytic site of a L,D-transpeptidase having the amino acid sequence starting at the amino acid located in position 119 and ending at the amino acid located in position 466 of the amino acid sequence of SEQ ID NO: 13 comprising:
generating a three-dimensional model of said catalytic site using a set of data corresponding to the relative structural coordinates according to Table 3; and
employing said three-dimensional model to design or select a compound, from a serial of compounds, that interacts with said catalytic site of a crystallized L,D-transpeptidase having the amino acid sequence starting at the amino acid located in position 119 and ending at the amino acid located in position 466 of the amino acid sequence of SEQ ID NO: 13.

71. The method of claim 70, further comprising:
obtaining a compound designed or selected; and
assaying to determine whether the compound is an antibacterial substance.

72. The method of claim 70, wherein said compound is selected from a library of compounds.

73. The method of claim 70, wherein said compound is selected from a database.

74. The method of claim 70, wherein said compound is designed de novo.

75. A method for selecting a compound that interacts with a catalytic site of a L,D-transpeptidase having the amino acid sequence starting at the amino acid located in position 119 and ending at the amino acid located in position 466 of the amino acid sequence of SEQ ID NO: 13 comprising selecting or designing a candidate L,D-transpeptidase inhibitor by performing computer fitting analysis of the candidate agonist or antagonist compound with a three-dimensional model of said catalytic site using a set of data corresponding to the relative structural coordinates according to Table 3.

76. The method of claim 75, further comprising:
obtaining a compound designed or selected; and
assaying to determine whether the compound is an antibacterial substance.

77. The method of claim 75, wherein said compound is selected from a library of compounds.

78. The method of claim 75, wherein said compound is selected from a database.

79. The method of claim 75, wherein said compound is designed de novo.

80. A method for selecting a compound that interacts with a catalytic site of a L,D-transpeptidase having the amino acid sequence starting at the amino acid located in position 119 and ending at the amino acid located in position 466 of the amino acid sequence of SEQ ID NO: 13 comprising:
generating a three-dimensional model of a L,D-transpeptidase having the amino acid sequence starting at the amino acid located in position 119 and ending at the amino acid located in position 466 of the amino acid sequence of SEQ ID NO: 13 using a set of data corresponding to the relative structural coordinates of amino acid residues HIS421, SER439, HIS440 and CYS442 according to Table 3±a root mean square deviation from the backbone atoms of said amino acids of not more than 1.5 Å;
performing, for each candidate compound, a computer fitting analysis of said candidate inhibitor compound with three-dimensional model generated; and
selecting, as an inhibitor compound, every candidate compound having a chemical structure inducing: (i) hydrogen bonds with at least two of the HIS421, SER439, HIS440 and CYS442 amino acid residues of a L,D-transpeptidase having the amino acid sequence starting at the amino acid located in position 119 and ending at the amino acid located in position 466 of the amino acid sequence of SEQ ID NO: 13; or (ii) steric constraints with at least one of the amino acid residues comprised in the 368-450 polypeptide portion of a L,D-transpeptidase having the amino acid sequence starting at the amino acid located in position 119 and ending at the amino acid located in position 466 of the amino acid sequence of SEQ ID NO: 13.

81. The method of claim 80, further comprising:
obtaining a compound designed or selected; and
assaying to determine whether the compound is an antibacterial substance.

82. The method of claim 80, wherein said compound is selected from a library of compounds.

83. The method of claim 80, wherein said compound is selected from a database.

84. The method of claim 80, wherein said compound is designed de novo.

85. A machine-readable data storage medium, comprising a data storage material encoded with machine-readable data, wherein said machine-readable data consist of the X-ray structural coordinate data of a L,D-transpeptidase having the amino acid sequence starting at the amino acid located in position 119 and ending at the amino acid located in position 466 of the amino acid sequence of SEQ ID NO: 13, according to Table 3.

86. A machine-readable data storage medium, comprising a data storage material encoded with machine-readable data which, when using a machine programmed with instructions for using said data, displays a graphical three-dimensional representation of a crystal of a L,D-transpeptidase having the amino acid sequence starting at the amino acid located in position 119 and ending at the amino acid located in position 466 of the amino acid sequence of SEQ ID NO: 13, according to Table 3.

87. A system for generating a three-dimensional model of at least a portion of the L,D-transpeptidase of SEQ ID NO: 13, said system comprising:
a data storage device storing data comprising a set of structure coordinates defining at least a portion of the three-dimensional structure of said L,D-transpeptidase according to Table 3; and
a processing unit for generating the three-dimensional model from said data stored in said data-storage device.

88. A D-asparte ligase selected from the group consisting of the D-aspartate ligases of SEQ ID NOs: 1 to 10, or a biologically active peptide fragment thereof.

89. A nucleic acid encoding a D-aspartate ligase according to claim 88, or a biologically active peptide fragment thereof.

90. The nucleic acid of claim 89, comprising a sequence of any of SEQ ID NOs: 22 to 31.

91. An antibody directed against a D-aspartate ligase of claim 39 or against a biologically active peptide fragment thereof.

92. A kit for the screening of an antibacterial substance comprising at least one container comprising a purified D-aspartate ligase of claim 88.

93. The kit of claim 92, further comprising one or more reagents necessary for assessing the enzyme activity of said D-aspartate ligase.

94. A L,D-transpeptidase of SEQ ID NO: 13, or a biologically active peptide fragment thereof.

95. A nucleic acid encoding a L,D-transpeptidase of claim 94, or a biologically active peptide fragment thereof.

96. The nucleic acid of claim 95, comprising the sequence of SEQ ID NO: 32.

97. An antibody directed against a L,D-transpeptidase of claim 95, or against a biologically active peptide fragment thereof.

98. A kit for the screening of an antibacterial substance, wherein said kit comprises at least one container comprising a purified L,D-transpeptidase of claim 94.

99. The kit of claim 98, further comprising one or more reagents necessary for assessing the enzyme activity of said L,D-transpeptidase.


SEQUENCE LISTING (33 sequences)

SEQ ID NO: 1 - protein, 421 aa, Enterococcus faecium
MMNSIENEEFIPILLGSDMNVYGMARSFNEAYGKICQAYASDQLAPTRYSKIVNVEVIPGFDKDPVFIETMLRLAKERYSDKSKKYLLIACGDGYAELISQHKQELSEYFICPYIDYSLFERLINKVSFYEVCEEYDLPYPKTLIVREEMLVNGHLEQELPFEFPVALKPANSVEYLSVQFEGRKKAFILETREEFDLILGRIYEAGYKSEMIVQDFIPGDDSNMRVLNAYVDEDHQVRMMCLGHPLLEDPTPASIGNYVVIMPDYNEKIYQTIKAFLEKIEYTGFANFDMKYDPRDGEYKLFEINLRQGRSSFFVTLNGLNLARFVTEDRVFNKPFVETTYGTNQSDKARLWMGVPKKIFLEYARENEDKKLAEQMIKENRYGTTVFYEKDRSIKRWLLMKYMFHNYIPRFKKYFHVKEG

SEQ ID NO: 2 - protein, 408 aa, Lactobacillus lactis
MSQTNEFQPILLGSDINVYGMARSFHEEFGITSLALSASGLAPTKYSKIVEVQTHENFDHEETFIQVLRDFASKHKDSKKKYLLVACGDGYALLASKYREELSEFFEVPYIDYALYRQLENKPDFYNICEQYNLPYPKTVLVNPKSEIDTLVDELTFPYPMILKPGNSISYLDVHFEGQKKAFTIASNEEMKLMLHRCYEAGYPDEMIIQDFIPGGDENMRVLNAYVDKNHKVRMMCLGHPLLEDPSPKAIGNYVAILPDYNQQVYQQIQDFLEKIEYTGFANFDMKYDPRDGSYKLFEINLRQGRSSFYVTLNGYNLTRYLVADRINNEPFTETIYGKGSKLWLGVPKKVLLKYVNPSFKEEIRRFINSGNYGTTLFYSKDNSPKRYLLMKYAFHRYIGAFKTYFHK

SEQ ID NO: 3 - protein, 408 aa, Lactobacillus cremoris
MSQTIEFQPILLGSDINVYGMARSFHEEFGITSLALSASGLAPTKYSKIVEVQTHENFDHEETFIQVLRDFASKHKDDHKKYLLVACGDGYALLASKYRDELSEFFEVPYIEYSLYQQLENKPDFYNICEQYGLPYPKTVLVNPESEIDSLVDSLTFPYPMILKPGNSISYLDVHFEGQKKAFTIESNEEMKLMLHRCYEAGYPDEMIIQDFIPGGDENMRVLNAYVDKNHKVRMMCLGHPLLEDPSPKAIGNYVAILPDYNEQVYQQIQEFLEKIEYTGFANFDMKYDPRDGSYKLFEINLRQGRSSFYVTLNGYNLTRYLVADRIYNEAFTETVYGKGTKLWLGVPKKVLLKYVNPSFKEEIRRFVSSGNYGTTLFYSKDNSPKRYLLMKYAFHRYIGAFKTYFHK

SEQ ID NO: 4 - protein, 443 aa, Lactobacillus gasseri
MVQAKFTPILLGSDINVYGMARSFNEAYGIKVQAWAASQLAATRYSKIVDVEVHEGFEEDPGFMNVMKQKIEEYKNHPEPVILIACGDGYAELLAKHKDELKDTFVVPYIDYDLLEKLISKEGFYEIAEKYGLPYPHTKIVTMDDYKAENYLDLPFDYPIELKPEDPVSWLNCQFEGRKKAFTIHDETELVDIVGKIYTNGYTEDLILQDFIPGDDSNMRVLNAYVDKDHKVKMMCLGHPLLEDPTPQSIGNYMAILPAFSQKLYDTVQSFLEKLNYTGMANFDIKYDPRDGEYKFFEINLRQGRSSFYVTLNGYNLAKWYIDDYVEDNLKDKPTVYGNKDGANYMLWLGVPKKIFKEYAYENGSKRLAEKLIDEGHYGTTVFYDKDRSFKRWLLMRYMFHNYYARYKKYYEVNKGQYFEEEAKKLKKQALRDGQQENHENEI

SEQ ID NO: 5 - protein, 443 aa, Lactobacillus johnsonii
MVQAKFTPILLGSDINVYGMARSFNEAYGIKVQAWAASQLAATRYSKIVDVEVHEGFEEDPGFMNVMKQKIEEYKNHPEPVILIACGDGYAELLAKHKGELKDTFVVPYIDYDLLEKLISKEGFYEIAEKYGLPYPHTKIVTMDDYKAENYLNLPFDYPVELKPEDPVSWLNCQFEGRKKAFTIHDEAELVDIVGKIYTNGYTEDLILQDFIPGDDSNMRVLNAYVDKDHKVKMMCLGHPLLEDPTPQSIGNYMAILPAFSQKLYDTVQSFLEKLNYTGMANFDIKYDPRDGEYKFFEINLRQGRSSFYVTLNGYNLAKWYVDDYVEDNLKDKPTVYGNKDGANYMLWLGVPKKIFKEYAYDNGSKRLAEKLIDEGHYGTTVFYDKDRSLKRWLLMHYMFHNYYARYKKYYQVNKGQYFEEEAKKLEKQALRDGQQENHENEI

SEQ ID NO: 6 - protein, 418 aa, Lactobacillus delbrueckii
MQQFTPILLGSDINVYGMARSFHEAYGIKVQAWGASTLAPTRYSKIVDIEVHPGFTEDPEFIKVMREKIKEYKKHPEPVILISCGDGYSELLAKHKQELEEAFIVPYIDYALLEKLISKEGFYEVCEEYGLPYPKTKIITMSAYKDGSYADVPFPFPVALKPEDPVSWLDVQFEGRKKAFVIKDLDEFKDIVGKIYTNGYKEDLILQDFIPGDDSNMRVLNAYVDKNHQVKMMCLGHPLLEDPDPIAIGNYMVIAPDYDEKLYQTVQAFLEKINYTGMANFDIKYDVRDGQYKFFEINLRQGRSSFYVTLNGYNLAKWYVDDYVEDSLKDQPVVYGNQDPAKHKLWLGVPDKIFEQYAVDSLAKKEALALLRAGKVGTTVFYKPDMNVKRWLLMKHMYHNYYKDYQKYFKVNKEQYFE

SEQ ID NO: 7 - protein, 419 aa, Lactobacillus casei
MKDAPSFTPILLGSDMNVYGMARSFHEMIGGAIDVYAREQLAPTRFSRIVNVHLIEHFDSDPTFIENMRQVAKVHADAPGKLLLIACGDTYAQLVSKHKDELSQWFICPYVDYELLKRLNSKESFYQVCEEYHLPYPATKIVTKSMYESNATITAPFEFPVALKPTDSVEWLNIRFEGRKKAFTIHSQVELTNTIAKIYDNGYTSDLILQDFVPGDDSNMRTLNCYVDQYHHVKMMCLGHPLLEDPTPSSIGNYVAIMPAFDQDLYDQIKTFLEAIKFTGFANFDMKYDHRDKTFKLFEINIRQGRSSFFVTLNGYNLASWPVRDYIVGDLKDAPTVYGNDDHSKYKLWLGVPERVFKQYSADNEAKKEALQLLKEKRYGTTVFYKPDMTFKRWLLMKYMFHNYVGRFKQYFAENKGDM

SEQ ID NO: 8 - protein, 422 aa, Lactobacillus acidophilus
MTKEFTPILLGSDINVYGMARSFNEAYGIRVKALADVQLAATRYSKIVDVELHHGFSADPTFMDVMRKKMEIYKDHKEPVILIACGDGYAELLSKHKDELSKVFIVPYIEYDLLEKLISKEGFYEVAEEYGLPYPKTKIITMDDYKSGKYKDVPFQFPVELKPEDPVSWLNCQFEGRKKTFTLHDENEFEDIVTKIYTHGYTEDLILQDFIPGDDSNMRTLNAYVDKNHQVKMMCLGHPLLEDPTPQSIGNYMTILPAYDEKLYEQVQSFLEKLNYTGMANFDIKYDERDGQYKFFEINLRQGRSSFYVTLNGYNLAKWYIDDYVEDNLSDKKTVYGNKLKSKHVLWMGVPVKIFKEYAFENEAKDTAEELIHEGRYGTTVFYDKDRSLKRWLLMKYMFHNYFARYKKYYEVNKGQYFKKNK

SEQ ID NO: 9 - protein, 418 aa, Lactobacillus brevis
METNGVKFTPVLLGSDFNVYGMARSFYELYHEPVQAFASVQLAPTRYTKVVNLELIPGFDEDPVWIQTMRKLKERYADHPEPVILIGCGDGYAELISKHKAELTDVFICPYIDYDKLRHLNNKENFYKVCDQYGLPYPATRIITKDMATAAEPVEQPFDYPVALKPANSVEWLDVHFEGRKKAFRIQSEAEFRDIVQKIYANGYQSDLILQDFVPGDDSNMRVLNAYVDQYHHVKMMCLGHPLLEDPAPSAIGNYVAIIPEYNETIYQNIQKFLEAIEFTGYANFDMKYDVRDQTFKLFEINLRQGRSSFFVTLNGYNLADWVVKDYVKQELVDAPTVYGNQDESQHALWLGVPVKVFKQYTRENVDKQRALKLIAAGKYGTTYHYDQDMNLKRWALLTWMQHNYTVNFKKYFAENKG

SEQ ID NO: 10 - protein, 418 aa, Pediococcus pentosaceus
MERPTNNFTPILLGSDFNAYGMARSFYEITGRRVQAYAGTVLAPTRFTKIVNVDIIPGFSEDPVFIESMRKIAKKYENSDEKVVLLGMGDGYAELISKHKEELSKTFICPYIDYDLLKQLNNKERFYSICDEYNLPYPATHFITKAQYESGEKFISPFEFPVALKPADSVEWLDIHFEGRKKAFRIKTQAELDEVIGKIYDNGYTSDLILQDFIPGDDSNMRVLNAYVDQNHQVKMMCLGHPLLEDPAPSAIGNYVAIIPEFNQEIYDTVKNFLESINYVGYANFDMKYDSRDGKFKLFEINIRQGRSSFFVTLNGYNLARWVVEDRVNNALADRETVYANDVPGNWKLWLGVPVKVFKKYAKNNDDKQKALELINNDQYGFTAIYDEDMNFKRSLLMWWMNHNYVKNFRKYFKENKG

SEQ ID NO: 11 - protein, 127 aa, Enterococcus faecium
IEDTYIEVDLENQHMWYYKDGKVALETDIVSGKPTTPTPAGVFYVWNKEEDATLKGTNDDGTPYESPVNYWMPIDWTGVGIHDSDWQPEYGGDLWKTRGSHGCINTPPSVMKELFGMVEKGTPVLVF

SEQ ID NO: 12 - protein, 251 aa, Enterococcus faecium
LKEQLASMNAIANVKATYSINGETFQIPSSDIMSWLTYNDGKVDLDTEQVRQYVTDLGTKYNTSTNDTKFKSTKRGEVTVPVGTYSWTIQTDSETEALKKAILAGQDFTRSPIVQGGTTADHPLIEDTYIEVDLENQHMWYYKDGKVALETDIVSGKPTTPTPAGVFYVWNKEEDATLKGTNDDGTPYESPVNYWMPIDWTGVGIHDSDWQPEYGGDLWKTRGSHGCINTPPSVMKELFGMVEKGTPVLVF

SEQ ID NO: 13 - protein, 466 aa, Enterococcus faecium
MTRSSHRKKSKHVALLSVLGVLLVLISFYTYRSTYYTKHFLPNTEINGINVSNLTVEKANEKLKEAYSDKKLAIKEDGKVWQEVPKSELGYKDDFTSELSPILNEQNGWTWGMTYVSAAEKQEIDPVSQNHQKLDTTVQTLTTKLTDLNKDRTPTTDATIEKAGDSFVIKPEVNGNTIDVDAAVKQLKSAVNSGKDTIELTEFKEKPKVTSEDSSLKEQLASMNAIANVKATYSINGETFQIPSSDIMSWLTYNDGKVDLDTEQVRQYVTDLGTKYNTSTNDTKFKSTKRGEVTVPVGTYSWTIQTDSETEALKKAILAGQDFTRSPIVQGGTTADHPLIEDTYIEVDLENQHMWYYKDGKVALETDIVSGKPTTPTPAGVFYVWNKEEDATLKGTNDDGTPYESPVNYWMPIDWTGVGIHDSDWQPEYGGDLWKTRGSHGCINTPPSVMKELFGMVEKGTPVLVF

SEQ ID NO: 14 - DNA, 33 bp, artificial sequence
  primer
gagagaccatggtgaacagtattgaaaatgaag

SEQ ID NO: 15 - DNA, 42 bp, artificial sequence
  primer
ctccatggctaggatccttctttcacatgaaaatactttttg

SEQ ID NO: 16 - DNA, 44 bp, artificial sequence
  primer
gggagctcaaggaggagactgaccatggactttaataaaattga

SEQ ID NO: 17 - DNA, 63 bp, artificial sequence
  primer
catctagattaagatctcaatggtgatgatggtgatgggatccctattataaaagccagtcat

SEQ ID NO: 18 - DNA, 30 bp, artificial sequence
  primer
ttccatggcagaaaaacaagaaatagatcc

SEQ ID NO: 19 - DNA, 25 bp, artificial sequence
  primer
ttggatccgaagaccaatacaggcg

SEQ ID NO: 20 - protein, 20 aa, artificial sequence
  N-terminal peptide
AEKQEIDPVSQNHQKLDTTV

SEQ ID NO: 21 - DNA, 21 bp, artificial sequence
  transcriptional initiation site
acttaaggagttgtcgatatg

SEQ ID NO: 22 - DNA, 1266 bp, Enterococcus faecium
atgatgaacagtattgaaaatgaagaatttatccctatattattaggaagcgacatgaacgtttatggtatggcacgttcttttaatgaagcatacggcaaaatctgccaagcatatgcttcggatcagttagcccctacccgctacagtaaaattgtcaatgtagaagttattccaggatttgataaagaccctgtatttatcgagacgatgcttcgtttggcaaaagaaagatattctgataagagtaaaaaatatcttttgattgcttgtggtgatggatatgcagaattgatctcccaacataaacaagagctgtcagaatattttatctgtccatatattgattattctttatttgaaaggctgatcaataaagtaagcttttatgaagtttgtgaagaatatgatcttccgtatcctaaaacattgatcgtcagagaagaaatgttagtaaatggacatttggaacaggaattgccttttgaattcccagttgctttgaaacctgcaaacagtgtggagtatctttctgtccaatttgaaggaagaaaaaaagcttttattttagagactagagaagagttcgatttaattttaggccggatatacgaagccggttataaaagtgaaatgattgtacaagattttatcccgggtgatgatagtaatatgcgtgtgctaaatgcgtatgtagatgaagatcatcaagttcggatgatgtgccttggtcatccactattagaagatcctacaccagcatcgattggtaattacgttgtgatcatgccagattataacgaaaaaatctatcaaaccattaaggcatttttggaaaaaatcgaatatacaggctttgccaatttcgatatgaagtatgatcctagagatggagaatataaattatttgaaatcaatttgagacaaggtcgtagcagtttctttgtgacattgaacggcttgaatctggctcgttttgtcacagaagatcgagtgttcaacaaaccctttgttgaaacgacctatggaacgaatcaatcagataaagcaagattatggatgggtgtgcccaaaaaaatctttttagaatatgctcgggaaaatgaagacaaaaaactagctgagcaaatgatcaaagaaaatcgttacggtactactgtattttatgaaaaagaccgatcaatcaagcgctggttgttgatgaaatatatgttccataactatattccaagattcaaaaagtattttcatgtgaaagaaggctag

SEQ ID NO: 23 - DNA, 1266 bp, Lactobacillus lactis; identical to SEQ ID NO: 22

SEQ ID NO: 24 - DNA, 1227 bp, Lactobacillus cremoris
gtgtcacaaacaattgaatttcaacctattctgttaggttctgatatcaacgtatatggaatggctcgttcatttcatgaagaatttgggattacttcccttgctttaagtgcttctggattggcacccacaaagtattcgaaaatcgttgaagtgcaaacacatgaaaattttgaccatgaggaaacttttatccaagttcttcgtgatttcgcaagtaaacataaggatgatcataaaaaatatcttttagtggcttgtggggatggctatgctttacttgcttcgaaatatcgtgatgaactttccgaattttttgaagtgccttatattgagtactctctttatcaacaattagaaaataaacctgatttttacaatatctgtgagcagtatggcttgccatatcctaaaacggtattggttaatcctgaatcagaaatagattctctagtggacagtttaactttcccttacccaatgattttaaaaccagggaattccatttcttacttggacgttcattttgaaggacaaaagaaggctttcacgattgaatcaaatgaagaaatgaagttaatgcttcatcgttgttatgaagccggttatcctgatgaaatgattatccaagactttattcctggtggcgatgaaaatatgcgcgttttgaatgcttatgtagataaaaatcataaggtaagaatgatgtgtttaggtcatcctttgcttgaagatccaagtccaaaagcaattggaaactatgttgcgattttgcctgattataatgaacaagtctatcagcaaattcaggaatttcttgagaaaattgagtacacaggctttgcgaatttcgatatgaagtatgatccgcgtgatggttcttataaactttttgaaattaacttacgtcaagggcgcagttcattttatgttaccttaaatgggtataatttgacccgttatttggtggctgaccgtatttacaacgaagcttttactgaaacagtttatggtaaagggactaagctttggttgggagttcctaaaaaagtattgcttaaatatgtgaatccgagctttaaagaggaaattcgtcgttttgttagctcaggaaactatggaacaactttattttattccaaggataactctcctaagcgttaccttttgatgaagtatgctttccatcgttatattggggcctttaaaacatattttcacaaataa

SEQ ID NO: 25 - DNA, 1332 bp, Lactobacillus gasseri
atggtacaagcaaaatttactcctattttattaggtagcgacatcaatgtttatggaatggcacgttcattcaatgaagcctatgggatcaaagttcaagcatgggcagctagccaattagcagcaacgcgttactctaaaattgttgacgtagaagttcatgaaggctttgaagaagatccaggctttatgaacgtgatgaagcaaaagattgaagaatacaagaatcacccagaaccagttattttaattgcttgtggggatggttatgctgaattacttgctaaacacaaggatgaattaaaggatactttcgttgttccttatattgactatgacttgctagagaagttaatttctaaagagggattctacgaaattgctgaaaaatatggtcttccatatccacatactaagattgtgaccatggatgattacaaggccgaaaattacttagatttaccatttgattatccaattgaattgaaaccagaagatcctgtttcttggttgaattgtcaatttgaaggtcgtaaaaaggcatttactattcatgacgaaactgaattagtagatattgtgggcaagatttatactaacggttatactgaagacttgattttgcaagactttatcccaggtgatgattctaacatgcgtgttcttaacgcctatgttgataaggatcacaaggttaagatgatgtgcttaggtcacccacttcttgaagatcctacgccacaatcaatcggtaactacatggcaattttgccagcatttagtcaaaaactttatgatacagttcaatcattccttgagaagttgaactacacagggatggctaactttgacattaagtatgatccgagagatggagaatacaagttctttgaaattaacttgcgtcaaggtcgttcaagtttctatgtaactttaaatggctataacttagctaagtggtatatagatgattatgtagaagataatttaaaagataagccaactgtatatggtaataaagatggcgctaattacatgctttggcttggtgttcctaagaaaatctttaaggaatatgcttatgaaaatggctcaaagcgtttagctgaaaaattaattgatgaaggtcattatggtactactgtcttttacgataaagatcgtagcttcaaacgctggttattaatgcgttatatgttccacaactactatgctagatacaagaagtactatgaagtaaacaagggacaatactttgaagaagaagctaagaagttaaaaaagcaagcacttcgtgatggtcaacaagaaaaccatgaaaatgaaatttag

SEQ ID NO: 26 - DNA, 1332 bp, Lactobacillus johnsonii
atggtacaagcaaaatttactcctattttactcggtagtgacatcaatgtttacggaatggctcgctccttcaacgaagcttatggaattaaggttcaagcttgggctgctagtcaattagcagcaacgcgctattctaaaattgttgacgtagaagttcatgaaggctttgaagaagatccaggctttatgaacgttatgaagcaaaagattgaagaatacaagaatcatccagaaccagttattttaattgcttgtggagatggatacgcagaattattggctaagcataagggtgagttgaaggatacttttgttgttccttacattgattatgacttgctagagaagctaatttctaaagaaggtttctacgaaattgctgaaaaatatggtcttccatatccacatactaagattgtgactatggatgactataaggctgaaaattatttgaatttaccatttgattatccagttgaattgaagccagaagatccagtttcatggttgaattgtcaatttgaaggccgtaaaaaagcctttactattcacgatgaggcagaattagtagatatcgttggtaaaatctataccaatggttatactgaagacttgattttacaagatttcattccaggcgatgattctaacatgcgtgttcttaatgcttatgttgataaagatcacaaggttaagatgatgtgcttaggtcacccacttcttgaagacccaactcctcaatctatcggtaattacatggcaattttgccagccttcagtcaaaagctctacgatactgttcaatcattccttgaaaaattgaactatactggtatggctaactttgacattaagtacgatccaagagatggagaatacaaattctttgagatcaacctccgtcaaggtcgttcaagtttctatgtaactttaaatggctataacttagctaagtggtatgtagatgattatgttgaagataatttgaaagataagccaacagtttatggaaataaagacggtgctaactatatgttgtggcttggtgttccaaagaagatctttaaggaatatgcttacgacaacggttcaaaacgcctagctgaaaagttaattgacgagggtcactatggtacaactgtcttctatgataaagatcgtagcttaaaacgttggttattgatgcactacatgttccataactactatgctagatataagaagtactaccaagtaaataagggacaatattttgaagaagaagctaaaaaattagaaaagcaagctcttcgtgatggtcaacaagaaaaccatgagaatgaaatttaa

SEQ ID NO: 27 - DNA, 1260 bp, Lactobacillus delbrueckii
atggctaaatttacaccaattttgcttggcagcgacatcaatgtctacgggatggcgcggtcttttaatgaagcatacggcatcaaggtccaggcttgggcggcaacgcaattggcggccacccgctattctaagatcgtggacgttgaagtgcaccctggtttttccgaagacccgggcttcatggaagtcatgcaggaaaaaatcaaggaataccagaatcatgaagagccagtcattctgatcgcttgtggggacggctatgcagaacttttggctaagcacaagcaagaactggaagggaccttcgtcgttccttacatcgactaccagcttttggaaaagctgatctctaaggaaggcttctacgaagtctgtgaagaatacggcctgccttacccgaagaccaagatcatcaagctggctgactaccaggatgggtcatacgccgacgtgccttttgccttcccggttgccttgaagccggaagacccggtttcctggctggatgtccagtttgaagggcggaagaaggcttttgtcatcaaggacctggctgaattcaaggacattgtgggcaagatctacactaatggctacaaggaagacttgattctgcaggacttcatcccgggcgatgactccaacatgcgggtcttgaacgcttatgtggacaagaaccaccaggtcaagatgatgtgcctgggccacccgcttttggaagacccgactccgcagtcaatcgggaactacatggctattttgccggcctatgatgaaaagctttacgagaccgtccagaccttcctggagaagatcaactatgtcggcatggccaacttcgacatcaagtacgatgcccgcgacggccagtacaagttctttgaaatcaacctccgccaaggccggtccagcttctacgtgaccttgaacggctacaacctggccaagtggtacgttgacgactacgctgaagactccttggccggccaggagaccgtctatgccaacaagctggagagcagccacatgctctggctgggcgtgccggagaagatcttcaaggaatacgcggtcgacaacgaggccaagcaggcggccttgaaactgatcgatgccggccaggtggggacgaccgtcttctacgaccaagacagctcctttaagcgctggctcttgatgaagtacatgttccacaactactacaagcgcttcaagacctacttccaggtcaacaagggccagtacttcgataaatag

SEQ ID NO: 28 - DNA, 1260 bp, Lactobacillus casei
atgaaagatgcacccagttttacccccatcctgcttggcagtgacatgaatgtgtatggtatggcgcgttcgttccacgagatgatcggtggggcgattgatgtttatgcgcgcgaacaacttgctccaacgcgtttttcgcggatcgtgaatgtccatctgatcgagcattttgattccgatccgacttttattgaaaatatgcggcaggtagccaaggtacatgcagatgctccaggcaaactgttgttaatcgcttgcggggacacctatgcccaacttgtttccaaacataaggatgagttgagccagtggttcatctgcccatatgttgattatgaattgttgaagcgtctgaatagtaaggagagcttctatcaagtttgcgaagagtatcatttgccatatccggcaaccaaaatcgtcaccaagtcgatgtatgaaagcaacgcaaccatcacggcaccgtttgaatttccagtagcactcaagccgactgattccgttgaatggttgaacattcgctttgaaggccgcaaaaaggcgtttacgattcactcgcaagttgaactgacgaatacgattgccaaaatctatgataacggctatacgtctgatctcatcttgcaggactttgtaccaggtgatgacagtaacatgcggactttgaactgttatgttgatcaatatcatcacgttaaaatgatgtgcttaggtcatccgctacttgaggatcctacgccttcttcaatcggtaactatgtcgcgatcatgccagctttcgatcaggatttgtacgatcagatcaaaacattcctcgaagcaattaagttcaccggttttgctaactttgatatgaagtatgatcatcgtgacaagacgttcaagctgtttgaaattaatattcgtcaaggccggagtagctttttcgtcactttaaatgggtataatcttgccagctggcctgttcgcgattacatcgtgggagatttgaaggacgcgccaacggtttatggtaatgatgaccacagcaagtataaattgtggctaggggttccagagcgtgttttcaagcagtattctgcagataacgaagctaaaaaggaagccttgcagttacttaaagagaaacgctatggtacgacggtcttttataaaccggacatgacattcaagcgctggttattgatgaaatatatgttccataattatgttggtcggtttaagcagtattttgcggaaaataagggcgacatgtga

SEQ ID NO: 29 - DNA, 1269 bp, Lactobacillus acidophilus
atgactaaagaatttacgccaattttacttggtagtgatattaatgtttatggtatggctcgttcatttaatgaagcctatggtattcgtgttaaggcgcttgctgatgtacagcttgctgcaactcgttattccaaaattgttgatgtagaacttcatcatggttttagcgcagatccaacatttatggacgttatgcgcaagaagatggaaatctacaaggatcacaaggaaccagtcattttgattgcttgtggtgatggttatgcagagcttctttccaagcataaggatgaattgagcaaggtatttatcgtaccttacattgaatatgacttgcttgaaaaattgatctctaaggaaggcttctatgaagtagctgaagaatatggtttgccatatcctaagactaagatcattactatggatgattacaagtcaggaaaatataaggatgtacctttccaattcccagtagaacttaaaccagaagacccagtttcatggcttaactgccaatttgaaggccgtaagaagacttttactcttcatgatgaaaacgaatttgaggacattgtaactaaaatttatactcacggttatactgaagatcttattttacaagactttatccctggcgatgattcaaacatgagaactttgaatgcttatgttgataagaatcaccaagttaaaatgatgtgcttgggtcacccacttttggaagatccaactccacaatcaatcggtaactacatgactatcttgccagcatacgatgaaaaattgtatgaacaagttcaatcattccttgaaaagttaaactacactggtatggctaactttgatatcaagtacgatgaacgtgatggccaatacaagttcttcgaaatcaatttacgtcaaggtcgttcaagtttttacgtaactttgaatggttacaacttagctaagtggtacattgatgattatgttgaagataacttgagtgacaaaaagacagtttacggtaataaattgaagtctaagcatgttttatggatgggtgttccagttaaaatcttcaaggaatatgcattcgaaaatgaagcaaaagacactgcagaagaattaattcatgaaggccgctatggtacaactgttttttatgataaagatcgcagccttaaacgttggcttttgatgaagtatatgttccataattacttcgcaagatacaagaagtactatgaagtaaataagggtcaatactttaagaagaacaaataa

SEQ ID NO: 30 - DNA, 1257 bp, Lactobacillus brevis
atggaaaccaatggcgtcaagtttacgcccgtattactcgggagtgattttaacgtttacgggatggctcgctccttttatgagctataccatgaacccgttcaagcttttgccagtgtgcaattagcaccaacgcgctatacaaaagttgttaatttagagttgattcctggatttgatgaagatccagtttggattcaaacaatgcgtaaacttaaggagcggtacgctgatcatcccgaacctgtgattttaattgggtgtggggatggttatgccgaattgattagtaagcataaagccgaactcactgatgtgtttatttgcccctatatcgactacgataaattacggcatttgaacaataaagagaatttctacaaagtctgtgaccagtacggattaccctatccggctacccgtattatcaccaaggacatggcgacagccgctgaaccggtggaacaaccctttgattatccggtagccctgaaaccagctaacagtgtggaatggttggatgtccacttcgaggggcgtaagaaggcctttcggattcagtctgaagcagagttccgcgatattgtgcagaagatttatgccaacggctaccagtccgatctgatcttacaagattttgttcctggtgatgatagtaacatgcgggtactaaatgcttacgtcgatcaatatcaccatgtgaagatgatgtgcctcggccatcctttgttggaagacccagcaccatctgcgattggtaactatgttgcgatcattcctgaatataacgagacaatttatcagaacattcagaaattcttggaagccattgaatttactgggtacgcgaactttgatatgaagtacgatgtgcgtgaccaaacgtttaagttatttgaaattaacttacgtcagggacggagtagtttctttgtgaccttaaatggctataatctggctgattgggttgttaaggactacgttaaacaggaattggttgatgctcccaccgtttacggtaatcaagatgagtcacaacatgcgctatggttaggtgtgccggtgaaagtctttaagcaatatacgcgtgagaatgtggacaagcaacgggcgttgaagttgattgccgctggcaagtatggaacaacgtatcactacgatcaggatatgaacttaaaacggtgggcattgctaacctggatgcaacacaactatacggtgaactttaagaagtatttcgctgaaaataaggggtaa

SEQ ID NO: 31 - DNA, 1257 bp, Pediococcus pentosaceus
atggaaagaccaactaacaattttacgccaattctattaggtagtgattttaatgcttatgggatggctcgttcattttacgaaattacaggaagacgtgttcaagcgtatgccgggaccgtgctagcacccacacgttttactaaaatcgtgaatgttgatattattcctggtttttcagaagaccctgtttttattgaatcaatgcgtaaaattgctaaaaaatacgaaaattctgacgagaaggttgttttactagggatgggcgatggctatgctgaattgatttcaaagcataaagaagagttgtcaaaaactttcatctgtccctacatcgattatgatttattgaaacaattgaataataaagaacgtttttattcaatttgtgatgaatataatttaccatatccagcaacccattttattacgaaggcacaatatgaaagtggtgagaaattcatctcaccttttgaatttccagttgctttgaagccagctgacagtgtagaatggctcgatattcattttgaaggtcgtaaaaaagctttcagaattaaaactcaagcagaacttgatgaagtcattggtaaaatctatgacaatggctatacttctgatttgattttacaagattttattccaggtgatgatagcaacatgcgagttttgaatgcttatgttgatcaaaatcatcaagtaaaaatgatgtgtttaggtcatccattattggaggatccagctccatcagcaattggaaactacgtagcaattattcctgaatttaaccaagaaatttatgacacggttaaaaactttcttgaaagtatcaattacgttggatatgccaactttgatatgaaatatgacagtcgggatggtaagtttaaattatttgaaatcaatattcgtcaaggccgtagtagcttctttgttacattaaatggatacaacctagctcgttgggtggttgaagaccgagttaacaatgcgttggctgatcgtgaaactgtatacgctaatgatgttccaggcaactggaagttatggttgggcgttccagttaaggtcttcaaaaagtatgctaaaaataatgatgataagcaaaaggcgcttgaactaattaacaatgatcaatatggatttaccgctatctacgatgaggatatgaactttaagcgtagcttattaatgtggtggatgaaccataattatgttaagaactttaggaagtattttaaggaaaataaggggtaa

SEQ ID NO: 32 - DNA, 1401 bp, Enterococcus faecium
atgacaagatcttcgcatcgaaaaaaatcgaaacatgttgctttgctttcagttcttggtgttctcttagtgctgatcagcttttatacctatcgcagtacatactacacaaagcattttttgccgaatacagaaataaatggaataaatgtcagcaatctaacagtcgaaaaagcaaatgaaaaattaaaagaagcttattctgataaaaaactggctattaaagaagatggaaaagtctggcaggaagtaccaaaatccgagctcggctacaaagatgatttcacttcggagctttctccgatcctaaatgaacaaaacggatggacttggggaatgacttatgtctcagcggcagaaaaacaagaaatagatcctgtcagtcaaaaccaccaaaaactagacactaccgttcagacattgacaactaagctgactgatttgaacaaagaccggacacctactactgatgcaacgatcgaaaaagcaggtgactctttcgttatcaagcctgaagtcaatggaaatacgatcgacgtcgatgctgcggtcaaacaattaaaaagtgcggtaaactctggtaaagatacaattgaattgactgaattcaaagaaaaaccaaaagtaacatctgaagacagctcactgaaagaacaactcgcatccatgaatgctatcgcaaatgtaaaagctacttacagtataaacggtgagactttccagatcccatcaagtgatattatgagttggctgacctacaatgatggaaaagtcgatttagatacagaacaagtacgtcaatatgtaacagatttaggcacaaagtataacacaagtacgaatgacacaaaatttaaaagtacaaaaagaggagaagttacggtcccagttggcacttacagttggacgatccaaacagacagtgagacagaagcattgaaaaaagcgattctggcaggccaagattttactcgttcacccattgtgcaaggtggcacaaccgctgatcatccattgatcgaagatacgtatatcgaagttgatctggagaaccagcatatgtggtattacaaagacggaaaagtggcccttgaaacagatatcgtttccggaaagccaactacacctacacctgctggagtcttctatgtttggaataaagaagaagatgcaactttaaaaggaacaaatgacgatggaacaccttacgagagtccggtaaactactggatgccgattgactggacaggtgttggtatccacgattcagactggcaacctgaatacggtggagacttatggaaaacccgcggttcacacggctgcatcaacaccccaccaagtgtaatgaaggaattattcgggatggtcgaaaaaggaacgcctgtattggtcttctaa

SEQ ID NO: 33 - protein, 348 aa, Enterococcus faecium
AEKQEIDPVSQNHQKLDTTVQTLTTKLTDLNKDRTPTTDATIEKAGDSFVIKPEVNGNTIDVDAAVKQLKSAVNSGKDTIELTEFKEKPKVTSEDSSLKEQLASMNAIANVKATYSINGETFQIPSSDIMSWLTYNDGKVDLDTEQVRQYVTDLGTKYNTSTNDTKFKSTKRGEVTVPVGTYSWTIQTDSETEALKKAILAGQDFTRSPIVQGGTTADHPLIEDTYIEVDLENQHMWYYKDGKVALETDIVSGKPTTPTPAGVFYVWNKEEDATLKGTNDDGTPYESPVNYWMPIDWTGVGIHDSDWQPEYGGDLWKTRGSHGCINTPPSVMKELFGMVEKGTPVLVF